SEQUENCE LISTING (63 sequences)

SEQ ID NO: 1 - protein, 18 aa, synthetic construct
EGRGSLLTCGDVEENPGP

SEQ ID NO: 2 - protein, 19 aa, synthetic construct
ATNFSLLKQAGDVEENPGP

SEQ ID NO: 3 - protein, 20 aa, synthetic construct
QCTNYALLKLAGDVESNPGP

SEQ ID NO: 4 - protein, 22 aa, synthetic construct
VKQTLNFDLLKLAGDVESNPGP

SEQ ID NO: 5 - protein, 27 aa, synthetic construct
FWVLVVVGGVLACYSLLVTVAFIIFWV

SEQ ID NO: 6 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 7 - protein, 22 aa, synthetic construct
VSAVTLAYLIFCLCSLVGILHL

SEQ ID NO: 8 - DNA, 66 bp, synthetic construct
  source 1..66: mol_type = other DNA
gtctcagctgtgactttggcttatctgatcttctgcctgtgttcccttgtgggcattcttcatctt

SEQ ID NO: 9 - protein, 21 aa, synthetic construct
IYIWAPLAGTCGVLLLSLVIT

SEQ ID NO: 10 - DNA, 71 bp, synthetic construct
  source 1..71: mol_type = other DNA
atctacatctgggcgcccttggccgggacttgtggggtccttctcctgtcactggttatcaccctttactg

SEQ ID NO: 11 - protein, 22 aa, synthetic construct
MALIVLGGVAGLLLFIGLGIFF

SEQ ID NO: 12 - DNA, 66 bp, synthetic construct
  source 1..66: mol_type = other DNA
atggccctgatcgtgctgggcggagtggctggcctgctgctgtttatcggcctgggcatattcttt

SEQ ID NO: 13 - protein, 45 aa, synthetic construct
TTTPAPRPPTPAPTIASQPLSLRPEACRPAAGGAVHTRGLDFACD

SEQ ID NO: 14 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 15 - protein, 22 aa, synthetic construct
MLLLVTSLLLCELPHPAFLLIP

SEQ ID NO: 16 - DNA, 66 bp, synthetic construct
  source 1..66: mol_type = other DNA
atgctgctgctcgtgacctctctgctgctgtgcgagctgccccaccctgcctttctgctgatccct

SEQ ID NO: 17 - protein, 20 aa, synthetic construct
EFGLSWLFLVAILKGVQCSR

SEQ ID NO: 18 - DNA, 60 bp, synthetic construct
  source 1..60: mol_type = other DNA
gagtttgggctgagctggctttttcttgtggctattttaaaaggtgtccagtgctctaga

SEQ ID NO: 19 - protein, 269 aa, synthetic construct
MSAALFSLDGPARGAPWPAEPAPFYEPGRAGKPGRGAEPGALGEPGAAAPAMYDDESAIDFSAYIDSMAAVPTLELCHDELFADLFNSNHKAGGAGPLELLPGGPARPLGPGPAAPRLLKREPDWGDGDAPGSLLPAQVAACAQTVVSLAAAGQPTPPTSPEPPRSSPRQTPAPGPAREKSAGKRGPDRGSPEYRQRRERNNIAVRKSRDKAKRRNQEMQQKLVELSAENEKLHQRVEQLTRDLAGLRQFFKQLPSPPFLPAAGTADCR

SEQ ID NO: 20 - DNA, 807 bp, synthetic construct
  source 1..807: mol_type = other DNA
atgtcagcagctcttttttctcttgatggtcctgcccggggcgcgccatggcccgctgagccagctcccttttacgagccaggtagagctggaaaaccagggagaggggcggagcccggggctctcggggagcctggagctgcagctccggccatgtatgacgatgaatcagcaatcgatttctcagcgtacattgattccatggccgccgttccaacgttggaactctgtcatgatgagcttttcgcagacctctttaactctaatcacaaagctggcggcgctggtccactggaacttcttcctgggggccccgctcggccgctcgggcccggtcccgccgccccgagattgctgaaaagggaaccggattggggagatggtgacgcgccaggaagcttgctgcctgcacaggtagcggcatgcgcccagaccgtggtgagtctggctgcagcgggccaaccaacccctccaacgtcaccggaaccaccacgatcatcacctagacaaacaccagcgcccggcccggcgcgcgaaaagtccgcagggaaaagagggcctgacagaggcagtccggaatacagacaacgccgggagaggaacaatatagctgtgagaaagagtcgcgataaggcgaaacggcggaaccaagaaatgcagcagaagctggtagaactcagcgcagagaacgagaagctccatcaacgggttgagcaactgactagggacctcgcgggcctgagacaatttttcaagcagctgccgagtccaccctttcttcccgcagcggggactgccgactgccgc

SEQ ID NO: 21 - DNA, 810 bp, Homo sapiens
  source 1..810: mol_type = other DNA
atgagcgccgcgctcttcagcctggacggcccggcgcgcggcgcgccctggcctgcggagcctgcgcccttctacgaaccgggccgggcgggcaagccgggccgcggggccgagccaggggccctaggcgagccaggcgccgccgcccccgccatgtacgacgacgagagcgccatcgacttcagcgcctacatcgactccatggccgccgtgcccaccctggagctgtgccacgacgagctcttcgccgacctcttcaacagcaatcacaaggcgggcggcgcggggcccctggagcttcttcccggcggccccgcgcgccccttgggcccgggccctgccgctccccgcctgctcaagcgcgagcccgactggggcgacggcgacgcgcccggctcgctgttgcccgcgcaggtggccgcgtgcgcacagaccgtggtgagcttggcggccgcagggcagcccaccccgcccacgtcgccggagccgccgcgcagcagccccaggcagacccccgcgcccggccccgcccgggagaagagcgccggcaagaggggcccggaccgcggcagccccgagtaccggcagcggcgcgagcgcaacaacatcgccgtgcgcaagagccgcgacaaggccaagcggcgcaaccaggagatgcagcagaagttggtggagctgtcggctgagaacgagaagctgcaccagcgcgtggagcagctcacgcgggacctggccggcctccggcagttcttcaagcagctgcccagcccgcccttcctgccggccgccgggacagcagactgccggtaa

SEQ ID NO: 22 - DNA, 1590 bp, synthetic construct
  source 1..1590: mol_type = other DNA
atgtcagcagctcttttttctcttgatggtcctgcccggggcgcgccatggcccgctgagccagctcccttttacgagccaggtagagctggaaaaccagggagaggggcggagcccggggctctcggggagcctggagctgcagctccggccatgtatgacgatgaatcagcaatcgatttctcagcgtacattgattccatggccgccgttccaacgttggaactctgtcatgatgagcttttcgcagacctctttaactctaatcacaaagctggcggcgctggtccactggaacttcttcctgggggccccgctcggccgctcgggcccggtcccgccgccccgagattgctgaaaagggaaccggattggggagatggtgacgcgccaggaagcttgctgcctgcacaggtagcggcatgcgcccagaccgtggtgagtctggctgcagcgggccaaccaacccctccaacgtcaccggaaccaccacgatcatcacctagacaaacaccagcgcccggcccggcgcgcgaaaagtccgcagggaaaagagggcctgacagaggcagtccggaatacagacaacgccgggagaggaacaatatagctgtgagaaagagtcgcgataaggcgaaacggcggaaccaagaaatgcagcagaagctggtagaactcagcgcagagaacgagaagctccatcaacgggttgagcaactgactagggacctcgcgggcctgagacaatttttcaagcagctgccgagtccaccctttcttcccgcagcggggactgccgactgccgcggctctggggaaggacgcggtagccttctgacttgcggcgatgtggaggagaatcctgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 23 - protein, 345 aa, Homo sapiens
MQRLVAWDPACLPLPPPPPAFKSMEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 24 - DNA, 1038 bp, Homo sapiens
  source 1..1038: mol_type = other DNA
atgcaacgcctggtggcctgggacccagcatgtctccccctgccgccgccgccgcctgcctttaaatccatggaagtggccaacttctactacgaggcggactgcttggctgctgcgtacggcggcaaggcggcccccgcggcgccccccgcggccagacccgggccgcgcccccccgccggcgagctgggcagcatcggcgaccacgagcgcgccatcgacttcagcccgtacctggagccgctgggcgcgccgcaggccccggcgcccgccacggccacggacaccttcgaggcggctccgcccgcgcccgcccccgcgcccgcctcctccgggcagcaccacgacttcctctccgacctcttctccgacgactacgggggcaagaactgcaagaagccggccgagtacggctacgtgagcctggggcgcctgggggccgccaagggcgcgctgcaccccggctgcttcgcgcccctgcacccaccgcccccgccgccgccgccgcccgccgagctcaaggcggagccgggcttcgagcccgcggactgcaagcggaaggaggaggccggggcgccgggcggcggcgcaggcatggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcgcccccgaccgcctgctacgcgggggccgcgccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctag

SEQ ID NO: 25 - protein, 322 aa, Homo sapiens
MEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 26 - DNA, 969 bp, Homo sapiens
  source 1..969: mol_type = other DNA
atggaagtggccaacttctactacgaggcggactgcttggctgctgcgtacggcggcaaggcggcccccgcggcgccccccgcggccagacccgggccgcgcccccccgccggcgagctgggcagcatcggcgaccacgagcgcgccatcgacttcagcccgtacctggagccgctgggcgcgccgcaggccccggcgcccgccacggccacggacaccttcgaggcggctccgcccgcgcccgcccccgcgcccgcctcctccgggcagcaccacgacttcctctccgacctcttctccgacgactacgggggcaagaactgcaagaagccggccgagtacggctacgtgagcctggggcgcctgggggccgccaagggcgcgctgcaccccggctgcttcgcgcccctgcacccaccgcccccgccgccgccgccgcccgccgagctcaaggcggagccgggcttcgagcccgcggactgcaagcggaaggaggaggccggggcgccgggcggcggcgcaggcatggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcgcccccgaccgcctgctacgcgggggccgcgccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctag

SEQ ID NO: 27 - protein, 147 aa, Homo sapiens
MAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 28 - DNA, 444 bp, Homo sapiens
  source 1..444: mol_type = other DNA
atggcggcgggcttcccgtacgcgctgcgcgcttacctcggctaccaggcggtgccgagcggcagcagcgggagcctctccacgtcctcctcgtccagcccgcccggcacgccgagccccgctgacgccaaggcgcccccgaccgcctgctacgcgggggccgcgccggcgccctcgcaggtcaagagcaaggccaagaagaccgtggacaagcacagcgacgagtacaagatccggcgcgagcgcaacaacatcgccgtgcgcaagagccgcgacaaggccaagatgcgcaacctggagacgcagcacaaggtcctggagctcacggccgagaacgagcggctgcagaagaaggtggagcagctgtcgcgcgagctcagcaccctgcggaacttgttcaagcagctgcccgagcccctgctcgcctcctccggccactgctag

SEQ ID NO: 29 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 30 - DNA, 486 bp, synthetic construct
  source 1..486: mol_type = other DNA
atgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 31 - protein, 162 aa, synthetic construct
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 32 - DNA, 486 bp, synthetic construct
  source 1..486: mol_type = other DNA
atgaggagcagtccaggcaatatggagcggatagtaatttgtctcatggtaatattcctcggtactctggtacataaatcttcctctcaaggtcaggaccgccatatgattcgaatgcggcagctgattgacatagtcgatcaactgaagaactatgtgaatgatcttgtgcccgagtttttgccagcccctgaagacgtagaaactaattgtgagtggagtgccttttcctgctttcaaaaggcacagctgaaatccgccaacacgggcaataacgaacggataattaacgtatccattaagaagctgaagcggaagccgccctcaaccaatgcgggacggcggcaaaagcatcgcttgacctgtccgtcatgcgacagctacgagaaaaagcccccgaaggagttcttggaacgcttcaagagtctccttcagaaaatgattcaccagcacctgtcctcacggacgcacggaagcgaggacagt

SEQ ID NO: 33 - protein, 132 aa, Homo sapiens
GQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 34 - DNA, 396 bp, Homo sapiens
  source 1..396: mol_type = other DNA
ggccaggaccggcacatgatccggatgagacagctgatcgacatcgtggaccagctgaagaactacgtgaacgacctggtgcccgagttcctgcctgcccccgaggacgtggaaacaaactgcgagtggagcgccttcagctgcttccagaaggcccagctgaaaagcgccaacaccggcaacaacgagcggatcatcaacgtgtccatcaagaagctgaagcggaagccccccagcaccaacgccggaagaaggcagaagcacagactgacctgccccagctgcgacagctacgagaagaagccccctaaagagttcctggaacggttcaagagcctgctgcagaagatgatccaccagcacctgagcagccggacccacggctctgaggacagc

SEQ ID NO: 35 - protein, 272 aa, synthetic construct
EPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKETTPNKGSGTTSGTTRLLSGHTCFTLTGLLGTLVTMGLLT

SEQ ID NO: 36 - DNA, 816 bp, synthetic construct
  source 1..816: mol_type = other DNA
gagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagaaacaaccccaaataaaggaagtggaaccacttcaggtactacccgtcttctatctgggcacacgtgtttcacgttgacaggtttgcttgggacgctagtaaccatgggcttgctgact

SEQ ID NO: 37 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - DNA, 687 bp, synthetic construct
  source 1..687: mol_type = other DNA
gagtctaagtacggccctccctgccctccttgtccagcccctgaatttctgggcggacccagcgtgttcctgttccccccaaagcccaaggacaccctgatgatcagcaggacccccgaagtgacctgcgtggtggtggatgtgtcccaggaagatcccgaggtgcagtttaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaggtgtccaacaagggcctgcccagcagcatcgagaaaaccatcagcaaggccaagggccagcctcgggaaccccaggtgtacacactgccccctagccaggaagagatgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagccgcctgaccgtggacaagagcagatggcaggaaggcaacgtgttcagctgcagcgtgatgcacgaggccctgcacaaccactacacccagaagtccctgtctctgagcctgggcaag

SEQ ID NO: 39 - protein, 313 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHL

SEQ ID NO: 40 - DNA, 939 bp, synthetic construct
  source 1..939: mol_type = other DNA
atgccacctcctcgcctcctcttcttcctcctcttcctcacccctatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcactatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctt

SEQ ID NO: 41 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 42 - DNA, 816 bp, Homo sapiens
  source 1..816: mol_type = other DNA
cccgaggagcccctggtggtgaaggtggaggagggcgacaacgccgtgctgcagtgcctgaagggcaccagcgacggccccacccagcagctgacctggagcagagagagccccctgaagcccttcctgaagctgagcctgggcctgcccggcctgggcatccacatgagacccctggccatctggctgttcatcttcaacgtgagccagcagatgggcggcttctacctgtgccagcccggcccccccagcgagaaggcctggcagcccggctggaccgtgaacgtggagggcagcggcgagctgttcagatggaacgtgagcgacctgggcggcctgggctgcggcctgaagaacagaagcagcgagggccccagcagccccagcggcaagctgatgagccccaagctgtacgtgtgggccaaggacagacccgagatctgggagggcgagcccccctgcctgccccccagagacagcctgaaccagagcctgagccaggacctgaccatggcccccggcagcaccctgtggctgagctgcggcgtgccccccgacagcgtgagcagaggccccctgagctggacccacgtgcaccccaagggccccaagagcctgctgagcctggagctgaaggacgacagacccgccagagacatgtgggtgatggagaccggcctgctgctgcccagagccaccgcccaggacgccggcaagtactactgccacagaggcaacctgaccatgagcttccacctggagatcaccgccagacccgtgctgtggcactggctgctgagaaccggcggctggaag

SEQ ID NO: 43 - DNA, 2271 bp, synthetic construct
  source 1..2271: mol_type = other DNA
atgctcgagtttgggctgagctggctttttcttgtggctattttaaaaggtgtccagtgctctagacccgaggagcccctggtggtgaaggtggaggagggcgacaacgccgtgctgcagtgcctgaagggcaccagcgacggccccacccagcagctgacctggagcagagagagccccctgaagcccttcctgaagctgagcctgggcctgcccggcctgggcatccacatgagacccctggccatctggctgttcatcttcaacgtgagccagcagatgggcggcttctacctgtgccagcccggcccccccagcgagaaggcctggcagcccggctggaccgtgaacgtggagggcagcggcgagctgttcagatggaacgtgagcgacctgggcggcctgggctgcggcctgaagaacagaagcagcgagggccccagcagccccagcggcaagctgatgagccccaagctgtacgtgtgggccaaggacagacccgagatctgggagggcgagcccccctgcctgccccccagagacagcctgaaccagagcctgagccaggacctgaccatggcccccggcagcaccctgtggctgagctgcggcgtgccccccgacagcgtgagcagaggccccctgagctggacccacgtgcaccccaagggccccaagagcctgctgagcctggagctgaaggacgacagacccgccagagacatgtgggtgatggagaccggcctgctgctgcccagagccaccgcccaggacgccggcaagtactactgccacagaggcaacctgaccatgagcttccacctggagatcaccgccagacccgtgctgtggcactggctgctgagaaccggcggctggaagtcgcgagagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagaaacaaccccaaataaaggaagtggaaccacttcaggtactacccgtcttctatctgggcacacgtgtttcacgttgacaggtttgcttgggacgctagtaaccatgggcttgctgactttcggaccgcagtgtactaattatgctctcttgaaattggctggagatgttgagagcaatcccgggcccatgcgcatgatgaggagcagtccaggcaatatggagcggatagtaatttgtctcatggtaatattcctcggtactctggtacataaatcttcctctcaaggtcaggaccgccatatgattcgaatgcggcagctgattgacatagtcgatcaactgaagaactatgtgaatgatcttgtgcccgagtttttgccagcccctgaagacgtagaaactaattgtgagtggagtgccttttcctgctttcaaaaggcacagctgaaatccgccaacacgggcaataacgaacggataattaacgtatccattaagaagctgaagcggaagccgccctcaaccaatgcgggacggcggcaaaagcatcgcttgacctgtccgtcatgcgacagctacgagaaaaagcccccgaaggagttcttggaacgcttcaagagtctccttcagaaaatgattcaccagcacctgtcctcacggacgcacggaagcgaggacagttga

SEQ ID NO: 44 - DNA, 2268 bp, synthetic construct
  source 1..2268: mol_type = other DNA
atgctcgagtttgggctgagctggctttttcttgtggctattttaaaaggtgtccagtgctctagacccgaggagcccctggtggtgaaggtggaggagggcgacaacgccgtgctgcagtgcctgaagggcaccagcgacggccccacccagcagctgacctggagcagagagagccccctgaagcccttcctgaagctgagcctgggcctgcccggcctgggcatccacatgagacccctggccatctggctgttcatcttcaacgtgagccagcagatgggcggcttctacctgtgccagcccggcccccccagcgagaaggcctggcagcccggctggaccgtgaacgtggagggcagcggcgagctgttcagatggaacgtgagcgacctgggcggcctgggctgcggcctgaagaacagaagcagcgagggccccagcagccccagcggcaagctgatgagccccaagctgtacgtgtgggccaaggacagacccgagatctgggagggcgagcccccctgcctgccccccagagacagcctgaaccagagcctgagccaggacctgaccatggcccccggcagcaccctgtggctgagctgcggcgtgccccccgacagcgtgagcagaggccccctgagctggacccacgtgcaccccaagggccccaagagcctgctgagcctggagctgaaggacgacagacccgccagagacatgtgggtgatggagaccggcctgctgctgcccagagccaccgcccaggacgccggcaagtactactgccacagaggcaacctgaccatgagcttccacctggagatcaccgccagacccgtgctgtggcactggctgctgagaaccggcggctggaagtcgcgagagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagaaacaaccccaaataaaggaagtggaaccacttcaggtactacccgtcttctatctgggcacacgtgtttcacgttgacaggtttgcttgggacgctagtaaccatgggcttgctgactttcggaccgcagtgtactaattatgctctcttgaaattggctggagatgttgagagcaatcccgggcccatgcgcatgatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 45 - DNA, 1260 bp, synthetic construct
  source 1..1260: mol_type = other DNA
atggatgctgctgctcgtgacctctctgctgctgtgcgagctgccccaccctgcctttctgctgatccctcagggccaggaccggcacatgatccggatgagacagctgatcgacatcgtggaccagctgaagaactacgtgaacgacctggtgcccgagttcctgcctgcccccgaggacgtggaaacaaactgcgagtggagcgccttcagctgcttccagaaggcccagctgaaaagcgccaacaccggcaacaacgagcggatcatcaacgtgtccatcaagaagctgaagcggaagccccccagcaccaacgccggaagaaggcagaagcacagactgacctgccccagctgcgacagctacgagaagaagccccctaaagagttcctggaacggttcaagagcctgctgcagaagatgatccaccagcacctgagcagccggacccacggctctgaggacagcgagtctaagtacggccctccctgccctccttgtccagcccctgaatttctgggcggacccagcgtgttcctgttccccccaaagcccaaggacaccctgatgatcagcaggacccccgaagtgacctgcgtggtggtggatgtgtcccaggaagatcccgaggtgcagtttaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaggtgtccaacaagggcctgcccagcagcatcgagaaaaccatcagcaaggccaagggccagcctcgggaaccccaggtgtacacactgccccctagccaggaagagatgaccaagaaccaggtgtccctgacctgtctcgtgaagggcttctaccccagcgacattgccgtggaatgggagagcaacggccagcccgagaacaactacaagaccaccccccctgtgctggacagcgacggctcattcttcctgtacagccgcctgaccgtggacaagagcagatggcaggaaggcaacgtgttcagctgcagcgtgatgcacgaggccctgcacaaccactacacccagaagtccctgtctctgagcctgggcaagatggccctgatcgtgctgggcggagtggctggcctgctgctgtttatcggcctgggcatattcttttgacgcgtcaattgcgcgtcatcatcgatccggattag

SEQ ID NO: 46 - DNA, 2970 bp, synthetic construct
  source 1..2970: mol_type = other DNA
atgctcgagggagtgcaggtggaaaccatctccccaggcgacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagatcatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagcttcgcgagccgagggcaggggaagtcttctaacatgcggggacgtggaggaaaatcccgggcccatggatggccttaccagtgaccgccttgctcctgccgctggccttgctgctccacgccgccaggccgcccataaatcttcctctcaaggtcaggaccgccatatgattcgaatgcggcagctgattgacatagtcgatcaactgaagaactatgtgaatgatcttgtgcccgagtttttgccagcccctgaagacgtagaaactaattgtgagtggagtgccttttcctgctttcaaaaggcacagctgaaatccgccaacacgggcaataacgaacggataattaacgtatccattaagaagctgaagcggaagccgccctcaaccaatgcgggacggcggcaaaagcatcgcttgacctgtccgtcatgcgacagctacgagaaaaagcccccgaaggagttcttggaacgcttcaagagtctccttcagaaaatgattcaccagcacctgtcctcacggacgcacggaagcgaggacagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcggaccgcagtgtactaattatgctctcttgaaattggctggagatgttgagagcaatcccgggcctatggggatgccacctcctcgcctcctcttcttcctcctcttcctcacccctatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcactatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctttag

SEQ ID NO: 47 - DNA, 2847 bp, synthetic construct
  source 1..2847: mol_type = other DNA
atgctcgagggagtgcaggtggaaaccatctccccaggcgacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagatcatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagcttcgcgagccgagggcaggggaagtcttctaacatgcggggacgtggaggaaaatcccgggcccatggatggccttaccagtgaccgccttgctcctgccgctggccttgctgctccacgccgccaggccgcccataaatcttcctctcaaggtcaggaccgccatatgattcgaatgcggcagctgattgacatagtcgatcaactgaagaactatgtgaatgatcttgtgcccgagtttttgccagcccctgaagacgtagaaactaattgtgagtggagtgccttttcctgctttcaaaaggcacagctgaaatccgccaacacgggcaataacgaacggataattaacgtatccattaagaagctgaagcggaagccgccctcaaccaatgcgggacggcggcaaaagcatcgcttgacctgtccgtcatgcgacagctacgagaaaaagcccccgaaggagttcttggaacgcttcaagagtctccttcagaaaatgattcaccagcacctgtcctcacggacgcacggaagcgaggacagtttttgggtgctggtggtggttggtggagtcctggcttgctatagcttgctagtaacagtggcctttattattttctgggtgcccggaccgcagtgtactaattatgctctcttgaaattggctggagatgttgagagcaatcccgggcctatggggatgccacctcctcgcctcctcttcttcctcctcttcctcacccctatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcactatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctttag

SEQ ID NO: 48 - DNA, 1767 bp, synthetic construct
  source 1..1767: mol_type = other DNA
atggccttaccagtgaccgccttgctcctgccgctggccttgctgctccacgccgccaggccgatgaggagcagtccaggcaatatggagcggatagtaatttgtctcatggtaatattcctcggtactctggtacataaatcttcctctcaaggtcaggaccgccatatgattcgaatgcggcagctgattgacatagtcgatcaactgaagaactatgtgaatgatcttgtgcccgagtttttgccagcccctgaagacgtagaaactaattgtgagtggagtgccttttcctgctttcaaaaggcacagctgaaatccgccaacacgggcaataacgaacggataattaacgtatccattaagaagctgaagcggaagccgccctcaaccaatgcgggacggcggcaaaagcatcgcttgacctgtccgtcatgcgacagctacgagaaaaagcccccgaaggagttcttggaacgcttcaagagtctccttcagaaaatgattcaccagcacctgtcctcacggacgcacggaagcgaggacagtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgctcgcgagccgagggcaggggaagtcttctaacatgcggggacgtggaggaaaatcccgggcctatggggatgccacctcctcgcctcctcttcttcctcctcttcctcacccctatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcactatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctttag

SEQ ID NO: 49 - DNA, 1641 bp, synthetic construct
  source 1..1641: mol_type = other DNA
atggccttaccagtgaccgccttgctcctgccgctggccttgctgctccacgccgccaggccgatgaggagcagtccaggcaatatggagcggatagtaatttgtctcatggtaatattcctcggtactctggtacataaatcttcctctcaaggtcaggaccgccatatgattcgaatgcggcagctgattgacatagtcgatcaactgaagaactatgtgaatgatcttgtgcccgagtttttgccagcccctgaagacgtagaaactaattgtgagtggagtgccttttcctgctttcaaaaggcacagctgaaatccgccaacacgggcaataacgaacggataattaacgtatccattaagaagctgaagcggaagccgccctcaaccaatgcgggacggcggcaaaagcatcgcttgacctgtccgtcatgcgacagctacgagaaaaagcccccgaaggagttcttggaacgcttcaagagtctccttcagaaaatgattcaccagcacctgtcctcacggacgcacggaagcgaggacagtttttgggtgctggtggtggttggtggagtcctggcttgctatagcttgctagtaacagtggcctttattattttctgggtgtcgcgagccgagggcaggggaagtcttctaacatgcggggacgtggaggaaaatcccgggcctatggggatgccacctcctcgcctcctcttcttcctcctcttcctcacccctatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcactatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatctttag

SEQ ID NO: 50 - DNA, 1590 bp, synthetic construct; identical to SEQ ID NO: 22
  source 1..1590: mol_type = other DNA

SEQ ID NO: 51 - DNA, 2136 bp, synthetic construct
  source 1..2136: mol_type = other DNA
atgtcagcagctcttttttctcttgatggtcctgcccggggcgcgccatggcccgctgagccagctcccttttacgagccaggtagagctggaaaaccagggagaggggcggagcccggggctctcggggagcctggagctgcagctccggccatgtatgacgatgaatcagcaatcgatttctcagcgtacattgattccatggccgccgttccaacgttggaactctgtcatgatgagcttttcgcagacctctttaactctaatcacaaagctggcggcgctggtccactggaacttcttcctgggggccccgctcggccgctcgggcccggtcccgccgccccgagattgctgaaaagggaaccggattggggagatggtgacgcgccaggaagcttgctgcctgcacaggtagcggcatgcgcccagaccgtggtgagtctggctgcagcgggccaaccaacccctccaacgtcaccggaaccaccacgatcatcacctagacaaacaccagcgcccggcccggcgcgcgaaaagtccgcagggaaaagagggcctgacagaggcagtccggaatacagacaacgccgggagaggaacaatatagctgtgagaaagagtcgcgataaggcgaaacggcggaaccaagaaatgcagcagaagctggtagaactcagcgcagagaacgagaagctccatcaacgggttgagcaactgactagggacctcgcgggcctgagacaatttttcaagcagctgccgagtccaccctttcttcccgcagcggggactgccgactgccgcggctctggggaaggacgcggtagccttctgacttgcggcgatgtggaggagaatcctgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 52 - RNA, 20 nt, synthetic construct
  source 1..20: mol_type = other RNA
gccgtccaggctgaagagcg

SEQ ID NO: 53 - RNA, 20 nt, synthetic construct
  source 1..20: mol_type = other RNA
cccggttcgtagaagggcgc

SEQ ID NO: 54 - RNA, 20 nt, synthetic construct
  source 1..20: mol_type = other RNA
ctctcgtcgtcgtacatggc

SEQ ID NO: 55 - DNA, 17 bp, synthetic construct
  source 1..17: mol_type = other DNA
gacagcctcgcttggac

SEQ ID NO: 56 - DNA, 18 bp, synthetic construct
  source 1..18: mol_type = other DNA
caagctcaccacggtctg

SEQ ID NO: 57 - DNA, 20 bp, synthetic construct
  source 1..20: mol_type = other DNA
ggtttcaccatgttgaccag

SEQ ID NO: 58 - DNA, 20 bp, synthetic construct
  source 1..20: mol_type = other DNA
agaatgggctttgtcatgtg

SEQ ID NO: 59 - RNA, 20 nt, synthetic construct
  source 1..20: mol_type = other RNA
aatcttgactctcaatccaa

SEQ ID NO: 60 - RNA, 20 nt, synthetic construct
  source 1..20: mol_type = other RNA
agctgtcactgtagagctga

SEQ ID NO: 61 - RNA, 20 nt, synthetic construct
  source 1..20: mol_type = other RNA
attttagcaggatggcccaa

SEQ ID NO: 62 - DNA, 20 bp, synthetic construct
  source 1..20: mol_type = other DNA
gggtgcccctttatctcctg

SEQ ID NO: 63 - DNA, 20 bp, synthetic construct
  source 1..20: mol_type = other DNA
gggagaaagaagcagggtcc

(19) United States
(12) Patent Application Publication
(10) Pub. No.: US 2026/0258360 A1
(43) Pub. Date: Sep. 3, 2026

(54) ENGINEERED NATURAL KILLER CELLS WITH ENHANCED ANTITUMOR MEMORY RESPONSES

(71) Applicant: BOARD OF REGENTS, THE UNIVERSITY OF TEXAS SYSTEM, Austin, TX (US)

(72) Inventors: Katy REZVANI, Houston, TX (US); Mayra SHANLEY, Houston, TX (US); Ken CHEN, Houston, TX (US); Merve DEDE, Houston, TX (US); Vakul MOHANTY, Houston, TX (US); Jinzhuang DOU, Houston, TX (US)

(73) Assignee: BOARD OF REGENTS, THE UNIVERSITY OF TEXAS SYSTEM, Austin, TX (US)

(21) Appl. No.: 19/166,168

(22) PCT Filed: Mar. 22, 2024
(86) PCT No.: PCT/US2024/021157

§ 371 Date: Sep. 17, 2025

Related U.S. Application Data
(60) Provisional application No. 63/492,056, filed on Mar. 24, 2023.

(51) Int. Cl. C12N 5/0783; A61K 40/15; A61K 40/35; A61K 40/42; A61P 35/00; C07K 14/47; C07K 14/54; C07K 14/705; C07K 14/715; C12N 9/12; C12N 9/64

(52) U.S. Cl. CPC C12N 5/0646; A61K 40/15; A61K 40/35; A61K 40/4234; A61P 35/00; C07K 14/47; C07K 14/4702; C07K 14/54; C07K 14/5443; C07K 14/70503; C07K 14/7051; C07K 14/70535; C07K 14/7056; C07K 14/715; C12N 9/12; C12N 9/6467; C12Y 207/10002; C12Y 304/21078; C12Y 304/21079; C12N 2501/2302; C12N 2510/00

(57) ABSTRACT

Embodiments of the disclosure include methods and compositions in which engineered NK cells are modified by the hand of man to overexpress a CCAAT/enhancer binding protein (C/EBP) family member. Such engineered NK cells have improved antitumor immune memory-like features, increased long term cytotoxicity, and metabolic fitness in vivo and in vitro.

### CROSS REFERENCE TO RELATED APPLICATIONS

This application claims the benefit of priority of U.S. Provisional Patent Application 63/492,056 filed on Mar. 24, 2023, the contents of which are hereby incorporated by reference in their entirety.

### GOVERNMENT SUPPORT CLAUSE

This invention was made with government support under CA127001 awarded by the National Institutes of Health. The government has certain rights in the invention.

### SEQUENCE LISTING

The instant application contains a Sequence Listing which has been submitted in ST26 format and is hereby incorporated by reference in its entirety. Said ST26 copy, created on Mar. 17, 2024, is named MDAC_1355WO_Sequence_Listing.xml and is 87,687 bytes in size.

### I. TECHNICAL FIELD

This disclosure relates at least to the fields of immunology, cell biology, molecular biology, and medicine, including at least cancer medicine.

### II. BACKGROUND

Natural killer (NK) cells have been studied as potential anti-tumor effectors, yet a number of barriers limit their therapeutic exploitation, such as an apparent lack of memory responses relative to other immune cells.

Glioblastoma multiforme (GBM) is the most common and aggressive primary brain tumor and presents a significant therapeutic challenge. Current treatments, including surgery, radiotherapy and chemotherapy¹, offer limited efficacy, with a median survival of only 18 to 21 months². Glioblastoma stem cell-like cells (GSCs) play a crucial role in tumor development and recurrence, and are resistant to conventional chemotherapy and radiotherapy³. Natural killer (NK) cells have an innate ability to recognize and kill GSCs^{4,5} and may therefore offer a promising immunotherapeutic strategy against this disease.

Cytokines can be used to further enhance the potency and in vivo persistence of NK cells against cancer. To date, IL-15 has been the primary focus of the translational and clinical work, as it has been shown to promote the cytotoxicity, proliferation and persistence of NK cells^{6,7}. Numerous preclinical studies and early clinical trials in patients with lymphoid malignancies support the overall safety and promising activity of systemically administered NK cells engineered to express IL-15 and a chimeric antigen receptor (CAR)⁸⁻¹². In addition, several studies have explored the safety and efficacy of locoregionally administered CAR-T cells and NK cells in GBM¹³⁻¹⁵ However, the safety and activity of locoregionally administered IL-15 NK cells in the context of GBM has not been explored. IL-21 is another attractive cytokine for cancer immunotherapy, and is known to induce metabolic reprogramming and mitochondrial biogenesis in T cells¹⁶. IL-21 also promotes NK cell proliferation, maturation and metabolic fitness¹⁷⁻¹⁹. Recombinant IL-21 was tested in several clinical trials of metastatic cancer, and despite its acceptable safety profile, its short half-life and the need for repeated dosing limited its clinical application^{20,21}.

The present disclosure satisfies a need in the art to improve upon therapies for treating cancers, such as brain cancers, through use of immunotherapies that comprise engineered NK cells.

### BRIEF SUMMARY

Using multiple patient-derived GSCs and orthotopic in vivo models, the inventors herein show that locoregionally administered IL-15 NK cells (NK cells engineered to express secreted IL-15, see FIG. 1) are highly toxic and ineffective at GBM tumor control. In contrast, the data showed that IL-21 NK cells (NK cells engineered to express secreted IL-21, see FIG. 1) administered similarly were safe and resulted in long-term antitumor activity. IL-21 NK cells exhibited distinct epigenetic and transcriptional signatures, with the CCAAT/Enhancer-Binding Proteins (C/EBP, CEBP) family of transcription factors (TFs) emerging as critical regulators of IL-21 NK cell response against GBM. Deletion of CEBPD, the most differentially expressed member of the CEBP TFs in IL-21 NK cells, impaired their potency and long-term antitumor response, while CEBPD overexpression in NK cells increased their functional capacity and metabolic fitness. The data showed that STAT3 functioned as a key signaling pathway for CEBPD-mediated gene expression regulation. These data identify C/EBP TFs, particularly CEBPD, as important transcriptional and epigenetic coordinators of NK cell responses to cancer, and support arming NK cells with IL-21 as an immunotherapeutic approach for treatment of GBM.

Embodiments of the disclosure include at least methods and compositions for treatment of an individual with a disorder (e.g., cancer) using adoptive cell therapy. In certain embodiments, provided herein are engineered Natural Killer (NK) cells modified to overexpress a CCAAT/Enhancer-binding protein (CEBP) transcription factor family protein. In certain embodiments, a CEBP transcription factor expression is relative to a non-engineered NK cell and/or relative to an NK cell engineered to express IL-15. In certain embodiments, the CEBP protein is CEBPA (CEBP-alpha, CEBPα), CEBPB (CEBP-beta, CEBPβ), CEBPG (CEBP-gamma, CEBPγ), CEBPD (CEBP-delta, CEBPδ), CEBPE (CEBP-epsilon, CEBPε) and/or CEBP Homologous Protein (CHOP). In certain embodiments, the CEBP protein is CEBPD and/or CEBPB. In certain embodiments, engineered NK cells overexpresses a transgenic CEBPB and/or CEBPD protein. In certain embodiments, NK cell transgenically expresses and/or are subjected to a CEBP protein transcriptional and/or translational activator. In certain embodiments, NK cell transgenically express and/or are subjected to an inhibitor of a CEBP protein transcriptional and/or translational inhibitor.

In certain embodiments, provided herein are engineered NK cells overexpressing a CEBP protein, wherein the CEBP protein comprises a sequence at least 80%, 85%, 90%, 95%, 99%, or 100% identical to SEQ ID NO: 19. In certain embodiments, the CEBP protein is encoded by a sequence comprising a sequence at least 80%, 85%, 90%, 95%, 99%, or 100% identical to SEQ ID NO: 20 or 21. In certain embodiments, the CEBP protein comprises a sequence at least 80%, 85%, 90%, 95%, 99%, or 100% identical to any one of SEQ ID NOs: 23, 25, or 27. In certain embodiments, the CEBP protein is encoded by a sequence comprising a sequence at least 80%, 85%, 90%, 95%, 99%, or 100% identical to any one of SEQ ID NOs: 24, 26, or 28.

In certain embodiments, an engineered CEBP overexpressing NK cell has enhanced mitochondrial fitness and/or memory-like features relative to a non-engineered NK cell and/or relative to an NK cell engineered to express IL-15. In certain embodiments, the enhanced mitochondrial fitness comprises increased basal and/or maximal oxygen consumption rate (OCR) compared to a non-engineered NK cell and/or an NK cell engineered to express IL-15, and/or a reduction in glycolysis (e.g., wherein glycolysis is measured by extracellular acidification rate (ECAR)) compared to a non-engineered NK cell and/or an NK cell engineered to express IL-15.

In certain embodiments, an engineered CEBP overexpressing NK cell has enhanced anti-tumor cytotoxicity relative to a non-engineered NK cell and/or relative to an NK cell engineered to express IL-15. In certain embodiments, the NK cell has enhanced anti-tumor memory relative to a non-engineered NK cell and/or relative to an NK cell engineered to express IL-15. In certain embodiments, the NK cell has high expression of functional markers, low expression of inhibitory markers, high expression of survival genes, low expression of exhaustion genes, and/or upregulation of activation receptors and markers, relative to a non-engineered NK cell and/or relative to an NK cell engineered to express IL-15. In certain embodiments, the functional markers comprise Granzyme A (GrA), Granzyme B (GrB), Perforin, and/or Zap70. In certain embodiments, the inhibitory markers comprise LAG3 and/or KLRG1. In certain embodiments, the activation receptors and markers comprise NKp30, CD25, DNAM, Ki67, CD3ζ, T-bet, and/or FCεRG. In certain embodiments, the survival genes comprise KLRD1, ITGA1 and/or GZMK. In certain embodiments, the exhaustion genes comprise DUSP2, CISH, and/or BAX. In certain embodiments, the NK cells comprise high expression levels of cytotoxicity markers GrA, GrB, Perforin, TRAIL, and/or CD95, and/or high expression levels of activation markers/receptors CD25, CD69, DNAM, NKG2D, NKp44 and/or NKp46, relative to a non-engineered NK cell and/or relative to an NK cell engineered to express IL-15. In certain embodiments, the NK cells comprise high expression levels of transcription factors important for NK cell maturation, NK cell immune function, NK cell cytotoxicity, IFN-γ response, memory formation, and/or AP-1 complex members, relative to a non-engineered NK cell and/or relative to an NK cell engineered to express IL-15. In certain embodiments, the transcription factors comprise CEBPD, CEBPB, ETS1, IRF1, TBX21, EOMES, IRF9, STAT1, ETS1, JUN, JUNB, JUND, FOS, and/or FOSL1.

Also provided herein are engineered CEBP overexpressing NK cells that are further engineered to express one or more interleukins (IL). In certain embodiments, the IL is IL-2, IL-7, IL-12, IL-15, IL-17, IL-18, IL-21, and/or the p35 and p40 subunits of IL-12 artificially linked together. In certain embodiments, the IL is IL-21 and/or IL-15. In certain embodiments, the IL is secreted, tethered, or membrane bound in the cell. In certain embodiments, NK cells described herein gain increased tumor cell apoptosis, apoptosis, and/or cytotoxicity capacity. In certain embodiments, the NK cell has increased levels and/or activity of ERK1/2, NFκB, IFNG, TNFSF10, FASLG, and/or Nfat.

In certain embodiments, an NK cell described herein is derived from cord blood (CB), peripheral blood (PB), bone marrow, stem cells, NK cell lines, or a combination thereof. In certain embodiments, NK cells are primary NK cells, and are not derived from stem cells and/or induced pluripotent stem cells (iPSCs). In certain embodiments, NK cells are complexed to one or more monospecific, bispecific, and/or multi-specific antibodies. In certain embodiments, an NK cell expresses one or more antibody. In certain embodiments, an NK cell is further modified to express one or more additional heterologous proteins selected from the group consisting of an antigen receptor, a cytokine, a homing receptor, a chemokine receptor, and a combination thereof. In certain embodiments, an engineered receptor is an engineered antigen receptor. In certain embodiments, a target antigen is a cancer antigen. In certain embodiments, an NK cell comprises a suicide gene. In certain embodiments, an NK cell further comprises one or more engineered mutations in an endogenous gene. In certain embodiments, the endogenous gene is TGFBR2, CISH, GR, and/or CD38. In certain embodiments, NK cells are pre-activated with one or more cytokines. In certain embodiments, one or more cytokines comprises IL-2.

Also provided herein are compositions comprising engineered NK cells described herein. In some embodiments, a composition comprises a pharmaceutically acceptable excipient. In some embodiments, a composition is comprised in a delivery device.

Also provided herein are methods of treating disease in an individual, wherein the method can comprise the step of administering to the individual a therapeutically effective amount of any one of the engineered NK cells or compositions described herein. In some embodiments, a disease is an autoimmune disease, infection, and/or cancer. In some embodiments, a disease is cancer. In some embodiments, a cancer is of the lung, brain, breast, blood, skin, pancreas, liver, colon, head and neck, kidney, thyroid, stomach, spleen, gallbladder, bone, ovary, testes, endometrium, prostate, rectum, anus, cervix, or is hematological. In some embodiments, a cancer is glioblastoma. In some embodiments, a glioblastoma is a TCGA mesenchymal, neural, classical, or Proneural subtype. In some embodiments, a glioblastoma is a mesenchymal, or Proneural subtype. In some embodiments, a glioblastoma has an MGMT unmethylated, methylated, or indeterminate status. In some embodiments, a glioblastoma is primary or recurrent.

In some embodiments, engineered NK cells as described herein utilized in methods described herein display increased in vivo antitumor functionality relative to a non-engineered NK cell and/or an NK cell engineered to express IL-15. In some embodiments, increased in vivo antitumor functionality comprises long-term tumor eradication, significant prolongation of subject survival, and/or reduced toxicity or weight loss.

Also provided herein are methods of providing a subject with immune memory against cancer, the method comprising administering to an individual a therapeutically effective amount of an engineered NK cell and/or composition described herein.

Also provided herein are isolated nucleic acids encoding a CEBP protein fused to a heterologous transcriptional regulatory element. In some embodiments, a CEBP protein comprises a sequence at least 80%, 85%, 90%, 95%, 99%, or 100% identical to SEQ ID NO: 19. In some embodiments, a CEBP protein is encoded by a sequence comprising a sequence at least 80%, 85%, 90%, 95%, 99%, or 100% identical to SEQ ID NO: 20 or 21. In some embodiments, a heterologous transcriptional regulatory element is a promoter.

In some embodiments, also provided herein are methods of treating glioblastoma, the methods comprising administering to an individual a therapeutically effective amount of an NK cell engineered to constitutively express secreted IL-21. In some embodiments, the method of treating glioblastoma comprises administering the NK cells through intracranial injection. In some embodiments, the administering is through intratumoral injection. In some embodiments, the NK cell engineered to constitutively and/or autonomously express secreted IL-21 comprises a transgenic polynucleotide sequence encoding and/or comprising a polynucleotide sequence at least 80%, 85%, 90%, 95%, or 100% identical to SEQ ID NOs: 31-32. In some embodiments, the method of treating glioblastoma provides the subject with immune memory against glioblastoma. In some embodiments, the method of treating glioblastoma provides the subject with immune memory against glioblastoma stem cells. In certain embodiments, the NK cells are engineered to stably secrete IL-21 at a rate suitable for reaching an extracellular concentration of greater than or equal to 200, 250, 300, 350, 400, 450, 500, 550, 600, 650, 700, or 750 pg/mL. In certain embodiments, the NK cells are engineered to stably secrete IL-21 at a rate suitable for reaching an extracellular concentration of greater than or equal to 200, 250, 300, 350, 400, 450, 500, 550, 600, 650, 700, or 750 pg/mL within 1 day, 2 days, or 3 days of culture. In certain embodiments, the NK cells are engineered to stably secrete IL-21 at a rate suitable for reaching an extracellular concentration of greater than or equal to 200, 250, 300, 350, 400, 450, 500, 550, 600, 650, 700, 750, 800, 850, 900, 950, 1000, 1050, 1100, 1200, 1300, 1400, 1500, 1600, 1700, 1800, 1900, 2000, 2100, 2200, 2300, 2400, 2500, 2600, 2700, 2800, 2900, 3000 or greater than 3000 pg/mL within 3 days, 4 days, or 5 days of culture. In certain embodiments, the NK cells are engineered to stably IL-21 at a rate suitable for reaching an extracellular concentration of equal to or about 200-800 pg/mL, 250-750 pg/mL, 300-700 pg/mL, or 350-650 pg/mL, or any value derivable therein. In certain embodiments, the NK cells are engineered to stably IL-21 at a rate suitable for reaching an extracellular concentration of equal to or about 200-800 pg/mL, 250-750 pg/mL, 300-700 pg/mL, or 350-650 pg/mL, or any value derivable therein, and maintaining said concentration through media changes that occur every 1 day, 2 days, 3 days, 4 days, or 5 days.

Certain embodiments of the present disclosure and associated inventions can be characterized through the following enumerated Aspects.

Aspect 1 is an engineered Natural Killer (NK) cell modified to overexpress a CCAAT/Enhancer-binding protein (CEBP) transcription factor family protein.

Aspect 2 is the engineered NK cell of aspect 1, wherein CEBP transcription factor expression is relative to a non-engineered NK cell and/or relative to an NK cell engineered to express IL-15.

Aspect 3 is the engineered NK cell of aspect 1, wherein the CEBP protein is CEBPD (CEBP-delta, CEBPδ), CEBPA (CEBP-alpha, CEBPα), CEBPB (CEBP-beta, CEBPβ), CEBPG (CEBP-gamma, CEBPγ), CEBPE (CEBP-epsilon, CEBPε) and/or CEBP Homologous Protein (CHOP).

Aspect 4 is the engineered NK cell of aspect 1 or 3, wherein the CEBP protein is CEBPD and/or CEBPB.

Aspect 5 is the engineered NK cell of any one of aspects 1 to 4, wherein the NK cell overexpresses a transgenic CEBPB and/or CEBPD protein.

Aspect 6 is the engineered NK cell of any one of aspects 1 to 5, wherein the NK cell transgenically expresses and/or are subjected to a CEBP protein transcriptional and/or translational activator.

Aspect 7 is the engineered NK cell of any one of aspects 1 to 6, wherein the NK cell transgenically expresses and/or are subjected to an inhibitor of a CEBP protein transcriptional and/or translational inhibitor.

Aspect 8 is the engineered NK cell of any one of aspects 1 to 7, wherein the CEBP protein comprises a sequence at least 80%, 85%, 90%, 95%, 99%, or 100% identical to SEQ ID NO: 19.

Aspect 9 is the engineered NK cell of any one of aspects 1 to 8, wherein the CEBP protein is encoded by a sequence comprising a sequence at least 80%, 85%, 90%, 95%, 99%, or 100% identical to SEQ ID NO: 20 or 21.

Aspect 10 is the engineered NK cell of any one of aspects 1 to 9, wherein the CEBP protein comprises a sequence at least 80%, 85%, 90%, 95%, 99%, or 100% identical to any one of SEQ ID NOs: 23, 25, or 27.

Aspect 11 is the engineered NK cell of any one of aspects 1 to 10, wherein the CEBP protein is encoded by a sequence comprising a sequence at least 80%, 85%, 90%, 95%, 99%, or 100% identical to any one of SEQ ID NOs: 24, 26, or 28.

Aspect 12 is the engineered NK cell of any one of aspects 1 to 11, wherein the NK cell has enhanced mitochondrial fitness and/or memory-like features relative to a non-engineered NK cell and/or relative to an NK cell engineered to express IL-15.

Aspect 13 is the engineered NK cell of aspect 12, wherein the enhanced mitochondrial fitness comprises increased basal and/or maximal oxygen consumption rate (OCR) compared to a non-engineered NK cell and/or an NK cell engineered to express IL-15, and/or a reduction in glycolysis (e.g., wherein glycolysis is measured by extracellular acidification rate (ECAR)) compared to a non-engineered NK cell and/or an NK cell engineered to express IL-15.

Aspect 14 is the engineered NK cell of any one of aspects 1 to 13, wherein the NK cell has enhanced anti-tumor cytotoxicity relative to a non-engineered NK cell and/or relative to an NK cell engineered to express IL-15.

Aspect 15 is the engineered NK cell of any one of aspects 1 to 14, wherein the NK cell has enhanced anti-tumor memory relative to a non-engineered NK cell and/or relative to an NK cell engineered to express IL-15.

Aspect 16 is the engineered NK cell of any one of aspects 1 to 15, wherein the NK cell has high expression of functional markers, low expression of inhibitory markers, high expression of survival genes, low expression of exhaustion genes, and/or upregulation of activation receptors and markers, relative to a non-engineered NK cell and/or relative to an NK cell engineered to express IL-15.

Aspect 17 is the engineered NK cell of aspect 16, wherein the functional markers comprises Granzyme A (GrA), Granzyme B (GrB), Perforin, and/or Zap70.

Aspect 18 is the engineered NK cell of aspect 16, wherein the inhibitory markers comprises LAG3 and/or KLRG1.

Aspect 19 is the engineered NK cell of aspect 16, wherein the activation receptors and markers comprises NKp30, CD25, DNAM, Ki67, CD3ζ, T-bet, and/or FCεRG.

Aspect 20 is the engineered NK cell of aspect 16, wherein the survival genes comprises KLRD1, ITGA1 and/or GZMK.

Aspect 21 is the engineered NK cell of aspect 16, wherein the exhaustion genes comprises DUSP2, CISH, and/or BAX.

Aspect 22 is the engineered NK cell of any one of aspects 1 to 21, wherein the NK cells comprise high expression levels of cytotoxicity markers GrA, GrB, Perforin, TRAIL, and/or CD95, and/or high expression levels of activation markers/receptors CD25, CD69, DNAM, NKG2D, NKp44 and/or NKp46, relative to a non-engineered NK cell and/or relative to an NK cell engineered to express IL-15.

Aspect 23 is the engineered NK cell of any one of aspects 1 to 22, wherein the NK cells comprise high expression levels of transcription factors important for NK cell maturation, NK cell immune function, NK cell cytotoxicity, IFN-γ response, memory formation, and/or AP-1 complex members, relative to a non-engineered NK cell and/or relative to an NK cell engineered to express IL-15.

Aspect 24 is the engineered NK cell of aspect 23, wherein the transcription factors comprise CEBPD, CEBPB, ETS1, IRF1, TBX21, EOMES, IRF9, STAT1, ETS1, JUN, JUNB, JUND, FOS, and/or FOSL1.

Aspect 25 is the engineered NK cell of any one of aspects 1 to 24, wherein the cell is further engineered to provide one or more interleukins (IL).

Aspect 26 is the engineered NK cell of aspect 25, wherein the IL is IL-2, IL-7, IL-12, IL-15, IL-17, IL-18, IL-21, and/or the p35 and p40 subunits of IL-12 artificially linked together.

Aspect 27 is the engineered NK cell of aspect 26, wherein the IL is IL-21 and/or IL-15.

Aspect 28 is the engineered NK cell of aspect 26 or 27, wherein the NK cell gains increased tumor cell apoptosis, apoptosis, and/or cytotoxicity capacity.

Aspect 29 is the engineered NK cell of any one of aspects 26 to 28, wherein the NK cell has increased levels and/or activity of ERK1/2, NFκB, IFNG, TNFSF10, FASLG, and/or Nfat.

Aspect 30 is the engineered NK cell of any one of aspects 25 to 29, wherein the IL is secreted, tethered, or membrane bound in the cell.

Aspect 31 is the engineered NK cell of any one of aspects 1 to 30, wherein the NK cells are derived from cord blood (CB), peripheral blood (PB), bone marrow, stem cells, NK cell lines, or a combination thereof.

Aspect 32 is the engineered NK cell of any one of aspects 1 to 31, wherein the NK cells are primary NK cells, and are not derived from stem cells and/or induced pluripotent stem cells (iPSCs).

Aspect 33 is the engineered NK cell of any one of aspects 1 to 32, wherein the NK cells are complexed to one or more monospecific, bispecific, and/or multi-specific antibodies.

Aspect 34 is the engineered NK cell of aspect 33, wherein the NK cell expresses one or more antibody.

Aspect 35 is the engineered NK cell of any one of aspects 1 to 34, wherein the NK cell is further modified to express one or more additional heterologous proteins selected from the group consisting of an antigen receptor, a cytokine, a homing receptor, a chemokine receptor, and a combination thereof.

Aspect 36 is the engineered NK cell of aspect 35, wherein the engineered receptor is an engineered antigen receptor.

Aspect 37 is the engineered NK cell of any one of aspects 33 to 36, wherein a target antigen is a cancer antigen.

Aspect 38 is the engineered NK cell of any one of aspects 1 to 37, wherein the NK cell comprises a suicide gene.

Aspect 39 is the engineered NK cell of any one of aspects 1 to 38, wherein the NK cell further comprises one or more engineered mutations in an endogenous gene.

Aspect 40 is the engineered NK cell of aspect 39, wherein the endogenous gene is TGFBR2, CISH, GR, and/or CD38.

Aspect 41 is the engineered NK cell of any one of aspects 1 to 40, wherein the NK cells are pre-activated with one or more cytokines.

Aspect 42 is the engineered NK cell of aspect 41, wherein the one or more cytokines comprises IL-2.

Aspect 43 is a composition comprising the engineered NK cell of any one of aspects 1 to 42.

Aspect 44 is the composition of aspect 43, further comprising a pharmaceutically acceptable excipient.

Aspect 45 is the composition of aspect 43 or 44, wherein the composition is comprised in a delivery device.

Aspect 46 is a method of treating a disease in an individual, the method comprising the step of administering to the individual a therapeutically effective amount of any one of the engineered NK cells or compositions of any one of the preceding aspects.

Aspect 47 is the method of aspect 46, wherein the disease is an autoimmune disease, infection, and/or cancer.

Aspect 48 is the method of aspect 46 or 47, wherein the disease is cancer.

Aspect 49 is the method of aspect 48, wherein the cancer is of the lung, brain, breast, blood, skin, pancreas, liver, colon, head and neck, kidney, thyroid, stomach, spleen, gallbladder, bone, ovary, testes, endometrium, prostate, rectum, anus, cervix, or is hematological.

Aspect 50 is the method of aspect 48 or 49, wherein the cancer is glioblastoma.

Aspect 51 is the method of aspect 50, where the glioblastoma is a TCGA mesenchymal, neural, classical, or Proneural subtype.

Aspect 52 is the method of aspect 51, where the glioblastoma is a mesenchymal, or Proneural subtype.

Aspect 53 is the method of aspect 50 or 51, wherein the glioblastoma has an MGMT unmethylated, methylated, or indeterminate status.

Aspect 54 is the method of any one of aspects 50 to 53, wherein the glioblastoma is primary or recurrent.

Aspect 55 is the method of any one of aspects 48 to 54, wherein the engineered NK cells display increased in vivo antitumor functionality relative to a non-engineered NK cell and/or an NK cell engineered to express IL-15.

Aspect 56 is the method of aspect 55, wherein the increased in vivo antitumor functionality comprises long-term tumor eradication, significant prolongation of subject survival, and/or reduced toxicity or weight loss.

Aspect 57 is the method of any one of aspects 50-56, wherein the administering is through intracranial injection.

Aspect 58 is the method of any one of aspects 50-57 wherein the administering is through intratumoral injection.

Aspect 59 is the method of any one of aspects 50-58, wherein the method provides immune memory against glioblastoma.

Aspect 60 is the method of any one of aspects 50-59, wherein the method provides immune memory against glioblastoma stem cells.

Aspect 61 is a method of providing a subject with immune memory against cancer, the method comprising administering to an individual a therapeutically effective amount of the engineered NK cells of any one of aspects 1 to 42, or compositions of any one of aspects 43 to 45.

Aspect 62 is an isolated nucleic acid encoding a CEBP protein fused to a heterologous transcriptional regulatory element.

Aspect 63 is the isolated nucleic acid of aspect 62, wherein the CEBP protein comprises a sequence at least 80%, 85%, 90%, 95%, 99%, or 100% identical to SEQ ID NO: 19.

Aspect 64 is the isolated nucleic acid of aspect 62 or 63, wherein the CEBP protein is encoded by a sequence comprising a sequence at least 80%, 85%, 90%, 95%, 99%, or 100% identical to SEQ ID NO: 20 or 21.

Aspect 65 is the isolated nucleic acid of any one of aspects 62 to 64, wherein the heterologous transcriptional regulatory element is a promoter.

Aspect 66 is a method of treating glioblastoma, the method comprising administering to an individual a therapeutically effective amount of an NK cell engineered to autonomously and/or constitutively express secreted IL-21.

Aspect 67 is the method of aspect 66, wherein the administering is through intracranial injection.

Aspect 68 is the method of aspect 66 or 67, wherein the administering is through intratumoral injection.

Aspect 69 is the method of any one of aspects 66-68, wherein the NK cell engineered to constitutively and/or autonomously express secreted IL-21 comprises a transgenic polynucleotide sequence encoding and/or comprising a sequence at least 80%, 85%, 90%, 95%, or 100% identical to SEQ ID NOs: 31-32.

Aspect 70 is the method of any one of aspects 66-69, wherein the method provides immune memory against glioblastoma.

Aspect 71 is the method of any one of aspects 66-70, wherein the method provides immune memory against glioblastoma stem cells.

Aspect 72 is the method of any one of aspects 66-71, wherein the NK cells are engineered to stably secrete IL-21 a rate suitable for reaching an extracellular concentration of greater than or equal to 200, 250, 300, 350, 400, 450, 500, 550, 600, 650, 700, or 750 pg/mL.

Aspect 73 is the method of any one of aspects 66-72, wherein the NK cells are engineered to stably secrete IL-21 a rate suitable for reaching an extracellular concentration of greater of equal to or about 200-800 pg/mL, 250-750 pg/mL, 300-700 pg/mL, or 350-650 pg/mL.

Aspect 74 is the method of any one of aspects 66-73, wherein the glioblastoma is a TCGA mesenchymal, neural, classical, or Proneural subtype.

Aspect 75 is the method of any one of aspects 66-74, wherein the glioblastoma is a mesenchymal, or Proneural subtype.

Aspect 76 is the method of any one of aspects 66-75, wherein the glioblastoma has an MGMT unmethylated, methylated, or indeterminate status.

Aspect 77 is the method of any one of aspects 66-76, wherein the glioblastoma is primary or recurrent.

Aspect 78 is the method of any one of aspects 66-77, wherein the engineered NK cells display increased in vivo antitumor functionality relative to a non-engineered NK cell and/or an NK cell engineered to express IL-15.

Aspect 79 is the method of aspect 78, wherein the increased in vivo antitumor functionality comprises long-term tumor eradication, significant prolongation of subject survival, and/or reduced toxicity or weight loss.

Other objects, features and advantages of the present inventions will become apparent from the following detailed description. It should be understood, however, that the detailed description and the specific examples, while indicating specific embodiments of the inventions, are given by way of illustration only, since various changes and modifications within the spirit and scope of the inventions will become apparent to those skilled in the art from this detailed description.

### BRIEF DESCRIPTION OF THE DRAWINGS

The following drawings form part of the present specification and are included to further demonstrate certain aspects of the present inventions. The inventions may be better understood by reference to one or more of these drawings in combination with the detailed description of specific embodiments presented herein.

FIGS. 1A-1I—Cytokine arming improved NK cell antitumor activity against glioblastoma stem cells (GSC). (FIG. 1A) Schematic representation of two retroviral vectors used to transduce NK cells to secrete IL-15 or IL-21. (FIG. 1B) NK cells were expanded with uAPC feeder cells and IL-2 for 5 days and then transduced with a retroviral vector encoding IL-15 or IL-21 (SEQ ID NO: 29 or 31). The transduction efficiency was determined by assessing IgG expression using an antibody against human IgG1 (n=5-6 donors) by flow cytometry. (FIG. 1C) Total IL-15 (n=3 donors) and IL-21 (n=6 donors) cytokine levels (pg/mL; ELISA) in supernatants from non-transduced (NT) NK (NT NK), IL-15-armed NK (IL-15 NK), or IL-21-armed NK (IL-21 NK) cells 5 days after transduction. Error bars denote standard deviation. (FIG. 1D) Percentage (%) of GSC20 killing by NT NK, IL-15 NK, or IL-21 NK cells over time as measured by real-time killing assay (E:T ratio of 1:1); asterisks depict statistical significance for the comparisons. IL-15 NK vs NT NK=***; IL-21 NK vs NT NK=**. (FIGS. 1E-1F) K562 killing by NT NK, IL-15 NK, or IL-21 NK cells that were cultured either alone (FIG. 1E) or for 48 hours with GSC20 (FIG. 1F) at an E:T ratio of 1:1. NK cells were then purified and their ability to kill K562 targets was assessed by real time killing assays (n=6 donors). IL-15 NK vs NT NK=***; IL-21 NK vs NT NK=***. (FIGS. 1G-1I) 3D killing assay of GSC20 and GSC272 spheroids by NT NK, IL-15 NK, or IL-21 NK cells (n=3 donors). Total red object integrated intensity (y axis, RCU×μm²/image) revealed the growth signal of the GSC272 (FIG. 1G) or GSC20 (FIG. 1H) spheroids over time (x axis). IL-21 NK cells and IL-15 NK cells induced significant levels (P=***) of GSC272 and GSC20 apoptosis relative to NT NK controls. (FIG. 1I) Images depict NT NK, IL-15 NK, or IL-21 NK cell with GSC272 co-culture, or control GSC272 cells (x axis) over time (y axis), with red signals from GSC272 spheroid 3D killing assays monitored over time, IL-21 NK cells induced significant levels of GSC272 apoptosis within 24 hours of co-culture. Images depict the red signals from live imaging of GSC272 spheroid 3D killing assays. Statistical significance was determined using two-way ANOVA with Dunnet correction for multiple comparisons. ***p≤0.001.

FIGS. 2A-2P—IL-21-armed NK cells exhibited long-term cytotoxicity against GSCs and displayed greater metabolic fitness than IL-15-armed or unarmed NK cells. Cytokine-armed NK cells or non-transduced (NT) controls, and m-cherry transduced GSC20 (red) were co-cultured at an E:T ratio of 1:1. Every 2-3 days, fresh GSCs were added to the co-cultures without adding new NK cells. Red (GSC) signal was followed with real time imaging. (FIGS. 2A-2C) NK cell surface and intracellular markers expression were measured by mass cytometry for IL-15 NK, IL-21 NK, and NT NK cells (n=3 donors for each) cultured either alone or in the presence of GSC20 for 48 hours. (FIG. 2A) tSNE plots showing the cluster distribution. (FIG. 2B) Analysis of the tSNE plots showing cluster distribution, expression, and fraction, where color scale and circle size represent expression and size of cluster for each group. (FIG. 2C) Comparative heatmap showing expression of NK cell markers at the cluster level. Heatmap column clustering was identified by FlowSOM analysis. Color scale shows the expression level for each marker, with red representing higher expression and blue lower expression (n=3 donors). (FIGS. 2D, 2F, and 2G) Graphs showing the red object count per image over time, which correlated with the proportion of live cancer cells after each (FIG. 2D) GSC20 rechallenge (n=3 donors), (FIG. 2F) GSC272 rechallenge (n=3 donors), and (FIG. 2G) GSC267 rechallenge (n=3 donors). The data showed IL-21 NK cells maintained high levels of cytotoxicity against GSC20, GSC272, and GSC267 cells over 5 rechallenge events, a trait not observed in NT NK or IL-15 NK cells. Asterisks represent statistical differences between groups. Error bars denote standard deviation. (FIG. 2E) Summary red object count per image after each GSC20 rechallenge from 2 separate experiments (n=6 donors). Error bars denote standard deviation. (FIG. 2H) Heatmap showing levels of different cytokines measured by multiplex ELISA in supernatants collected from co-cultures of NK cells with GSCs in the experiment presented in FIG. 2D. (FIGS. 2I-2J) Polyfunctionality scores (FIG. 2I) and polyfunctionality strength index (FIG. 2J) of NT NK, IL-15 NK, or IL-21 NK cells in response to GSC20 (n=4 donors). Error bars denote standard deviation. (FIG. 2K) Representative measurements of oxygen consumption rate (OCR) upon addition of oligomycin (Oligo), FCCP, and rotenone and antimycin A (R/A). Quantified basal respiration (FIG. 2L) and maximal respiration (FIG. 2M) of purified NT NK, IL-15 NK, and IL-21 NK cells after 48 hours of co-culture with GSC20 (n=3 donors). (FIG. 2N) Representative measurements of extracellular acidification rate (ECAR) upon addition of glucose, Oligo, and 2-deoxy-D-glucose (2-DG) and quantified basal glycolysis (FIG. 2O) and glycolytic capacity (FIG. 2P) of purified NT NK, IL-15 NK, and IL-21 NK cells after 48 hours of co-culture with GSC20 (n=3 donors). Error bars denote standard error of mean. Statistical significance was determined using two-way ANOVA with Bonferroni correction for multiple comparisons. *p≤0.05, **p≤0.01, ***p≤0.001.

FIGS. 3A-3G—IL-21-armed NK cells displayed significantly superior in vivo anti-tumor activities in orthotopic PDX mouse models of patient-derived GSCs when compared to controls. (FIG. 3A) Displays a schematic diagram showing the injection timeline, and representative Bioluminescence imaging (BLI) images displaying in vivo tumor status. BLI was utilized to monitor growth of FFluc-labeled GSC20 tumor cells (0.5×10⁶) that were intracranially (I.C.) injected into NSG (NOD scid gamma) mice. The tumors were allowed to grow for seven days, and mice were then treated intratumorally (I.T. or IT) with injection of (0.1×10⁶) NT NK, IL-15 NK, or IL-21 NK cells (n=3-5 mice per group). The BLI imaging showed that IL-21 NK cells could effectively eliminate and maintain the absence of FFluc-labeled GSC20 tumor cell signal until at least Day 176 following tumor cell inoculation, significantly longer than the control groups. (FIG. 3B) Shows quantification of individual (fine lines) and average (thick lines) radiance (BLI) data (p/sec/cm2/sr) from mice shown in FIG. 3A. Error bars denote standard error of mean. Statistical significance was determined using unpaired t-test. **p≤0.01, ns=not significant. (FIG. 3C) Kaplan-Meier plot showing mouse survival in each group (n=3-5 mice per group). Animals treated with IL-21 NK cells had significantly better survival compared with GSC20 alone (p=0.0042), IL-15 NK cells (p=0.0027) or NT NK cell controls (p=0.0042) based on log-rank test. *p≤0.05, **p≤0.01, ns=not significant. (FIG. 3D) Is a graph showing the body weight change (%) of mice over time in the different groups described in FIG. 3C. IL-21 NK cells maintained and/or slightly increased in bodyweight throughout the experimental timeline, and IL-21 NK cells had significantly higher body weights than the control groups. Statistical significance was determined using unpaired t-test. **p≤0.01, ***p≤0.001, ns=not significant. (FIG. 3E) Schematic diagram showing GSC20 in vivo rechallenge. IL-21 NK-treated mice with no sign of tumor at day 400 after tumor inoculation were then re-injected with 0.25×10⁶ GSC20 (n=4 mice), as was a new cohort of control NSG mice; brain tissue was collected 17 days after GSC rechallenge (n=4 test mice, or n=5 control mice). Flow cytometry showing NK cells in brain tissue (CD56+CD3-CD16+ cells) from IL-21 NK cell treated (n=3 out of 4 mice with available tissue) or tumor only control (n=5 mice) mice after GSC20 rechallenge (FIG. 3F) and immunohistochemical (IHC) CD16 staining (FIG. 3G). The arrows show CD16+NK cells that had infiltrated the brain. Representative IHC images from brain sections from two mice (#1 and #2) treated with IL-21 NK cells and rechallenged with GSC20 (left panels) compared to tumor only control (top right panel) and tonsil (positive control; bottom right panel) are shown. Images were taken at 10×.

FIGS. 4A-4J—Epigenetic and transcriptomic profiles of IL-21 NK cells were characterized over time after GSC co-culture. (FIG. 4A) displays a schematic representation of scRNA-seq and scATAC-seq characterization experiments, and a UMAP plot of scATAC-seq data showing the cluster level epigenetic evolution of IL-15 NK (top) and IL-21 NK (bottom) cells over time from baseline (day 0) to days 3 and 9 following co-culture with GSCs. (FIG. 4B) Fish plots showing the prevalence of different scATAC-seq clusters in IL-15 NK cells and IL-21 NK cells over time, from day 0 (baseline) to days 3 and 9 after co-culture with GSC20. (FIG. 4C) Transcription factor enrichment of Cluster 6 (mostly from day 9 IL-21 NK cells) specific peaks. (FIG. 4D) Volcano plots showing transcription factor enrichments of IL-15 NK cell and IL-21 NK cell specific peaks at day 3 (left figure) and day 9 (right figure) post-co-culture with GSC20. The red dots represent transcription factors (TFs) with motifs that were highly enriched in product-specific peaks. (FIG. 4E) IL-21 motif enrichment at day 9. Motifs of members of the CCAAT/enhancer binding proteins (C/EBP) family were among the top 20 most enriched TFs discovered, and select members binding motifs are shown. (FIG. 4F) UMAP plot of scRNA-seq data showing the transcriptomic clusters and their evolution over time in IL-15 NK (top) and IL-21 NK (bottom) cells at baseline (day 0) and at days 3 and 9 following co-culture with GSC20. (FIG. 4G) Fish plots showing the prevalence of clusters from scRNA-seq data of IL-15 NK and IL-21 NK cells over time from day 0 (baseline) to days 3 and 9 after co-culture with GSC20. (FIG. 4H) Volcano plot showing genes with significant upregulation in IL-15 NK cell cluster 3 (left) and IL-21 NK cell cluster 4 (right) at day 9 after co-culture with GSC20. The red dots represent TFs with higher gene expression levels. (FIG. 4I) Venn diagram showing the DEGs in cluster 4 by scRNA-seq overlapping with the associated genes with a chromatin open region in Cluster 6 by scATAC-seq. These two clusters were unique to IL-21 NK cells upon multiple rechallenges with GSC. (FIG. 4J) scATAC and scRNA profiling-derived genomic coverage plots showed higher chromatin accessibility peaks in the CEBPD coding region of IL-21 NK cells compared with IL-15 NK cells after co-culture with GBM cancer cells for 9 days.

FIGS. 5A-5J—Distinct regulon activity was observed in IL-21 and IL-15 NK cells after GSC co-culture. (FIG. 5A) Venn diagram showing the overlapping regulons between IL-21 NK vs IL-15 NK cells at each time point (baseline, Day 3, Day 9). (FIG. 5B) Heatmap showing regulon activity in IL-21 NK and IL-15 NK cells at baseline (day 0) and days 3 and 9 following co-culture with GSCs. Color bar denotes scaled regulon activity score (AUC) from pySCENIC. (FIGS. 5C-5D) Violin plots showing the scaled gene-level chromatin accessibility of target genes of CEBPB (FIG. 5C) and CEBPD (FIG. 5D) inferred from gene expression profiles using pySCENIC. There were 106 target genes for CEBPD and 85 target genes for CEBPβ. (FIGS. 5E-5F) Top enriched Hallmark pathways of target genes of CEBPB (FIG. 5E) and CEBPD (FIG. 5F) inferred from pySCENIC. (FIG. 5G) UMAP showing the distribution of clusters for each NK cell product based on scRNA-seq data from samples at baseline (day 0), and at days 3 and 9 after challenge and/or rechallenge with GSC20. (FIG. 5H) UMAP plot of the expression level of CEBPD across all cell population, which was highest in cluster 4 (IL-21 cluster). (FIG. 5I) UMAP plot showing the gene set score of CEBPD regulons at the transcriptomic level. (FIG. 5J) Select downstream gene targets of interest regulated by CEBPB and CEBPD identified through pySCENIC.

FIGS. 6A-6K—CEBPD was required for robust and long-lived antitumor activity and metabolic fitness of IL-21 NK cells. NT, Cas9 IL-21 control and CEBPD-KO IL-21 NK cells were co-cultured with m-cherry transduced GSC20 (red) at 1:1 E:T ratio. After 2-3 days, fresh GSC20 were added to the co-cultures (arrows) without disturbing or adding new NK cells. Red (tumor) signal was followed with real time imaging. (FIG. 6A) Graph showing the red object count per image in real time killing analysis (n=4 donors) of NT NK, IL-21 Cas9 NK, and CEBPD-KO IL-21 NK cells. (FIG. 6B) Representative measures of oxygen consumption rate (OCR) upon addition of Oligo, FCCP, and rotenone and antimycin A (R/A). Quantified basal respiration (FIG. 6C) and maximal respiration (FIG. 6D) of purified NT, IL-21 and CEBPD-KO IL-21 NK cells after 48 hours of co-culture with GSC20 (n=3 donors). Error bars denote standard error of mean. (FIG. 6E) Graph showing the red object count per image in a real time killing analysis (n=4 donors) of NT NK and CEBPD-KI (knock in; also known as CEBPD-OE (overexpression)) NT NK cells, and IL-21 NK cells. Arrows indicate addition of GSCs, i.e., rechallenges. (FIG. 6F) Representative measures of oxygen consumption rate (OCR) upon addition of Oligo, FCCP, and R/A. Quantified basal respiration (FIG. 6G) and maximal respiration (FIG. 6H) of purified NT NK, CEBPD-KI NT NK and IL-21 NK cells after 48 hours of co-culture with GSC20 (n=4 donors). Error bars denote standard error of mean. (FIG. 6I) Schematic diagram showing the timeline of the in vivo experiment, and representative BLI images used to monitor the growth of FFluc-labeled GSC272 tumor cells at the indicated time points in NSG mice injected with GSC272 alone, or GSC272 plus NT NK, CEBPD-KI NT NK, IL-21-Cas9 control, or CEBPD-KO IL-21 NK cells. (FIG. 6J) Average radiance (BLI) data (p/sec/cm2/sr) for the denoted treatment conditions. Statistical significance was determined using two-way ANOVA for multiple comparisons between NK treated groups with Bonferroni correction. ***p≤0.001, ns=not significant. (FIG. 6K) Kaplan-Meier plot showing mouse survival in each group (n=5 mice). The data showed that NSG mice receiving CEBPD-KINT NK cells lived significantly longer than mice receiving NT NK cells or tumor cells only, and that CEBPD was required for IL-21 mediated survival. Error bars denote standard error of mean. Statistical significance based on log-rank test. *p≤0.05, **p≤0.01, ***p≤0.001, ns=not significant.

FIGS. 7A-7L—Proliferation and long-term cytotoxicity of cytokine-armed NK cells against GSCs. (FIG. 7A) Fold expansion of IL-15 NK, IL-21 NK, and NT NK cells cultured with uAPC feeder cells at 2:1 ratio (uAPC:NK) and IL-2 (200 IU/mL) over time (n=4 donors). (FIG. 7B) Percentage (%) of killing of GSC8-11 cells by NT NK, IL-15 NK, or IL-21 NK cells as measured by real-time killing assay (n=3). Asterisks represent statistical difference between groups, IL-15 NK vs. NT NK; IL-21 NK vs. NT NK. Error bars denote standard deviation. Statistical significance was determined using 2-way ANOVA with Dunnet correction for multiple comparisons. ***p≤0.001. (FIG. 7C) Graph showing the red object count per image over time, which correlates with the proportion of live cancer cells after each GSC8-11 rechallenge (n=3 donors). Cytokine-armed NK cells were co-cultured with mCherry transduced GSC8-11 (red) at an ET ratio of 1:1. Every 2-3 days, fresh GSCs were added to the co-cultures without adding new NK cells. Red signal (tumor) was followed with real time imaging. Asterisks represent statistical differences between groups. Error bars denote standard deviation. Statistical significance was determined using 2-way ANOVA with Bonferroni correction for multiple comparisons. ***p≤0.001. (FIG. 7D) Absolute counts of NK cells after co-culture with GSC20 at 1:1 ratio over time. Error bars denote standard deviation. (FIG. 7E) Bar graph showing percentage (%) of live NK cells gated on Annexin V- and live/dead-population. Error bars denote standard deviation. Statistical significance was determined using 2-way ANOVA with Bonferroni correction for multiple comparisons. (FIG. 7F) NT NK cells after IL-21 priming and GSCs were co-cultured at an ET ratio of 1:1. Every 2-3 days, fresh GSCs were added to the co-cultures without adding new NK cells. Red signal (tumor) was followed with real-time imaging. Asterisks represent statistical differences between groups. Error bars denote standard deviation. Statistical significance was determined using 2-way ANOVA with Bonferroni correction for multiple comparisons. ***p≤0.001. (FIGS. 7G-7K) Graphs showing IL-21 (FIG. 7G), TNF-α (FIG. 7H), IFN-γ (FIG. 7I), Granzyme B (FIG. 7J), and Perforin (FIG. 7K) concentrations in supernatant samples of NK cells co-cultured with GSC20 at 1:1 ratio. Every 2-3 days, fresh GSCs were added to the co-cultures without adding new NK cells. Cell supernatant samples were collected at different time points and cytokine levels were measured by multiplex ELISA. Asterisks represent statistical differences between groups. Error bars denote standard deviation. Statistical significance was determined using 2-way ANOVA with Bonferroni correction for multiple comparisons. **p≤0.01, ***p≤0.001. IL-15 NK vs NT NK; IL-21 NK vs NT NK. (FIG. 7L) Polyfunctionality heatmap of NT vs IL-15 vs IL-21 NK cells in response to GSC20.

FIGS. 8A-8B—IL-21-armed NK cells exhibited long-term cytotoxicity against GSCs. Cytokine-armed NK cells were co-cultured with m-cherry transduced GSC20 (red) at an E:T ratio of 1:1. Every 2-3 days, fresh GSCs were added to the co-cultures without adding new NK cells. GSC20 killing percentage (%) was calculated by the ratio of red and green (death cell) overlapping signals (counts per image) divided by the red signal (GSC). Signal was followed with real time imaging. (FIG. 8A) Percentage (%) of GSC20 killing by NT NK, IL-15 NK, or IL-21 NK cells after each tumor rechallenge as measured by real-time killing assay (n=3 donors). (FIG. 8B) Bar graph showing percentage (%) of GSC20 killing by the indicated NK cells after each tumor rechallenge from two separate experiments (n=6 donors). Asterisks represent statistical difference between groups, IL-21 NK vs. NT NK; IL-21 NK vs. IL-15 NK. Error bars denote standard deviation. Statistical significance was determined using 2-way ANOVA with Bonferroni correction for multiple comparisons. ***p≤0.001.

FIGS. 9A-9I—IL-15 NK cells showed increased proliferation and toxicity in an orthotopic mouse model of GSC. NSG mice were implanted orthotopically with GSC20 and treated intratumorally (IT) with 0.5×10⁶ IL-15 NK or IL-21 NK cells. (FIG. 9A) Bioluminescence imaging (BLI) was used to monitor the growth of FFluc-labeled GSC20 tumor cells over time in NSG mice treated with IL-15 NK or IL-21 NK cells. (FIG. 9B) Average radiance (BLI) data (p/sec/cm2/sr). Statistical significance was determined using unpaired t-test at day 14 time point. *p≤0.05. (FIG. 9C) Graph showing the percentage change in body weight as a measure of toxicity for the different groups described. Statistical significance was determined using unpaired t-test at day 22. *p≤0.05. (FIG. 9D) Kaplan-Meier plot showing the survival for IL-15 NK (n=3) vs IL-21 NK (n=4) treated mice. Mice treated with IL-15 NK cells died significantly faster than those receiving IL-21 NK cells (p=0.01) based on log-rank test; **p≤0.01. Statistical significance was determined using unpaired t-test at day 22 time point. (FIG. 9E) Immunohistochemical staining for human granzyme B (GrB) (left), Iba1 (middle), and glial fibrillary acidic protein (GFAP) in brain from mice that died after IL-15 NK treatment showed profuse NK cell proliferation, microgliosis and astrocytosis. Images were taken at 20× magnification. Tonsil used as positive control for GrB and mouse brain for Iba1 and GFAP. (FIG. 9F) Representative images of immunohistochemical staining for human granzyme B (GrB) in brain sections collected from NSG mice implanted orthotopically with GSC20 cells and treated with 0.1×10⁶ IL-15 NK cells (n=4 mice), IL-21 NK cells (n=4 mice), or NT NK cells (n=3 mice). NSG mice implanted with GSC20 cells (n=3 mice) were used as negative control. Images acquired at 10× magnification. Insets in IL-15 NK panels show NK cells (arrows) that had infiltrated the brain of IL-15 NK cell treated mice (at 20× magnification; brown signals). Staining from two mice per treatment condition is shown. (FIG. 9G) Granzyme B (GrB)-positive cells per mm² in the brain sections of NT NK, IL-15 NK, and IL-21 NK cell treated mice. Error bars denote standard deviations. Statistical significance was determined using 2-way ANOVA with Bonferroni correction for multiple comparisons. ***p≤0.001; ns=not significant. (FIG. 9H) Representative images of immunohistochemical staining for mouse GFAP in brain sections collected from NSG mice implanted orthotopically with GSC20 cells and treated with IL-15 NK cells (n=4 mice), IL-21 NK cells (n=4 mice) or NT NK cells (n=3 mice). Images were acquired at 20× magnification. (FIG. 9I) Brain histopathology measuring infiltration with human granzyme B⁺ cells (as a measure of NK cells) and the gliosis score in human IL-15 NK and IL-21 NK cell treated mice. The frontal lobe was examined for the presence or absence of granzyme B+ cells and for increased glial cells (gliosis). Gliosis was scored semi-quantitatively using the following scale: 0=no apparent increase in glial cells, 1=minimal gliosis (cellularity increase 1-10%), 2=mild gliosis (cellularity increase 11-30%), 3=moderate gliosis (cellularity increase 31-50%) and 4=marked gliosis (cellularity increase greater than 50%).

FIGS. 10A-10F—The route of administration influenced the efficacy and degree of toxicity caused by IL-15 NK cells. (FIG. 10A) Mice were implanted orthotopically with GSC262 and treated with IL-15 NK cells administered through the tail vein (IV), or intratumorally (IT). Mice treated with IT NT NK cells were used as controls. Kaplan-Meier plot showing the survival for each group of mice (n=5 mice per group). Mice treated with IT IL-15 NK cells died significantly faster than those receiving IL-15 NK cells IV (p=0.0027) based on log-rank test; **p≤0.01. (FIG. 10B) Graph showing the percentage change in body weight as a measure of toxicity for the different groups described. Statistical significance was determined using 2-way ANOVA with Bonferroni correction for multiple comparisons. ns=not significant. (FIG. 10C) Flow cytometry panels showing massive infiltration of NK cells in the brain tissue from IT (top panels) vs IV (bottom panels) IL-15 NK cell-treated mice. (FIG. 10D) Percentage of NK cells (CD56+CD3−) gated on hCD45+ cells in the brain tissue of mice treated with IL-15 NK cells administered IT (n=4 mice) vs. IV (n=4 mice). Error bars denote standard deviations. Statistical significance was determined using t-test. ***p≤0.001. (FIG. 10E) Mice were implanted orthotopically with GSC20 and treated with IL-21 NK cells administered through tail vein (IV) or untreated (control). Kaplan-Meier plot showing the survival for each group of mice (n=5 mice per group). No significant difference was observed between the groups based on log-rank test. (FIG. 10F) Average radiance (BLI) data (p/sec/cm2/sr). Error bars denote standard error of mean. Statistical significance was determined using paired t-test at each time point. ns=not significant.

FIGS. 11A-11H—Superior in vivo antitumor activity was displayed by IL-21 NK cells. (FIG. 11A) Schematic diagram showing the timeline of the in vivo experiment. Bioluminescence imaging (BLI) was used to monitor the growth of FFluc-labeled GSC8-11 tumor cells over time in NSG mice treated with GSC8-11 alone, or GSC8-11 plus NT NK or IL-21 NK cells. (FIG. 11B) Average radiance (BLI) data (p/sec/cm2/sr). Error bars denote standard error of mean (s.e.m.). Statistical significance was determined using paired t-test at each time point. *p≤0.05. (FIG. 11C) Kaplan-Meier plot showing survival for each group (n=5 mice per group). Animals treated with IL-21 NK cells had significantly better survival compared with tumor controls (p=0.0163). In contrast, there was no significant difference in the survival of mice treated with NT-NK cells vs tumor alone (P=0.135) based on log-rank test; *p≤0.05, ns=not significant. (FIG. 11D) Graph showing the body weight change (%) of mice over time in the different groups related to GSC8-11. (FIG. 11E) Schematic diagram showing the timeline of the in vivo experiment. BLI was used to monitor the growth of FFluc-labeled GSC267 tumor cells at different time points in NSG mice treated with GSC267 alone, or GSC267 plus NT-NK or IL-21 NK cells. (FIG. 11F) average radiance (BLI) data (p/sec/cm2/sr). Statistical significance was determined using paired t-test at each time point. *p≤0.05. (FIG. 11G) Kaplan-Meier plot showing survival for each group (n=3-4 mice per group). Animals treated with IL-21 NK cells had significantly better survival compared with tumor controls (p=0.0091). In contrast, there was no significant difference in the survival of mice treated with NT-NK vs tumor controls (P=0.762); based on the log-rank test. (FIG. 11H) Graph showing the percentage (%) change in body weight in the different groups related to GSC267.

FIGS. 12A-12D—NK cells emerged after GSC rechallenge in IL-21 NK treated mice. (FIG. 12A) Representative images of immunohistochemical staining for Ki-67 in brain sections collected from NSG mice treated four hundred days prior with IL-21 NK cells (n=4) and rechallenged with GSC20 (0.25×10⁶ GSC20 cells) and naïve NSG mice (n=5_not previously treated with IL-21 NK cells that also received GSC20 (GSC20 alone control), sacrificed 17 days later. Mouse lymph nodes were used as a positive control. Staining from four mice in each group is shown. Images were taken at 10×. (FIG. 12B) Quantification of total Ki-67+ and Ki-67 tumor cells in brain sections collected from NSG mice treated with IL-21 NK cells rechallenged with GSC20 (n=4 mice) vs GSC only controls (n=5 mice), indicating less proliferative and non-proliferative tumor cells in IL-21 NK cell treated mice. Error bars denote standard deviations. Statistical significance was determined using unpaired t-test. *p≤0.05. (FIG. 12C) Gating strategy to identify NK cells by flow cytometry in brain tissue collected from mice after rechallenge with GSC20. Four hundred days after treatment with IL-21 NK cells, mice were rechallenged with additional GSC20 (0.25×10⁶ GSC20) and sacrificed 17 days later (a representative FACS plot from n=4 mice is shown in the top panel). A group of naïve NSG mice (n=5 mice) not previously treated with IL-21 NK cells also received GSC20 and were used as a positive tumor control (bottom panel). (FIG. 12D) Quantification of human CD16-positive cells by IHC per mm², in the brain sections of IL-21 NK cell-treated mice rechallenged with GSC20 (n=4 mice) vs GSC only controls (n=5 mice). Error bars denote standard deviations. Statistical significance was determined using unpaired t-test. **p≤0.01.

FIGS. 13A-13B—IL-21 NK cells may have resided in the brain. (FIG. 13A) Representative images from 4 mice of immunohistochemical granzyme B (GrB) staining of liver, spleen and lung sections from NSG mice treated four hundred days prior with IL-21 NK cells and rechallenged with GSC20. Human tonsil was used as a positive control. Images were taken at 10× and 20× magnifications. (FIG. 13B) Representative flow cytometry panel showing the absence of human CD45+ cells in lung, spleen and bone marrow in mice treated with IL-21 NK cells. Cord blood NK cells were used as positive control.

FIG. 14—Single cell RNA sequencing profiling of IL-15 NK and IL-21 NK cells after GSC20 rechallenge. (FIG. 14) Heatmap showing gene expression at the cluster level on day 9 after rechallenge. Genes in red font represented particularly intriguing genes (e.g., CEBPD, CLIC3, EOMES, BIRC3, NFκBIA) that were upregulated in cluster 4.

FIGS. 15A-15B—CEBPD expression at the RNA level and regulon score. (FIG. 15A) Violin plots showing the CEBPD scRNA-seq expression level and (FIG. 15B) CEBPD regulon score by each cluster.

FIGS. 16A-16F—IL-21 NK cells expressed CEBPD in vivo and in vitro. CEBPD expression in NK cells by qPCR (FIG. 16A) and by flow cytometry in Mean Fluorescence Intensity (MFI) and percentage CD56+ and CEBPD+ cells (FIGS. 16B-16D) after 21 days of in vitro rechallenge with GSC20. Error bars denote standard deviation. Statistical significance was determined using 2-way ANOVA with Bonferroni correction for multiple comparisons. *p≤0.05, ***p≤0.001; ns=not significant. (FIG. 16E) Representative flow panel showing NK cells in brain tissue collected from NSG mice implanted with GSC272 and treated with IL-15 NK, IL-21 NK, or NT NK cells. The flow panels show CEBPD expression in IL-21 NK vs NT NK cells. Gating based in CEBPD KO NK cells was used as a control. (FIG. 16F) Histogram showing the percentage of CD56 CEBPD+ cells gated on CD56+CD3-cells by flow cytometry in brain tissues pooled from three different GSC models: GSC20, GSC8-11, and GSC272, (n=3-8 mice per group). Error bars denote standard deviation. Statistical significance was determined using ordinary one-way ANOVA with Bonferroni correction for multiple comparisons. ns=not significant; *p≤0.05, ***p≤0.001.

FIGS. 17A-17C—Enrichment analysis of the IL-21 vs IL-15 WT response. (FIG. 17A) Top enriched canonical pathways differentially activated with IL-21 when compared with IL-15 NK cells. (FIG. 17B) Activity prediction and expression of antitumoral-related functions and molecules involved in NK Cell Signaling Pathway. (FIG. 17C) Network analysis of regulators differentially activated or inhibited with IL-15 and IL-21 responses. Significant enrichments shown have a Z score <−2 or >2 with BH-corrected p-values (Q-values)<0.05. Expression values in log 2 fold-changes (Log 2FC) from corresponding DEGs are overlapped from bulk RNA seq, and network regulators were cross-validated from bulk and single cell ATAC-seq.

FIGS. 18A-18D—Genetic deletion of CEBPD by CRISPR/Cas9 in IL-21 NK cells. (FIG. 18A) The CRISPR targeted CEBPD locus was PCR amplified from genomic DNA from NT NK, IL-21 Cas9 control, and IL-21 CEBPD knock-out (KO) NK cells using forward and reverse primers (SEQ ID NOs: 55 and 56) and the PCR products were size-separated by electrophoresis on an agarose gel. PCR products from two CB donors are shown. (FIG. 18B) CEBPD expression by flow cytometry in NK cells after 30 days of rechallenge with GSC20; MFI=mean fluorescence intensity (FIG. 18C) Fold proliferation of NT NK, IL-21 Cas9 NK, and IL-21 CEBPD-KO NK cells over time with culture with IL-2 and UAPCs. (FIG. 18D) Cell viability of NT NK, IL-21 Cas9 NK, and IL-21 CEBPD KO NK cells in culture over time. Error bars denote standard deviation. Statistical significance was determined using 2-way ANOVA with Bonferroni correction for multiple comparisons. ns=not significant; ***p≤0.001.

FIGS. 19A-19C—Deletion of CEBPD impaired the long-term cytotoxicity of IL-21 NK cells against GSCs. NT NK, IL-21 Cas9 NK, and IL-21 CEBPD-KO NK cells were co-cultured with m-cherry transduced GSC20 (red) at 1:1 E:T ratio, GSC20 cells were cultured alone as a control. After 2-3 days, fresh GSC20 were added to the co-culture without disturbing or adding new NK cells. Red (tumor) signal was followed with real time imaging. (FIG. 19A) Representative image on day 9 of NK cell rechallenge with GSCs, showing that CEBPD-KO impairs the cytotoxicity of IL-21 NK cells against GSC20. Scale bar indicate 400 μm. (FIG. 19B) Bar graphs from multiplex ELISA showing cytokine and chemokine production by NT NK (left columns), IL-21 Cas9 NK (middle columns), and IL-21 CEBPD-KO NK cells (right columns) after 5 rechallenges with GSC20 (n=4 donors). Error bars denote standard deviation. Statistical significance was determined using 2-way ANOVA with Bonferroni correction for multiple comparisons. ns=not significant; *p≤0.05, **p≤0.01, ***p≤0.001. (FIG. 19C) Heatmap showing Normalized Enrichment Scores (NES) of selected Hallmark pathways (y-axis) for transcriptional changes across three comparisons of interest (x-axis). Color represents the direction of the enrichment (top of scale (red) represents upregulation of the pathway in CEBPD KO or overexpression (OE) NK cells while bottom of scale (blue) represents upregulation of the pathway in CEBPD (wild-type) NK cells). Asterisks denote significantly enriched pathways, defined as FDR adjusted p-value<0.05.

FIGS. 20A-20B—CEBPD knock-in (KI) efficiency. (FIG. 20A) Flow panel showing CEBPD expression in NK cells after transduction with a retroviral vector encoding full-length CEBPD (SEQ ID NO: 19) for its overexpression (“OE”, or knock-in “KI”). NT NK cells (WT) were used as negative control. (FIG. 20B) Histogram showing the CEBPD-KINK cell transduction efficiency at day 5 after transduction (n=5 donors). Error bars denote standard deviation. Statistical significance was determined using unpaired t test. ***p≤0.001.

FIGS. 21A-21C—CEBPD-KI reduced mitochondrial reactive oxygen species (ROS) production. (FIG. 21A) Representative flow cytometry plot and histogram (FIG. 21B) showing percentage of MitoSOX™ production in NT NK, CEBPD-KINT NK, IL-21 NK, and IL-21 CEBPD-KO NK cells (CD56⁺Mitosox⁺). Error bars denote standard deviation. Statistical significance was determined using one-way ANOVA for multiple comparisons with Bonferroni correction test. ns=not significant; *p≤0.05, **p≤0.01. (FIG. 21C) The CRISPR targeted STAT3 locus was PCR amplified from genomic DNA from NK cells obtained from two CB donors using forward (SEQ ID NO: 62) and reverse (SEQ ID NO: 63) primers, and the PCR products were size-separated by agarose gel electrophoresis.

FIGS. 22A-22B—C/EBP family and AP-1 complex gene expression in tumor infiltrating (TI)-NK cells from patients with GBM. (FIG. 22A) UMAP of NK cells from healthy control (HC-NK) PBMC samples and GBM tumor-infiltrating NK (TI-NK) (n=10 patients). The HC-NK PBMC samples were downloaded from 10× genomic database with around 3,000 cells. (FIG. 22B) Violin plots showing C/EBP family (Top, CEBPA, CEBPβ, CEBPD, CEBPE, and CEBPG) and AP-1 complex (Bottom, JUND, JUNB, FOS, FOSL1, and FOSL2) gene expression comparison between healthy control PBMC (HC-NK) and high-grade glioma (TI-NK) surgical samples from 10 patients. The P value is indicated on the top of each plot. The significance was calculated using unpaired T-test.

FIGS. 23A-23S—Cytokine arming improved NK cell antitumor activity against GSCs. (FIGS. 23A-23S) 3D killing assay of GSC272 spheroids by NT NK, IL-15 NK, or IL-21 NK cells over 7 days. Images were acquired every 2 hours, and representative images from every 4 hours for the first 24 hours, and every 12 hours thereafter are shown. Red signal revealed the growth of the GSC272 spheroids and green signal indicated apoptosis (Caspase 3/7). Scale bar indicates 600 μm.

FIGS. 24A-24H—STAT3 functioned as a mediator of CEBPD expression in IL-21 NK cells, and validation of CEBPD as a target gene. (FIG. 24A) Relative expression levels of CEBPD by qPCR in IL-21 Cas9 NK control cells compared with IL-21 STAT3 KO NK cells (n=3 donors). Error bars denote standard deviation. Statistical significance was determined using unpaired t-test. *p≤0.05. (FIG. 24B) Chromatin immunoprecipitation (ChIP) of pSTAT3 followed by qPCR analysis of CEBPD normalized to IgG (n=3 donors). CEBPD-BS (CEBPD-biding site): Primers for the STAT3 binding site at the promotor region (SEQ ID NOs: 57-58); CEBPD-NC (CEBPD-negative control): Primers designed in a region with no potential STAT3 binding site. Error bars denote standard deviation. Statistical significance was determined using two-way Anova for multiple comparisons with Bonferroni correction test. ***p≤0.001. (FIG. 24C) Barcode Enrichment Plot for CEBPD downstream genes; All genes from the dataset were ranked along x-axis according to enrichment score (y-axis), and the ranked position of each gene within a signature is shown in the x-axis. Statistical significance was determined by a rank-based test, in which the enrichment score (ES) reflects the degree to which genes in a set were over-represented at the extremes of a ranked list, utilizing a Kolmogorov-Smirnov-like statistic. Then the statistical significance of the ES was determined through a permutation test based on phenotypic labels, generating a null distribution for comparison to ascertain the gene set's dependency on these labels (n=4 donors). (FIG. 24D) Heatmap showing the average log 2 fold change in two comparisons (left: NT CEBPD OE vs NT; right: IL-21 CEBPD KO vs IL-21). Listed are genes in the CEBPD downstream signaling pathway which were hierarchically clustered (n=4 donors). (FIG. 24E) CEBPD occupancy of CEBPD-specific regulon gene targets (KLF2, BNIP3L, IRF1 and ETS1) in IL-21 NK cells vs NT NK cells (n=2 donors). Profile plots of CEBPD ChIP-seq normalized tags around promoter regions (−300, +100 bp) of transcription start sites (TSS). (FIG. 24F) Boxplot of CEBPD ChIP-seq normalized tag densities across gene target sites. Statistical significance was determined by unpaired, two-tailed t-test between NT and IL-21 NK cells. (FIG. 24G) Relative expression levels of KLF2 (left) and BNIP3L (right) by qPCR in IL-21 Cas9 NK control cells compared with IL-21 CEBPD KO NK cells (n=4 donors). Error bars denote standard deviation. Statistical significance was determined using unpaired t-test. *p≤0.05, ***p≤0.001. (FIG. 24H) Schematic diagram depicting how IL-21 induced CEBPD which in turn regulated downstream target genes (e.g., KLF2 and BNIP3L) in NK cells.

FIGS. 25A-25J. IT injection of IL-15 NK cells with low transduction (low IL-15 production) also increased NK infiltration and gliosis in brain. NSG mice were implanted orthotopically with GSC20 and treated with NT, low transduced IL-15 NK cells, or IL-21 NK cells. After 2 weeks, brains were collected and analyzed by flow cytometry and IHC staining. (FIG. 25A) NK cells were expanded with uAPC feeder cells and IL-2 for 5 days and then transduced with a low dose of retroviral vector encoding IL-15. (FIG. 25B) Flow cytometry plots showing massive NK infiltration in brain of NK cells with low IL-15 transduction/expression compared with NT and IL-21 NK cell treated mice. (FIG. 25C) Absolute count of hCD45⁺CD56⁺CD3⁻ (NK cells) in brain tissue (n=4-5 mice per group). Error bars denote standard deviation. Statistical significance was determined using one-way ANOVA with Bonferroni correction for multiple comparisons. *p≤0.05; ns=not significant. (FIG. 25D) Representative images of immunohistochemical staining for human granzyme B, glial fibrillary acidic protein (GFAP) and Iba1 in brain sections collected from NSG mice implanted orthotopically with GSC20 cells and treated with low transduced IL-15 NK cells (n=3 mice), IL-21 NK cells (n=3 mice) or NT NK cells (n=3 mice). NSG mice implanted with GSC20 cells alone (n=3 mice) were used as control. (FIG. 25E) Granzyme B-positive cells per mm² in brain sections of NT NK, IL-15 NK, and IL-21 NK cell treated mice (n=3 mice per group). Error bars denote standard deviation. Statistical significance was determined using one-way ANOVA with Bonferroni correction for multiple comparisons. ***p≤0.001. (FIG. 25F) GFAP-positive cells per mm² in brain sections collected from NSG mice implanted orthotopically with GSC20 cells and treated with IL-15 NK, IL-21 NK or NT NK cells (n=3 mice per group). Statistical significance was determined using one-way ANOVA with Bonferroni correction for multiple comparisons. *p≤0.05. (FIG. 25G) Iba1-positive cells per mm² in brain sections collected from NSG mice implanted orthotopically with GSC20 cells and treated with IL-15 NK, IL-21 NK or NT NK cells (n=3 mice per group). (FIG. 25H and FIG. 25I) Representative image showing multiplex immunofluorescence of Iba1 (light blue), Luciferase (green), Granzyme B (yellow), Caspase-3 (red), Ki-67 (orange), GFAP (purple) and DAPI (dark blue) signal in mice brain tissue treated with low transduced IL-15 NK cells and IL-21 NK cells 2 weeks after GSC injection. Images were acquired at 4× and 20× magnification. White arrow in FIG. 25H indicates microglia (Iba1) and NK cells (Granzyme B) in close contact. (FIG. 25J) Representative immunofluorescence images showing green tumor cell signal (GSC20 luciferase) in mice brain from NSG mice treated with low transduced IL-15 NK cells and IL-21 NK cells. Mice injected with GSC20 alone were used as a control. Images were acquired at 2× magnification.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present inventions. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, the term “CD3 receptor complex” or “CD3 co-receptor complex” refers to the protein complex that in nature acts as a T cell co-receptor and is comprised of CD3ζ chain, CD3γ chain, a CD3δ chain, and two CD3ε chains (although in alternatives only one CD3ε chain is used).

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector. Cells may be engineered to express heterologous proteins that are not naturally expressed by the cells, or overexpress a heterologous proteins that can be naturally expressed by the cell, either because the heterologous proteins and/or transcripts encoding the same are recombinant or synthetic or because the cells do not naturally express the proteins.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “subject,” as used herein, generally refers to an individual having a that has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of one or more symptoms of the disease or condition, or the delaying of the progression of the disease or condition. “Treatment” does not necessarily indicate complete eradication or cure of the disease or condition, or associated symptoms thereof. Treating may mean alleviation of at least one symptom of the disease or condition.

### I. EMBODIMENTS OF THE DISCLOSURE

Natural killer (NK) cells are innate immune cells with strong antitumor activity. While NK cells have been described as exhibiting adaptive-like memory responses to previously encountered viruses, the factors that promote memory against cancer have remained unaccounted for and elusive. Provided herein are data and analyses that show how NK cells engineered to express IL-21 (IL-21 NK) acquired memory-like features, with the ability to respond to glioblastoma (GBM) rechallenge in vitro and in vivo. Furthermore, using single-cell multiomics platforms, the inventors show herein that IL-21 NK cells have a unique signature of chromatin accessibility, with CCAAT/Enhancer-Binding Protein (C/EBP, or CEBP), especially CEBPD, serving as key transcription factors regulating NK cell memory response. Deletion of CEBPD resulted in loss of IL-21 NK cell potency, while overexpression of CEBPD in NK cells increased their long-term cytotoxicity, metabolic fitness and anti-GBM potency in vivo. The results presented herein show that IL-21 epigenetically reprograms NK cells to acquire an antitumor immune memory. Furthermore, the results provided herein show that this antitumor immune memory is highly dependent on C/EBP family members, particularly CEBPD, and that overexpression of CEBPD is sufficient to recapitulate IL-21 mediated phenotypes such as long-term cytotoxicity, metabolic fitness, and anti-cancer (e.g., anti-GBM) potency in vivo. Thus, provided herein in some embodiments are compositions and methods comprising engineered NK cells modified to overexpress one or more CEBP family protein members.

Until recently, NK cells were believed to lack memory features that are characteristic of adaptive immune cells such as T and B cells. Recently, this traditional dogma has been challenged, with a number of studies reporting NK memory-like responses against haptens and viruses, and following exposure to inflammatory cytokines such as interleukin (IL)-12, IL-15 and IL-18. Although subsets of NK cells with memory-like features following murine and human cytomegalovirus (CMV) infection have been described, evidence for the presence of such subsets after tumor challenge is absent, and the molecular mechanisms that regulate a memory response to cancer remain undescribed and unclear.

The potency and in vivo persistence of NK cells against cancer can be enhanced by arming NK cells with cytokines (cytokine-armed), with most of the translational and clinical work having focused on IL-15. IL-15 is released by numerous cell types, including macrophages and dendritic cells, and promotes NK cell cytotoxicity and proliferation. A number of preclinical studies and early clinical trials support the overall safety and promising activity of IL-15-armed NK cells.

IL-21, another attractive cytokine for cancer immunotherapy, induces metabolic reprogramming and mitochondrial biogenesis in T cells, and is produced by a number of immune cells such as activated CD4⁺ T cells and natural killer T (NKT) cells. IL-21 also promotes NK cell maturation and metabolic fitness and modulates NK cell function. Recombinant IL-21 was tested in several clinical trials of metastatic cancer with an acceptable safety profile, but its short half-life and need for repeated dosing have severely limited its clinical application. Thus, these data reveal critical roles for IL-15 and IL-21 in modulating diverse NK cell functions, and support cytokine engineering as a strategy to augment NK cell antitumor efficacy.

As shown herein, multiple orthotopic models of patient-derived glioblastoma stem cells (GSCs) were utilized to compare the antitumor activity of engineered IL-21-armed NK cells (IL-21 NK) and engineered IL-15-armed NK cells (IL-15 NK). The data presented herein showed engineered IL-21 NK cells exerted superior antitumor activity with memory-like features, associated with distinct transcriptional and epigenetic signatures when compared to IL-15 NK cells and/or non-armed NK cells. Furthermore, the data presented herein was utilized to identify the C/EBP family of transcription factors (TF) and the activator protein 1 (AP-1) complex as important regulators of NK cell memory. Surprisingly, deletion of CEBPD, which the inventors discovered is the most differentially expressed TF in IL-21 NK cells, impaired IL-21 NK cell potency and recall response, while CEBPD overexpression in wild-type NK cells increased NK cell functional capacity and metabolic fitness. Finally, as shown herein, the inventors confirmed expression of CEBPD in tumor-infiltrating NK cells from patients with GBM. These data have identified the CEBP protein family, particularly CEBPD, as important transcriptional and epigenetic coordinators of NK cell memory to cancer. Furthermore, the data support arming NK cells with IL-21 (cytokine-armed) and/or with overexpression of a CEBP family protein as novel immunotherapeutic approaches for treatment of cancers, such as GBM.

The present disclosure specifically relates to NK cells that have been modified to render the NK cells to have enhanced function as an immunotherapy compared to NK cells not so modified. The modifications allow for the NK cells to have greater versatility and functionality when used alone and/or with other therapeutic agents.

### II. COMPOSITIONS OF THE DISCLOSURE

The disclosure concerns at least compositions and methods that comprise engineered NK cells that have been modified to overexpress and/or upregulate the activity of one or more members of the CCAAT/Enhancer-binding protein (CEBP) family. In certain embodiments, the engineered NK cells may be transgenically modified to overexpress a nucleic acid encoding a CEBP protein family member. In certain embodiments, an engineered NK cell transgenically expresses and/or is subjected to a CEBP protein transcriptional and/or translational activator. In certain embodiments, an engineered NK cell transgenically expresses and/or is subjected to an inhibitor of a CEBP protein transcriptional and/or translational inhibitor.

### A. NK Cell Modifications

In particular embodiments, provided herein are compositions that comprise engineered NK cells that have been modified by the hand of man to overexpress part or all of a CEBP protein.

In certain embodiments, an engineered Natural Killer (NK) cell is modified to overexpress a CEBP protein relative to a non-engineered NK cell and/or an NK cell engineered to express IL-15. In certain embodiments, the CEBP protein is CEBPA (CEBP-alpha, CEBPα), CEBPB (CEBP-beta, CEBPβ), CEBPG (CEBP-gamma, CEBPγ), CEBPD (CEBP-delta, CEBPδ), CEBPE (CEBP-epsilon, CEBPε) and/or CEBP Homologous Protein (CHOP). In certain embodiments, the CEBP protein is CEBPD. In certain embodiments, the CEBP protein is CEBPB. In certain embodiments, the CEBP protein is CEBPD and CEBPβ. In certain embodiments, an engineered natural killer cell is modified to express a heterologous CEBP transcription factor family protein (e.g., overexpress a CEBP transcription factor family protein that is not normally expressed, or that is expressed naturally at a relatively low level).

In cases wherein part of a CEBP protein is utilized, the part may be at least 50, 75, 100, 125, 150, 175, 200, 225, 250, 275, 300, 325, 350, 375, or 400 amino acids, including contiguous and/or non-contiguous amino acids. In certain embodiments, a CEBP protein may comprise at least 50, 55, 60, 65, 70, 75, 80, 85, 90, 91, 92, 93, 94, 95, 96, 97, 98, 99, or 100% of the amino acids of wild type CEBP protein.

In certain embodiments, a CEBP protein and/or transcript encoding a CEBP protein is overexpressed by at least 1.0, 1.25, 1.5, 1.75, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, or greater than 10 fold, or any range derivable therein, relative to a control cell. In certain embodiments, a CEBP protein and/or transcript encoding a CEBP protein is overexpressed by at least 1.0, 1.25, 1.5, 1.75, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, or greater than 10 fold, or any range derivable therein, relative to a non engineered cell. In certain embodiments, a CEBP protein and/or transcript encoding a CEBP protein is overexpressed by at least 1.0, 1.25, 1.5, 1.75, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5, 8.75, 9.0, 9.25, 9.5, 9.75, 10.0, or greater than 10 fold, or any range derivable therein, relative to an IL-15 expressing cell.

In specific cases, any sequences encompassed herein are utilized to modify the NK cells, although in other cases sequences that are related to these in identity are utilized. For example, related sequences that are at least 80, 85, 90, 95, 96, 97, 98, 99% identical to any sequence encompassed herein may be utilized in the disclosure.

In specific cases, one or more vectors utilized for transduction of an NK cell may or may not be multicistronic, and may or may not be able to provide a template for a transcript that encodes more than one separate polypeptide. In cases wherein one or more multicistronic vectors are employed, they may utilize one or more internal ribosome entry sites (IRES) and/or one or more 2A self-cleaving peptide sites. In cases wherein one or more 2A sequences are utilized, the following may be used, where GSG is an optional linker:

T2A
(SEQ ID NO: 1)
(GSG) EGRGSLLTCGDVEENPGP
P2A
(SEQ ID NO: 2)
(GSG) ATNFSLLKQAGDVEENPGP
E2A
(SEQ ID NO: 3)
(GSG) QCTNYALLKLAGDVESNPGP
F2A
(SEQ ID NO: 4)
(GSG) VKQTLNFDLLKLAGDVESNPGP

In situations wherein multiple protein components are expressed from a multicistronic vector, the order in a 5′ to 3′ direction on the polynucleotide vector may be of any order, although in alternative cases they are present on the vector in a particular order. A multicistronic vector may express multiple heterologous proteins. In certain embodiments, a multicistronic vector includes one or multiple marker proteins (e.g., a fluorescent tag, etc.) and one or multiple functional proteins (e.g., a CEBP protein, a cytokine, an engineered antigen receptor, etc.).

In certain embodiments, an engineered Natural Killer (NK) cell described herein is modified such that it develops and/or develops the capacity to have memory (e.g., immune memory) and/or memory-like phenotypes relative to a target (e.g., an antigen, a cell, a cancer cell, a virus, an amoeba, a parasite, etc.). In certain embodiments, an engineered NK cell obtains a memory and/or memory-like phenotype directed to a target, such that the NK cell can recognize and activate against the target at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, or greater than 400 days after exposure to a target. In certain embodiments, an engineered NK cell obtains a memory and/or memory-like phenotype directed to a target, such that the NK cell can recognize and activate against the target for at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, or greater than 400 days after clearance of the target.

In certain embodiments, an engineered NK cell obtains a memory and/or memory-like phenotype directed to a target, such that the NK cell can provide immunity against the target for at least or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, or greater than 400 days after exposure to a target.

### B. NK Cells

In certain embodiments, NK cells that are engineered to overexpress and/or upregulate the activity of one or more members of the CEBP family may be obtained from any suitable source, including fresh or frozen. In certain embodiments, NK cells are not NK cells obtained from iPSC differentiation. In certain embodiments, NK cells are derived from human peripheral blood mononuclear cells (PBMC), unstimulated leukapheresis products (PBSC), NK cell lines (e.g., NK-92), human embryonic stem cells (hESCs), induced pluripotent stem cells (iPSCs), bone marrow, or umbilical cord blood by methods well known in the art. Specifically, the NK cells may be isolated from cord blood (CB), peripheral blood (PB), bone marrow, stem cells, NK cell lines, or a mixture thereof. In particular embodiments, the NK cells are isolated from pooled CB. The CB may be pooled from 2, 3, 4, 5, 6, 7, 8, 9, 10, or more units. The NK cells may be autologous or allogeneic with respect to a recipient individual. The isolated NK cells may or may not be haplotype matched for the subject to be administered the cell therapy. NK cells can be detected by specific surface markers, such as CD16 and CD56 in humans, for example. In some cases, the source of the NK cells is cord blood and the NK cells may be in the cord blood in a heterogeneous mixture of cells and may be depleted of certain cells expressing CD3. In other methods, umbilical CB is used to derive NK cells by the isolation of CD34+ cells.

The NK cells may be pre-activated with one or more inflammatory cytokines, and they may be expanded or non-expanded. In some cases, the NK cells are pre-activated prior to engineering to overexpress and/or upregulate the activity of one or more members of the CEBP family, and/or following such engineering. In specific embodiments, pre-activation of the NK cells may comprise culturing the isolated NK cells in the presence of one or more cytokines. The NK cells may be stimulated with IL-2, or other cytokines that bind the common gamma-chain (e.g., IL-7, IL-12, IL-15, IL-18, IL-21, and others). In particular embodiments, the pre-activation cytokines may be selected from the group consisting of IL-12, IL-15, IL-18, and a combination thereof. One or more additional cytokines may be used for the pre-activation step. The pre-activation may be for a short period of time such as 5-72 hours, such as 10-50 hours, particularly 10-20 hours, such as 12, 13, 14, 15, 16, 17, 18, 19, or 20 hours, specifically about 16 hours. The pre-activation culture may comprise IL-12 at a concentration of 0.1-150 ng/ml, such as 0.5-50 ng/mL, particularly 1-20 ng/ml, such as 2, 3, 4, 5, 6, 7, 8, 9, 10, 11, 12, 13, 14, or 15 ng/mL, specifically about 10 ng/ml. The pre-activation culture may comprise IL-18 and/or IL-15 at a concentration of 10-100 ng/ml, such as 40-60 ng/ml, particular 45, 46, 47, 48, 49, 50, 51, 52, 53, 54, or 55 ng/mL, specifically about 50 ng/ml.

In some cases, the NK cells are expanded either prior to modification to overexpress and/or upregulate the activity of one or more members of the CEBP family, and/or following said modification. Pre-activated NK cells may be expanded in the presence of artificial antigen presenting cells (aAPCs) and/or feeders/fragments or NK activating beads. The pre-activated NK cells may be washed prior to expansion, such as 2, 3, 4, or 5 times, specifically 3 times. The aAPCs may be engineered to express CD137 ligand and/or a membrane-bound cytokine. The membrane-bound cytokine may be membrane-bound IL-21 (mIL-21) or membrane-bound IL-15 (mIL-15). In particular embodiments, the aAPCs are engineered to express CD137 ligand and mIL-21. The aAPCs may be derived from cancer cells, such as leukemia cells. The aAPCs may not express endogenous HLA class I, II, or CD1d molecules. They may express ICAM-1 (CD54) and LFA-3 (CD58). In particular, the aAPCs may be K562 cells, such as K562 cells engineered to express CD137 ligand and mIL-21. The aAPCs may be irradiated. In some embodiments, fragments of APC can be used to expand the NK cells. The engineering may be by any method known in the art, such as retroviral transduction. Retroviral transduction may be at least, at most, or about 1, 2, 3, 4, 5, 6, 7, 8, 9, 10, 11, 12, 13, 14, 15, 16, 17, 18, 19, or 20 days following NK co-culturing with an antigen presenting cell. In some embodiments, retroviral transduction comprises co-transduction of more than one construct. In some embodiments, retroviral transduction occurs after or at about 5 days of co-culturing with an antigen presenting cell. In some embodiments, co-culturing with an antigen presenting cell continues following transduction of an NK cell. The expansion may be for about 2-30 days, such as 3-20 days, particularly 12-16 days, such as 12, 13, 14, 15, 16, 17, 18, or 19 days, specifically about 14 days. The pre-activated NK cells and aAPCs may be present at a ratio of about 3:1-1:3, such as 2:1, 1:1, 1:2, specifically about 1:2. The expansion culture may further comprise cytokines to promote expansion, such as IL-2. The IL-2 may be present at a concentration of about 10-500 U/mL, such as 100-300 U/mL, particularly about 200 U/mL. The IL-2 may be replenished in the expansion culture, such as every 2-3 days. The aAPCs may be added to the culture at least a second time, such as at about 7 days of expansion.

In particular embodiments, the NK cells are transfected or transduced with one or more membrane bound cytokines, including IL-21, IL-12, IL-18, IL-23, IL-7, or IL-15, either secreted by NK cells or tethered to the NK cell membrane. In such cases, the membrane bound cytokine may be tethered to the NK cell membrane with a particular transmembrane domain, such as the transmembrane domain of CD8, CD28, CD27, B7H3, IgG1, IgG4, CD4, DAP10, DAP12, for example. In certain embodiments, a cytokine is tethered to the NK cell membrane with a polypeptide comprising a sequence at least 80, 81, 82, 83, 84, 85, 86, 87, 88, 89, 90, 91, 92, 93, 94, 95, 96, 97, 98, 99, or 100% identical to a peptide represented by and/or encoded by any of SEQ ID NOs: 5-13. In certain embodiments, a cytokine is fused to an extracellular domain of a polypeptide comprising a sequence at least 80, 81, 82, 83, 84, 85, 86, 87, 88, 89, 90, 91, 92, 93, 94, 95, 96, 97, 98, 99, or 100% identical to a peptide represented by and/or encoded by any of SEQ ID NOs: 13-14. In certain embodiments, an extracellular domain of a polypeptide may be N terminal or C terminal of the cytokine. In certain embodiments, a cytokine comprises a signal peptide fused to the N terminal domain, wherein the signal peptide comprises a sequence at least 80, 81, 82, 83, 84, 85, 86, 87, 88, 89, 90, 91, 92, 93, 94, 95, 96, 97, 98, 99, or 100% identical to a peptide represented by and/or encoded by any of SEQ ID NOs: 15-18.

SEQ ID NO: 5-CD28 derived transmembrane domain amino acid sequence
(SEQ ID NO: 5)
FWVLVVVGGVLACYSLLVTVAFIIFWV
SEQ ID NO: 6-CD28 derived transmembrane domain nucleic acid sequence
(SEQ ID NO: 6)
TTTTGGGTGCTGGTGGTGGTTGGTGGAGTCCTGGCTTGCTATAGCTTGCTAGTAACAGTGGC
CTTTATTATTTTCTGGGTG
SEQ ID NO: 7-CD19 derived transmembrane domain amino acid sequence
(SEQ ID NO: 7)
VSAVTLAYLIFCLCSLVGILHL
SEQ ID NO: 8-CD19 derived transmembrane domain nucleic acid sequence
(SEQ ID NO: 8)
GTCTCAGCTGTGACTTTGGCTTATCTGATCTTCTGCCTGTGTTCCCTTGTGGGCATTCTTCA
TCTT
SEQ ID NO: 9-CD8 derived transmembrane domain amino acid sequence
(SEQ ID NO: 9)
IYIWAPLAGTCGVLLLSLVIT
SEQ ID NO: 10-CD8 derived transmembrane domain nucleic acid sequence
(SEQ ID NO: 10)
ATCTACATCTGGGCGCCCTTGGCCGGGACTTGTGGGGTCCTTCTCCTGTCACTGGTTATCAC
CCTTTACTG
SEQ ID NO: 11-CD4 derived transmembrane domain amino acid sequence
(SEQ ID NO: 11)
MALIVLGGVAGLLLFIGLGIFF
SEQ ID NO: 12-CD4 derived transmembrane domain nucleic acid sequence
(SEQ ID NO: 12)
ATGGCCCTGATCGTGCTGGGCGGAGTGGCTGGCCTGCTGCTGTTTATCGGCCTGGGCATATT
CTTT
SEQ ID NO: 13-CD8 derived extracellular domain amino acid sequence
(SEQ ID NO: 13)
TTTPAPRPPTPAPTIASQPLSLRPEACRPAAGGAVHTRGLDFACD
SEQ ID NO: 14-CD8 derived extracellular domain nucleic acid sequence
(SEQ ID NO: 14)
ACCACGACGCCAGCGCCGCGACCACCAACACCGGCGCCCACCATCGCGTCGCAGCCCCTGTC
CCTGCGCCCAGAGGCGTGCCGGCCAGCGGCGGGGGGCGCAGTGCACACGAGGGGGCTGGACT
TCGCCTGTGAT
SEQ ID NO: 15-GMCSF-R signal peptide amino acid sequence
(SEQ ID NO: 15)
MLLLVTSLLLCELPHPAFLLIP
SEQ ID NO: 16-GMCSF-R signal peptide nucleic acid sequence
(SEQ ID NO: 16)
ATGCTGCTGCTCGTGACCTCTCTGCTGCTGTGCGAGCTGCCCCACCCTGCCTTTCTGCTGAT
CCCT
SEQ ID NO: 17-Exemplary signal peptide amino acid sequence
(SEQ ID NO: 17)
EFGLSWLELVAILKGVQCSR
SEQ ID NO: 18-Exemplary signal peptide nucleic acid sequence
(SEQ ID NO: 18)
GAGTTTGGGCTGAGCTGGCTTTTTCTTGTGGCTATTTTAAAAGGTGTCCAGTGCTCTAGA

Following preparation, the engineered NK cells may be immediately infused (optionally, in certain embodiments, including with an effective amount of one or more monospecific, bispecific, or multi-specific antibodies), or the NK cells may be stored, such as by cryopreservation. In some cases, when the NK cells are sourced from cryopreservation, the NK cells were deactivated pre-cryopreservation using a deactivating agent (e.g., a kinase inhibitor, e.g., Dasatinib, nilotinib, rapamycin, etc.). In certain aspects, the cells may be propagated for days, weeks, or months ex vivo as a bulk population within about 1, 2, 3, 4, or 5 days.

### C. Loading of NK Cells

In particular embodiments, engineered NK cells are loaded with antibodies prior to use. The NK cells may be loaded in any specific manner, including in culture or immediately before infusion, for example, to produce a complex of NK cells with the antibodies. The conditions are suitable enough to allow for an effective amount of antibody to bind to the surface of the NK cells. In the case of use of monospecific antibodies, the Fc region of the monospecific antibody binds the NK cell while the antigen binding domain of the monospecific antibody is free to bind its target antigen. In certain embodiments of cases of use of multispecific antibodies, one or more antigen binding domains of the antibody can bind the surface of the NK cells, such as through an antigen on the surface of the NK cells, (for example but not limited to, CD3, NKp30, NKp44, NKp46, CD16, CD32, CD64, KIRs, and the like), and the other antigen binding domain is free to bind its target antigen. In certain embodiments of cases of use of multispecific antibodies, one or more antigen binding domains of the antibody can bind one or more target antigens.

The culture conditions by which the NK cells become loaded may or may not be of a particular type having one or more specific parameters. In particular embodiments, the loading of the NK cells occurs in culture at a specific temperature, such as 37° C., although in alternative embodiments the temperature is 36° C. or 38° C., or lower or higher. The duration of the loading step may be for any suitable amount of time, such as in a range of one minute to 24 hours or longer. For example, the range may be in the range of 1 min to 24 hrs, 1 min to 18 hrs, 1 min to 12 hours, 1 min to 6 hrs, 1 min to 1 hr, 30 min to 24 hrs, 30 min to 18 hrs, 30 min to 12 hrs, 30 min to 6 hrs, 30 min to 1 hr, 1-24 hrs, 1-18 hrs, 1-12 hrs, 1-6 hrs, 6-24 hrs, 6-18 hrs, 6-12 hrs, 12-24 hrs, 12-18 hrs, or 18-24 hrs. In some embodiments, the duration of the loading step may be greater than or equal to approximately 6, 8, 10, 12, 14, 16, 18, 20, 22, 24, 26, 28, 30, 32, 34, 36, 38, 40, 42, 44, 46, or 48 hours, or any range derivable therein. In specific embodiments, the cell culture media is basal media or complex media. In some cases, the culture comprises one or more reagents that were utilized during pre-activation and/or expansion steps, while in other cases the culture does not. In specific embodiments, the culture comprises one or more cytokines, including one or more of IL-12, IL-15, IL-2, and IL-18, for example. In some embodiments, the culture comprises APCs of any kind.

In certain embodiments, antibodies of compositions described herein are subjected in an effective amount to an effective amount of NK cells of the disclosure, thereby producing a complex that is “chimeric antigen receptor-like.” In particular, an antigen binding domain of the antibody binds to the NK cells, such as through the antigen that is a cell surface protein. A plurality of antibodies may be subjected to a plurality of NK cells such that there are multiple complexes of cell/antibody. The antibodies may be of any type, including monospecific, bispecific, or multispecific, and in specific cases the antibody engages both the NK cell and a target antigen through an antigen binding domain of the antibody (such as with engagers in the art that are fusion proteins consisting of two single-chain variable fragments (scFvs) of different antibodies). In examples wherein the antibody is monospecific, an antigen binding domain of the antibody binds a target antigen, such as a cancer antigen, and another part of the antibody binds the NK cells, such as an Fc region of the antibody. In cases wherein the antibody is multispecific, one or more antigen binding domains of the antibody binds the NK cell (such as through an NK cell surface antigen, either naturally occurring, or transgenic, e.g., CD3) and one or more antigen binding domains of the antibody binds one or more target antigens. The multispecific antibody may be bispecific, trispecific, or tetraspecific, for example. In cases wherein the antibody is trispecific or tetraspecific, the additional antigen binding domains may bind other cells, such as stem cells.

In particular embodiments, the antibodies may bind any NK cell surface antigen (that may or may not be receptors) on NK cells, such as CD16 (including CD16a or CD16b), CD32, CD56, CD64, a c-type lectin such as NKG2D, NKG2C, a costimulatory molecule such as CS1, DNAM, 2B4, CD2, an NCR, NKp30, NKp44, NKp46, or KIR, and redirect the NK cells to a target, thus increasing the response and specificity against different tumors. In certain embodiments, the antibodies may bind to a transgenic NK cell surface antigen, such as CD3.

In some embodiments, the antibodies may bind any suitable antigen (e.g., antigens described herein). In particular embodiments, an antibody targets CD19. In particular embodiments, an antibody targets CD20. In particular embodiments, an antibody targets CD123. In particular embodiments, an antibody targets EGFR. In particular embodiments, an antibody targets EGFR2.

In certain embodiments, generation of loaded NK cells may be by any suitable means, such that the conditions are sufficient for the appropriate region of the antibody to bind the appropriate surface region of the NK cell. Any particular medium may be utilized, in certain instances. In specific cases, Plasma-Lyte A and/or human serum albumin are utilized, wherein in other cases they are not. Once the complexes are formed in culture, they may or may not be washed prior to administration to the subject, such as through infusion. In alternative embodiments, the NK cells and the antibodies are administered separately, and the complexes form in vivo.

### D. Pre-Activation

In some embodiments, the NK cells are pre-activated prior to administration to a recipient individual. The pre-activation step may or may not occur before any expansion step. In specific embodiments, the NK cells are pre-activated with one or more cytokines, and in specific embodiments, the NK cells are pre-activated with one or more of IL-12, IL-15, IL-2, and IL-18 and including two, three, or more. In cases wherein less than all three of IL-12, IL-15, IL-2, and IL-18 are utilized, it may be that IL-12 and IL-15 but not IL-18; or IL-12 and IL-18 but not IL-15; or IL-15 and IL-18 but not IL-12. IL-2 may or may not be substituted for IL-15.

In particular embodiments, the pre-activation cytokines may be IL-12, IL-15, and IL-18. One or more additional cytokines may be used for the pre-activation step. The pre-activation may be for a short period of time such as 5-72 hours, such as 10-50 hours, particularly 10-20 hours, such as 12, 13, 14, 15, 16, 17, 18, 19, or 20 hours, and specifically about 16 hours in some cases. The pre-activation culture may comprise IL-18 and/or IL-15 at a concentration of 10-100 ng/mL, such as 40-60 ng/mL, particular 45, 46, 47, 48, 49, 50, 51, 52, 53, 54, or 55 ng/ml, specifically about 50 ng/mL. In some cases, the pre-activation culture comprises IL-12 at a concentration of 0.1-150 ng/ml, including at a concentration of 1-20 ng/mL, such as a concentration of 10 ng/mL. In alternative embodiments the NK cells may be stimulated with IL-2, or other cytokines that bind the common gamma-chain (e.g., IL-7, IL-21, and others), and this may be in addition to IL-12, IL-15, and IL-18 or as an alternative to one or more of them. In such cases, the pre-activation culture may comprise IL-12 at a concentration of 0.1-150 ng/ml, such as 0.5-50 ng/ml, particularly 1-20 ng/ml, such as 2, 3, 4, 5, 6, 7, 8, 9, 10, 11, 12, 13, 14, or 15 ng/mL, specifically about 10 ng/mL.

### E. Expansion

In particular embodiments, NK cells are expanded to increase their quantity prior to administration to an individual in need thereof. The expanded cells may or may not be derived from pre-activated NK cells such that a pre-activation step may occur before an expansion step. The NK cell expansion step may be of any suitable such that the NK cell population is expanded, but in specific cases the expansion step utilizes particular one or more reagents, such as in culture, to enhance their expansion. In certain cases the NK cells may not be expanded. IL-2 or IL-15 or IL-18 or any combination of the cytokines may be added to the expansion culture before or during expansion. The NK cells can be expanded ex vivo in flasks or in one of several different bioreactor configurations with continuous perfusion of media/additives, in specific embodiments.

In specific cases, the NK cells (whether pre-activated or not) may be washed (e.g., with PBS or Plasma Lyte or human serum albumin or culture media or combinations thereof) prior to and/or after expansion, such as 1, 2, 3, 4, or 5 times. In some embodiments, cells are washed specifically 3 times. In particular embodiments, the NK cells are expanded in the presence of artificial antigen presenting cells (aAPCs). In particular embodiments, the NK cells are expanded in the presence of fragments of aAPCs. The aAPCs may be engineered to express CD137 ligand and/or a membrane-bound cytokine. The membrane-bound cytokine may be membrane-bound IL-21 (mIL-21) or membrane-bound IL-15 (mIL-15). In particular embodiments, the aAPCs are engineered to express CD137 ligand and mIL-21. The aAPCs may be derived from cancer cells, such as leukemia cells. The aAPCs may not express endogenous HLA class I, II, or CD1d molecules. They may express ICAM-1 (CD54) and LFA-3 (CD58) or CD48. In particular, the aAPCs may be K562 cells, such as K562 cells engineered to express CD137 ligand and mIL-21. The engineering may be by any method known in the art, such as retroviral transduction, although any viral or non-viral vector may be utilized. The aAPCs may or may not be irradiated. The expansion may be for a particular duration in time, such as for about 2-30 days, such as 3-20 days, particularly 12-16 days, such as 12, 13, 14, 15, 16, 17, 18, or 19 days, specifically about 14 days. The pre-activated NK cells and aAPCs may be present at a ratio of about 3:1-1:3, such as 2:1, 1:1, 1:2, specifically about 1:2. The expansion culture may further comprise one or more cytokines to promote expansion, such as IL-2. The IL-2 may be present at a concentration of about 10-500 U/mL, such as 100-300 U/mL, particularly about 200 U/mL. The IL-2 may be replenished in the expansion culture, including at a certain frequency, such as every 2-3 days. The aAPCs may be added to the culture at least a second time, such as at about 7 days of expansion. Any cytokine(s) used in the pre-activation and/or expansion steps may be recombinant human cytokines.

In some embodiments, following expansion, the NK cells may be immediately utilized in any manner, such as complexed with one or more antibodies, or they may be stored, such as by cryopreservation. In certain aspects, the cells may be propagated for days, weeks, or months ex vivo as a bulk population within about 1, 2, 3, 4, or 5 days.

Activated and/or expanded NK cells can secrete type I cytokines, such as interferon-γ, tumor necrosis factor-α and granulocyte-macrophage colony-stimulating factor (GM-CSF), which activate both innate and adaptive immune cells as well as other cytokines and chemokines. The measurement of these cytokines can be used to determine the activation status of NK cells. In addition, other methods known in the art for determination of NK cell activation may be used for characterization of the NK cells of the present disclosure.

Thus, with respect to particular pre-activation and expansion aspects of the disclosure, in specific embodiments the NK cells pre-activated with any combination of IL-12, IL15, and/or IL-18 followed by expansion with aAPCs, such as K562 cells expressing mIL-21 and CD137 ligand, provide a highly potent cellular product. Thus, methods are provided using the present NK cells for the treatment of various diseases, such as immunotherapy of patients with cancer. In an exemplary method, the isolated NK cells may be subjected to a brief period, such as about 16 hours, of pre-activation with a combination of cytokines, such as interleukin-12 (IL-12), IL-15, and/or IL-18, followed by expansion using artificial antigen presenting cells (aAPCs), such as K562 feeder cells expressing membrane-bound IL-21 and CD137 ligand, and/or exogenous IL-2. IL-2 or IL-15 or IL-18 or any combination of the cytokines may be added to the expansion culture at least a second time.

### F. Cryopreservation

In particular cases, NK cells and/or antibodies of the disclosure are preserved in a cryopreservation medium composition comprising at least one cryoprotectant, a serum (human or animal serum) or a non-serum alternative to serum (not human serum or animal serum), and at least one cytokine and/or at least one growth factor. In some cases, the cryoprotectant is dimethyl sulfoxide (DMSO), glycerin, glycerol, hydroxyethyl starch, or a combination thereof. The non-serum alternative may be of any kind, including at least platelet lysate and/or a blood product lysate (for example, human serum albumin). In embodiments of the composition wherein one or more (including two or more) cytokines are utilized, the cytokine may be a natural or a recombinant or a synthetic protein. At least one of the cytokines may be an Food and Drug Administration (FDA)-approved cytokine. Examples of cytokines and growth factors include at least IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, thrombopoietin, erythropoietin, or a combination thereof. For serum embodiments, the serum may be an animal-derived serum, such as human serum (including human AB serum) or bovine serum. DMSO and other cryoprotectants, when utilized may comprise 4-10%, 4-6%, 4-8%, 5-10%, 5-8%, 6-10%, 6-8%, 8-10%, and so forth, of the composition. For embodiments wherein serum is employed, the serum may comprise 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%, 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of serum. In specific embodiments, the composition comprises platelet lysate that may be at any concentration in the composition, but in certain embodiments the platelet lysate comprises 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%, 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of platelet lysate.

The composition may have certain concentrations of components, including cytokines and/or growth factors. In specific cases, any cytokine, including IL-2, IL-21, and/or IL-15, for example, are present in the composition in a particular concentration. The IL-2 may be present at a concentration of 1-5000, 1-1000, 1-500, 1-100, 100-5000, 100-500, 500-5000, 500-1000, or 1000-5000 U/mL, for example. In a specific case, the IL-2 is present at a concentration in the composition of at least or no more than 100, 200, 300, 400, 500, 600, 700, 800, 900, or 1000 U/mL. In specific embodiments, IL-21 is present in the composition at a concentration of 10-3000, 10-2000, 10-1000, 10-500, 10-100, 100-3000, 100-2000, 100-1000, 500-3000, 500-2000, 500-1000, 1000-3000, 1000-2000, or 2000-3000 ng/mL. The IL-21 may be in a concentration in the composition of at least or nor more than 1, 5, 10, 20, 30, 40, 50, 60, 70, 80, 90, 100, 150, 200, 250, 300, 350, 400, 450, 500, 750, 1000, 1250, 1500, 1750, 2000, 2250, 2500, 2750, or 3000 ng/ml. IL-15 may be present in the composition at a concentration of 1-2000, 1-1000, 1-500, 1-100, 100-2000, 100-1000, 100-500, 500-2000, 500-1000, or 1000-2000 ng/mL. IL-15 may be present in the composition at a concentration of at least or no more than 10, 50, 100, 500, 1000, 1500, or 2000 ng/mL.

Compositions as encompassed herein that comprise at least one cryoprotectant, a serum or a non-serum alternative to serum, and at least one cytokine and/or at least one growth factor may further comprise a plurality of immune cells and/or stem cells, each of any kind. In specific embodiments, the cells are NK cells, T cells, B cells, NKT cells derived from mature bone marrow or peripheral blood cells, cell lines such as tumor cell lines (e.g., NK92 or other NK lines), hematopoietic stem cells, induced pluripotent stem cells, MSCs (a population of cells alternatively called “mesenchymal stem cells” and “mesenchymal stromal cells” in the literature), or a mixture thereof, which can be derived from bone marrow, peripheral blood, skin, adipose tissue, or a combination thereof. In embodiments wherein NK cells are utilized, the NK cells may or may not be expanded NK cells. Embodiments of the disclosure also encompass pharmaceutical compositions that comprise any composition of the disclosure and a suitable pharmaceutically acceptable carrier.

In certain embodiments, cells and/or antibodies are treated with one or more deactivating agents (e.g., a kinase inhibitor, e.g., Dasatinib, Nilotinib, Rapamycin, etc.) pre-cryopreservation.

In some embodiments, technologies described herein comprise deactivating a NK cell, comprising treating an NK cell with an effective amount of one or more deactivating agents under conditions to produce a deactivated NK cell. In some embodiments, a deactivating agent is a kinase inhibitor. In some embodiments, a deactivating agent is a mechanistic target of rapamycin (mTOR) inhibitor. In some embodiments, the mTOR inhibitor is rapamycin, everolimus, and/or temsirolimus. In some embodiments, the mTOR inhibitor is rapamycin. In some embodiments, the deactivating agent is a tyrosine kinase (TK) inhibitor. In some embodiments, the TK inhibitor is Lorlatinib, Brigatinib, Ceritinib, Alectinib, Crizotinib, Bosutinib, Ponatinib, Nilotinib, Dasatinib, Imatinib, Zanubrutinib, Acalabrutinib, Ibrutinib, Capmatinib, Pexidartinib, Dacomitinib, Osimertinib, Erlotinib, Gefitinib, Lapatinib, Afatinib, Pemigatinib, Erdafitinib, Nintedanib, Gilteritinib, Midostaurin, Tucatinib, Neratinib, Baricitinib, Ruxolitinib, Fedratinib, Tofacitinib, Ripretinib, Selumetinib, Binimetinib, Cobimetinib, Trametinib, Upadacitinib, Avapritinib, Selpercatinib, Cabozantinib, Fostamatinib, Larotrectinib, Entrectinib, Axitinib, Regorafenib, Pazopanib, Sorafenib, Lenvatinib, Vandetanib, and/or Sunitinib. In some embodiments, the TK inhibitor is a BCR-Abl inhibitor. In some embodiments, the TK inhibitor is Bosutinib, Ponatinib, Nilotinib, Dasatinib, and/or Imatinib. In some embodiments, the TK inhibitor is Dasatinib and/or Nilotinib. In some embodiments, the TK inhibitor is Dasatinib.

In some embodiments, treatment with a deactivating agent is at any point during culturing of the NK cell. In some embodiments, the treatment is for about 24 to about 96 hours, about 36 to about 84 hours, or about 48 to about 72 hours. In some embodiments, the treatment is for about 24 hours, about 48 hours, or about 72 hours. In some embodiments, the NK cell is treated with the deactivating agent at a concentration of about 1 to about 1000 nM. In some embodiments, the NK cell is treated with the deactivating agent at a concentration of about 5 to about 500 nM. In some embodiments, the NK cell is treated with the deactivating agent at a concentration of about 20 to about 200 nM. In some embodiments, the NK cell is treated with the deactivating agent at a concentration of about 30 to about 100 nM. In some embodiments, the deactivated NK cell has an increased expression of one or more of C-kit, CCR-5, CD62L and/or CXCR4, and/or decreased expression of one or more of NKG2D, DNAM, OX-40, TRAIL, HLA-DR, CD2, CD25, ICOS, and/or CD95 relative to an activated NK cell. In some embodiments, technologies described herein comprise methods of maintaining the viability of a population of cells over at least 50% percent following cryopreservation of the population, comprising the step of subjecting the population to an effective amount of one or more deactivating agents (e.g., a tyrosine kinase inhibitor) to deactivate the cells prior to cryopreservation, cryopreserving the cells, and thawing the population, wherein upon thawing the viability of the population is over at least 50%. In some cases, upon thawing of the cells the viability of the population of cells is over at least 55, 60, 65, 70, 75, 80, 85, 90, 91, 92, 93, 94, 95, 96, 97, 98, or 99% following cryopreservation of the population.

### III. HETEROLOGOUS PROTEINS AND MUTATIONS

In specific embodiments, NK cells are engineered to overexpress and/or upregulate the activity of one or more members of the CEBP family (e.g., heterologous expression), and may also be modified to express one or more other heterologous proteins. In certain embodiments, NK cells are engineered to express one or more heterologous proteins. The heterologous proteins may facilitate activity of the NK cells in any manner, including at least their activation, persistence, expansion, homing, and/or cytotoxicity.

The oligonucleotides, polypeptides, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, exactly, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 or more contiguous amino acids or nucleic acids, or any range derivable therein, of SEQ ID NOs: 1-63. In specific aspects, the nucleic acid encoding the peptide or polypeptide is codon optimized for expression in a mammal. In certain aspects, the peptide or polypeptide is not naturally occurring and/or is in a combination of peptides or polypeptides.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, or 422 substitutions (or any range derivable therein). In some aspects, the substitution is with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

In some aspects, the polypeptide comprises one or more substitutions at one or more amino acid positions selected from amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, or 529 of any of SEQ ID NOs: 1-5, 7, 9, 11, 13, 15, 17, 19, 23, 25, 27, 29, 31, 33, 35, 37, 39, or 41, wherein each substitution is independently chosen from an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine; and wherein the polypeptide is or is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOs: 1-5, 7, 9, 11, 13, 15, 17, 19, 23, 25, 27, 29, 31, 33, 35, 37, 39, or 41.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, or 529 of SEQ ID NOs: 1-5, 7, 9, 11, 13, 15, 17, 19, 23, 25, 27, 29, 31, 33, 35, 37, 39, or 41.

In some aspects, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, or 529 of SEQ ID NOs: 1-5, 7, 9, 11, 13, 15, 17, 19, 23, 25, 27, 29, 31, 33, 35, 37, 39, or 41 and have or have at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) sequence identity to one of SEQ ID NOs: 1-63.

In some aspects, the protein, polypeptide, or nucleic acid may comprise, comprise at least, or comprise at most 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 (or any derivable range therein) contiguous amino acids or nucleic acids of SEQ ID NOs: 1-63.

In some aspects, the polypeptide, protein, or nucleic acid may comprise at least, at most, or exactly 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 (or any derivable range therein) contiguous amino acids or nucleic acids of SEQ ID NOs: 1-63 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOs: 1-63.

In some aspects there is a nucleic acid molecule or polypeptide starting at position 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, or 422, of any of SEQ ID NOs: 1-63 and comprising at least, at most, or exactly 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 425, 450, 475, 500, 525, 550, 575, 600, 625, 650, 675, 700, 725, 750, 775, 800, 825, 850, 875, 900, 925, 950, 1000, 1200, 1266, 1400, 1600, 1800, or 2000 (or any derivable range therein) contiguous amino acids or nucleic acids of any of SEQ ID NOs: 1-63.

### A. Transcriptional Regulators

In specific embodiments, the NK cells are engineered to express one or more transcriptional regulators. In some embodiments, a transcriptional regulator is a transcription factor (TF). In some embodiments, a transcriptional regulator is a positive and/or negative regulator of a transcription factor.

### 1. CCAAT/Enhancer-Binding Proteins (C/EBP)

In some embodiments, a transcription factor is a CEBP (also referred to herein as C/EBP) protein family transcription factor. In certain embodiments, an engineered Natural Killer (NK) cell is modified to overexpress a CEBP protein relative to a non-engineered NK cell and/or an NK cell engineered to express IL-15. In certain embodiments, the CEBP protein is CEBPA (CEBP-alpha, CEBPα), CEBPB (CEBP-beta, CEBPβ), CEBPG (CEBP-gamma, CEBPγ), CEBPD (CEBP-delta, CEBPδ), CEBPE (CEBP-epsilon, CEBPε) and/or CEBP Homologous Protein (CHOP). In certain embodiments, the CEBP protein is CEBPD. In certain embodiments, the CEBP protein is CEBPB. In certain embodiments, the CEBP protein is CEBPD and CEBPB.

In certain embodiments, an engineered NK cell is transgenically modified to express a nucleic acid sequence encoding and/or a protein comprising, consisting essentially of, or consisting of a sequence at least, or exactly 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 19-28. In certain embodiments, a CEBP family member may be expressed as part of a multicistronic construct. In certain embodiments, a CEBP family member is transcriptionally linked to one or more marker proteins. In certain embodiments, a CEBP family member is operably linked to a heterologous transcriptional regulatory sequence, such as but not limited to, one or more of: a promoter, an enhancer, a 5′ UTR, a 3′ UTR, an RNA interference machinery target site (e.g., a target of one or more miRNA, siRNA, shRNA, etc.), and/or a polyadenylation signal.

SEQ ID NO: 19-CEBP Delta (CEBPD, CEBPδ) amino acid sequence
(SEQ ID NO: 19)
MSAALFSLDGPARGAPWPAEPAPFYEPGRAGKPGRGAEPGALGEPGAAAPAMYDDESAIDFS
AYIDSMAAVPTLELCHDELFADLFNSNHKAGGAGPLELLPGGPARPLGPGPAAPRLLKREPD
WGDGDAPGSLLPAQVAACAQTVVSLAAAGQPTPPTSPEPPRSSPRQTPAPGPAREKSAGKRG
PDRGSPEYRQRRERNNIAVRKSRDKAKRRNQEMQQKLVELSAENEKLHQRVEQLTRDLAGLR
QFFKQLPSPPFLPAAGTADCR
SEQ ID NO: 20-CEBP Delta (CEBPD, CEBPδ) codon optimized nucleic
acid sequence
(SEQ ID NO: 20)
ATGTCAGCAGCTCTTTTTTCTCTTGATGGTCCTGCCCGGGGCGCGCCATGGCCCGCTGAGCC
AGCTCCCTTTTACGAGCCAGGTAGAGCTGGAAAACCAGGGAGAGGGGCGGAGCCCGGGGCTC
TCGGGGAGCCTGGAGCTGCAGCTCCGGCCATGTATGACGATGAATCAGCAATCGATTTCTCA
GCGTACATTGATTCCATGGCCGCCGTTCCAACGTTGGAACTCTGTCATGATGAGCTTTTCGC
AGACCTCTTTAACTCTAATCACAAAGCTGGCGGCGCTGGTCCACTGGAACTTCTTCCTGGGG
GCCCCGCTCGGCCGCTCGGGCCCGGTCCCGCCGCCCCGAGATTGCTGAAAAGGGAACCGGAT
TGGGGAGATGGTGACGCGCCAGGAAGCTTGCTGCCTGCACAGGTAGCGGCATGCGCCCAGAC
CGTGGTGAGTCTGGCTGCAGCGGGCCAACCAACCCCTCCAACGTCACCGGAACCACCACGAT
CATCACCTAGACAAACACCAGCGCCCGGCCCGGCGCGCGAAAAGTCCGCAGGGAAAAGAGGG
CCTGACAGAGGCAGTCCGGAATACAGACAACGCCGGGAGAGGAACAATATAGCTGTGAGAAA
GAGTCGCGATAAGGCGAAACGGCGGAACCAAGAAATGCAGCAGAAGCTGGTAGAACTCAGCG
CAGAGAACGAGAAGCTCCATCAACGGGTTGAGCAACTGACTAGGGACCTCGCGGGCCTGAGA
CAATTTTTCAAGCAGCTGCCGAGTCCACCCTTTCTTCCCGCAGCGGGGACTGCCGACTGCCG
C
SEQ ID NO: 21-CEBP Delta (CEBPD, CEBPδ) nucleic acid sequence
(SEQ ID NO: 21)
ATGAGCGCCGCGCTCTTCAGCCTGGACGGCCCGGCGCGCGGCGCGCCCTGGCCTGCGGAGCC
TGCGCCCTTCTACGAACCGGGCCGGGCGGGCAAGCCGGGCCGCGGGGCCGAGCCAGGGGCCC
TAGGCGAGCCAGGCGCCGCCGCCCCCGCCATGTACGACGACGAGAGCGCCATCGACTTCAGC
GCCTACATCGACTCCATGGCCGCCGTGCCCACCCTGGAGCTGTGCCACGACGAGCTCTTCGC
CGACCTCTTCAACAGCAATCACAAGGCGGGCGGCGCGGGGCCCCTGGAGCTTCTTCCCGGCG
GCCCCGCGCGCCCCTTGGGCCCGGGCCCTGCCGCTCCCCGCCTGCTCAAGCGCGAGCCCGAC
TGGGGCGACGGCGACGCGCCCGGCTCGCTGTTGCCCGCGCAGGTGGCCGCGTGCGCACAGAC
CGTGGTGAGCTTGGCGGCCGCAGGGCAGCCCACCCCGCCCACGTCGCCGGAGCCGCCGCGCA
GCAGCCCCAGGCAGACCCCCGCGCCCGGCCCCGCCCGGGAGAAGAGCGCCGGCAAGAGGGGC
CCGGACCGCGGCAGCCCCGAGTACCGGCAGCGGCGCGAGCGCAACAACATCGCCGTGCGCAA
GAGCCGCGACAAGGCCAAGCGGCGCAACCAGGAGATGCAGCAGAAGTTGGTGGAGCTGTCGG
CTGAGAACGAGAAGCTGCACCAGCGCGTGGAGCAGCTCACGCGGGACCTGGCCGGCCTCCGG
CAGTTCTTCAAGCAGCTGCCCAGCCCGCCCTTCCTGCCGGCCGCCGGGACAGCAGACTGCCG
GTAA
SEQ ID NO: 22-Construct comprising CEBP Delta (CEBPD, CEBPδ)
nucleic acid sequence
(SEQ ID NO: 22)
ATGTCAGCAGCTCTTTTTTCTCTTGATGGTCCTGCCCGGGGCGCGCCATGGCCCGCTGAGCC
AGCTCCCTTTTACGAGCCAGGTAGAGCTGGAAAACCAGGGAGAGGGGCGGAGCCCGGGGCTC
TCGGGGAGCCTGGAGCTGCAGCTCCGGCCATGTATGACGATGAATCAGCAATCGATTTCTCA
GCGTACATTGATTCCATGGCCGCCGTTCCAACGTTGGAACTCTGTCATGATGAGCTTTTCGC
AGACCTCTTTAACTCTAATCACAAAGCTGGCGGCGCTGGTCCACTGGAACTTCTTCCTGGGG
GCCCCGCTCGGCCGCTCGGGCCCGGTCCCGCCGCCCCGAGATTGCTGAAAAGGGAACCGGAT
TGGGGAGATGGTGACGCGCCAGGAAGCTTGCTGCCTGCACAGGTAGCGGCATGCGCCCAGAC
CGTGGTGAGTCTGGCTGCAGCGGGCCAACCAACCCCTCCAACGTCACCGGAACCACCACGAT
CATCACCTAGACAAACACCAGCGCCCGGCCCGGCGCGCGAAAAGTCCGCAGGGAAAAGAGGG
CCTGACAGAGGCAGTCCGGAATACAGACAACGCCGGGAGAGGAACAATATAGCTGTGAGAAA
GAGTCGCGATAAGGCGAAACGGCGGAACCAAGAAATGCAGCAGAAGCTGGTAGAACTCAGCG
CAGAGAACGAGAAGCTCCATCAACGGGTTGAGCAACTGACTAGGGACCTCGCGGGCCTGAGA
CAATTTTTCAAGCAGCTGCCGAGTCCACCCTTTCTTCCCGCAGCGGGGACTGCCGACTGCCG
CGGCTCTGGGGAAGGACGCGGTAGCCTTCTGACTTGCGGCGATGTGGAGGAGAATCCTGGGC
CCatggtgagcaagggcgaggagctgttcaccggggtggtgcccatcctggtcgagctggac
ggcgacgtaaacggccacaagttcagcgtgtccggcgagggcgagggcgatgccacctacgg
caagctgaccctgaagttcatctgcaccaccggcaagctgcccgtgccctggcccaccctcg
tgaccaccctgacctacggcgtgcagtgcttcagccgctaccccgaccacatgaagcagcac
gacttcttcaagtccgccatgcccgaaggctacgtccaggagcgcaccatcttcttcaagga
cgacggcaactacaagacccgcgccgaggtgaagttcgagggcgacaccctggtgaaccgca
tcgagctgaagggcatcgacttcaaggaggacggcaacatcctggggcacaagctggagtac
aactacaacagccacaacgtctatatcatggccgacaagcagaagaacggcatcaaggtgaa
cttcaagatccgccacaacatcgaggacggcagcgtgcagctcgccgaccactaccagcaga
acacccccatcggcgacggccccgtgctgctgcccgacaaccactacctgagcacccagtcc
gccctgagcaaagaccccaacgagaagcgcgatcacatggtcctgctggagttcgtgaccgc
cgccgggatcactctcggcatggacgagctgtacaagTGA
SEQ ID NO: 23-CEBP Beta (CEBPB, CEBPβ) isoform A amino acid sequence
(SEQ ID NO: 23)
MQRLVAWDPACLPLPPPPPAFKSMEVANFYYEADCLAAAYGGKAAPAAPPAARPGPRPPAGE
LGSIGDHERAIDFSPYLEPLGAPQAPAPATATDTFEAAPPAPAPAPASSGQHHDFLSDLFSD
DYGGKNCKKPAEYGYVSLGRLGAAKGALHPGCFAPLHPPPPPPPPPAELKAEPGFEPADCKR
KEEAGAPGGGAGMAAGFPYALRAYLGYQAVPSGSSGSLSTSSSSSPPGTPSPADAKAPPTAC
YAGAAPAPSQVKSKAKKTVDKHSDEYKIRRERNNIAVRKSRDKAKMRNLETQHKVLELTAEN
ERLQKKVEQLSRELSTLRNLFKQLPEPLLASSGHC
SEQ ID NO: 24-CEBP Beta (CEBPB, CEBPβ) transcript variant 2 (encoding
isoform A) nucleic acid sequence
(SEQ ID NO: 24)
ATGCAACGCCTGGTGGCCTGGGACCCAGCATGTCTCCCCCTGCCGCCGCCGCCGCCTGCCTT
TAAATCCATGGAAGTGGCCAACTTCTACTACGAGGCGGACTGCTTGGCTGCTGCGTACGGCG
GCAAGGCGGCCCCCGCGGCGCCCCCCGCGGCCAGACCCGGGCCGCGCCCCCCCGCCGGCGAG
CTGGGCAGCATCGGCGACCACGAGCGCGCCATCGACTTCAGCCCGTACCTGGAGCCGCTGGG
CGCGCCGCAGGCCCCGGCGCCCGCCACGGCCACGGACACCTTCGAGGCGGCTCCGCCCGCGC
CCGCCCCCGCGCCCGCCTCCTCCGGGCAGCACCACGACTTCCTCTCCGACCTCTTCTCCGAC
GACTACGGGGGCAAGAACTGCAAGAAGCCGGCCGAGTACGGCTACGTGAGCCTGGGGCGCCT
GGGGGCCGCCAAGGGCGCGCTGCACCCCGGCTGCTTCGCGCCCCTGCACCCACCGCCCCCGC
CGCCGCCGCCGCCCGCCGAGCTCAAGGCGGAGCCGGGCTTCGAGCCCGCGGACTGCAAGCGG
AAGGAGGAGGCCGGGGCGCCGGGCGGCGGCGCAGGCATGGCGGCGGGCTTCCCGTACGCGCT
GCGCGCTTACCTCGGCTACCAGGCGGTGCCGAGCGGCAGCAGCGGGAGCCTCTCCACGTCCT
CCTCGTCCAGCCCGCCCGGCACGCCGAGCCCCGCTGACGCCAAGGCGCCCCCGACCGCCTGC
TACGCGGGGGCCGCGCCGGCGCCCTCGCAGGTCAAGAGCAAGGCCAAGAAGACCGTGGACAA
GCACAGCGACGAGTACAAGATCCGGCGCGAGCGCAACAACATCGCCGTGCGCAAGAGCCGCG
ACAAGGCCAAGATGCGCAACCTGGAGACGCAGCACAAGGTCCTGGAGCTCACGGCCGAGAAC
GAGCGGCTGCAGAAGAAGGTGGAGCAGCTGTCGCGCGAGCTCAGCACCCTGCGGAACTTGTT
CAAGCAGCTGCCCGAGCCCCTGCTCGCCTCCTCCGGCCACTGCTAG
SEQ ID NO: 25-CEBP Beta (CEBPB, CEBPβ) isoform B amino acid sequence
(SEQ ID NO: 25)
MEVANFYYEADCLAAAYGGKAAPAAPPAARPGPRPPAGELGSIGDHERAIDFSPYLEPLGAP
QAPAPATATDTFEAAPPAPAPAPASSGQHHDFLSDLFSDDYGGKNCKKPAEYGYVSLGRLGA
AKGALHPGCFAPLHPPPPPPPPPAELKAEPGFEPADCKRKEEAGAPGGGAGMAAGFPYALRA
YLGYQAVPSGSSGSLSTSSSSSPPGTPSPADAKAPPTACYAGAAPAPSQVKSKAKKTVDKHS
DEYKIRRERNNIAVRKSRDKAKMRNLETQHKVLELTAENERLQKKVEQLSRELSTLRNLFKQ
LPEPLLASSGHC
SEQ ID NO: 26-CEBP Beta (CEBPB, CEBPβ) transcript variant 1 (encoding
isoform B) nucleic acid sequence
(SEQ ID NO: 26)
ATGGAAGTGGCCAACTTCTACTACGAGGCGGACTGCTTGGCTGCTGCGTACGGCGGCAAGGC
GGCCCCCGCGGCGCCCCCCGCGGCCAGACCCGGGCCGCGCCCCCCCGCCGGCGAGCTGGGCA
GCATCGGCGACCACGAGCGCGCCATCGACTTCAGCCCGTACCTGGAGCCGCTGGGCGCGCCG
CAGGCCCCGGCGCCCGCCACGGCCACGGACACCTTCGAGGCGGCTCCGCCCGCGCCCGCCCC
CGCGCCCGCCTCCTCCGGGCAGCACCACGACTTCCTCTCCGACCTCTTCTCCGACGACTACG
GGGGCAAGAACTGCAAGAAGCCGGCCGAGTACGGCTACGTGAGCCTGGGGCGCCTGGGGGCC
GCCAAGGGCGCGCTGCACCCCGGCTGCTTCGCGCCCCTGCACCCACCGCCCCCGCCGCCGCC
GCCGCCCGCCGAGCTCAAGGCGGAGCCGGGCTTCGAGCCCGCGGACTGCAAGCGGAAGGAGG
AGGCCGGGGCGCCGGGCGGCGGCGCAGGCATGGCGGCGGGCTTCCCGTACGCGCTGCGCGCT
TACCTCGGCTACCAGGCGGTGCCGAGCGGCAGCAGCGGGAGCCTCTCCACGTCCTCCTCGTC
CAGCCCGCCCGGCACGCCGAGCCCCGCTGACGCCAAGGCGCCCCCGACCGCCTGCTACGCGG
GGGCCGCGCCGGCGCCCTCGCAGGTCAAGAGCAAGGCCAAGAAGACCGTGGACAAGCACAGC
GACGAGTACAAGATCCGGCGCGAGCGCAACAACATCGCCGTGCGCAAGAGCCGCGACAAGGC
CAAGATGCGCAACCTGGAGACGCAGCACAAGGTCCTGGAGCTCACGGCCGAGAACGAGCGGC
TGCAGAAGAAGGTGGAGCAGCTGTCGCGCGAGCTCAGCACCCTGCGGAACTTGTTCAAGCAG
CTGCCCGAGCCCCTGCTCGCCTCCTCCGGCCACTGCTAG
SEQ ID NO: 27-CEBP Beta (CEBPB, CEBPβ) isoform C amino acid sequence
(SEQ ID NO: 27)
MAAGFPYALRAYLGYQAVPSGSSGSLSTSSSSSPPGTPSPADAKAPPTACYAGAAPAPSQVK
SKAKKTVDKHSDEYKIRRERNNIAVRKSRDKAKMRNLETQHKVLELTAENERLQKKVEQLSR
ELSTLRNLFKQLPEPLLASSGHC
SEQ ID NO: 28-CEBP Beta (CEBPB, CEBPβ) transcript variant 3 (encoding
isoform C) nucleic acid sequence
(SEQ ID NO: 28)
ATGGCGGCGGGCTTCCCGTACGCGCTGCGCGCTTACCTCGGCTACCAGGCGGTGCCGAGCGG
CAGCAGCGGGAGCCTCTCCACGTCCTCCTCGTCCAGCCCGCCCGGCACGCCGAGCCCCGCTG
ACGCCAAGGCGCCCCCGACCGCCTGCTACGCGGGGGCCGCGCCGGCGCCCTCGCAGGTCAAG
AGCAAGGCCAAGAAGACCGTGGACAAGCACAGCGACGAGTACAAGATCCGGCGCGAGCGCAA
CAACATCGCCGTGCGCAAGAGCCGCGACAAGGCCAAGATGCGCAACCTGGAGACGCAGCACA
AGGTCCTGGAGCTCACGGCCGAGAACGAGCGGCTGCAGAAGAAGGTGGAGCAGCTGTCGCGC
GAGCTCAGCACCCTGCGGAACTTGTTCAAGCAGCTGCCCGAGCCCCTGCTCGCCTCCTCCGG
CCACTGCTAG

### B. Bispecific or Multi-Specific Antibodies

In some embodiments, the NK cells are modified to express one or more bispecific or multi-specific antibodies, although in other cases the NK cells do not express the antibodies but the antibodies are utilized in conjunction with the NK cells.

In cases wherein the NK cells are modified to express the antibodies, the antibodies may be engagers that bridge a particular immune effector cell with a particular target cell for destruction of the target cell. In some embodiments, engineered NK cells are complexed with and/or utilized in conjunction with T-cell engagers (BiTEs) and/or bispecific NK engagers (BiKEs). In certain embodiments, a BiKE comprises an antibody that binds a surface protein on the NK cell, including a naturally expressed surface protein on NK cells (for example but not limited to, NKp30, NKp44, NKp46, CD16, CD32, CD64, KIRs, and the like), and also comprises an antibody that binds a desired target antigen. The BiKE may target the NK cells through an antibody an NK surface protein such as CD16, CS1, CD32, CD64, CD56, NKG2D, NKG2C, DNAM, 2B4, CD2, an NCR, NKp30, NKp44, NKp46, or KIR, for example. In such cases, the BiKE used in the inventions may also target a cancer or viral antigen that may be tailored to the medical condition of an intended recipient individual. For example, the BiKE may be tailored to bind a cancer antigen that is characteristic of the cancer cells of a cancer of the individual.

In certain embodiments, an antibody is Blinatumomab. In certain embodiments, an antibody is Tebentafusp. In certain embodiments, an antibody is Mosunetuzumab. In certain embodiments, an antibody is Teclistamab. In certain embodiments, an antibody is Glofitamab. In certain embodiments, an antibody is Epcoritamab. In some embodiments, an antibody is Flotetuzumab. In some embodiments, an antibody is APVO436. In some embodiments, an antibody is TNB383B. In certain embodiments of cases of use of multispecific antibodies, one or more antigen binding domains of the antibody can bind one or more target antigens.

### C. Cytokines

In some embodiments, the cells expressing the NK cells are engineered to express one or more heterologous cytokines and/or are engineered to upregulate normal expression of one or more heterologous cytokines. The cells may or may not be transduced or transfected for one or more cytokines on the same vector as other genes. In certain embodiments, NK cells may be modified to express one or more cytokines, cytokine receptors, chemokines, chemokine receptors, and/or suicide genes.

One or more cytokines may be co-expressed from a vector, including as a separate polypeptide from any component involved in overexpression and/or upregulation of the activity of one or more CEBP family proteins. In some embodiments, interleukin-15 (IL-15), for example, is tissue restricted and only under pathologic conditions is it observed at any level in the serum, or systemically. In some embodiments, IL-15 possesses several attributes that are desirable for adoptive therapy. In some embodiments, IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, in some embodiments, other cytokines are envisioned. These include, but are not limited to, cytokines (e.g., IL-2, IL-7, IL-12, IL-15, IL-17, IL-18, IL-21, chemokines, and other molecules that contribute to the activation and proliferation of cells used for human application. In some embodiments, NK cells expressing IL-15 are capable of continued supportive cytokine signaling, which is useful for their survival post-infusion. In certain embodiments, an engineered NK cell is not transgenically modified to express cytokine IL-15. In some embodiments, engineered NK cells autonomously and/or constitutively expressing and secreting IL-21 are capable of continued supportive cytokine signaling, which is useful for their survival post-infusion. In certain embodiments, engineered NK cells expressing IL-21 are capable of enhanced immune memory to glioblastoma stem cells relative to cells engineered to express IL-15. In certain embodiments, the NK cells are engineered to autonomously express IL-21. In certain embodiments, the NK cells are engineered to constitutively express IL-21. In certain embodiments, the NK cells are engineered to secrete IL-21. In certain embodiments, the NK cells are engineered to stably secrete IL-21 at levels sufficient to maintain extracellular concentration at or above 150, 175, 200, 225, 250, 275, 300, 325, 350, 375, 400, 425, 450, 475, 500, 525, 550, 575, 600, 625, 650, 675, 700, 725, 750, 775, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000, or greater than 3000 pg/mL. In certain embodiments, the NK cells are engineered to stably secrete IL-21, and thus create an extracellular concentration of IL-21, that is greater than or equal to, exactly or about, 150, 175, 200, 225, 250, 275, 300, 325, 350, 375, 400, 425, 450, 475, 500, 525, 550, 575, 600, 625, 650, 675, 700, 725, 750, 775, or 800 pg/mL, or any valuable derivable therein. In certain embodiments, the NK cells are engineered to stably secrete IL-21, and thus create an extracellular concentration of IL-21, at a concentration of equal to or about 200-800 pg/mL, 250-750 pg/mL, 300-700 pg/mL, 350-650 pg/mL, or 400-600 pg/mL, or any value or range derivable therein.

In some embodiments, the cells express one or more exogenously provided engineered receptors, wherein the engineered receptor comprises a chemokine receptor and/or a cytokine receptor. In some embodiments, a cytokine receptor is an IL-15 receptor. In some embodiments, a cytokine receptor is a non-naturally occurring variant of a cytokine receptor. In some embodiments, a cytokine receptor is an IL-15, IL-12, IL-2, IL-18, IL-21, IL-23, or GMCSF receptor, or a combination thereof.

In specific embodiments, the cells express one or more exogenously provided cytokines. As one example, the cytokine is IL-15, IL-12, IL-2, IL-18, IL-21, IL-23, GMCSF, or a combination thereof. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as one or more other components described herein.

In certain embodiments, an engineered NK cell is transgenically modified to express a nucleic acid sequence encoding and/or a cytokine comprising, consisting essentially of, or consisting of a sequence at least, or exactly 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 29-34.

In some embodiments, a specific sequence of IL-15 is utilized, such as those that follow (underlining refers to signal peptide sequence, which may be included or omitted):

SEQ ID NO: 29-IL-15 amino acid sequence (signal peptide underlined)
(SEQ ID NO: 29)
MRISKPHLRSISIQCYLCLLLNSHELTEAGIHVFILGCFSAGLPKTEANWVNVISDLKKIED
LIQSMHIDATLYTESDVHPSCKVTAMKCFLLELQVISLESGDASIHDTVENLIILANNSLSS
NGNVTESGCKECEELEEKNIKEFLQSFVHIVqMFINTS
SEQ ID NO: 30-IL-15 nucleic acid sequence (signal peptide underlined)
(SEQ ID NO: 30)
ATGCGCATTAGCAAGCCCCACCTGCGGAGCATCAGCATCCAGTGCTACCTGTGCCTGCTGCT
GAACAGCCACTTCCTGACCGAGGCCGGCATCCACGTGTTCATCCTGGGCTGCTTCAGCGCCG
GACTGCCCAAGACCGAGGCCAACTGGGTGAACGTGATCAGCGACCTGAAGAAGATCGAGGAC
CTGATCCAGAGCATGCACATCGACGCCACCCTGTACACCGAGAGCGACGTGCACCCCAGCTG
CAAGGTGACCGCCATGAAGTGCTTTCTGCTGGAACTGCAGGTGATCAGCCTGGAAAGCGGCG
ACGCCAGCATCCACGACACCGTGGAGAACCTGATCATCCTGGCCAACAACAGCCTGAGCAGC
AACGGCAACGTGACCGAGAGCGGCTGCAAAGAGTGCGAGGAACTGGAAGAGAAGAACATCAA
AGAGTTTCTGCAGAGCTTCGTGCACATCGTGCAGATGTTCATCAACACCAGC
In some embodiments, a specific sequence of IL-21 is utilized, such as those
that follow (underlining refers to signal peptide sequence, which may be
included or omitted):
SEQ ID NO: 31-Codon optimized IL-21 amino acid sequence (signal peptide
underlined)
(SEQ ID NO: 31)
MRSSPGNMERIVICLMVIFLGTLVHKSSSQGQDRHMIRMRQLIDIVDQLKNYVNDLVPEFLP
APEDVETNCEWSAFSCFQKAQLKSANTGNNERIINVSIKKLKRKPPSTNAGRRQKHRLTCPS
CDSYEKKPPKEFLERFKSLLQKMIHqHLSSRTHGSEDS
SEQ ID NO: 32-Codon optimized IL-21 nucleic acid sequence (signal peptide
underlined)
(SEQ ID NO: 32)
ATGAGGAGCAGTCCAGGCAATATGGAGCGGATAGTAATTTGTCTCATGGTAATATTCCTCGG
TACTCTGGTACATAAATCTTCCTCTCAAGGTCAGGACCGCCATATGATTCGAATGCGGCAGC
TGATTGACATAGTCGATCAACTGAAGAACTATGTGAATGATCTTGTGCCCGAGTTTTTGCCA
GCCCCTGAAGACGTAGAAACTAATTGTGAGTGGAGTGCCTTTTCCTGCTTTCAAAAGGCACA
GCTGAAATCCGCCAACACGGGCAATAACGAACGGATAATTAACGTATCCATTAAGAAGCTGA
AGCGGAAGCCGCCCTCAACCAATGCGGGACGGCGGCAAAAGCATCGCTTGACCTGTCCGTCA
TGCGACAGCTACGAGAAAAAGCCCCCGAAGGAGTTCTTGGAACGCTTCAAGAGTCTCCTTCA
GAAAATGATTCACCAGCACCTGTCCTCACGGACGCACGGAAGCGAGGACAGT
SEQ ID NO: 33-IL-21 amino acid sequence
(SEQ ID NO: 33)
GQDRHMIRMRQLIDIVDQLKNYVNDLVPEFLPAPEDVETNCEWSAFSCFQKAQLKSANTGNN
ERIINVSIKKLKRKPPSTNAGRRQKHRLTCPSCDSYEKKPPKEFLERFKSLLqKMIHQHLSS
RTHGSEDS
SEQ ID NO: 34-IL-21 nucleic acid sequence
(SEQ ID NO: 34)
GGCCAGGACCGGCACATGATCCGGATGAGACAGCTGATCGACATCGTGGACCAGCTGAAGAA
CTACGTGAACGACCTGGTGCCCGAGTTCCTGCCTGCCCCCGAGGACGTGGAAACAAACTGCG
AGTGGAGCGCCTTCAGCTGCTTCCAGAAGGCCCAGCTGAAAAGCGCCAACACCGGCAACAAC
GAGCGGATCATCAACGTGTCCATCAAGAAGCTGAAGCGGAAGCCCCCCAGCACCAACGCCGG
AAGAAGGCAGAAGCACAGACTGACCTGCCCCAGCTGCGACAGCTACGAGAAGAAGCCCCCTA
AAGAGTTCCTGGAACGGTTCAAGAGCCTGCTGCAGAAGATGATCCACCAGCACCTGAGCAGC
CGGACCCACGGCTCTGAGGACAGC

In certain embodiments, a cytokine is expressed as part of a multicistronic construct with one or more marker proteins. In certain embodiments, an engineered NK cell is transgenically modified to express a nucleic acid encoding and/or a marker protein comprising, consisting essentially of, or consisting of a sequence at least, or exactly 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 35-42.

In certain embodiments, an engineered NK cell is transgenically modified to express a nucleic acid comprising, consisting essentially of, or consisting of a sequence at least, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 43-51.

SEQ ID NO: 35-IgG Fc amino acid sequence
(SEQ ID NO: 35)
EPKSPDKTHTCPPCPAPELLGGPSVFLFPPKPKDTLMISRTPEVTCVVVDVSHEDPEVKfNW
YVDGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGKETTPNKGSGTTSGTTR
LLSGHTCFTLTGLLGTLVIMGLLT
SEQ ID NO: 36-IgG Fc nucleic acid sequence
(SEQ ID NO: 36)
GAGCCCAAATCTCCTGACAAAACTCACACATGCCCACCGTGCCCAGCACCTGAACTCCTGGG
GGGACCGTCAGTCTTCCTCTTCCCCCCAAAACCCAAGGACACCCTCATGATCTCCCGGACCC
CTGAGGTCACATGCGTGGTGGTGGACGTGAGCCACGAAGACCCTGAGGTCAAGTTCAACTGG
TACGTGGACGGCGTGGAGGTGCATAATGCCAAGACAAAGCCGCGGGAGGAGCAGTACAACAG
CACGTACCGTGTGGTCAGCGTCCTCACCGTCCTGCACCAGGACTGGCTGAATGGCAAGGAGT
ACAAGTGCAAGGTCTCCAACAAAGCCCTCCCAGCCCCCATCGAGAAAACCATCTCCAAAGCC
AAAGGGCAGCCCCGAGAACCACAGGTGTACACCCTGCCCCCATCCCGGGATGAGCTGACCAA
GAACCAGGTCAGCCTGACCTGCCTGGTCAAAGGCTTCTATCCCAGCGACATCGCCGTGGAGT
GGGAGAGCAATGGGCAACCGGAGAACAACTACAAGACCACGCCTCCCGTGCTGGACTCCGAC
GGCTCCTTCTTCCTCTACAGCAAGCTCACCGTGGACAAGAGCAGGTGGCAGCAGGGGAACGT
CTTCTCATGCTCCGTGATGCATGAGGCTCTGCACAACCACTACACGCAGAAGAGCCTCTCCC
TGTCTCCGGGTAAAGAAACAACCCCAAATAAAGGAAGTGGAACCACTTCAGGTACTACCCGT
CTTCTATCTGGGCACACGTGTTTCACGTTGACAGGTTTGCTTGGGACGCTAGTAACCATGGG
CTTGCTGACT
SEQ ID NO: 37-IgG4 amino acid sequence
(SEQ ID NO: 37)
ESKYGPPCPPCPAPEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
SEQ ID NO: 38-IgG4 nucleic acid sequence
(SEQ ID NO: 38)
GAGTCTAAGTACGGCCCTCCCTGCCCTCCTTGTCCAGCCCCTGAATTTCTGGGCGGACCCAG
CGTGTTCCTGTTCCCCCCAAAGCCCAAGGACACCCTGATGATCAGCAGGACCCCCGAAGTGA
CCTGCGTGGTGGTGGATGTGTCCCAGGAAGATCCCGAGGTGCAGTTTAATTGGTACGTGGAC
GGCGTGGAAGTGCACAACGCCAAGACCAAGCCCAGAGAGGAACAGTTCAACAGCACCTACCG
GGTGGTGTCCGTGCTGACCGTGCTGCACCAGGACTGGCTGAACGGCAAAGAGTACAAGTGCA
AGGTGTCCAACAAGGGCCTGCCCAGCAGCATCGAGAAAACCATCAGCAAGGCCAAGGGCCAG
CCTCGGGAACCCCAGGTGTACACACTGCCCCCTAGCCAGGAAGAGATGACCAAGAACCAGGT
GTCCCTGACCTGTCTCGTGAAGGGCTTCTACCCCAGCGACATTGCCGTGGAATGGGAGAGCA
ACGGCCAGCCCGAGAACAACTACAAGACCACCCCCCCTGTGCTGGACAGCGACGGCTCATTC
TTCCTGTACAGCCGCCTGACCGTGGACAAGAGCAGATGGCAGGAAGGCAACGTGTTCAGCTG
CAGCGTGATGCACGAGGCCCTGCACAACCACTACACCCAGAAGTCCCTGTCTCTGAGCCTGG
GCAAG
SEQ ID NO: 39-CD19 extracellular domain with signal peptide amino acid
sequence
(SEQ ID NO: 39)
MPPPRLLFFLLFLTPMEVRPEEPLVVKVEEGDNAVLQCLKGTSDGPTQQLTWSRESPLKPFL
KLSLGLPGLGIHMRPLAIWLFIFNVSQQMGGFYLCQPGPPSEKAWQPGWTVNVEGSGELFRW
NVSDLGGLGCGLKNRSSEGPSSPSGKLMSPKLYVWAKDRPEIWEGEPPCLPPRDSLNqSLSQ
DLTMAPGSTLWLSCGVPPDSVSRGPLSWTHVHPKGPKSLLSLELKDDRPARDMWVMETGLLL
PRATAQDAGKYYCHRGNLTMSFHLEITARPVLWHWLLRTGGWKVSAVTLAYLIFCLCSLVGI
LHL
SEQ ID NO: 40-CD19 extracellular domain with signal peptide nucleic acid
sequence
(SEQ ID NO: 40)
ATGCCACCTCCTCGCCTCCTCTTCTTCCTCCTCTTCCTCACCCCTATGGAAGTCAGGCCCGA
GGAACCTCTAGTGGTGAAGGTGGAAGAGGGAGATAACGCTGTGCTGCAGTGCCTCAAGGGGA
CCTCAGATGGCCCCACTCAGCAGCTGACCTGGTCTCGGGAGTCCCCGCTTAAACCCTTCTTA
AAACTCAGCCTGGGGCTGCCAGGCCTGGGAATCCACATGAGGCCCCTGGCCATCTGGCTTTT
CATCTTCAACGTCTCTCAACAGATGGGGGGCTICTACCTGTGCCAGCCGGGGCCCCCCTCTG
AGAAGGCCTGGCAGCCTGGCTGGACAGTCAATGTGGAGGGCAGCGGGGAGCTGTTCCGGTGG
AATGTTTCGGACCTAGGTGGCCTGGGCTGTGGCCTGAAGAACAGGTCCTCAGAGGGCCCCAG
CTCCCCTTCCGGGAAGCTCATGAGCCCCAAGCTGTATGTGTGGGCCAAAGACCGCCCTGAGA
TCTGGGAGGGAGAGCCTCCGTGTCTCCCACCGAGGGACAGCCTGAACCAGAGCCTCAGCCAG
GACCTCACTATGGCCCCTGGCTCCACACTCTGGCTGTCCTGTGGGGTACCCCCTGACTCTGT
GTCCAGGGGCCCCCTCTCCTGGACCCATGTGCACCCCAAGGGGCCTAAGTCATTGCTGAGCC
TAGAGCTGAAGGACGATCGCCCGGCCAGAGATATGTGGGTAATGGAGACGGGTCTGTTGTTG
CCCCGGGCCACAGCTCAAGACGCTGGAAAGTATTATTGTCACCGTGGCAACCTGACCATGTC
ATTCCACCTGGAGATCACTGCTCGGCCAGTACTATGGCACTGGCTGCTGAGGACTGGTGGCT
GGAAGGTCTCAGCTGTGACTTTGGCTTATCTGATCTTCTGCCTGTGTTCCCTTGTGGGCATT
CTTCATCTT
SEQ ID NO: 41-CD19 amino acid sequence
(SEQ ID NO: 41)
PEEPLVVKVEEGDNAVLQCLKGTSDGPTQQLTWSRESPLKPFLKLSLGLPGLGIHMRPLAIW
LFIFNVSQQMGGFYLCQPGPPSEKAWQPGWTVNVEGSGELFRWNVSDLGGLGCGLKNRSSEG
PSSPSGKLMSPKLYVWAKDRPEIWEGEPPCLPPRDSLNQSLSQDLTMAPGSTLWLSCGVPPD
SVSRGPLSWTHVHPKGPKSLLSLELKDDRPARDMWVMETGLLLPRATAQDAGKYYCHRGNLT
MSFHLEITARPVLWHWLLRTGGWK
SEQ ID NO: 42-CD19 nucleic acid sequence
(SEQ ID NO: 42)
CCCGAGGAGCCCCTGGTGGTGAAGGTGGAGGAGGGCGACAACGCCGTGCTGCAGTGCCTGAA
GGGCACCAGCGACGGCCCCACCCAGCAGCTGACCTGGAGCAGAGAGAGCCCCCTGAAGCCCT
TCCTGAAGCTGAGCCTGGGCCTGCCCGGCCTGGGCATCCACATGAGACCCCTGGCCATCTGG
CTGTTCATCTTCAACGTGAGCCAGCAGATGGGCGGCTTCTACCTGTGCCAGCCCGGCCCCCC
CAGCGAGAAGGCCTGGCAGCCCGGCTGGACCGTGAACGTGGAGGGCAGCGGCGAGCTGTTCA
GATGGAACGTGAGCGACCTGGGCGGCCTGGGCTGCGGCCTGAAGAACAGAAGCAGCGAGGGC
CCCAGCAGCCCCAGCGGCAAGCTGATGAGCCCCAAGCTGTACGTGTGGGCCAAGGACAGACC
CGAGATCTGGGAGGGCGAGCCCCCCTGCCTGCCCCCCAGAGACAGCCTGAACCAGAGCCTGA
GCCAGGACCTGACCATGGCCCCCGGCAGCACCCTGTGGCTGAGCTGCGGCGTGCCCCCCGAC
AGCGTGAGCAGAGGCCCCCTGAGCTGGACCCACGTGCACCCCAAGGGCCCCAAGAGCCTGCT
GAGCCTGGAGCTGAAGGACGACAGACCCGCCAGAGACATGTGGGTGATGGAGACCGGCCTGC
TGCTGCCCAGAGCCACCGCCCAGGACGCCGGCAAGTACTACTGCCACAGAGGCAACCTGACC
ATGAGCTTCCACCTGGAGATCACCGCCAGACCCGTGCTGTGGCACTGGCTGCTGAGAACCGG
CGGCTGGAAG
SEQ ID NO: 43-Exemplary SPCD19IgG1COIL21 Vector-nucleic acid sequence (signal
peptide underlined)
(SEQ ID NO: 43)
ATGCTCGAGTTTGGGCTGAGCTGGCTTTTTCTTGTGGCTATTTTAAAAGGTGTCCAGTGCTC
TAGACCCGAGGAGCCCCTGGTGGTGAAGGTGGAGGAGGGCGACAACGCCGTGCTGCAGTGCC
TGAAGGGCACCAGCGACGGCCCCACCCAGCAGCTGACCTGGAGCAGAGAGAGCCCCCTGAAG
CCCTTCCTGAAGCTGAGCCTGGGCCTGCCCGGCCTGGGCATCCACATGAGACCCCTGGCCAT
CTGGCTGTTCATCTTCAACGTGAGCCAGCAGATGGGCGGCTTCTACCTGTGCCAGCCCGGCC
CCCCCAGCGAGAAGGCCTGGCAGCCCGGCTGGACCGTGAACGTGGAGGGCAGCGGCGAGCTG
TTCAGATGGAACGTGAGCGACCTGGGCGGCCTGGGCTGCGGCCTGAAGAACAGAAGCAGCGA
GGGCCCCAGCAGCCCCAGCGGCAAGCTGATGAGCCCCAAGCTGTACGTGTGGGCCAAGGACA
GACCCGAGATCTGGGAGGGCGAGCCCCCCTGCCTGCCCCCCAGAGACAGCCTGAACCAGAGC
CTGAGCCAGGACCTGACCATGGCCCCCGGCAGCACCCTGTGGCTGAGCTGCGGCGTGCCCCC
CGACAGCGTGAGCAGAGGCCCCCTGAGCTGGACCCACGTGCACCCCAAGGGCCCCAAGAGCC
TGCTGAGCCTGGAGCTGAAGGACGACAGACCCGCCAGAGACATGTGGGTGATGGAGACCGGC
CTGCTGCTGCCCAGAGCCACCGCCCAGGACGCCGGCAAGTACTACTGCCACAGAGGCAACCT
GACCATGAGCTTCCACCTGGAGATCACCGCCAGACCCGTGCTGTGGCACTGGCTGCTGAGAA
CCGGCGGCTGGAAGTCGCGAGAGCCCAAATCTCCTGACAAAACTCACACATGCCCACCGTGC
CCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGACAAAGCCG
CGGGAGGAGCAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTACACCCTGCCCCCA
TCCCGGGATGAGCTGACCAAGAACCAGGTCAGCCTGACCTGCCTGGTCAAAGGCTTCTATCC
CAGCGACATCGCCGTGGAGTGGGAGAGCAATGGGCAACCGGAGAACAACTACAAGACCACGC
CTCCCGTGCTGGACTCCGACGGCTCCTTCTTCCTCTACAGCAAGCTCACCGTGGACAAGAGC
AGGTGGCAGCAGGGGAACGTCTTCTCATGCTCCGTGATGCATGAGGCTCTGCACAACCACTA
CACGCAGAAGAGCCTCTCCCTGTCTCCGGGTAAAGAAACAACCCCAAATAAAGGAAGTGGAA
CCACTTCAGGTACTACCCGTCTTCTATCTGGGCACACGTGTTTCACGTTGACAGGTTTGCTT
GGGACGCTAGTAACCATGGGCTTGCTGACTTTCGGACCGCAGTGTACTAATTATGCTCTCTT
GAAATTGGCTGGAGATGTTGAGAGCAATCCCGGGCCCATGCGCATGATGAGGAGCAGTCCAG
GCAATATGGAGCGGATAGTAATTTGTCTCATGGTAATATTCCTCGGTACTCTGGTACATAAA
TCTTCCTCTCAAGGTCAGGACCGCCATATGATTCGAATGCGGCAGCTGATTGACATAGTCGA
TCAACTGAAGAACTATGTGAATGATCTTGTGCCCGAGTTTTTGCCAGCCCCTGAAGACGTAG
AAACTAATTGTGAGTGGAGTGCCTTTTCCTGCTTTCAAAAGGCACAGCTGAAATCCGCCAAC
ACGGGCAATAACGAACGGATAATTAACGTATCCATTAAGAAGCTGAAGCGGAAGCCGCCCTC
AACCAATGCGGGACGGCGGCAAAAGCATCGCTTGACCTGTCCGTCATGCGACAGCTACGAGA
AAAAGCCCCCGAAGGAGTTCTTGGAACGCTTCAAGAGTCTCCTTCAGAAAATGATTCACCAG
CACCTGTCCTCACGGACGCACGGAAGCGAGGACAGTTGA
SEQ ID NO: 44-Exemplary IL15 Vector-nucleic acid sequence (signal peptide
underlined)
(SEQ ID NO: 44)
ATGCTCGAGTTTGGGCTGAGCTGGCTTTTTCTTGTGGCTATTTTAAAAGGTGTCCAGTGCTC
TAGACCCGAGGAGCCCCTGGTGGTGAAGGTGGAGGAGGGCGACAACGCCGTGCTGCAGTGCC
TGAAGGGCACCAGCGACGGCCCCACCCAGCAGCTGACCTGGAGCAGAGAGAGCCCCCTGAAG
CCCTTCCTGAAGCTGAGCCTGGGCCTGCCCGGCCTGGGCATCCACATGAGACCCCTGGCCAT
CTGGCTGTTCATCTTCAACGTGAGCCAGCAGATGGGCGGCTICTACCTGTGCCAGCCCGGCC
CCCCCAGCGAGAAGGCCTGGCAGCCCGGCTGGACCGTGAACGTGGAGGGCAGCGGCGAGCTG
TTCAGATGGAACGTGAGCGACCTGGGCGGCCTGGGCTGCGGCCTGAAGAACAGAAGCAGCGA
GGGCCCCAGCAGCCCCAGCGGCAAGCTGATGAGCCCCAAGCTGTACGTGTGGGCCAAGGACA
GACCCGAGATCTGGGAGGGCGAGCCCCCCTGCCTGCCCCCCAGAGACAGCCTGAACCAGAGC
CTGAGCCAGGACCTGACCATGGCCCCCGGCAGCACCCTGTGGCTGAGCTGCGGCGTGCCCCC
CGACAGCGTGAGCAGAGGCCCCCTGAGCTGGACCCACGTGCACCCCAAGGGCCCCAAGAGCC
TGCTGAGCCTGGAGCTGAAGGACGACAGACCCGCCAGAGACATGTGGGTGATGGAGACCGGC
CTGCTGCTGCCCAGAGCCACCGCCCAGGACGCCGGCAAGTACTACTGCCACAGAGGCAACCT
GACCATGAGCTTCCACCTGGAGATCACCGCCAGACCCGTGCTGTGGCACTGGCTGCTGAGAA
CCGGCGGCTGGAAGTCGCGAGAGCCCAAATCTCCTGACAAAACTCACACATGCCCACCGTGC
CCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGACAAAGCCG
CGGGAGGAGCAGTACAACAGCACGTACCGTGTGGTCAGCGTCCTCACCGTCCTGCACCAGGA
CTGGCTGAATGGCAAGGAGTACAAGTGCAAGGTCTCCAACAAAGCCCTCCCAGCCCCCATCG
AGAAAACCATCTCCAAAGCCAAAGGGCAGCCCCGAGAACCACAGGTGTACACCCTGCCCCCA
TCCCGGGATGAGCTGACCAAGAACCAGGTCAGCCTGACCTGCCTGGTCAAAGGCTTCTATCC
CAGCGACATCGCCGTGGAGTGGGAGAGCAATGGGCAACCGGAGAACAACTACAAGACCACGC
CTCCCGTGCTGGACTCCGACGGCTCCTTCTTCCTCTACAGCAAGCTCACCGTGGACAAGAGC
AGGTGGCAGCAGGGGAACGTCTTCTCATGCTCCGTGATGCATGAGGCTCTGCACAACCACTA
CACGCAGAAGAGCCTCTCCCTGTCTCCGGGTAAAGAAACAACCCCAAATAAAGGAAGTGGAA
CCACTTCAGGTACTACCCGTCTTCTATCTGGGCACACGTGTTTCACGTTGACAGGTTTGCTT
GGGACGCTAGTAACCATGGGCTTGCTGACTTTCGGACCGCAGTGTACTAATTATGCTCTCTT
GAAATTGGCTGGAGATGTTGAGAGCAATCCCGGGCCCATGCGCATGATGCGCATTAGCAAGC
CCCACCTGCGGAGCATCAGCATCCAGTGCTACCTGTGCCTGCTGCTGAACAGCCACTTCCTG
ACCGAGGCCGGCATCCACGTGTTCATCCTGGGCTGCTTCAGCGCCGGACTGCCCAAGACCGA
GGCCAACTGGGTGAACGTGATCAGCGACCTGAAGAAGATCGAGGACCTGATCCAGAGCATGC
ACATCGACGCCACCCTGTACACCGAGAGCGACGTGCACCCCAGCTGCAAGGTGACCGCCATG
AAGTGCTTTCTGCTGGAACTGCAGGTGATCAGCCTGGAAAGCGGCGACGCCAGCATCCACGA
CACCGTGGAGAACCTGATCATCCTGGCCAACAACAGCCTGAGCAGCAACGGCAACGTGACCG
AGAGCGGCTGCAAAGAGTGCGAGGAACTGGAAGAGAAGAACATCAAAGAGTTTCTGCAGAGC
TTCGTGCACATCGTGCAGATGTTCATCAACACCAGC
SEQ ID NO: 45-Exemplary 19ACLTBC mbIL21Fc Vector-nucleic acid sequence
(SEQ ID NO: 45)
ATGGATGCTGCTGCTCGTGACCTCTCTGCTGCTGTGCGAGCTGCCCCACCCTGCCTTTCTGC
TGATCCCTCAGGGCCAGGACCGGCACATGATCCGGATGAGACAGCTGATCGACATCGTGGAC
CAGCTGAAGAACTACGTGAACGACCTGGTGCCCGAGTTCCTGCCTGCCCCCGAGGACGTGGA
AACAAACTGCGAGTGGAGCGCCTTCAGCTGCTTCCAGAAGGCCCAGCTGAAAAGCGCCAACA
CCGGCAACAACGAGCGGATCATCAACGTGTCCATCAAGAAGCTGAAGCGGAAGCCCCCCAGC
ACCAACGCCGGAAGAAGGCAGAAGCACAGACTGACCTGCCCCAGCTGCGACAGCTACGAGAA
GAAGCCCCCTAAAGAGTTCCTGGAACGGTTCAAGAGCCTGCTGCAGAAGATGATCCACCAGC
ACCTGAGCAGCCGGACCCACGGCTCTGAGGACAGCGAGTCTAAGTACGGCCCTCCCTGCCCT
CCTTGTCCAGCCCCTGAATTTCTGGGCGGACCCAGCGTGTTCCTGTTCCCCCCAAAGCCCAA
GGACACCCTGATGATCAGCAGGACCCCCGAAGTGACCTGCGTGGTGGTGGATGTGTCCCAGG
AAGATCCCGAGGTGCAGTTTAATTGGTACGTGGACGGCGTGGAAGTGCACAACGCCAAGACC
AAGCCCAGAGAGGAACAGTTCAACAGCACCTACCGGGTGGTGTCCGTGCTGACCGTGCTGCA
CCAGGACTGGCTGAACGGCAAAGAGTACAAGTGCAAGGTGTCCAACAAGGGCCTGCCCAGCA
GCATCGAGAAAACCATCAGCAAGGCCAAGGGCCAGCCTCGGGAACCCCAGGTGTACACACTG
CCCCCTAGCCAGGAAGAGATGACCAAGAACCAGGTGTCCCTGACCTGTCTCGTGAAGGGCTT
CTACCCCAGCGACATTGCCGTGGAATGGGAGAGCAACGGCCAGCCCGAGAACAACTACAAGA
CCACCCCCCCTGTGCTGGACAGCGACGGCTCATTCTTCCTGTACAGCCGCCTGACCGTGGAC
AAGAGCAGATGGCAGGAAGGCAACGTGTTCAGCTGCAGCGTGATGCACGAGGCCCTGCACAA
CCACTACACCCAGAAGTCCCTGTCTCTGAGCCTGGGCAAGATGGCCCTGATCGTGCTGGGCG
GAGTGGCTGGCCTGCTGCTGTTTATCGGCCTGGGCATATTCTTTTGACGCGTCAATTGCGCG
TCATCATCGATCCGGATTAG
SEQ ID NO: 46-Exemplary 21AAMWXC IC9SP8mbIL21tmCD8 Vector-nucleic acid
sequence
(SEQ ID NO: 46)
ATGCTCGAGGGAGTGCAGGTGGAAACCATCTCCCCAGGCGACGGGCGCACCTTCCCCAAGCG
CGGCCAGACCTGCGTGGTGCACTACACCGGGATGCTTGAAGATGGAAAGAAAGTTGATTCCT
CCCGGGACAGAAACAAGCCCTTTAAGTTTATGCTAGGCAAGCAGGAGGTGATCCGAGGCTGG
GAAGAAGGGGTTGCCCAGATGAGTGTGGGTCAGAGAGCCAAACTGACTATATCTCCAGATTA
TGCCTATGGTGCCACTGGGCACCCAGGCATCATCCCACCACATGCCACTCTCGTCTTCGATG
TGGAGCTTCTAAAACTGGAATCTGGCGGTGGATCCGGAGTCGACGGATTTGGTGATGTCGGT
GCTCTTGAGAGTTTGAGGGGAAATGCAGATTTGGCTTACATCCTGAGCATGGAGCCCTGTGG
CCACTGCCTCATTATCAACAATGTGAACTTCTGCCGTGAGTCCGGGCTCCGCACCCGCACTG
GCTCCAACATCGACTGTGAGAAGTTGCGGCGTCGCTTCTCCTCGCTGCATTTCATGGTGGAG
GTGAAGGGCGACCTGACTGCCAAGAAAATGGTGCTGGCTTTGCTGGAGCTGGCGCAGCAGGA
CCACGGTGCTCTGGACTGCTGCGTGGTGGTCATTCTCTCTCACGGCTGTCAGGCCAGCCACC
TGCAGTTCCCAGGGGCTGTCTACGGCACAGATGGATGCCCTGTGTCGGTCGAGAAGATTGTG
AACATCTTCAATGGGACCAGCTGCCCCAGCCTGGGAGGGAAGCCCAAGCTCTTTTTCATCCA
GGCCTGTGGTGGGGAGCAGAAAGATCATGGGTTTGAGGTGGCCTCCACTTCCCCTGAAGACG
AGTCCCCTGGCAGTAACCCCGAGCCAGATGCCACCCCGTTCCAGGAAGGTTTGAGGACCTTC
GACCAGCTGGACGCCATATCTAGTTTGCCCACACCCAGTGACATCTTTGTGTCCTACTCTAC
TTTCCCAGGTTTTGTTTCCTGGAGGGACCCCAAGAGTGGCTCCTGGTACGTTGAGACCCTGG
ACGACATCTTTGAGCAGTGGGCTCACTCTGAAGACCTGCAGTCCCTCCTGCTTAGGGTCGCT
AATGCTGTTTCGGTGAAAGGGATTTATAAACAGATGCCTGGTTGCTTTAATTTCCTCCGGAA
AAAACTTTTCTTTAAAACATCAGCTTCGCGAGCCGAGGGCAGGGGAAGTCTTCTAACATGCG
GGGACGTGGAGGAAAATCCCGGGCCCATGGATGGCCTTACCAGTGACCGCCTTGCTCCTGCC
GCTGGCCTTGCTGCTCCACGCCGCCAGGCCGCCCATAAATCTTCCTCTCAAGGTCAGGACCG
CCATATGATTCGAATGCGGCAGCTGATTGACATAGTCGATCAACTGAAGAACTATGTGAATG
ATCTTGTGCCCGAGTTTTTGCCAGCCCCTGAAGACGTAGAAACTAATTGTGAGTGGAGTGCC
TTTTCCTGCTTTCAAAAGGCACAGCTGAAATCCGCCAACACGGGCAATAACGAACGGATAAT
TAACGTATCCATTAAGAAGCTGAAGCGGAAGCCGCCCTCAACCAATGCGGGACGGCGGCAAA
AGCATCGCTTGACCTGTCCGTCATGCGACAGCTACGAGAAAAAGCCCCCGAAGGAGTTCTTG
GAACGCTTCAAGAGTCTCCTTCAGAAAATGATTCACCAGCACCTGTCCTCACGGACGCACGG
AAGCGAGGACAGTACCACGACGCCAGCGCCGCGACCACCAACACCGGCGCCCACCATCGCGT
CGCAGCCCCTGTCCCTGCGCCCAGAGGCGTGCCGGCCAGCGGCGGGGGGCGCAGTGCACACG
AGGGGGCTGGACTTCGCCTGTGATATCTACATCTGGGCGCCCTTGGCCGGGACTTGTGGGGT
CCTTCTCCTGTCACTGGTTATCACCCTTTACTGCGGACCGCAGTGTACTAATTATGCTCTCT
TGAAATTGGCTGGAGATGTTGAGAGCAATCCCGGGCCTATGGGGATGCCACCTCCTCGCCTC
CTCTTCTTCCTCCTCTTCCTCACCCCTATGGAAGTCAGGCCCGAGGAACCTCTAGTGGTGAA
GGTGGAAGAGGGAGATAACGCTGTGCTGCAGTGCCTCAAGGGGACCTCAGATGGCCCCACTC
AGCAGCTGACCTGGTCTCGGGAGTCCCCGCTTAAACCCTTCTTAAAACTCAGCCTGGGGCTG
CCAGGCCTGGGAATCCACATGAGGCCCCTGGCCATCTGGCTTTTCATCTTCAACGTCTCTCA
ACAGATGGGGGGCTTCTACCTGTGCCAGCCGGGGCCCCCCTCTGAGAAGGCCTGGCAGCCTG
GCTGGACAGTCAATGTGGAGGGCAGCGGGGAGCTGTTCCGGTGGAATGTTTCGGACCTAGGT
GGCCTGGGCTGTGGCCTGAAGAACAGGTCCTCAGAGGGCCCCAGCTCCCCTTCCGGGAAGCT
CATGAGCCCCAAGCTGTATGTGTGGGCCAAAGACCGCCCTGAGATCTGGGAGGGAGAGCCTC
CGTGTCTCCCACCGAGGGACAGCCTGAACCAGAGCCTCAGCCAGGACCTCACTATGGCCCCT
GGCTCCACACTCTGGCTGTCCTGTGGGGTACCCCCTGACTCTGTGTCCAGGGGCCCCCTCTC
CTGGACCCATGTGCACCCCAAGGGGCCTAAGTCATTGCTGAGCCTAGAGCTGAAGGACGATC
GCCCGGCCAGAGATATGTGGGTAATGGAGACGGGTCTGTTGTTGCCCCGGGCCACAGCTCAA
GACGCTGGAAAGTATTATTGTCACCGTGGCAACCTGACCATGTCATTCCACCTGGAGATCAC
TGCTCGGCCAGTACTATGGCACTGGCTGCTGAGGACTGGTGGCTGGAAGGTCTCAGCTGTGA
CTTTGGCTTATCTGATCTTCTGCCTGTGTTCCCTTGTGGGCATTCTTCATCTTTAG
SEQ ID NO: 47-Exemplary 21AAMWYC IC9SP8mbIL21tm28 Vector-nucleic acid
sequence
(SEQ ID NO: 47)
ATGCTCGAGGGAGTGCAGGTGGAAACCATCTCCCCAGGCGACGGGCGCACCTTCCCCAAGCG
CGGCCAGACCTGCGTGGTGCACTACACCGGGATGCTTGAAGATGGAAAGAAAGTTGATTCCT
CCCGGGACAGAAACAAGCCCTTTAAGTTTATGCTAGGCAAGCAGGAGGTGATCCGAGGCTGG
GAAGAAGGGGTTGCCCAGATGAGTGTGGGTCAGAGAGCCAAACTGACTATATCTCCAGATTA
TGCCTATGGTGCCACTGGGCACCCAGGCATCATCCCACCACATGCCACTCTCGTCTTCGATG
TGGAGCTTCTAAAACTGGAATCTGGCGGTGGATCCGGAGTCGACGGATTTGGTGATGTCGGT
GCTCTTGAGAGTTTGAGGGGAAATGCAGATTTGGCTTACATCCTGAGCATGGAGCCCTGTGG
CCACTGCCTCATTATCAACAATGTGAACTTCTGCCGTGAGTCCGGGCTCCGCACCCGCACTG
GCTCCAACATCGACTGTGAGAAGTTGCGGCGTCGCTTCTCCTCGCTGCATTTCATGGTGGAG
GTGAAGGGCGACCTGACTGCCAAGAAAATGGTGCTGGCTTTGCTGGAGCTGGCGCAGCAGGA
CCACGGTGCTCTGGACTGCTGCGTGGTGGTCATTCTCTCTCACGGCTGTCAGGCCAGCCACC
TGCAGTTCCCAGGGGCTGTCTACGGCACAGATGGATGCCCTGTGTCGGTCGAGAAGATTGTG
AACATCTTCAATGGGACCAGCTGCCCCAGCCTGGGAGGGAAGCCCAAGCTCTTTTTCATCCA
GGCCTGTGGTGGGGAGCAGAAAGATCATGGGTTTGAGGTGGCCTCCACTTCCCCTGAAGACG
AGTCCCCTGGCAGTAACCCCGAGCCAGATGCCACCCCGTTCCAGGAAGGTTTGAGGACCTTC
GACCAGCTGGACGCCATATCTAGTTTGCCCACACCCAGTGACATCTTTGTGTCCTACTCTAC
TTTCCCAGGTTTTGTTTCCTGGAGGGACCCCAAGAGTGGCTCCTGGTACGTTGAGACCCTGG
ACGACATCTTTGAGCAGTGGGCTCACTCTGAAGACCTGCAGTCCCTCCTGCTTAGGGTCGCT
AATGCTGTTTCGGTGAAAGGGATTTATAAACAGATGCCTGGTTGCTTTAATTTCCTCCGGAA
AAAACTTTTCTTTAAAACATCAGCTTCGCGAGCCGAGGGCAGGGGAAGTCTTCTAACATGCG
GGGACGTGGAGGAAAATCCCGGGCCCATGGATGGCCTTACCAGTGACCGCCTTGCTCCTGCC
GCTGGCCTTGCTGCTCCACGCCGCCAGGCCGCCCATAAATCTTCCTCTCAAGGTCAGGACCG
CCATATGATTCGAATGCGGCAGCTGATTGACATAGTCGATCAACTGAAGAACTATGTGAATG
ATCTTGTGCCCGAGTTTTTGCCAGCCCCTGAAGACGTAGAAACTAATTGTGAGTGGAGTGCC
TTTTCCTGCTTTCAAAAGGCACAGCTGAAATCCGCCAACACGGGCAATAACGAACGGATAAT
TAACGTATCCATTAAGAAGCTGAAGCGGAAGCCGCCCTCAACCAATGCGGGACGGCGGCAAA
AGCATCGCTTGACCTGTCCGTCATGCGACAGCTACGAGAAAAAGCCCCCGAAGGAGTTCTTG
GAACGCTTCAAGAGTCTCCTTCAGAAAATGATTCACCAGCACCTGTCCTCACGGACGCACGG
AAGCGAGGACAGTTTTTGGGTGCTGGTGGTGGTTGGTGGAGTCCTGGCTTGCTATAGCTTGC
TAGTAACAGTGGCCTTTATTATTTTCTGGGTGCCCGGACCGCAGTGTACTAATTATGCTCTC
TTGAAATTGGCTGGAGATGTTGAGAGCAATCCCGGGCCTATGGGGATGCCACCTCCTCGCCT
CCTCTTCTTCCTCCTCTTCCTCACCCCTATGGAAGTCAGGCCCGAGGAACCTCTAGTGGTGA
AGGTGGAAGAGGGAGATAACGCTGTGCTGCAGTGCCTCAAGGGGACCTCAGATGGCCCCACT
CAGCAGCTGACCTGGTCTCGGGAGTCCCCGCTTAAACCCTTCTTAAAACTCAGCCTGGGGCT
GCCAGGCCTGGGAATCCACATGAGGCCCCTGGCCATCTGGCTTTTCATCTTCAACGTCTCTC
AACAGATGGGGGGCTTCTACCTGTGCCAGCCGGGGCCCCCCTCTGAGAAGGCCTGGCAGCCT
GGCTGGACAGTCAATGTGGAGGGCAGCGGGGAGCTGTTCCGGTGGAATGTTTCGGACCTAGG
TGGCCTGGGCTGTGGCCTGAAGAACAGGTCCTCAGAGGGCCCCAGCTCCCCTTCCGGGAAGC
TCATGAGCCCCAAGCTGTATGTGTGGGCCAAAGACCGCCCTGAGATCTGGGAGGGAGAGCCT
CCGTGTCTCCCACCGAGGGACAGCCTGAACCAGAGCCTCAGCCAGGACCTCACTATGGCCCC
TGGCTCCACACTCTGGCTGTCCTGTGGGGTACCCCCTGACTCTGTGTCCAGGGGCCCCCTCT
CCTGGACCCATGTGCACCCCAAGGGGCCTAAGTCATTGCTGAGCCTAGAGCTGAAGGACGAT
CGCCCGGCCAGAGATATGTGGGTAATGGAGACGGGTCTGTTGTTGCCCCGGGCCACAGCTCA
AGACGCTGGAAAGTATTATTGTCACCGTGGCAACCTGACCATGTCATTCCACCTGGAGATCA
CTGCTCGGCCAGTACTATGGCACTGGCTGCTGAGGACTGGTGGCTGGAAGGTCTCAGCTGTG
ACTTTGGCTTATCTGATCTTCTGCCTGTGTTCCCTTGTGGGCATTCTTCATCTTTAG
SEQ ID NO: 48-Exemplary CD8SPcoIL21CD8TMD Vector-nucleic acid sequence
(signal peptide underlined)
(SEQ ID NO: 48)
ATGGCCTTACCAGTGACCGCCTTGCTCCTGCCGCTGGCCTTGCTGCTCCACGCCGCCAGGCC
GATGAGGAGCAGTCCAGGCAATATGGAGCGGATAGTAATTTGTCTCATGGTAATATTCCTCG
GTACTCTGGTACATAAATCTTCCTCTCAAGGTCAGGACCGCCATATGATTCGAATGCGGCAG
CTGATTGACATAGTCGATCAACTGAAGAACTATGTGAATGATCTTGTGCCCGAGTTTTTGCC
AGCCCCTGAAGACGTAGAAACTAATTGTGAGTGGAGTGCCTTTTCCTGCTTTCAAAAGGCAC
AGCTGAAATCCGCCAACACGGGCAATAACGAACGGATAATTAACGTATCCATTAAGAAGCTG
AAGCGGAAGCCGCCCTCAACCAATGCGGGACGGCGGCAAAAGCATCGCTTGACCTGTCCGTC
ATGCGACAGCTACGAGAAAAAGCCCCCGAAGGAGTTCTTGGAACGCTTCAAGAGTCTCCTTC
AGAAAATGATTCACCAGCACCTGTCCTCACGGACGCACGGAAGCGAGGACAGTACCACGACG
CCAGCGCCGCGACCACCAACACCGGCGCCCACCATCGCGTCGCAGCCCCTGTCCCTGCGCCC
AGAGGCGTGCCGGCCAGCGGCGGGGGGCGCAGTGCACACGAGGGGGCTGGACTTCGCCTGTG
ATATCTACATCTGGGCGCCCTTGGCCGGGACTTGTGGGGTCCTTCTCCTGTCACTGGTTATC
ACCCTTTACTGCTCGCGAGCCGAGGGCAGGGGAAGTCTTCTAACATGCGGGGACGTGGAGGA
AAATCCCGGGCCtATGGggatgccacctcctcgcctcctcttcttcctcctcttcctcaccc
cTatggaagtcaggcccgaggaacctctagtggtgaaggtggaagagggagataacgctgtg
ctgcagtgcctcaaggggacctcagatggccccactcagcagctgacctggtctcgggagtc
cccgcttaaacccttcttaaaactcagcctggggctgccaggcctgggaatccacatgaggc
ccctggccatctggcttttcatcttcaacgtctctcaacagatggggggcttctacctgtgc
cagccggggcccccctctgagaaggcctggcagcctggctggacagtcaatgtggagggcag
cggggagctgttccggtggaatgtttcggacctaggtggcctgggctgtggcctgaagaaca
ggtcctcagagggccccagctccccttccgggaagctcatgagccccaagctgtatgtgtgg
gccaaagaccgccctgagatctgggagggagagcctccgtgtctcccaccgagggacagcct
gaaccagagcctcagccaggacctcacTatggcccctggctccacactctggctgtcctgtg
gggtaccccctgactctgtgtccaggggccccctctcctggacccatgtgcaccccaagggg
cctaagtcattgctgagcctagagctgaaggacgatcgcccggccagagatatgtgggtaat
ggagacgggtctgttgttgccccgggccacagctcaagacgctggaaagtattattgtcacc
gtggcaacctgaccatgtcattccacctggagatcactgctcggccagtactatggcactgg
ctgctgaggactggtggctggaaggtctcagctgtgactttggcttatctgatcttctgcct
gtgttcccttgtgggcattcttcatcttTAG
SEQ ID NO: 49-Exemplary CD8SPcoIL21CD28TMD Vector-nucleic acid sequence
(signal peptide underlined)
(SEQ ID NO: 49)
ATGGCCTTACCAGTGACCGCCTTGCTCCTGCCGCTGGCCTTGCTGCTCCACGCCGCCAGGCC
GATGAGGAGCAGTCCAGGCAATATGGAGCGGATAGTAATTTGTCTCATGGTAATATTCCTCG
GTACTCTGGTACATAAATCTTCCTCTCAAGGTCAGGACCGCCATATGATTCGAATGCGGCAG
CTGATTGACATAGTCGATCAACTGAAGAACTATGTGAATGATCTTGTGCCCGAGTTTTTGCC
AGCCCCTGAAGACGTAGAAACTAATTGTGAGTGGAGTGCCTTTTCCTGCTTTCAAAAGGCAC
AGCTGAAATCCGCCAACACGGGCAATAACGAACGGATAATTAACGTATCCATTAAGAAGCTG
AAGCGGAAGCCGCCCTCAACCAATGCGGGACGGCGGCAAAAGCATCGCTTGACCTGTCCGTC
ATGCGACAGCTACGAGAAAAAGCCCCCGAAGGAGTTCTTGGAACGCTTCAAGAGTCTCCTTC
AGAAAATGATTCACCAGCACCTGTCCTCACGGACGCACGGAAGCGAGGACAGTTTTTGGGTG
CTGGTGGTGGTTGGTGGAGTCCTGGCTTGCTATAGCTTGCTAGTAACAGTGGCCTTTATTAT
TTTCTGGGTGTCGCGAGCCGAGGGCAGGGGAAGTCTTCTAACATGCGGGGACGTGGAGGAAA
ATCCCGGGCCtATGGggatgccacctcctcgcctcctcttcttcctcctcttcctcaccccT
atggaagtcaggcccgaggaacctctagtggtgaaggtggaagagggagataacgctgtgct
gcagtgcctcaaggggacctcagatggccccactcagcagctgacctggtctcgggagtccc
cgcttaaacccttcttaaaactcagcctggggctgccaggcctgggaatccacatgaggccc
ctggccatctggcttttcatcttcaacgtctctcaacagatggggggcttctacctgtgcca
gccggggcccccctctgagaaggcctggcagcctggctggacagtcaatgtggagggcagcg
gggagctgttccggtggaatgtttcggacctaggtggcctgggctgtggcctgaagaacagg
tcctcagagggccccagctccccttccgggaagctcatgagccccaagctgtatgtgtgggc
caaagaccgccctgagatctgggagggagagcctccgtgtctcccaccgagggacagcctga
accagagcctcagccaggacctcacTatggcccctggctccacactctggctgtcctgtggg
gtaccccctgactctgtgtccaggggccccctctcctggacccatgtgcaccccaaggggcc
taagtcattgctgagcctagagctgaaggacgatcgcccggccagagatatgtgggtaatgg
agacgggtctgttgttgccccgggccacagctcaagacgctggaaagtattattgtcaccgt
ggcaacctgaccatgtcattccacctggagatcactgctcggccagtactatggcactggct
gctgaggactggtggctggaaggtctcagctgtgactttggcttatctgatcttctgcctgt
gttcccttgtgggcattcttcatcttTAG
SEQ ID NO: 50-Exemplary CEBPD-GFP Vector-nucleic acid sequence
ATGTCAGCAGCTCTTTTTTCTCTTGATGGTCCTGCCCGGGGCGCGCCATGGCCCGCTGAGCC
AGCTCCCTTTTACGAGCCAGGTAGAGCTGGAAAACCAGGGAGAGGGGCGGAGCCCGGGGCTC
TCGGGGAGCCTGGAGCTGCAGCTCCGGCCATGTATGACGATGAATCAGCAATCGATTTCTCA
GCGTACATTGATTCCATGGCCGCCGTTCCAACGTTGGAACTCTGTCATGATGAGCTTTTCGC
AGACCTCTTTAACTCTAATCACAAAGCTGGCGGCGCTGGTCCACTGGAACTTCTTCCTGGGG
GCCCCGCTCGGCCGCTCGGGCCCGGTCCCGCCGCCCCGAGATTGCTGAAAAGGGAACCGGAT
TGGGGAGATGGTGACGCGCCAGGAAGCTTGCTGCCTGCACAGGTAGCGGCATGCGCCCAGAC
CGTGGTGAGTCTGGCTGCAGCGGGCCAACCAACCCCTCCAACGTCACCGGAACCACCACGAT
CATCACCTAGACAAACACCAGCGCCCGGCCCGGCGCGCGAAAAGTCCGCAGGGAAAAGAGGG
CCTGACAGAGGCAGTCCGGAATACAGACAACGCCGGGAGAGGAACAATATAGCTGTGAGAAA
GAGTCGCGATAAGGCGAAACGGCGGAACCAAGAAATGCAGCAGAAGCTGGTAGAACTCAGCG
CAGAGAACGAGAAGCTCCATCAACGGGTTGAGCAACTGACTAGGGACCTCGCGGGCCTGAGA
CAATTTTTCAAGCAGCTGCCGAGTCCACCCTTTCTTCCCGCAGCGGGGACTGCCGACTGCCG
CGGCTCTGGGGAAGGACGCGGTAGCCTTCTGACTTGCGGCGATGTGGAGGAGAATCCTGGGC
CCatggtgagcaagggcgaggagctgttcaccggggtggtgcccatcctggtcgagctggac
ggcgacgtaaacggccacaagttcagcgtgtccggcgagggcgagggcgatgccacctacgg
caagctgaccctgaagttcatctgcaccaccggcaagctgcccgtgccctggcccaccctcg
tgaccaccctgacctacggcgtgcagtgcttcagccgctaccccgaccacatgaagcagcac
gacttcttcaagtccgccatgcccgaaggctacgtccaggagcgcaccatcttcttcaagga
cgacggcaactacaagacccgcgccgaggtgaagttcgagggcgacaccctggtgaaccgca
tcgagctgaagggcatcgacttcaaggaggacggcaacatcctggggcacaagctggagtac
aactacaacagccacaacgtctatatcatggccgacaagcagaagaacggcatcaaggtgaa
cttcaagatccgccacaacatcgaggacggcagcgtgcagctcgccgaccactaccagcaga
acacccccatcggcgacggccccgtgctgctgcccgacaaccactacctgagcacccagtcc
gccctgagcaaagaccccaacgagaagcgcgatcacatggtcctgctggagttcgtgaccgc
cgccgggatcactctcggcatggacgagctgtacaagTGA
SEQ ID NO: 51-Exemplary CEBPD-GFP with IL-15 Vector-nucleic acid sequence
ATGTCAGCAGCTCTTTTTTCTCTTGATGGTCCTGCCCGGGGCGCGCCATGGCCCGCTGAGCC
AGCTCCCTTTTACGAGCCAGGTAGAGCTGGAAAACCAGGGAGAGGGGCGGAGCCCGGGGCTC
TCGGGGAGCCTGGAGCTGCAGCTCCGGCCATGTATGACGATGAATCAGCAATCGATTTCTCA
GCGTACATTGATTCCATGGCCGCCGTTCCAACGTTGGAACTCTGTCATGATGAGCTTTTCGC
AGACCTCTTTAACTCTAATCACAAAGCTGGCGGCGCTGGTCCACTGGAACTTCTTCCTGGGG
GCCCCGCTCGGCCGCTCGGGCCCGGTCCCGCCGCCCCGAGATTGCTGAAAAGGGAACCGGAT
TGGGGAGATGGTGACGCGCCAGGAAGCTTGCTGCCTGCACAGGTAGCGGCATGCGCCCAGAC
CGTGGTGAGTCTGGCTGCAGCGGGCCAACCAACCCCTCCAACGTCACCGGAACCACCACGAT
CATCACCTAGACAAACACCAGCGCCCGGCCCGGCGCGCGAAAAGTCCGCAGGGAAAAGAGGG
CCTGACAGAGGCAGTCCGGAATACAGACAACGCCGGGAGAGGAACAATATAGCTGTGAGAAA
GAGTCGCGATAAGGCGAAACGGCGGAACCAAGAAATGCAGCAGAAGCTGGTAGAACTCAGCG
CAGAGAACGAGAAGCTCCATCAACGGGTTGAGCAACTGACTAGGGACCTCGCGGGCCTGAGA
CAATTTTTCAAGCAGCTGCCGAGTCCACCCTTTCTTCCCGCAGCGGGGACTGCCGACTGCCG
CGGCTCTGGGGAAGGACGCGGTAGCCTTCTGACTTGCGGCGATGTGGAGGAGAATCCTGGGC
CCatggtgagcaagggcgaggagctgttcaccggggtggtgcccatcctggtcgagctggac
ggcgacgtaaacggccacaagttcagcgtgtccggcgagggcgagggcgatgccacctacgg
caagctgaccctgaagttcatctgcaccaccggcaagctgcccgtgccctggcccaccctcg
tgaccaccctgacctacggcgtgcagtgcttcagccgctaccccgaccacatgaagcagcac
gacttcttcaagtccgccatgcccgaaggctacgtccaggagcgcaccatcttcttcaagga
cgacggcaactacaagacccgcgccgaggtgaagttcgagggcgacaccctggtgaaccgca
tcgagctgaagggcatcgacttcaaggaggacggcaacatcctggggcacaagctggagtac
aactacaacagccacaacgtctatatcatggccgacaagcagaagaacggcatcaaggtgaa
cttcaagatccgccacaacatcgaggacggcagcgtgcagctcgccgaccactaccagcaga
acacccccatcggcgacggccccgtgctgctgcccgacaaccactacctgagcacccagtcc
gccctgagcaaagaccccaacgagaagcgcgatcacatggtcctgctggagttcgtgaccgc
cgccgggatcactctcggcatggacgagctgtacaagCAGTGTACTAATTATGCTCTCTTGA
AATTGGCTGGAGATGTTGAGAGCAATCCCGGGCCCATGCGCATTAGCAAGCCCCACCTGCGG
AGCATCAGCATCCAGTGCTACCTGTGCCTGCTGCTGAACAGCCACTTCCTGACCGAGGCCGG
CATCCACGTGTTCATCCTGGGCTGCTTCAGCGCCGGACTGCCCAAGACCGAGGCCAACTGGG
TGAACGTGATCAGCGACCTGAAGAAGATCGAGGACCTGATCCAGAGCATGCACATCGACGCC
ACCCTGTACACCGAGAGCGACGTGCACCCCAGCTGCAAGGTGACCGCCATGAAGTGCTTTCT
GCTGGAACTGCAGGTGATCAGCCTGGAAAGCGGCGACGCCAGCATCCACGACACCGTGGAGA
ACCTGATCATCCTGGCCAACAACAGCCTGAGCAGCAACGGCAACGTGACCGAGAGCGGCTGC
AAAGAGTGCGAGGAACTGGAAGAGAAGAACATCAAAGAGTTTCTGCAGAGCTTCGTGCACAT
CGTGCAGATGTTCATCAACACCAGCTGA

### D. Antigens

The engineered NK cells of the disclosure can be utilized with monospecific, bispecific, or multi-specific antibodies that target one or more particular antigens.

Among the antigens targeted by the antibodies are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include NY-ESO, CD19, EBNA, CD123, HER2, CA-125, TRAIL/DR4, CD20, CD22, CD70, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGSS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGIB, SUNC1, and LRRN1. Examples of sequences for antigens are known in the art, for example, in the GENBANK® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRVIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### E. Suicide Gene

In particular embodiments, a suicide gene is utilized in conjunction with the NK cell therapy to control its use and allow for termination of the cell therapy at a desired event and/or time. The suicide gene is employed in transduced cells for the purpose of eliciting death for the transduced cells when needed. The cells of the present disclosure that have been modified to harbor one or more vectors encompassed by the disclosure that may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

In some cases, the cell therapy may be subject to utilization of one or more suicide genes of any kind when an individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Utilization of the suicide gene may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin.

Examples of suicide genes include engineered nonsecretable (including membrane bound) tumor necrosis factor (TNF)-alpha mutant polypeptides (see PCT/US19/62009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), EGFRv3, and Thymidine phosphorylase (TP), as examples.

### F. Knockout or Knockdown of Endogenous Genes

In certain embodiments, NK cells of the disclosure may include gene editing of the NK cells to remove 1, 2, 3, 4, 5, 6, 7, 8, 9, 10, or more endogenous genes in the NK cells. In some cases the gene editing occurs in NK cells expressing one or more heterologous transgenes (e.g., CEBPD, CEBPB, IL-21, IL-15, etc.), whereas in other cases the gene editing occurs in NK cells that do not express a heterologous transgene but that ultimately will express one or more heterologous transgenes, in at least some cases. In particular embodiments, the NK cells that are gene edited are expanded NK cells.

In particular cases, one or more endogenous genes of the NK cells are modified, such as disrupted in expression where the expression is reduced in part or in full. In specific cases, one or more genes are knocked down or knocked out using processes of the disclosure. In specific cases, multiple genes are knocked down or knocked out in the same step as processes of the disclosure. The genes that are edited in the NK cells may be of any kind, but in specific embodiments the genes are genes whose gene products inhibit activity and/or proliferation of NK cells. In specific cases the genes that are edited in the NK cells allow the NK cells to work more effectively in a tumor microenvironment. In specific cases, the genes are one or more of NKG2A, SIGLEC-7, LAG3, TIM3, CISH, FOXO1, TGFBR2, GR, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, CREB, CREM, ICER, and CD39. In specific embodiments, the TGFBR2 gene is knocked out or knocked down in the NK cells. In specific embodiments, the CISH gene is knocked out or knocked down in the NK cells. In specific embodiments, the CD38 gene is knocked out or knocked down in the NK cells. In specific embodiments, the Glucocorticoid receptor (GR) gene is knocked out or knocked down in the NK cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. Methods of utilizing a CRISPR system are well known in the art

### IV. ADMINISTRATION OF THERAPEUTIC COMPOSITIONS

In certain embodiments, engineered NK cells are administered to an individual in need thereof. In certain embodiments, the engineered NK cells are administered to an individual in need thereof in conjunction with one or more antibodies, including in such a way as to have the antibody be in proximity for interactions with the engineered NK cell. In some cases, the two components (engineered NK cells and antibodies) are administered separately to an individual, whereas in other cases the two components are complexed together prior to administration, such as in an ex vivo manner. In another embodiment, the NK cells express the antibodies. In some cases, the two components are not pre-complexed prior to administration, but are co-administered by any suitable route of administration, such as by co-infusion to the patient.

In certain embodiments, the present disclosure concern methods for the use of the compositions comprising NK cells and antibodies provided herein for treating or preventing a medical disease or disorder. The method includes administering to the subject a therapeutically effective amount of the engineered NK cells with the antibodies, thereby treating or preventing the disease in the subject, including reducing the risk of, reducing the severity of, and/or delaying the onset of the disease. In certain embodiments of the present disclosure, cancer or infection is treated by transfer of a composition comprising the NK cell population and corresponding antibodies. In at least some cases, because of their release of pro-inflammatory cytokines, NK cells may reverse the anti-inflammatory tumor microenvironment and increase adaptive immune responses by promoting differentiation, activation, and/or recruitment of accessory immune cell to sites of malignancy. In certain embodiments, a providing step may comprise culturing the engineered NK cells with antibody molecules for a specific duration of time (e.g., about 5 minutes to about 24 hours or more) and storing the NK cells and the antibody molecules for a period of time (e.g., about 1, 2, 3, 4, 5 days, or greater than 5 days) prior to infusion/administration.

Cancers for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition. Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed. Examples of therapies other than those of the present disclosure include surgery, chemotherapy, drug therapy, radiation, hormone therapy, immunotherapy (other than that of the present disclosure), or a combination thereof.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, a method comprises a subject receiving one or more doses of NK cells described herein, wherein at least one dose is at about 1×10⁶ to about 1×10¹⁰ cells, or any range derivable therein. In certain embodiments, at least one dose is at about 4×10⁵, 8×10⁵, 4×10⁶, 8×10⁶, 4×10⁷, 8×10⁷, 4×10⁸, 8×10⁸, 4×10⁹, 8×10⁹, 4×10¹⁰, or 8×10¹⁰, or any range derivable therein.

In certain embodiments, a composition comprising NK cells described herein comprises cells at a concentration of about 1×10⁶ to about 100×10⁶, or any range derivable therein. In certain embodiments, a composition comprising NK cells described herein comprises cells at a concentration of about 3×10⁶ to about 25×10⁶, or any range derivable therein.

In certain embodiments, the effective dose of the pharmaceutical composition is one which can provide a blood level of about 1 μM to 150 μM. In another embodiment, the effective dose provides a blood level of about 4 μM to 100 μM, or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 UM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain embodiments, engineered NK cells provided herein are utilized to treat a cancer. In certain embodiments, the cancer is glioblastoma. In certain embodiments, a glioblastoma has a TCGA annotated Mesenchymal subtype. In certain embodiments, a glioblastoma has a TCGA annotated Proneural subtype. In certain embodiments, a glioblastoma has an unmethylated MGMT status. In some embodiments, a glioblastoma has a methylated MGMT status. In some embodiments, a glioblastoma has an indeterminate MGMT status. In certain embodiments, a glioblastoma is EGFRVIII negative. In certain embodiments, a glioblastoma is EGFRVIII positive. In certain embodiments, a glioblastoma is a primary glioblastoma (a primary GBM status) In certain embodiments, a glioblastoma is a secondary glioblastoma (a secondary GBM status). In certain embodiments, a glioblastoma is classified as wild type Isocitrate dehydrogenase 1 (IDH-1) and/or wild type Isocitrate Dehydrogenase (NADP(+)) 2 (IDH-2). In certain embodiments, a glioblastoma is classified as mutant Isocitrate dehydrogenase 1 (IDH-1) and/or mutant Isocitrate Dehydrogenase (NADP(+)) 2 (IDH-2). In certain embodiments, a glioblastoma is Glioma CpG island methylator phenotype (GCIMP) negative. In certain embodiments, a glioblastoma is GCIMP positive.

In certain embodiments, provided herein are methods of immunizing a subject from cancer, comprising administration of engineered NK cells described herein. In certain embodiments, immunizing a subject from cancer comprises immunizing a subject from glioblastoma, and comprises administration of an NK cell engineered to transgenically express IL-21. In certain embodiments, immunizing a subject from cancer comprises immunizing a subject from glioblastoma, and comprises administration of an NK cell engineered to transgenically overexpress a CEBP family protein. In certain embodiments, immunizing a subject from cancer comprises immunizing a subject from glioblastoma, and comprises administration of an NK cell engineered to transgenically overexpress CEBPD.

### V. KITS

Certain aspects of the present disclosure also concern kits comprising compositions of the disclosure and/or compositions to implement methods of the disclosure. In particular embodiments, the kit comprises NK cells, fresh or frozen, and that may or may not have been pre-activated or expanded. The NK cells may or may not already be engineered to overexpress one or more CEBP proteins relative to a non-engineered NK cell and/or an NK cell engineered to express IL-15. In cases where the NK cells are not already engineered to overexpress one or more CEBP proteins, a kit may comprise reagents for corresponding transfection or transduction of the NK cells, including reagents such as vectors that express the component(s), primers for amplification of the component(s), and so forth. In some cases, the NK cells may or may not also express one or more heterologous proteins as defined herein, and when they do not, the kit may comprise vectors that express the heterologous protein(s), primers for amplification of the heterologous protein(s), and so forth.

In certain embodiments, a kit may be designed such that a subject may receive cells at a cell dose of about 1×10⁶ to about 1×10¹⁰ cells, or any range derivable therein. In certain embodiments, a kit may be designed such that a subject may receive cells at a cell dose of about 4×10⁵, 8×10⁵, 4×10⁶, 8×10⁶, 4×10⁷, 8×10⁷, 4×10⁸, 8×10⁸, 4×10⁹, 8×10⁹, 4×10¹⁰, or 8×10¹⁰, or any range derivable therein.

In certain embodiments, a kit comprises compositions of cells aliquoted at a cell concentration of about 1×10⁶ to about 100×10⁶, or any range derivable therein. In certain embodiments, a kit comprises compositions of cells aliquoted at a cell concentration of about 3×10⁶ to about 25×10⁶, or any range derivable therein.

Kits may comprise components which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

### VI. REFERENCES

All patents and publications mentioned in the specification are indicative of the level of those skilled in the art to which the inventions pertain. All patents and publications are herein incorporated by reference to the same extent as if each individual publication was specifically and individually indicated to be incorporated by reference.
1. Lim, M., Xia, Y., Bettegowda, C., and Weller, M. (2018). Current state of immunotherapy for glioblastoma. Nat Rev Clin Oncol 15, 422-442. 10.1038/s41571-018-0003-5.
2. Mccutcheon, I. E., and Preul, M. C. (2021). Historical Perspective on Surgery and Survival with Glioblastoma: How Far Have We Come? World Neurosurg 149, 148-168. 10.1016/j.wneu.2021.02.047.
3. Lathia, J. D., Mack, S. C., Mulkearns-Hubert, E. E., Valentim, C. L., and Rich, J. N. (2015). Cancer stem cells in glioblastoma. Genes Dev 29, 1203-1217. 10.1101/gad.261982.115.
4. Gras Navarro, A., Bjorklund, A. T., and Chekenya, M. (2015). Therapeutic potential and challenges of natural killer cells in treatment of solid tumors. Front Immunol 6, 202. 10.3389/fimmu.2015.00202.
5. Shaim H, H. S. M., Basar R, Wang F, Daher M et al (2020). Inhibition of the av integrin-TGF-b axis improves natural killer cell function against glioblastoma stem cells. bioRxiv. https://doi.org/10.1101/2020.03.30.016667.
6. Huntington, N. D., Puthalakath, H., Gunn, P., Naik, E., Michalak, E. M., Smyth, M. J., Tabarias, H., Degli-Esposti, M. A., Dewson, G., Willis, S. N., et al. (2007). Interleukin 15-mediated survival of natural killer cells is determined by interactions among Bim, Noxa and Mcl-1. Nature Immunology 8, 856-863. 10.1038/ni1487.
7. Ma, S., Caligiuri, M. A., and Yu, J. (2022). Harnessing IL-15 signaling to potentiate NK cell-mediated cancer immunotherapy. Trends Immunol 43, 833-847. 10.1016/j.it.2022.08.004.
8 Imamura, M., Shook, D., Kamiya, T., Shimasaki, N., Chai, S. M., Coustan-Smith, E., Imai, C., and Campana, D. (2014). Autonomous growth and increased cytotoxicity of natural killer cells expressing membrane-bound interleukin-15. Blood 124, 1081-1088. 10.1182/blood-2014-02-556837.
9. Li, Y., Basar, R., Wang, G., Liu, E., Moyes, J. S., Li, L., Kerbauy, L. N., Uprety, N., Fathi, M., Rezvan, A., et al. (2022). KIR-based inhibitory CARs overcome CAR-NK cell trogocytosis-mediated fratricide and tumor escape. Nature Medicine 28, 2133-2144. 10.1038/s41591-022-02003-x.
10. Liu, E., Marin, D., Banerjee, P., Macapinlac, H. A., Thompson, P., Basar, R., Nassif Kerbauy, L., Overman, B., Thall, P., Kaplan, M., et al. (2020). Use of CAR-Transduced Natural Killer Cells in CD19-Positive Lymphoid Tumors. N Engl J Med 382, 545-553. 10.1056/NEJMoa1910607.
11. Liu, E., Tong, Y., Dotti, G., Shaim, H., Savoldo, B., Mukherjee, M., Orange, J., Wan, X., Lu, X., Reynolds, A., et al. (2017). Cord blood NK cells engineered to express IL-15 and a CD19-targeted CAR show long-term persistence and potent antitumor activity. Leukemia. 10.1038/leu.2017.226.
12. Marin, D., Li, Y., Basar, R., Rafei, H., Daher, M., Dou, J., Mohanty, V., Dede, M., Nieto, Y., Uprety, N., et al. (2024). Safety, efficacy and determinants of response of allogeneic CD19-specific CAR-NK cells in CD19+ B cell tumors: a phase 1/2 trial. Nature Medicine. 10.1038/s41591-023-02785-8.
13. Brown, C. E., Alizadeh, D., Starr, R., Weng, L., Wagner, J. R., Naranjo, A., Ostberg, J. R., Blanchard, M. S., Kilpatrick, J., Simpson, J., et al. (2016). Regression of Glioblastoma after Chimeric Antigen Receptor T-Cell Therapy. N Engl J Med 375, 2561-2569. 10.1056/NEJMoa1610497.
14. Brown, C. E., Badie, B., Barish, M. E., Weng, L., Ostberg, J. R., Chang, W. C., Naranjo, A., Starr, R., Wagner, J., Wright, C., et al. (2015). Bioactivity and Safety of IL13Rα2-Redirected Chimeric Antigen Receptor CD8+ T Cells in Patients with Recurrent Glioblastoma. Clin Cancer Res 21, 4062-4072. 10.1158/1078-0432.Ccr-15-0428.
15. Burger, M. C., Forster, M.-T., Romanski, A., Straßheimer, F., Macas, J., Zeiner, P. S., Steidl, E., Herkt, S., Weber, K. J., Schupp, J., et al. (2023). Intracranial injection of natural killer cells engineered with a HER2-targeted chimeric antigen receptor in patients with recurrent glioblastoma. Neuro-Oncology 25, 2058-2071. 10.1093/neuonc/noad087.
16. Loschinski, R., Bottcher, M., Stoll, A., Bruns, H., Mackensen, A., and Mougiakakos, D. (2018). IL-21 modulates memory and exhaustion phenotype of T-cells in a fatty acid oxidation-dependent manner. Oncotarget 9, 13125-13138. 10.18632/oncotarget.24442.
17. Heinze, A., Grebe, B., Bremm, M., Huenecke, S., Munir, T. A., Graafen, L., Frueh, J. T., Merker, M., Rettinger, E., Soerensen, J., et al. (2019). The Synergistic Use of IL-15 and IL-21 for the Generation of NK Cells From CD3/CD19-Depleted Grafts Improves Their ex vivo Expansion and Cytotoxic Potential Against Neuroblastoma: Perspective for Optimized Immunotherapy Post Haploidentical Stem Cell Transplantation. Front Immunol 10, 2816. 10.3389/fimmu.2019.02816.
18. Ojo, E. O., Sharma, A. A., Liu, R., Moreton, S., Checkley-Luttge, M. A., Gupta, K., Lee, G., Lee, D. A., Otegbeye, F., Sekaly, R. P., et al. (2019). Membrane bound IL-21 based NK cell feeder cells drive robust expansion and metabolic activation of NK cells. Sci Rep 9, 14916. 10.1038/s41598-019-51287-6.
19. Parrish-Novak, J., Dillon, S. R., Nelson, A., Hammond, A., Sprecher, C., Gross, J. A., Johnston, J., Madden, K., Xu, W., West, J., et al. (2000). Interleukin 21 and its receptor are involved in NK cell expansion and regulation of lymphocyte function. Nature 408, 57-63. 10.1038/35040504.
20. Bhatia, S., Curti, B., Ernstoff, M. S., Gordon, M., Heath, E. I., Miller, W. H., Puzanov, I., Quinn, D. I., Flaig, T. W., VanVeldhuizen, P., et al. (2014). Recombinant interleukin-21 plus sorafenib for metastatic renal cell carcinoma: a phase 1/2 study. Journal for ImmunoTherapy of Cancer 2, 2. 10.1186/2051-1426-2-2.
21. Petrella, T. M., Tozer, R., Belanger, K., Savage, K. J., Wong, R., Smylie, M., Kamel-Reid, S., Tron, V., Chen, B. E., Hunder, N. N., et al. (2012). Interleukin-21 has activity in patients with metastatic melanoma: a phase II study. J Clin Oncol 30, 3396-3401. 10.1200/JCO.2011.40.0655.
22. Bell, M., and Gottschalk, S. (2021). Engineered Cytokine Signaling to Improve CAR T Cell Effector Function. Front Immunol 12, 684642. 10.3389/fimmu.2021.684642. 23. Lottaz, C., Beier, D., Meyer, K., Kumar, P., Hermann, A., Schwarz, J., Junker, M., Oefner, P. J., Bogdahn, U., Wischhusen, J., et al. (2010). Transcriptional profiles of CD133+ and CD133-glioblastoma-derived cancer stem cell lines suggest different cells of origin. Cancer Res 70, 2030-2040. 10.1158/0008-5472.Can-09-1707.
24. Shaim, H., Shanley, M., Basar, R., Daher, M., Gumin, J., Zamler, D. B., Uprety, N., Wang, F., Huang, Y., Gabrusiewicz, K., et al. (2021). Targeting the alphav integrin/TGF-beta axis improves natural killer cell function against glioblastoma stem cells. J Clin Invest 131. 10.1172/JCI142116.
25. Zhu, H., Blum, R. H., Bernareggi, D., Ask, E. H., Wu, Z., Hoel, H. J., Meng, Z., Wu, C., Guan, K. L., Malmberg, K. J., and Kaufman, D. S. (2020). Metabolic Reprograming via Deletion of CISH in Human iPSC-Derived NK Cells Promotes In Vivo Persistence and Enhances Anti-tumor Activity. Cell Stem Cell 27, 224-237 e226. 10.1016/j.stem.2020.05.008.
26. Menk, A. V., Scharping, N. E., Rivadeneira, D. B., Calderon, M. J., Watson, M. J., Dunstane, D., Watkins, S. C., and Delgoffe, G. M. (2018). 4-1BB costimulation induces T cell mitochondrial function and biogenesis enabling cancer immunotherapeutic responses. Journal of Experimental Medicine 215, 1091-1100. 10.1084/jem.20171068.
27. Reddaway, J., Richardson, P. E., Bevan, R. J., Stoneman, J., and Palombo, M. (2023). Microglial morphometric analysis: so many options, so little consistency. Front Neuroinform 17, 1211188. 10.3389/fninf.2023.1211188.
28. Eisenman, J., Ahdieh, M., Beers, C., Brasel, K., Kennedy, M. K., Le, T., Bonnert, T. P., Paxton, R. J., and Park, L. S. (2002). Interleukin-15 interactions with interleukin-15 receptor complexes: characterization and species specificity. Cytokine 20, 121-129. 10.1006/cyto.2002.1989.
29. Duncan, G. S., Mittrücker, H. W., Kägi, D., Matsuyama, T., and Mak, T. W. (1996). The transcription factor interferon regulatory factor-1 is essential for natural killer cell function in vivo. J Exp Med 184, 2043-2048. 10.1084/jem.184.5.2043.
30. Gordon, S. M., Chaix, J., Rupp, L. J., Wu, J., Madera, S., Sun, J. C., Lindsten, T., and Reiner, S. L. (2012). The transcription factors T-bet and Eomes control key checkpoints of natural killer cell maturation. Immunity 36, 55-67. 10.1016/j.immuni.2011.11.016.
31. Geary, C. D., Krishna, C., Lau, C. M., Adams, N. M., Gearty, S. V., Pritykin, Y., Thomsen, A. R., Leslie, C. S., and Sun, J. C. (2018). Non-redundant ISGF3 Components Promote NK Cell Survival in an Auto-regulatory Manner during Viral Infection. Cell Rep 24, 1949-1957 e1946. 10.1016/j.celrep.2018.07.060.
32. Larsen, S. B., Cowley, C. J., Sajjath, S. M., Barrows, D., Yang, Y., Carroll, T. S., and Fuchs, E. (2021). Establishment, maintenance, and recall of inflammatory memory. Cell Stem Cell 28, 1758-1774 e1758. 10.1016/j.stem.2021.07.001.
33. de Laval, B., Maurizio, J., Kandalla, P. K., Brisou, G., Simonnet, L., Huber, C., Gimenez, G., Matcovitch-Natan, O., Reinhardt, S., David, E., et al. (2020). C/EBPbeta-Dependent Epigenetic Memory Induces Trained Immunity in Hematopoietic Stem Cells. Cell Stem Cell 26, 657-674 e658. 10.1016/j.stem.2020.01.017.
34. Rapp, M., Lau, C. M., Adams, N. M., Weizman, O. E., O'Sullivan, T. E., Geary, C. D., and Sun, J. C. (2017). Core-binding factor beta and Runx transcription factors promote adaptive natural killer cell responses. Sci Immunol 2. 10.1126/sciimmunol.aan3796. 35. Bromley, S. K., Akbaba, H., Mani, V., Mora-Buch, R., Chasse, A. Y., Sama, A., and Luster, A. D. (2020). CD49a Regulates Cutaneous Resident Memory CD8 (+) T Cell Persistence and Response. Cell Rep 32, 108085. 10.1016/j.celrep.2020.108085.
36. Gunturi, A., Berg, R. E., and Forman, J. (2004). The role of CD94/NKG2 in innate and adaptive immunity. Immunol Res 30, 29-34. 10.1385/IR: 30:1:029.
37. Weng, N. P., Araki, Y., and Subedi, K. (2012). The molecular basis of the memory T cell response: differential gene expression and its epigenetic regulation. Nat Rev Immunol 12, 306-315. 10.1038/nri3173.
38. Dan, L., Liu, L., Sun, Y., Song, J., Yin, Q., Zhang, G., Qi, F., Hu, Z., Yang, Z., Zhou, Z., et al. (2020). The phosphatase PAC1 acts as a T cell suppressor and attenuates host antitumor immunity. Nat Immunol 21, 287-297. 10.1038/s41590-019-0577-9.
39. Delconte, R. B., Kolesnik, T. B., Dagley, L. F., Rautela, J., Shi, W., Putz, E. M., Stannard, K., Zhang, J. G., Teh, C., Firth, M., et al. (2016). CIS is a potent checkpoint in NK cell-mediated tumor immunity. Nat Immunol 17, 816-824. 10.1038/ni.3470.
40. Tripathi, P., Koss, B., Opferman, J. T., and Hildeman, D. A. (2013). Mcl-1 antagonizes Bax/Bak to promote effector CD4 (+) and CD8 (+) T-cell responses. Cell Death Differ 20, 998-1007. 10.1038/cdd.2013.25.
41. Araki, K., Turner, A. P., Shaffer, V. O., Gangappa, S., Keller, S. A., Bachmann, M. F., Larsen, C. P., and Ahmed, R. (2009). mTOR regulates memory CD8 T-cell differentiation. Nature 460, 108-112. 10.1038/nature08155.
42. Hasan, F., Chiu, Y., Shaw, R. M., Wang, J., and Yee, C. (2021). Hypoxia acts as an environmental cue for the human tissue-resident memory T cell differentiation program. JCI Insight 6. 10.1172/jci.insight. 138970.
43. Kuo, C. T., Veselits, M. L., and Leiden, J. M. (1997). LKLF: A transcriptional regulator of single-positive T cell quiescence and survival. Science 277, 1986-1990. 10.1126/science.277.5334.1986.
44. O'Sullivan, T. E., Johnson, L. R., Kang, H. H., and Sun, J. C. (2015). BNIP3- and BNIP3L-Mediated Mitophagy Promotes the Generation of Natural Killer Cell Memory. Immunity 43, 331-342. 10.1016/j.immuni.2015.07.012.
45. Schober, S. L., Kuo, C. T., Schluns, K. S., Lefrancois, L., Leiden, J. M., and Jameson, S. C. (1999). Expression of the transcription factor lung Kruppel-like factor is regulated by cytokines and correlates with survival of memory T cells in vitro and in vivo. J Immunol 163, 3662-3667.
46. Tu, W. J., Hardy, K., Sutton, C. R., McCuaig, R., Li, J., Dunn, J., Tan, A., Brezar, V., Morris, M., Denyer, G., et al. (2017). Priming of transcriptional memory responses via the chromatin accessibility landscape in T cells. Sci Rep 7, 44825. 10.1038/srep44825.
47. Pantaleon Garcia, J., Kulkarni, V. V., Reese, T. C., Wali, S., Wase, S. J., Zhang, J., Singh, R., Caetano, M. S., Kadara, H., Moghaddam, S. J., et al. (2022). OBIF: an omics-based interaction framework to reveal molecular drivers of synergy. NAR Genom Bioinform 4, lqac028. 10.1093/nargab/lqac028.
48. Ramji, D. P., and Foka, P. (2002). CCAAT/enhancer-binding proteins: structure, function and regulation. Biochem J 365, 561-575. 10.1042/bj20020508.
49. Tolomeo, M., and Grimaudo, S. (2020). The “Janus” Role of C/EBPs Family Members in Cancer Progression. Int J Mol Sci 21. 10.3390/ijms21124308.
50. Kaisho, T., Tsutsui, H., Tanaka, T., Tsujimura, T., Takeda, K., Kawai, T., Yoshida, N., Nakanishi, K., and Akira, S. (1999). Impairment of natural killer cytotoxic activity and interferon gamma production in CCAAT/enhancer binding protein gamma-deficient mice. J Exp Med 190, 1573-1582. 10.1084/jem. 190.11.1573.
51. Leonard, W. J., and Wan, C. K. (2016). IL-21 Signaling in Immunity. F1000Res 5. 10.12688/f1000research.7634.1.
52. Hart, G. T., Hogquist, K. A., and Jameson, S. C. (2012). Kruppel-like factors in lymphocyte biology. J Immunol 188, 521-526. 10.4049/jimmunol.1101530.
53. Rabacal, W., Pabbisetty, S. K., Hoek, K. L., Cendron, D., Guo, Y., Maseda, D., and Sebzda, E. (2016). Transcription factor KLF2 regulates homeostatic NK cell proliferation and survival. Proc Natl Acad Sci USA 113, 5370-5375. 10.1073/pnas. 1521491113.
54. Myers, J. A., Schirm, D., Bendzick, L., Hopps, R., Selleck, C., Hinderlie, P., Felices, M., and Miller, J. S. (2022). Balanced engagement of activating and inhibitory receptors mitigates human NK cell exhaustion. JCI Insight 7. 10.1172/jci.insight. 150079.
55. Li, M., Li, Z., Yao, Y., Jin, W. N., Wood, K., Liu, Q., Shi, F. D., and Hao, J. (2017). Astrocyte-derived interleukin-15 exacerbates ischemic brain injury via propagation of cellular immunity. Proc Natl Acad Sci USA 114, E396-E405. 10.1073/pnas. 1612930114.
56. Shi, S. X., Li, Y. J., Shi, K., Wood, K., Ducruet, A. F., and Liu, Q. (2020). IL (Interleukin)-15 Bridges Astrocyte-Microglia Crosstalk and Exacerbates Brain Injury Following Intracerebral Hemorrhage. Stroke 51, 967-974. 10.1161/STROKEAHA.119.028638.
57. Cui, A., Huang, T., Li, S., Ma, A., Pérez, J. L., Sander, C., Keskin, D. B., Wu, C. J., Fraenkel, E., and Hacohen, N. (2024). Dictionary of immune responses to cytokines at single-cell resolution. Nature 625, 377-384. 10.1038/s41586-023-06816-9.
58. Lau, C. M., Adams, N. M., Geary, C. D., Weizman, O. E., Rapp, M., Pritykin, Y., Leslie, C. S., and Sun, J. C. (2018). Epigenetic control of innate and adaptive immune memory. Nat Immunol 19, 963-972. 10.1038/s41590-018-0176-1.
59. Rückert, T., Lareau, C. A., Mashreghi, M. F., Ludwig, L. S., and Romagnani, C. (2022). Clonal expansion and epigenetic inheritance of long-lasting NK cell memory. Nat Immunol 23, 1551-1563. 10.1038/s41590-022-01327-7.
60. Wendt, K., Wilk, E., Buyny, S., Schmidt, R. E., and Jacobs, R. (2007). Interleukin-21 differentially affects human natural killer cell subsets. Immunology 122, 486-495. 10.1111/j.1365-2567.2007.02675.x.
61. Zhang, Y., Sif, S., and DeWille, J. (2007). The mouse C/EBPdelta gene promoter is regulated by STAT3 and Sp1 transcriptional activators, chromatin remodeling and c-Myc repression. J Cell Biochem 102, 1256-1270. 10.1002/jcb.21356.
62. Liu, E., Ang, S. O. T., Kerbauy, L., Basar, R., Kaur, I., Kaplan, M., Li, L., Tong, Y., Daher, M., Ensley, E. L., et al. (2021). GMP-Compliant Universal Antigen Presenting Cells (uAPC) Promote the Metabolic Fitness and Antitumor Activity of Armored Cord Blood CAR-NK Cells. Front Immunol 12, 626098. 10.3389/fimmu.2021.626098.
63. Stuart, T., Srivastava, A., Madad, S., Lareau, C. A., and Satija, R. (2021). Single-cell chromatin state analysis with Signac. Nat Methods 18, 1333-1341. 10.1038/s41592-021-01282-5.
64. Jain, A. K., Xi, Y., McCarthy, R., Allton, K., Akdemir, K. C., Patel, L. R., Aronow, B., Lin, C., Li, W., Yang, L., and Barton, M. C. (2016). LncPRESS1 Is a p53-Regulated LncRNA that Safeguards Pluripotency by Disrupting SIRT6-Mediated De-acetylation of Histone H3K56. Mol Cell 64, 967-981. 10.1016/j.molcel.2016.10.039.
65. Wang, W. J., Li, C. F., Chu, Y. Y., Wang, Y. H., Hour, T. C., Yen, C. J., Chang, W. C., and Wang, J. M. (2017). Inhibition of the EGFR/STAT3/CEBPD Axis Reverses Cisplatin Cross-resistance with Paclitaxel in the Urothelial Carcinoma of the Urinary Bladder. Clin Cancer Res 23, 503-513. 10.1158/1078-0432.CCR-15-1169.
66. Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589. 10.1016/j.molcel.2010.05.004.
67. Robinson, J. T., Thorvaldsdottir, H., Winckler, W., Guttman, M., Lander, E. S., Getz, G., and Mesirov, J. P. (2011). Integrative genomics viewer. Nat Biotechnol 29, 24-26. 10.1038/nbt. 1754.

### VII. EXAMPLES

The following examples are included to demonstrate preferred embodiments of the inventions. It should be appreciated by those of skill in the art that the techniques disclosed in the examples which follow represent techniques discovered by the inventor to function well in the practice of the inventions, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the inventions.

### Materials and Methods

Unless otherwise stated, assays, assay materials, and experiments described in the following examples were performed as described herein.

### Glioblastoma Stem Cells (GSCs)

GSCs were obtained from primary human GBM samples as previously described 24. The GSCs were cultured in Dulbecco's Modified Eagle Medium: Nutrient Mixture F-12 (DMEM/F12) supplemented with 20 ng/ml of epidermal growth factor and basic fibroblast growth factor (all from PEPROTech®, Cranbury, NJ), B27 supplement (1:50; Invitrogen®, Carlsbad, CA), 100 units of penicillin and 100 mg/ml streptomycin (Thermo Fisher Scientific®, Waltham, MA) and passaged every 4-5 days. All GSC cell lines in this study were generated at MD Anderson Cancer Center.

### NK Cell Expansion

NK cells were purified from umbilical cord blood (CB) using an NK cell isolation kit (Miltenyi Biotec, Inc., San Diego, CA). NK cells were stimulated on day 0 with irradiated (100 Gy) K562-based feeder cells engineered to express 4-1BB ligand and CD137 ligand (referred to as universal antigen-presenting cell [uAPC]) 62 at a 2:1 feeder cell:NK ratio and recombinant human IL-2 (Proleukin, 200 IU/ml; Chiron, Emeryville, CA), in complete CellGenix® GMP SCGM Stem Cell Growth Medium (CellGenix® GmbH, Freiburg, Germany). For IL-21 priming, NT NK cells were cultured for two days with 3 ng/ml of exogenous human IL-21 (PEPROTech®, Cranbury, NJ).

### Retrovirus Transfection and Transduction

Retroviral vectors encoding human IL-15 or IL-21 were used for 293T cell transfection and CB-derived NK cell transduction. NK cells were transduced on day +5 in human fibronectin-coated plates (Clontech® Laboratories, Inc., Mountain View, CA). Non-transfected 293T or non-transduced (NT) NK cells were used as negative controls. To generate NK cells overexpressing CEBPD (CEBPD OE, aka CEPBD KI), CEBPD cDNA (SEQ ID NO: 20) was cloned into pSFG retroviral expression vector for retrovirus production.

### Flow Cytometry

CB-NK cells were stained using Live/Dead-aqua (Thermo Fisher Scientific®) and anti-human CD56 (clone HCD56) antibodies, and anti-human CD3 (clone SK7) antibodies (BioLegend®, San Diego, CA). Anti-human IgG1 antibody (Jackson ImmunoResearch) was used to identify the transfection and transduction efficiency of 293T and NK cells, respectively. For cell viability, annexin V (Thermo Fisher Scientific®) and Live/Dead was used following manufacturer's instructions. For CEBPD staining, cells were first stained with surface antibodies for 20 minutes, washed, fixed/permeabilized and stained with anti-human CEBPD antibody (clone C6, Santa Cruz Biotechnology, Dallas, TX) for 30 minutes at room temperature. For mouse tissue cell suspension, cells were blocked using human Fc receptor-blocking solution (BD Biosciences, San Jose, CA) for 10 minutes followed by surface staining with anti-human CD45 (clone HI30, BioLegend®) and anti-mouse CD45 (clone 30-F11, BioLegend®), anti-human CD56, anti-human CD-16 (clone 3G-8, BioLegend®), and anti-human CD3. All the cells were acquired and analyzed using a BD LSR Fortessa™ instrument. Data was analyzed using FlowJo software version 10.7.1.

### Suppression Assay

NK cells were co-cultured either alone or with GSCs at a 1:1 ratio for 48 hours at 37° C. before performing the functional assays. After co-culture, NK cells were selected using human NK cell Isolation Kit and human CD105 microbeads from Miltenyi Biotec, Inc., (San Diego, CA, USA), following the manufacturer's instructions.

### Real Time NK Cell Cytotoxicity Assay

NK cells were co-cultured at a 1:1 ratio with K562 or GSC targets labeled with CellTracker® Deep Red Dye (Thermo Fisher Scientific®, Waltham, MA) or previously transduced with M-cherry expressing retroviral vector. For rechallenge experiments, fresh GSC targets previously transduced with mCherry were added to the plate every 2-3 days. Apoptosis was detected using the CellEvent™ Caspase-3/7 Green Detection Reagent (Thermo Fisher Scientific®). Frames were captured over a period of 24 hours at one-hour or two-hour intervals from four separate 1.75×1.29 mm² regions per well with a 10× objective using IncuCyte® S3 live-cell analysis system (Sartorius®, Goettingen, Germany). Values from all four regions of each well were pooled and averaged across all three replicates. Results were expressed graphically as percent cytotoxicity by calculating the ratio of red and green overlapping signals (counts per image) divided by the red signal (counts per image). The number of tumor cells (red signal) were plotted over time to follow tumor growth or elimination by NK cells (tumor cell index).

### 3D Real Time Killing Assay

GSC spheroids were made by placing 10,000 single cells in 100 μl CellGenix® GMP SCGM Stem Cell Growth Medium (CellGenix®) in a 96-well clear round bottom ultra-low attachment microplate (Corning®, Glendale, AR). After 48 hours the spheroids were formed (confirmed by microscopy) and 20,000 NK cells were added to the spheroid-containing well. Frames were captured with a 10× objective at two-hour intervals over a period of six days. Red signal (tumor) was quantified using IncuCyte® S3 live-cell analysis system (Sartorius®).

### Single Cell Secretome Assay (IsoPlexis®)

IL-15 NK, IL-21 NK, and NT-NK cells were labeled with a fluorescent dye (IsoPlexis® stain cell membrane 405), co-cultured with GSC20 cells for 2 hours, and then purified using positive selection with CD56⁺ microbeads (Miltenyi Biotec, Waltham, MA). A total of 3×10⁴ NK cells were loaded onto individual Isocode™ chips according to the manufacturer's protocol (IsoPlexis®, Branford, CT) and subjected to single cell 32-plex cytokine secretome profiling using a fully validated panel of cytokines on the IsoPlexis® single-cell platform. The IsoSpeak™ software was used to quantify the number of polyfunctional NK cells by measuring the percentage of cells in each sample secreting multiple cytokines (2, 3, 4 or 5+), assigning the category of cytokines (effector, stimulatory, regulatory or chemoattractive) and measuring the polyfunctionality strength index (PSI) defined as the percentage of polyfunctional cells, multiplied by mean fluorescence intensity (MFI) of the proteins secreted by the NK cells.

### ELISA and Multiplex ELISA

IL-15 and IL-21 levels were measured in the supernatant of cytokine transduced NK cells at five days after transduction using IL-15 or IL-21 ELISA kits from Invitrogen® per the manufacturer's instructions. For multiplex ELISA, supernatants from GSC and NK co-cultures were collected at different time points and cytokine and chemokine production was assessed using Milliplex® Human Cytokine/Chemokine Magnetic bead Premixed 41 Plex Kit (EMD Millipore Corporation, Burlington, MA). Samples were read using a Millipore MAGPIX™ instrument (EMD Millipore). Data acquisition was performed using the Luminex® xPONENT® software and analyzed with Belysa™ Analysis Software (EMD Millipore). Data was plotted using Morpheus, https://software.broadinstitute.org/morpheus.

### Metabolic Assays

The extracellular acidification rate (ECAR) was measured using the Glycolytic Stress Test, and oxygen consumption rate (OCR) was measured using the Mito Stress Test or the XF Substrate Oxidation Stress Test with an Agilent® Seahorse® XFe96 Analyzer (Agilent®) per the manufacturer's instructions. NK cells were assayed alone or purified after 48 hour co-culture with GSC20 cells. Cells were plated in respective assay medias at 250,000 cells per well in a 96 well microplate. Data was analysed using Wave Software (Agilent®).

### Measurement of Reactive Oxygen Species (ROS)

Cells were stained to identify reactive oxygen species (ROS) by NK cells using flow cytometry. For mitochondrial ROS staining, MitoSOX™ red (Cat #M36008, Life Technologies®, Grand Island, NY, USA) was used following the manufacturer's protocol. Cells were analysed using a BD LSRFortessa™ instrument. Results were expressed as percentage of MitoSOX™ positive NK cells.

### Mass Cytometry

The strategy for antibody conjugation is described elsewhere. Table 2 shows the list of antibodies used for the characterization of NK cells in the study.

TABLE 2
List of CyTOF antibody panel

| | TARGET | Clone | TAG | Source | Catalog number |
|---|---|---|---|---|---|
| 1 | CD45 | HI30 | 89Y | Fluidigm ® | 3089003B |
| 2 | CD57 | HCD57 | 115In | BioLegend ® | Discontinued |
| 3 | Eomes | WD1928 | 142Nd | Invitrogen ® | 14-4877-82 |
| 4 | ZAP70 | REA814 | 143Nd | Miltenyi Biotec | 130-095-212 |
| 5 | Singlec 7 | REA214 | 144Nd | Miltenyi Biotec | 130-112 |
| 6 | CD62L | REA615 | 145Nd | Miltenyi Biotec | 130-122-326 |
| 7 | CD8a | REA734 | 146Nd | Miltenyi Biotec | 130-122-281 |
| 8 | NKG2C | REA205 | 147Sm | Miltenyi Biotec | 130-122-278 |
| 9 | TRAIL | RIK-2 | 148Nd | BD Bioscience | 550515 |
| 10 | CD25 | 2A3 | 149Sm | Miltenyi | 130-122-302 |
| 11 | CD69 | FN50 | 150Nd | BioLegend ® | 310939 |
| 12 | CD2 | REA972 | 151Eu | Miltenyi Biotec | 130-122-348 |
| 13 | CD3z | 6B10.2 | 152Sm | BioLegend ® | 644102 |
| 14 | TIGIT | MBSA43 | 154Sm | Thermo Fisher Scientific ® | 16-9500-82 |
| 15 | CD27 | L128 | 155Gd | Bd Bioscience | Custom Order |
| 16 | KLRG1 | 13F12F2 | 156Gd | Thermo Fisher Scientific ® | 16-9488-85 |
| 17 | CD94 | REA113 | 158Gd | Miltenyi Biotec | 130-126-464 |
| 18 | NKP30 | AF29-4D12 | 159Tb | Miltenyi Biotec | 130-092-554 |
| 19 | T-BET | 4B10 | 161Dy | BioLegend ® | 644802 |
| 20 | NKP46 | BAB281 | 162Dy | Fluidigm ® | 3162021B |
| 21 | NKP44 | 2.29 | 163Dy | Miltenyi Biotec | Custom Order |
| 22 | CD95 | DX2 | 164Dy | Miltenyi Biotec | 130-108-066 |
| 23 | TIM3 | 344823 | 165Ho | R&D Systems | MAB2365 |
| 24 | NKG2D | ON72 | 166Er | Beckman Coulter | Custom Order |
| 25 | 2B4 | C1.7 | 167Er | Thermo Fisher Scientific ® | 16-5838-85 |
| 26 | KI67 | Ki67 | 168Er | BioLegend ® | 350502 |
| 27 | NKG2A | Z199 | 169Tb | Fluidigm ® | 3169013B |
| 28 | DNAM | DX11 | 170Er | BD Bioscience | 559787 |
| 29 | Perforin | dG9 | 172Yb | BioLegend ® | 308102 |
| 30 | Granzyme B | GB11 | 173 Yb | BD Bioscience | Custom Order |
| 31 | Granzyme A | REA162 | 174Yb | Miltenyi Biotec | 130-108-054 |
| 32 | LAG3 | REA351 | 175Lu | Miltenyi Biotec | 130-124-529 |
| 33 | CD56 | NCAM16.2 | 176Yb | BD Bioscience | 559043 |
| 34 | CD16 | 3G8 | 209Bi | Fluidigm ® | 3209002B |
| 35 | CD3 | UCHT-1 | 194PT | BioLegend ® | 300402 |
| 36 | Cisplatin | | 198Pt | Fluidigm ® | 201198 |

### Mass Cytometry—Sample Preparation, Staining and Acquisition

NK cells were cultured alone or co-cultured with GSC20 cells (1:1, E:T ratio) for 48 hours. Human Fc receptor blocking solution (Trustain FcX™, BioLegend®, San Diego, CA) was applied to NK cells after they had been washed with cell staining buffer (0.5% BSA/PBS) and incubated for 10 minutes at room temperature. Cells were then stained with a cytometry by time of flight (CyTOF®) antibody mix against cell surface markers (Table 2). The strategy for antibody conjugation was previously described. Cells were recorded at 300 events/second on a Helios instrument (Fluidigm®) using the Helios 6.5.358 acquisition software (Fluidigm®). Mass cytometry data were normalized based on EQ™ four element signal shift over time using the Fluidigm® normalization software 2. Initial data quality control inspection was performed using Flowjo version 10.7. Singlets were chosen based on iridium 193 staining and event duration, and calibration beads were gated out. Dead cells were excluded by the Pt195 channel and further gating was performed to select CD45+ cells and then the NK cell population of interest (CD3 CD56+). A total of 320,000 cells were proportionally extracted from each sample to perform automated clustering.

### Data Analysis

The mass cytometry data were merged using Principal Component Analysis (PCA), “RunPCA” function, from R package Seurat (v3). Dimensionality reduction was performed using the “RunUMAP” function from the R package Seurat (v3) with the top 20 principal components. The UMAP plots were generated using the R package ggplot2 (v3.2.1). Data were analyzed using automated dimension reduction including (viSNE) in combination with FlowSOM for clustering for the deep phenotyping of immune cells. Relevant cell clusters were further delineated using an in-house pipeline for cell clustering. To generate heatmaps, CD45⁺CD56⁺CD3⁻ gated FCS files were exported from FlowJo to R using function “read.FCS” from the R package flowCore (v3.10). The mean values of all markers were plotted as a heat map using the function “pheatmap” from R package pheatmap (v1.0.12). Markers with similar expression were hierarchically clustered.

### Xenogeneic Mouse Model of GBM

To assess the antitumor effect of cytokine transduced NK cells against GSCs in vivo, NOD/SCID IL-2Rγ^{null} (NSG) human xenograft model animals were utilized (Jackson Laboratories, Bar Harbor, ME). Patient-derived GSC mouse models were utilized due to their superior invasiveness and migratory ability relative to conventional glioma cell lines when implanted intracranially²⁴. A total of 0.5×10⁶ patient-derived GSC20, GSC262, GSC267, GSC8-11 or GSC272 cells were implanted intracranially into the right frontal lobe of five week old female NSG mice using a guide-screw system as previously described 24. For rechallenge experiments, 0.25×10⁶ GSC20 cells were implanted intracranially. To increase uniformity of xenograft uptake and growth, cells were injected into 10 animals simultaneously using a multiport Microinfusion Syringe Pump (Harvard Apparatus, Holliston, MA). Animals were anesthetized with xylazine/ketamine during the procedure. For in vivo bioluminescent imaging, GSCs were engineered to express luciferase by lentivirus transduction. Kinetics of tumor growth were monitored using weekly using bioluminescence imaging (BLI) (Xenogen-IVIS® 200 Imaging system; Caliper, Waltham, MA). Signal quantitation in photons/second (p/s) was performed by determining the photon flux rate within standardized regions of interest (ROI) using Living Image software (Caliper). At day 7 post tumor implantation, NK cells (0.5×10⁶ or 0.1×10⁶ NKs in 4 μl) were injected intratumorally via the guide-screw. Mice that presented neurological symptoms (e.g., hydrocephalus, seizures, or ataxia) or were moribund were euthanized. Brain and other tissues were then extracted and processed for analysis.

### Histopathology

Brain tissue specimens were collected from untreated control mice, mice treated with either IL-15 transduced NK cells or IL-21 transduced NK cells. Brain tissue was fixed in 10% neutral buffered formalin and then embedded in paraffin. Formalin-fixed, paraffin embedded tissues were cut into 4 μm sections, and stained routinely with hematoxylin and eosin. Brains were examined for the presence or absence of glioblastoma tumor cells, general gliosis, and presence of NK infiltration. A board-certified veterinary pathologist examined sections free of tumor for evidence of meningoencephalitis using a Leica® DM 2500 light microscope. One section was examined from each sample. Representative images were captured from comparable areas of cerebral hemispheres with a Leica® DFC495 camera using 10× objectives.

### Immunohistochemical Staining

Formalin-fixed, paraffin embedded tissue was routinely sectioned at 4 μm and placed on glass slides. After deparaffinization, Granzyme B (human-specific, rabbit monoclonal antibody clone D6E9W, Cell Signaling Technologies) or CD16 (human specific, rabbit polyclonal antibody, Thermo Fisher Scientific®) antibody was added to the tissue section to identify human NK cells. Anti-Ki-67 antibody (rabbit monoclonal antibody clone SP6, Abcam) was used to identify proliferating GSC tumor cells in the brain sections. Anti-GFP antibody was used to identify astrocytes and anti-Iba11 for microglia. Twenty minutes of antigen retrieval in citrate buffer at 95° C. was performed prior to staining. Primary antibody was diluted at 1:100 with an incubation time of 60 minutes. Hematoxylin was used to counterstain the nuclei and sections were routinely cover-slipped. Slides were examined using a Leica® DM2500 microscope and images were captured at 4×, 10×, 20×, and 40× with a Leica® DMC6200 camera and Leica® Application Suite (LAS) software. For multiplex immunofluorescence, six antibodies were used: anti-Granzyme B, anti-Iba1 (clone EPR16589, Abcam), anti-GFAP (Dako), anti-Cleaved Caspase-3 (BioCare Medical), anti-luciferase (Clone EPR17790, Abcam) and anti-Ki-67. DAPI was used to identify cell nuclei. Slides were scanned using Aperio AT2, image registration, and image fusion/merging using HALO v.3.6.

### Mice Brain Tissue Processing

Mononuclear cells were isolated from mice brain tissue using a Percoll® (GE HealthCare, Chicago, IL) gradient following the protocol described by Pino et al²⁴. Briefly, brain tissue was dissociated and passed through using a 70 μm cell strainer (Life Science, Durham, NC) to make homogenous cell suspension. A 70% isotonic Percoll® solution was put on top of a resuspended cell suspension in a 30% isotonic Percoll® solution. Cells were centrifuged at 500×g for 30 minutes at 18° C., with no brake. In a clean tube, 2-3 ml of the 70%-30% interface was collected and rinsed once with PBS 1×. After this procedure, cells were ready for immunostaining as described above.

### Single-Cell ATAC Sequencing

IL-15 transduced NK cells and IL-21 transduced NK cells were co-cultured with GSC20 (1:1, E:T ratio). Every two to three days fresh GSCs cells were added to the co-culture. Cells were collected and cryopreserved at day 3 (1×GSC), and day 9 (3×GSC). NK cells cultured in the absence of GSCs were collected at day 0 and were used as baseline control.

Cryopreserved cell suspensions were thawed in 37° C. water bath, and viability was measured. The cells were treated with nuclei lysis buffer (10× Genomics™, San Francisco, CA), nuclei were washed, resuspended in diluted nuclei buffer (10× Genomics™) and counted for loading at a volume to recover 5000 to 10000 nuclei. Single-nucleus libraries were generated using the 10× Genomics™ Chromium Next GEM ATAC Capture and Library (V1.1), following the manufacturer's protocol. Nine samples were pooled to give a final concentration of 10 nM. Pooled samples underwent further qPCR prior to sequencing with the NovaSeq 6000 sequencer using the S1, 100 cycles flow cell and sequenced with read 1 for 50 cycles, i7 8 cycles, i5 index 16 cycles, and read 2 for 49 cycles. Data available under accession number GSE227098.

Upstream Analysis of scATAC-Seq Data

For each sample, the fragment and peak profiles were generated using the 10× Genomics™ CellRanger™ pipeline with default parameters. Chromosome, start, end, cell barcode, and PCR duplicate count were the five columns that made up fragment files, which were specified as coordinate-sorted, block gzip-compressed (bgzip), and indexed browser-extensible data files. The sites of the two Tn5 integration events that generated the sequenced DNA fragment were indicated by the start and end fields of the fragment file.

Downstream Analysis of scATAC-Seq Data

The downstream analysis was done in R 4.0 applying Signac⁶³. Because peak calling was performed for each sample independently, we created a common set of peaks across all samples using the reduce function in Signac. The peaks with width<20 bp or >10,000 bp were removed. The fragment matrix for each sample was adjusted based on the common peak set using CreateFragmentObject function. The peak profile from multiple samples was combined into one unified matrix in which the peak coordinates are shared by each sample.

The QC step was performed by removing cells with fragments<500 and features with cells<500. To perform the dimension reduction, the RunTFIDF was performed on top peaks identified from FindTopFeatures function (min.cutoff=10). The RunSVD function was used to perform dimension reduction on peak assay (dimension was set to 30).

Joint clustering of single-cell ATAC peak profiles over 3 time points (baseline, day 3 and day 9) of 7,000-11,000 NK cells (Table 3) using the Signac pipeline led to the identification of 10 different NK cell clusters.

TABLE 3
Number of cells used for each product after quality
control at baseline (day 0) and days 3 and 9

| | Product | Baseline | Day 3 | Day 9 |
|---|---|---|---|---|
| scATAC-seq | IL-15 NK | 10,565 | 7,610 | 8,201 |
| | IL-21 NK | 10,903 | 8,436 | 8,403 |
| scRNA-seq | IL-15 NK | 18,230 | 7,248 | 7,121 |
| | IL-21 NK | 7,388 | 7,082 | 5,804 |

To identify differential peaks among samples/clusters, the differential accessibility (DA) test using the FindMarkers function was performed with minimum.pct being 5% and the number of fragments as the latent variable. To identify potentially important sample/cluster-specific regulatory sequences, DNA motifs (i.e., TFs) that were overrepresented in a set of peaks that were differentially accessible between samples was searched for using the FindMotifs function. This generated the probability of observing the motif at the given frequency by chance, compared with a background set of peaks matched for GC content. A motif's higher score indicated that its TF was much more abundant in the differential peaks. The EnhancedVolcano function was used to show the TF enrichment fold-change between two samples, https://github.com/kevinblighe/enhancedvolcano.

To identify accessibility levels of CEBPD and CEBPB targets, the gene level accessibility for each cell was calculated by quantifying ATAC-seq counts in the 2 kb-upstream region and gene body, using the GeneActivity function in the Signac package.

### Single-Cell RNA Sequencing

IL-15 NK cells or IL-21 NK cells were co-cultured with GSC20 (1:1, E:T ratio). Every two to three days fresh GSCs were added to the co-culture. Cells were collected and cryopreserved at day 3 (1×GSC), and day 9 (3×GSC). NK cells cultured without tumor (e.g., in the absence of GSCs) were collected at day 0 and used as baseline control.

Cryopreserved cell suspensions were thawed in 37° C. water bath, and the viability of the samples was determined using Trypan Blue Stain (0.4%) with the Countess™ II FL. Cells were resuspended in phosphate buffered saline (PBS) with 0.4% bovine serum albumin (BSA) within the recommended range of 500-1000 cells/μl for loading at a volume to recover 7000 to 10,000 cells for single cell RNA-sequencing (scRNA-seq). Single cell capture, barcoding and library preparation were performed following the 10× Genomics™ Single Cell Chromium 3′ protocols (CG000183, V3.1). Ten libraries were pooled to give a final concentration of 10 nM. Pooled samples underwent further qPCR to determine the final concentration before submission for sequencing with the NovaSeq 6000 sequencer using the S2 100 cycles flow cell and sequenced for 28 cycles for read 1, 8 cycles for i7 index, and 91 cycles for read 2 through the ATGC (Advanced Technology Genomics Core) core at MD Anderson. Data available under accession number GSE227098.

The downstream analysis was done in R 4.0 applying Seurat software⁶³. The QC step was performed by removing cells with feature<50 and features with cells<100. The resulting single-cell dataset included 52,873 cells over the three time points for IL-21 NK cells vs IL-15 NK cells cultured with or without GSCs (Table 3). To perform the dimension reduction, the RunPCA was performed on the top 2,500 variable gene identified from FindTopFeatures function. The DEGs among samples/clusters were performed using the FindMarkers function with minimum.pct being 5% and the number of genes as the latent variable.

The CEBPD regulon score in gene expression level was calculated by averaging expression levels of 106 CEBPD target genes inferred from pySCENIC on single cell level using AddModuleScore function.

The gene enriched pathways of CEBPB and CEBPD target genes were identified using Enricher tool by searching MSigDB Hallmark database. Only enriched pathways with adjusted p value higher than 10⁻⁵ were kept.

For the scATAC and scRNA profiling-derived genomic coverage, bam alignment mapping files were retained from the cellranger count or cellranger-atac count pipeline that processes the scRNA or scATAC sequencing data, respectively. Cell barcodes of the filtered scRNA or scATAC samples were exported from the Seurat or Signac package in R, respectively. The bam files of these filtered cells were extracted and generated using sinto filterbarcodes of the sinto software (https://timoast.github.io/sinto/). The resulting bam files were converted into the bigwig format using the bamCoverage command of the deepTools software and visualized in IGV (Integrative Genomics Viewer).

Method Describing the pySCENIC Figures

In order to infer key TFs and gene regulatory networks from scRNA-seq data, the pySCENIC v0.11.2 (Single-Cell Regulatory Network Inference and Clustering) pipeline was utilized. Briefly, in the initial step, pySCENIC was used with the default parameters on the high-performance computing system to infer regulatory interactions between a previously defined list of TFs and candidate target genes using the gradient boosting machine regression GRNBoost2 algorithm, and Arboreto using co-expression patterns from scRNA-seq data. As a result, an adjacencies matrix that connects each TF with a target gene and an importance score which separates high confidence interactions from weak ones was obtained. Next, candidate modules from these interactions were generated, which were composed of a TF regulator and the list of its target genes. In order to separate the direct targets from indirect targets of a given regulator, these co-expression modules were refined by choosing target genes which have the DNA motif that is specific to a certain TF in their promoter region. This was achieved by cis-regulatory module scoring with RcisTarget to look for modules with cisTarget motif enrichment using pre-computed whole-genome rankings of all motifs that are linked to a known TF in the pySCENIC database. Through this analysis, normalized enrichment scores (NES) were calculated, which represents a quantitative measure for individual motif enrichment. Only target genes with NES>4 were considered for downstream analysis. Finally, the Area Under the Curve (AUC) scores, that measure and quantify the relative biological activity of each regulon at a cellular level, were calculated. With the pySCENIC analysis, 456 regulons with their direct target genes exhibiting significant TF motif enrichment along with their AUC regulon activity scores were defined. these scores were overlayed on the scRNA-seq data using Seurat 4.1.0, and differential regulons between IL-21 NK cell and IL-15 NK cell samples were identified across different time points using the FindMarkers function in Seurat. Differential regulons of interest were visualized using the DoHeatmap function on the scaled AUC matrix in order to facilitate making comparisons across samples.

### Analysis of Bulk RNA-Seq

Gene expression was quantified from fastq files using RSEM (1.3.3), using the bowtie 2 (2.4.2) aligner. The R package tximport was used to read in gene counts from RSEM output files. Data available under accession number GSE227098. Differential Gene Expression Analysis (DEG) was performed to compare NT NK cells independently, IL-15 NK cells, or IL-21 NK cells, and in the presence (CEBPD KI (OE)) or absence of CEBPD (CEBPD KO) (n=4 donors) using 2-way ANOVA fitted models, with either NT NK cells, IL-15 NK cells, IL-21 NK cells, or WT groups as the reference level⁴⁷. Differentially expressed genes (DEGs) were defined as genes with Log 2 fold change<0.3 or >0.3 and p-value<0.05. DEG was performed in R using OBIF package in R⁴⁷ version 1.0.

Enrichment Analysis was performed using the Ingenuity Pathway Analysis Software (QIAGEN® Inc.,) by a QIAGEN® IPA Certified Analyst with the core analysis function for expression values. Unsupervised Enrichment for Pathways, Diseases and Functions, Regulators, and Networks was performed to evaluate the IL-21 vs IL-15 wild-type (WT) response with significant enrichments defined as Z-score<−2 or >2 and false discovery rate (FDR)<0.05. Expression values were overlayed from bulk RNA-seq differential analysis accordingly, and network analysis of regulators was trimmed using bulk and single-cell ATAC-seq accessible regions as a reference.

Differential Gene Expression Analysis (DEG) was performed separately in IL-21-transduced and Non-transduced NK cells adjusting for cord. CEBPD KI (OE), CEBPD KO and NT NK cells were compared using NT NK cells as the reference level. Differential expressed genes (DEGs) were defined as genes with FDR-adjusted p-value<0.05. DEG was performed in R using DESeq2 package in R version 4.1.1.

Pathway Enrichment Analysis (GSEA) was performed using pre-defined CEBPD downstream genes. GSEA was performed using the ranked wald test statistics for all genes in the DEG analysis with gsea function from the clusterProfiler package in R version 4.1.1.

### Analysis of Bulk ATAC-Seq

ATAC-seq library preparation was performed at the MDACC Epigenomics Profiling Core following the protocol as previously described 12 with some modifications. Briefly, 50,000 nuclei isolated from IL-15 NK cells, IL-21 NK cells, and NT NK cells with and without CEBPD (CEBPD KI (OE) and KO, respectively) from 4 donors were fragmented and the resulting libraries were purified using SPRISelect™ beads (Beckman Coulter). Libraries were sequenced 2×100 bp on an Illumina™ NovaSeq 6000 to obtain at least 50 million high quality mapping reads per sample. Data available under accession number GSE227098.

For each bulk ATAC-seq sample, the pair-end reads from fastq files were aligned to the human genome (GRCh38) using bwa mem mode with duplicated reads removed. The 5′ end of ATAC-seq reads were shifted to the actual cut-site of the Transposase using alignmentSieve module implemented in DeepTools. The peaks were called using Macs2 with using the pair-end read information. The minimum FDR (q-value) cutoff for peak detection was set as 0.05. The Macs2 outputs from multiple samples were loaded using DiffBind. The peak sets from multiple samples were identified as the overlapping ones among samples using bUseSummarizeOverlaps function in DiffBind. The TF activity level was then calculated using the function RunChromVAR in Signac, and differential TFs among different groups were identified using function FindMarkers (with min.pct-0.5 and logfc.threshold=2).

Chromatin Immunoprecipitation (ChIP) qPCR and ChIP Sequencing (ChIP-Seq)

Chromatin immunoprecipitation (ChIP) was performed at MD Anderson Cancer Center Epigenomic Profile Core. IL-15 NK, IL-21 NK, and NT NK cells (n=2-3 donors) were cross-linked for 10 minutes with 1% formaldehyde at room temperature followed by addition of glycine (125 mM final concentration) to quench for 5 minutes⁶⁴. Chromatin was sonicated to an average size of 600 bp. Chromatin and associated proteins were immunoprecipitated using anti-CEBPD antibody (Santa Cruz Biotechnology) and IgG antibody. Input and CEBPD ChIP DNA libraries were prepared using NEBNext® Ultra™ II DNA library prep kit (NEB). The ChIP-Seq libraries and the corresponding input libraries were sequenced using the 50 base single-read protocol on Illumina® NextSeq® 500 and HiSeq® 3000 instrument.

For ChIP-quantitative Polymerase Chain Reaction (ChIP-qPCR), chromatin was immunoprecipitated using antibodies recognizing pSTAT1 (Tyr701) or pSTAT3 (Tyr705) (both from Cell Signaling Technology), or control rabbit immunoglobulin G (IgG) at 4° C. for 12-16 hours. Quantitative real-time PCR assays were performed after cross-link reversal, using the precipitated DNA and primers corresponding to specific target gene regions. Primers used in the assay were as follows⁶⁵: CEBPD Forward: 5′-GGTTTCACCATGTTGACCAG-3′ (SEQ ID NO: 57), and CEBPD Reverse: 5′-AGAATGGGCTTTGTCATGTG-3′ (SEQ ID NO: 58).

The DNA region of interest was detected by SYBR real-time quantitative PCR and enrichment relative to input were calculated.

### Analysis of ChIP-Seq

Sequencing adapters and low-quality bases were trimmed using Trimmomatic 0.38. Cleaned reads were then aligned to the reference genome hg38 using bowtie2. Peak signal was visualized using Integrative Genomics Viewer Version 2.16.1.

### Peak and Tag Density Profiles of CEBPD ChIP-Seq

Combined tag directories per treatment conditions were created from aligned donor sample files (BAM) using HOMER⁶⁶ with makeTagDirectory function (-genome hg38-checkGC). Using the annotatePeaks.pl function, annotated regions list for CEBPD-specific gene targets were made around their transcription start sites (TSS) (tss hg38-list <gene list>) and normalized tag density profiles (per base per peak) was calculated around TSS all individual samples (<annotated region list> hg38-size-300,100-d<combined tag directories>). GraphPad's Prism 10 software was used to create box plots that represent the sequenced normalized tag density of the CEBPD-specific gene targets, and peak profiles plots (5 bp bins) that represent their sequenced CEBPD occupancy from all donors. Genomic visualization of specific CEBPD-regulated gene targets in GRCh38 human genome was done using Integrative Genomics Viewer 67 (IGV 2.16.0) software from the sample BAM files.

qPCR

RNA was extracted from NK cells and then reverse transcription was performed using Iscript™ cDNA Synthesis kit (Bio-Rad®, Mylan, Italy). The relative expression levels of the genes of interest were assessed using RT-qPCR using Applied Biosystems® Fast SYBR® green qPCR master mix (Thermo Fisher Scientific®). Premade CEBPD, KLF2 and BNIP3L primers were purchased from IDT™. The relative expression level was determined by comparison with the housekeeping gene S18.

### CRISPR Gene Editing

Pre-designed sgRNAs were used to target the CEBPD gene in NK cells. sgRNAs were ordered from SYNTHEGO™ (Gene Knockout Kit v2-human-CEBPD-1.5 nmol, and Gene Knockout Kit v2-human-STAT3-1.5 nmol)—while Cas9 was purchased from IDT™ in their proprietary Alt-R™ HiFi format (IDT™ cat #1081061).

The following 3 sgRNAs were used for CEBPD gene mutation:

sgRNA #1:
(SEQ ID NO: 52)
GCCGUCCAGGCUGAAGAGCG,
sgRNA #2:
(SEQ ID NO: 53)
CCCGGUUCGUAGAAGGGCGC,
and
sgRNA #3:
(SEQ ID NO: 54)
CUCUCGUCGUCGUACAUGGC.

The following 3 sgRNAs were used for human STAT3 gene mutation:

sgRNA #1:
(SEQ ID NO: 59)
AAUCUUGACUCUCAAUCCAA,
sgRNA #2:
(SEQ ID NO: 60)
AGCUGUCACUGUAGAGCUGA,
and
sgRNA #3:
(SEQ ID NO: 61)
AUUUUAGCAGGAUGGCCCAA.

Combined sgRNAs from the kit were resuspended in nuclease-free TE buffer at a concentration of 100 μM. The sgRNAs were first diluted with nuclease-free water to a concentration of 30 μM. sgRNAs were combined with Cas9 and T buffer (Neon™ Electroporation Kit, Invitrogen®) at sgRNAs:Cas9 ratio of 3:1. The ribonucleoprotein complex (RNP) was incubated for 10 minutes at room temperature (RT). T25 flasks were prepared during the incubation period by adding an appropriate volume of media and Universal APCs (1:2 ratio of effector to target cells) supplemented with 200 IU/ml of IL-2 (for NK cells only) into each flask. The flasks were then placed in an incubator at 37° C. until the time of electroporation. Effector cells were collected by centrifugation and washed twice with PBS in aliquots of 500,000 cells each. To prepare the cells for electroporation, the supernatant was removed as much as possible without disturbing the pellet and the cells were resuspended in Resuspension Buffer T for electroporation. The final concentration for each electroporation was 1.8 μM sgRNA, 0.62 μM Cas9 nuclease and 0.45 μM Cas9 electroporation enhancer. The cells were electroporated using Neon Transfection System, at 1600V, 10 ms pulse width and 3 pulses with 10 μl electroporation tips (Thermo Fisher Scientific®, cat #MPK5000)). After electroporation the cells were transferred into the prepared flasks and placed in the 37° C. incubator. The knockout efficiency was evaluated by PCR followed by agarose gel electrophoresis.

The following primers were utilized for CEBPD gene amplification: Forward: 5′-

(SEQ ID NO: 55)
GACAGCCTCGCTTGGAC-3′,
and
Reverse:
(SEQ ID NO: 56)
5′-CAAGCTCACCACGGTCTG-3′.

The following primers were utilized for STAT3 gene amplification: Forward 5′-

(SEQ ID NO: 62)
GGGTGCCCCTTTATCTCCTG-3′,
and
Reverse
(SEQ ID NO: 63)
5′-GGGAGAAAGAAGCAGGGTCC-3′.

### Statistical Analyses

Statistical analyses were performed and plotted using Prism 9.2.0 software (GraphPad, San Diego, CA). The Student's t-test was used to test for significance between 2 groups; one-way ANOVA was applied to determine the comparison among groups of a certain condition; two-way ANOVA was applied to determine the comparison among groups over a specific time. The Dunnett correction was used when comparing to a category of reference or control. Additionally, the Bonferroni correction was used to adjust for repeated measures. Mean values+s.e.m. are shown. Overall survival (OS) analysis was calculated using Kaplan-Meier methods and compared to the treatment group using log-rank tests with 95% confidence intervals (CI). Statistically significant p values<0.05 are reported as *p<0.05, **p<0.01, and *** p<0.001. The significance test used and sample sizes (n) are reported in each figure description.

### Study Approval

All Tumor tissue that were used for the generation of glioblastoma stem cells (GSCs) was resected from patients who had signed written informed consents, and samples were collected in accordance with the Institutional Review Board of The University of Texas MD Anderson Cancer Center in Houston, IRB Protocols LAB04-0001, LAB03-0687, and 2012-0441. All tissue samples were de-identified. All studies were performed in accordance with the Declaration of Helsinki. All animal experiments were performed in accordance with recommendations in the Guide for the Care and Use of Laboratory Animals of the National Institute of Health and approved by the Institutional Animal Care and Use Committee (IACUC) protocol number 00001263-RN01 at MD Anderson Cancer Center in Houston.

### Example 1

### Cytokine-Engineered NK Cells Showed Robust Anti-GSC Activity

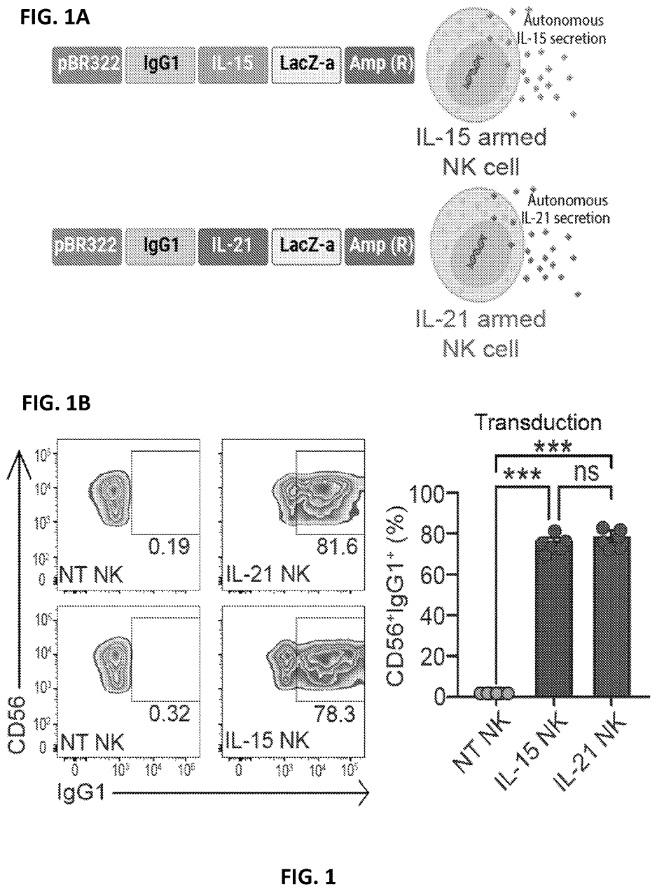

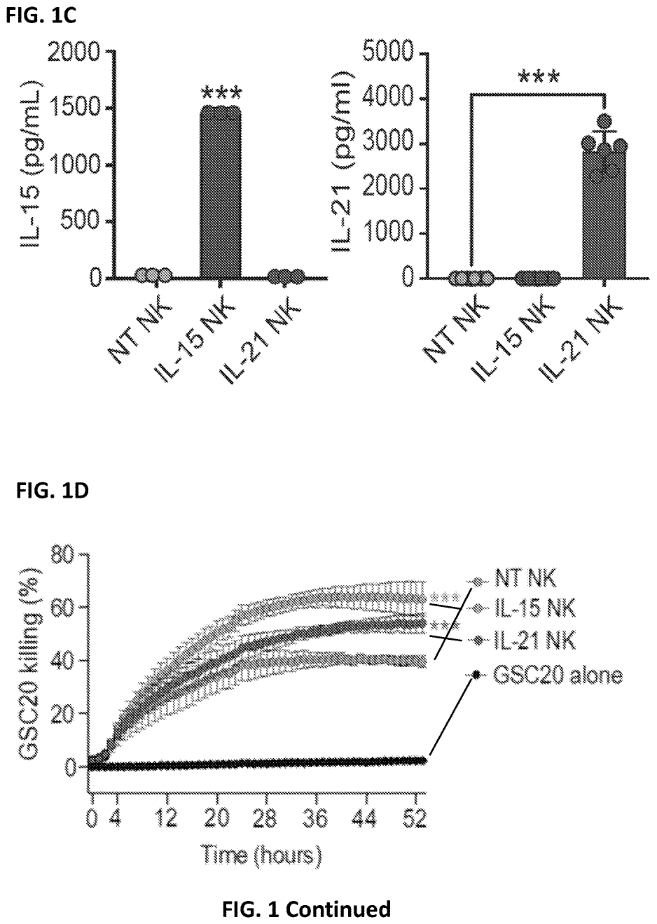

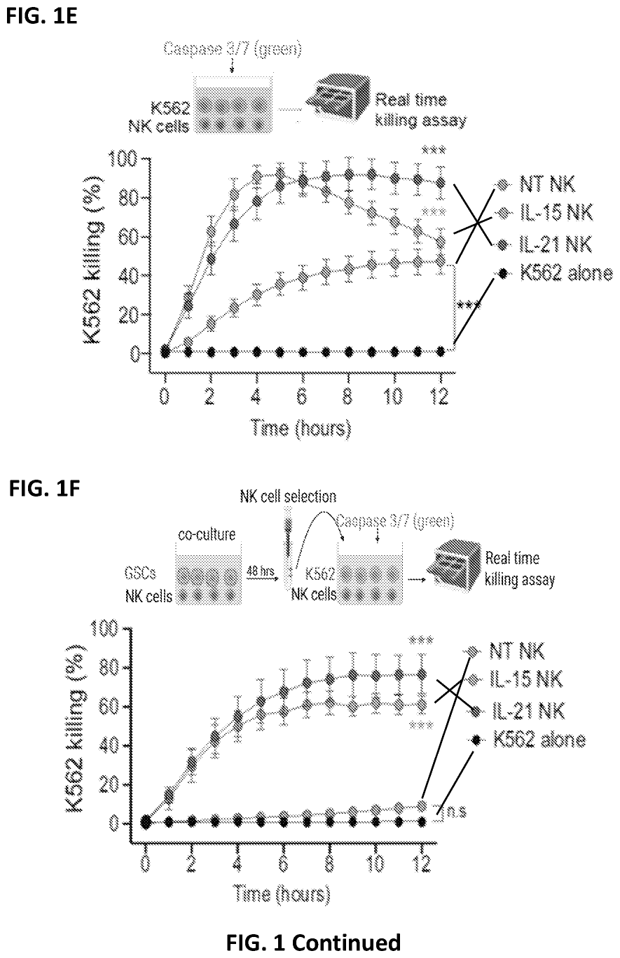

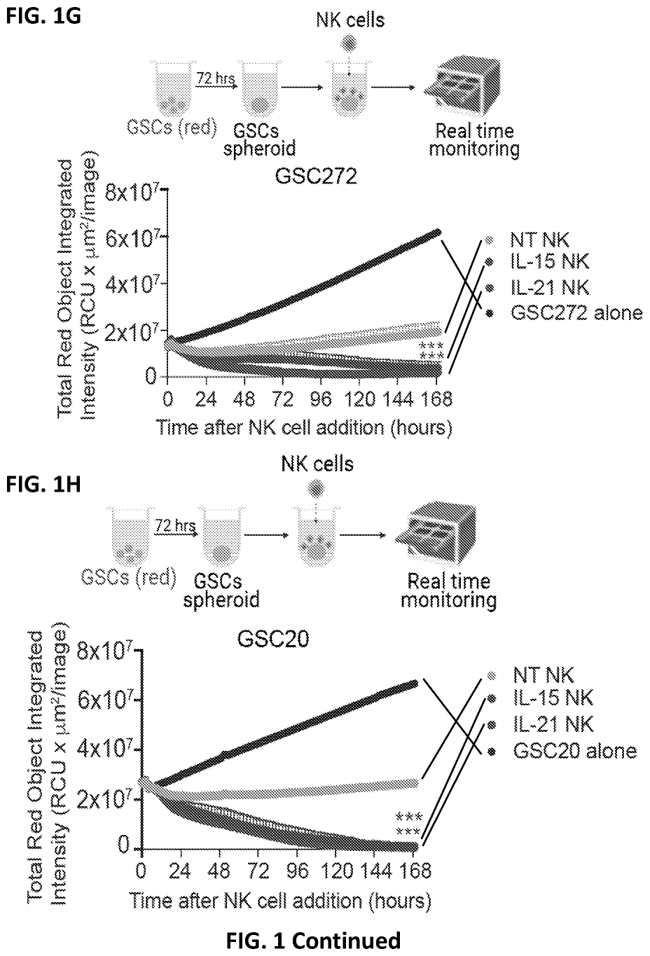

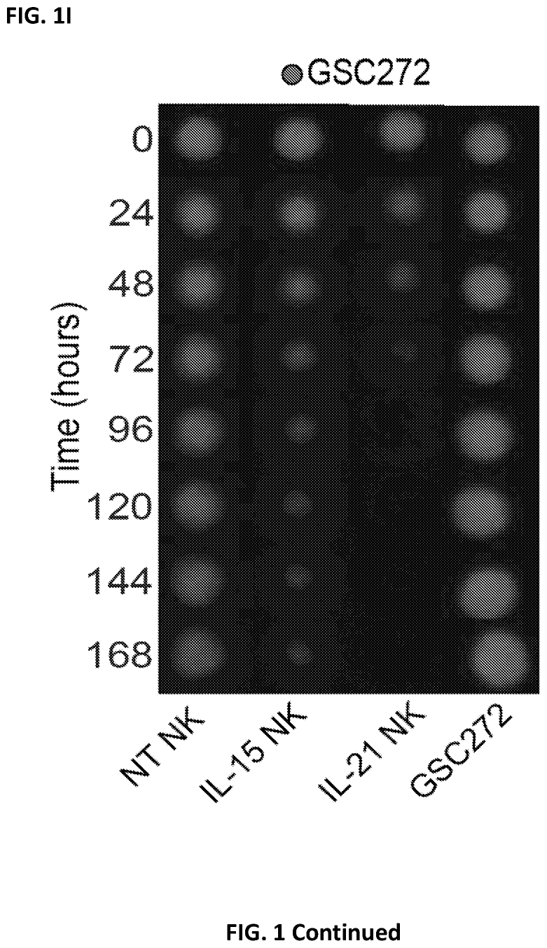

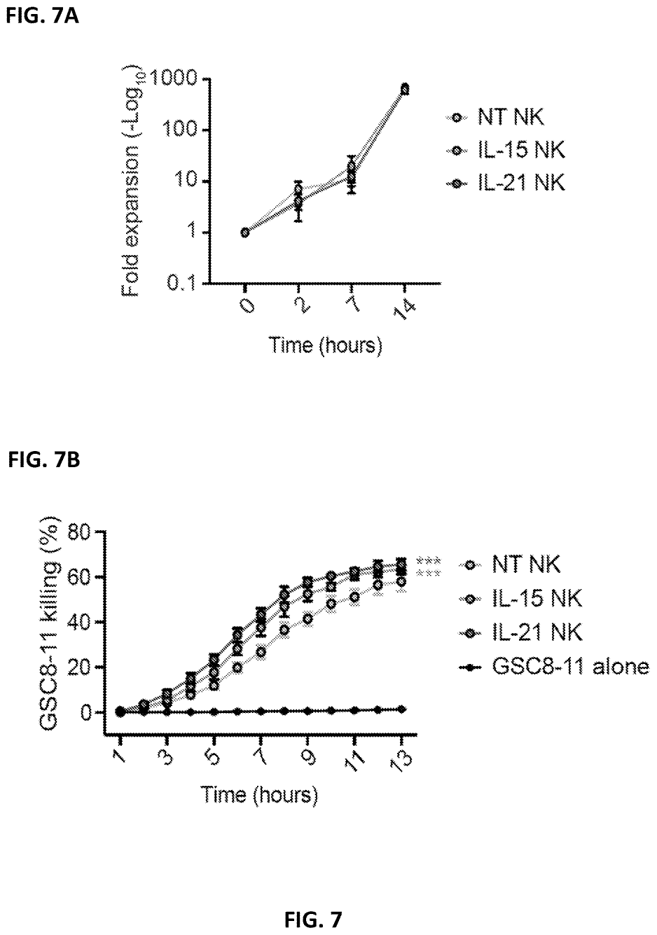

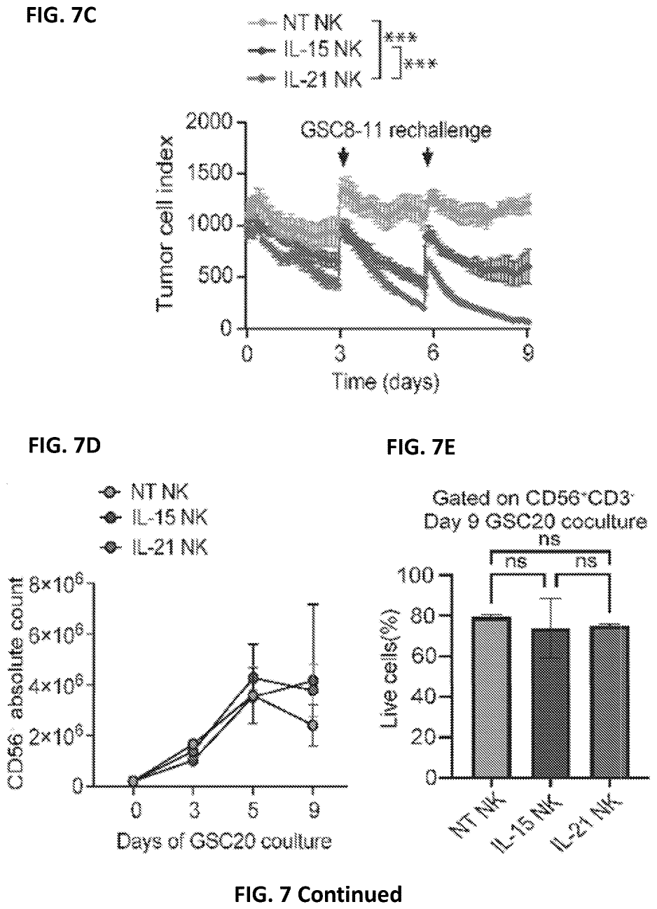

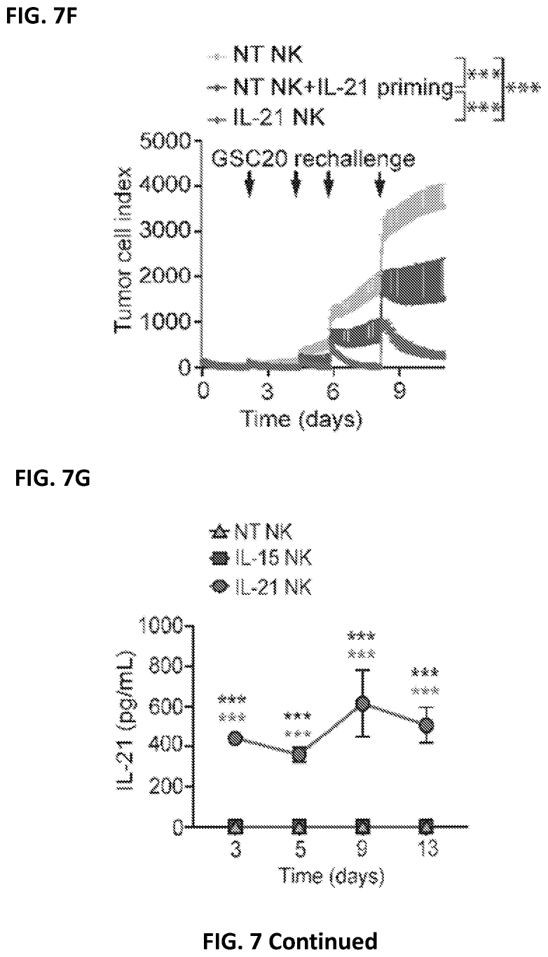

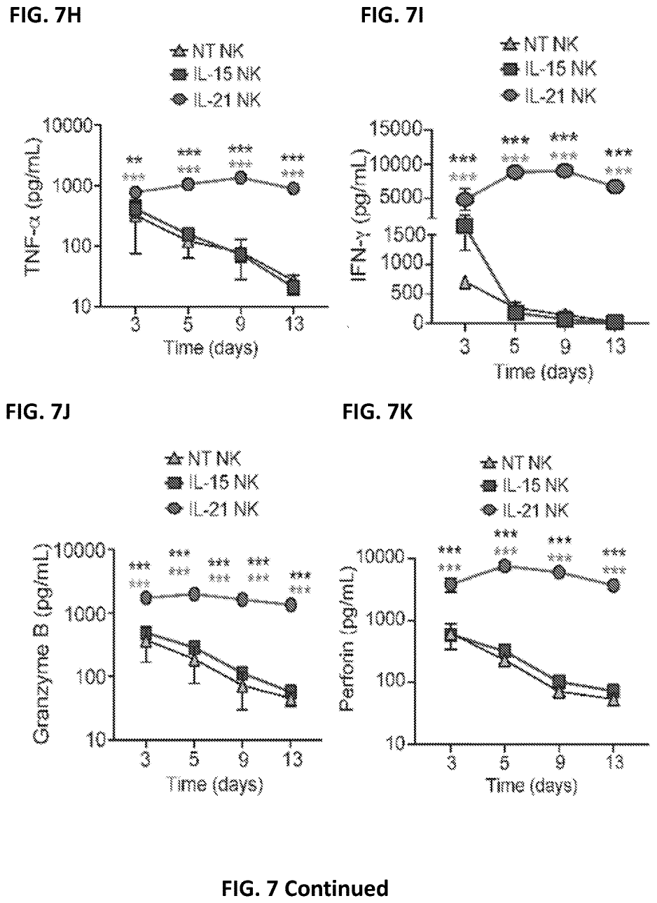

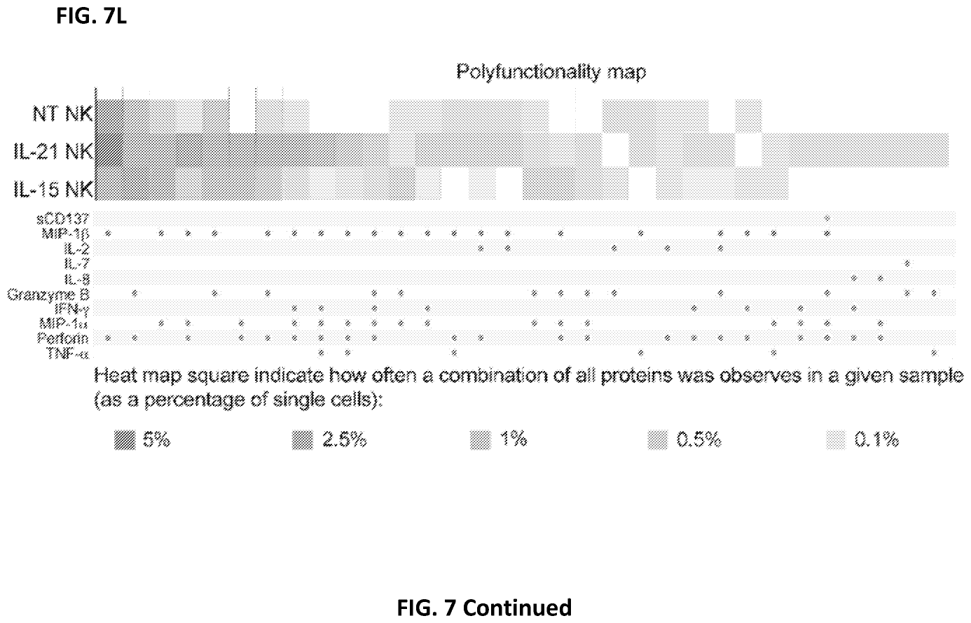

Cytokine engineering has been used as a tool to improve the proliferation, persistence and cytotoxicity of T and NK cells against cancer²². GSCs have tumor-regenerative potential with distinct transcriptional, epigenetic, and metabolic features²³. To determine the impact of cytokine-arming on NK cell antitumor responses, particularly responses against GSCs, cord blood (CB)-derived NK cells were engineered to express and autonomously release secreted IL-15 (SEQ ID NO: 29) or secreted IL-21 (SEQ ID NO: 31) (e.g., in constructs as represented by FIG. 1A (SEQ ID NOs: 44 and 43 respectively). Transduction efficiency was analyzed by flow cytometry, with CD56 expression utilized to identify NK cells, and expression of the co-transduced marker IgG1 Fc acting as a proxy for IL-15 or IL-21 expression, exemplary donor cell transduction efficiencies were approximately 84.7% for IL-15 NK and 89.1% for IL-21 NK (FIG. 1B). Secreted levels of IL-21 or IL-15 in the supernatants from non-transduced (NT) NK cells (NT NK), IL-21 NK cells (IL-21 NK), and IL-15 NK cells (IL-15 NK) was analyzed using ELISA assays (measured in pg/mL) 5 days after transduction. IL-15 NK cells were found to robustly express IL-15 (~1500 pg/mL; n=3 donors), while IL-21 NK cells were found to robustly express IL-21 (~2,200 pg/mL; n=5 donors) (FIG. 1C). The cytokine-armed NK cells response against GSCs was then assessed. NT NK cells utilized as controls. No significant differences in the short term in vitro proliferation of NK cells was observed among the three groups (FIG. 7A), following ex vivo culture with K562-based feeder cells and IL-2. both IL-15 NK, and IL-21 NK cells exerted significantly greater cytotoxicity against GSC20 (n=6 donors) and GSC8-11 (n=3 donors) cells in vitro when compared to NT NK cells (FIG. 1D and FIG. 7B).

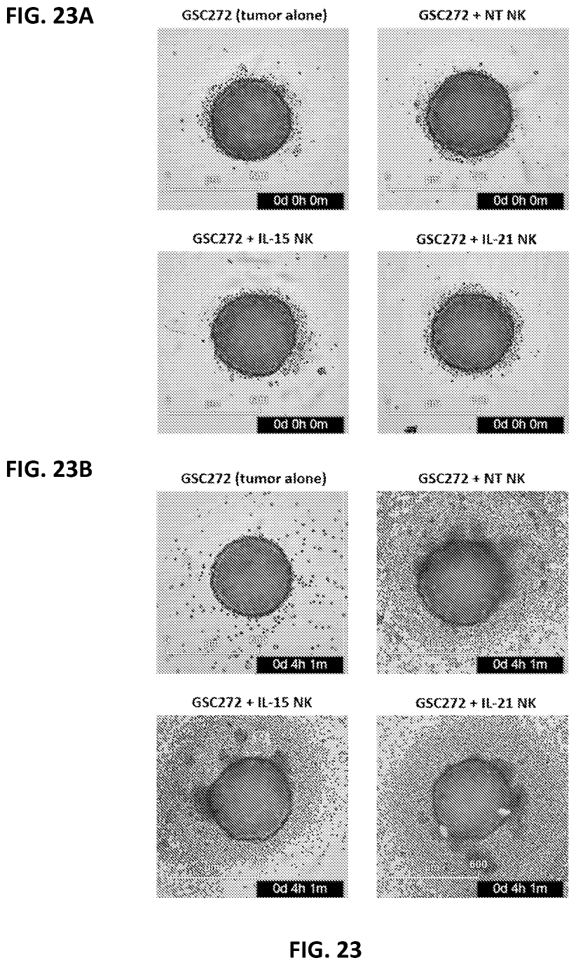

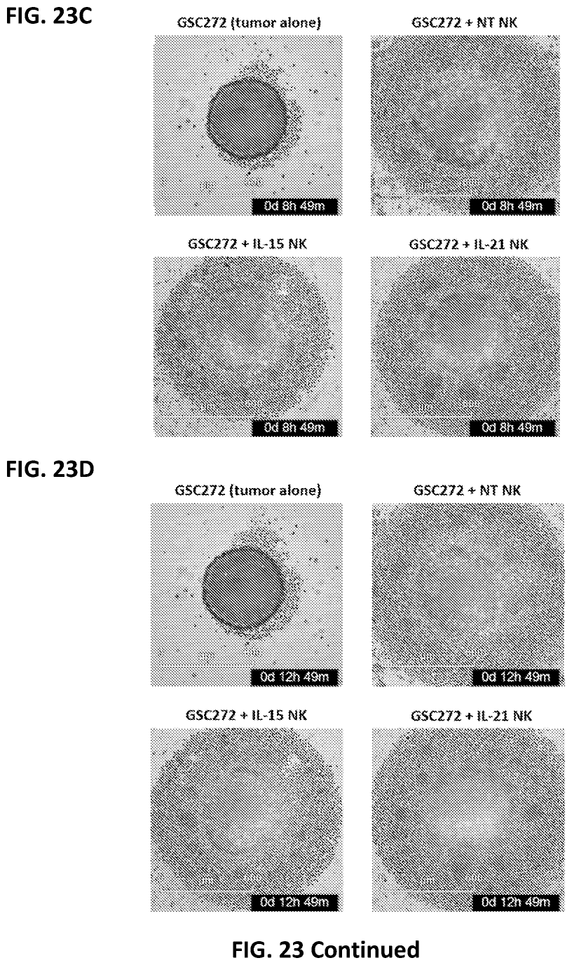

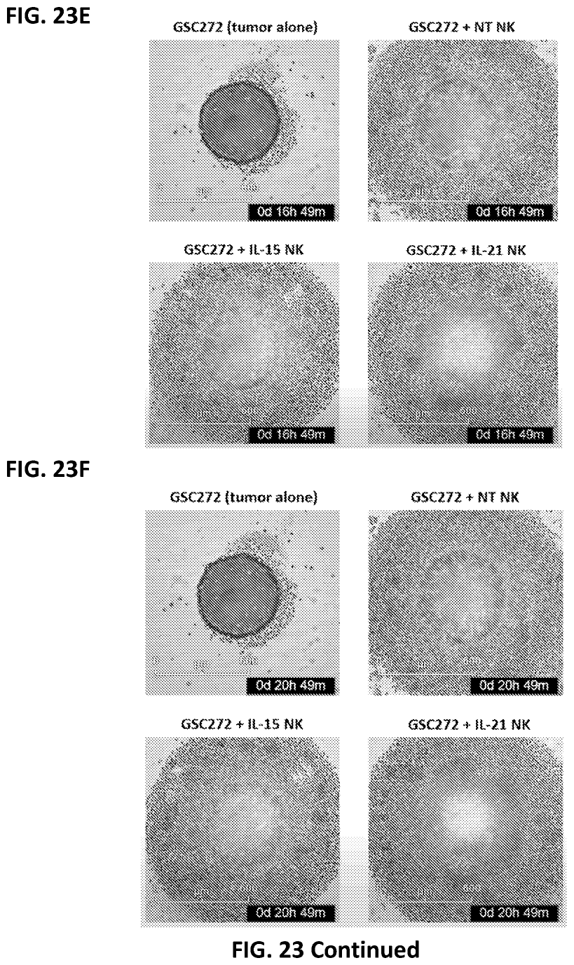

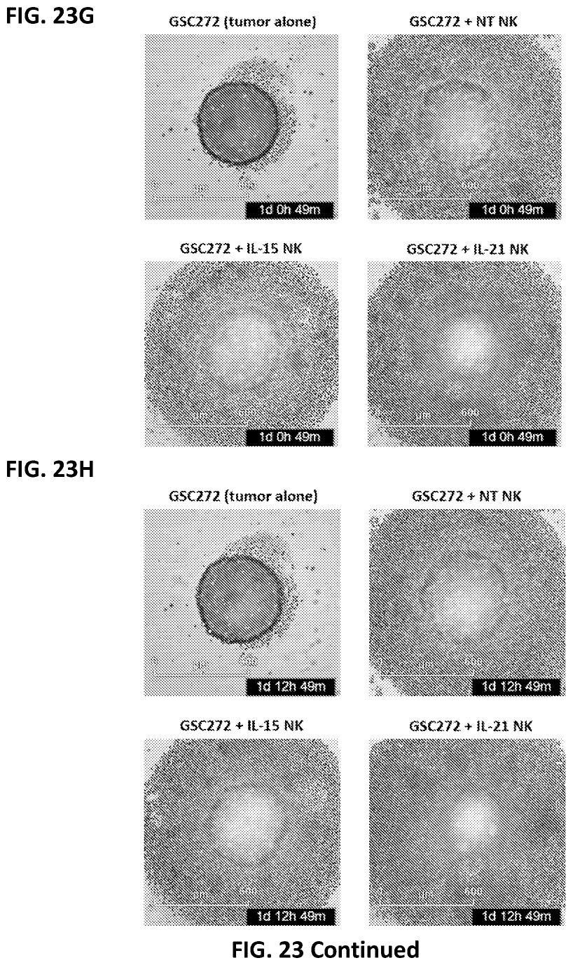

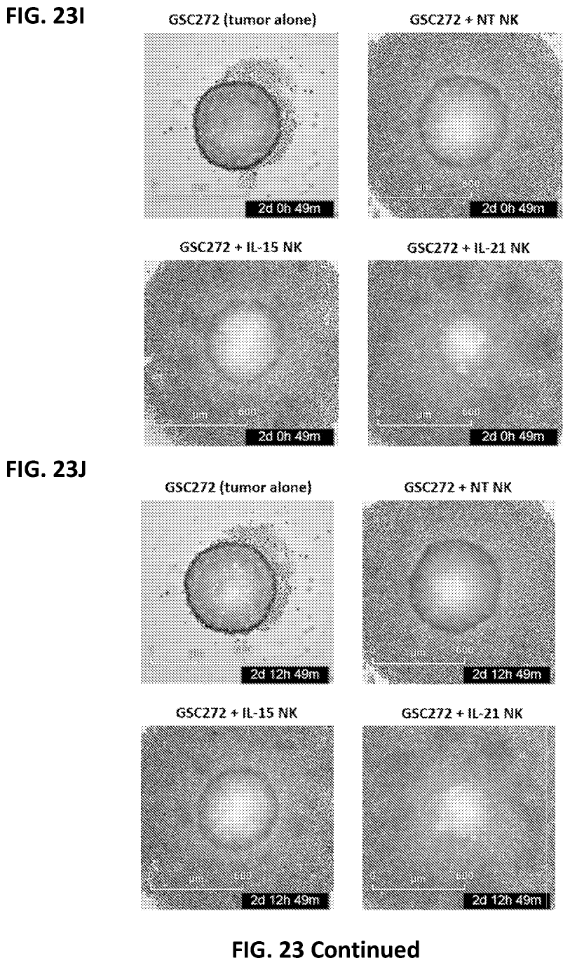

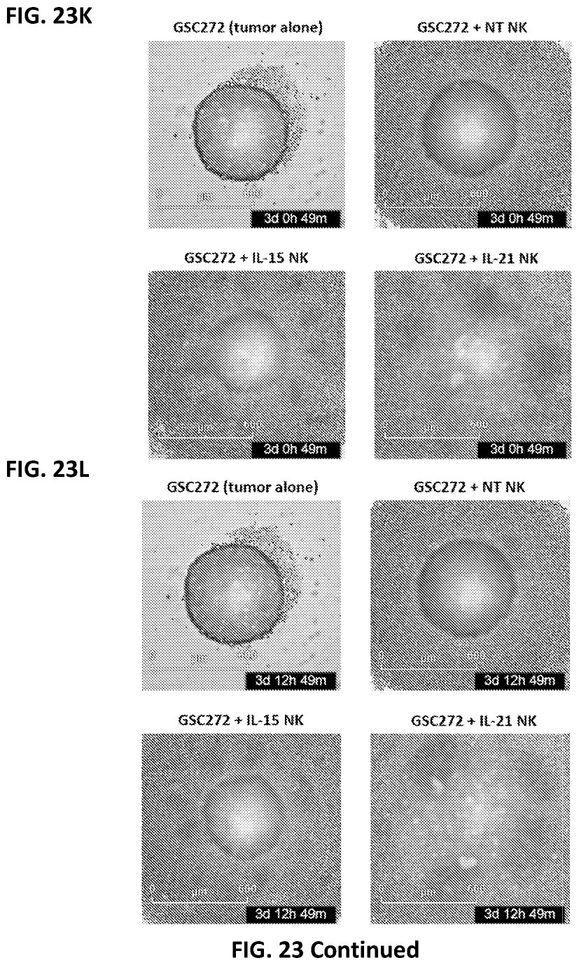

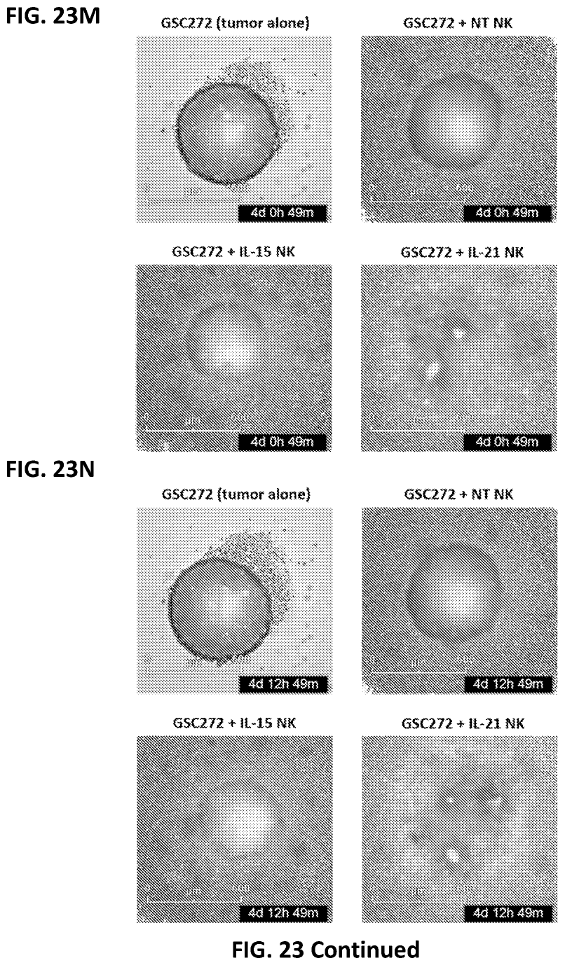

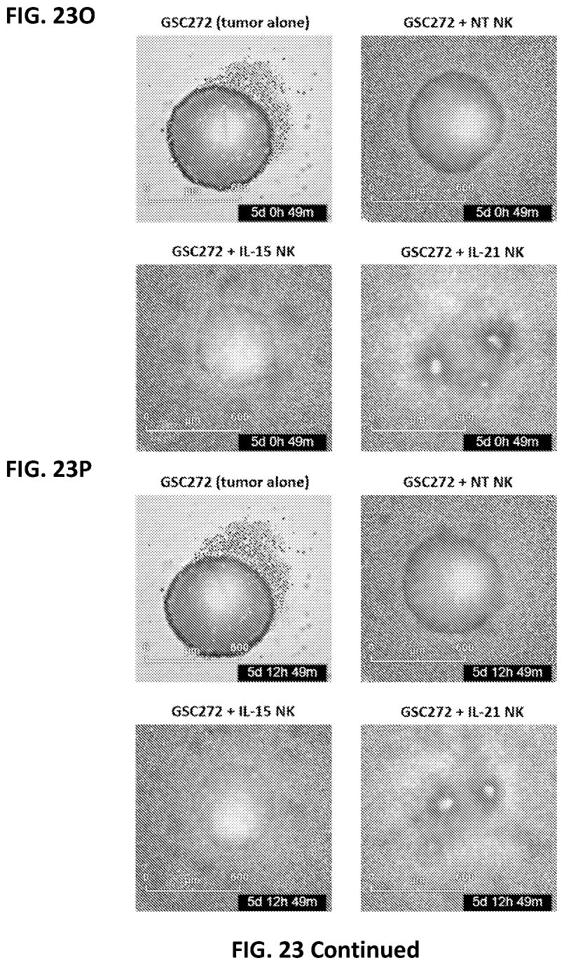

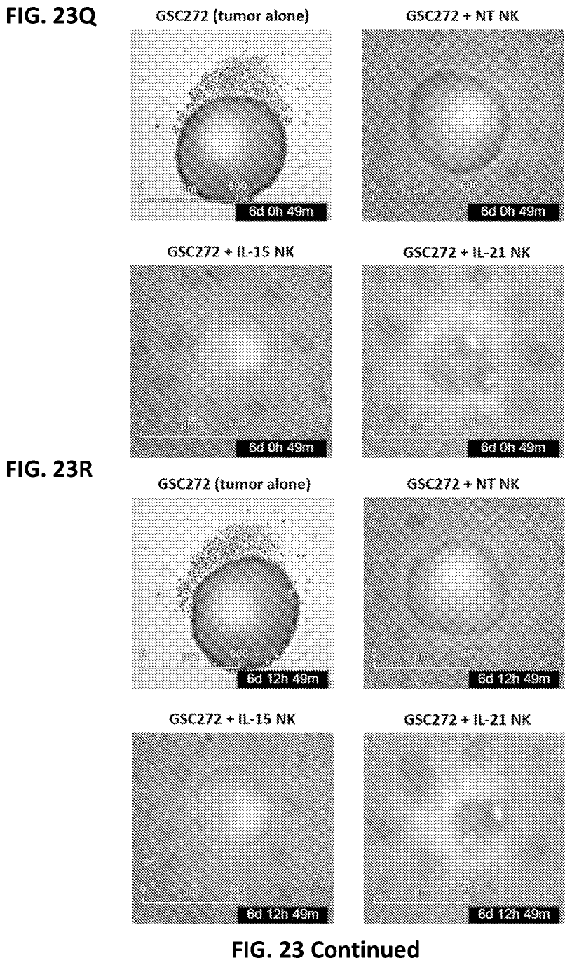

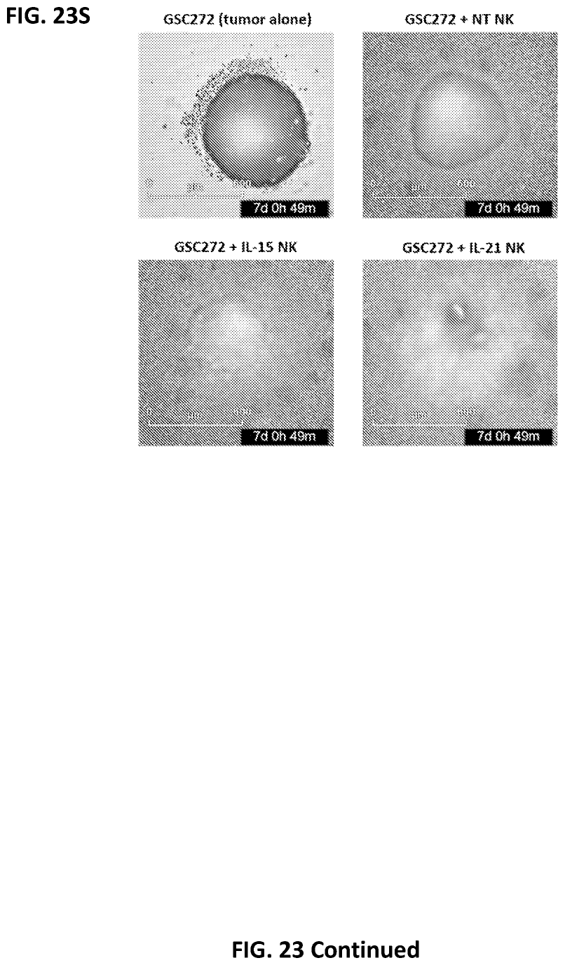

The Inventors have previously shown that GSCs suppress NK cell function through multiple mechanisms²⁴. To determine if arming NK cells with cytokines, such as IL-15 or IL-21 (“cytokine-armed”), could prevent GSC-induced suppression, NT NK cells and cytokine-transduced NK cells (IL-15 NK or IL-21 NK) were cultured with (FIG. 1E) or without (FIG. 1F) GSC20 for 48 hours. NK cells were then purified and their cytotoxicity tested against NK-sensitive K562 cells (lymphoblast cells isolated from the bone marrow of a male chronic myelogenous leukemia (CML) patient). Co-culture with GSC20 significantly impaired the cytotoxicity of NT NK cells against K562 cells, while IL-15 NK and IL-21 NK cells retained their cytotoxic function and ability to kill K562 cells (FIGS. 1E-1F). Next the potency of cytokine-armed NK cells was evaluated in a three-dimensional (3D) spheroid system using two GSC models, GSC20 and GSC272, which were cultured for 72 hours to form spheroids prior to introduction of NK cells (FIGS. 1G-1I, and FIGS. 23A-23S). The results confirmed that cytokine-transduced NK cells were more resistant to GSC-induced suppression than NT NK cells. These experiments demonstrated that NK cells expressing IL-15 or IL-21 killed GSC targets with greater potency than control NT NK cells, and were more resistant to tumor-induced dysfunction than NT NK cells.

### Example 2

IL-21 NK Cells Retained their Cytotoxicity Against Multiple GSC Rechallenges and Displayed Greater Metabolic Fitness

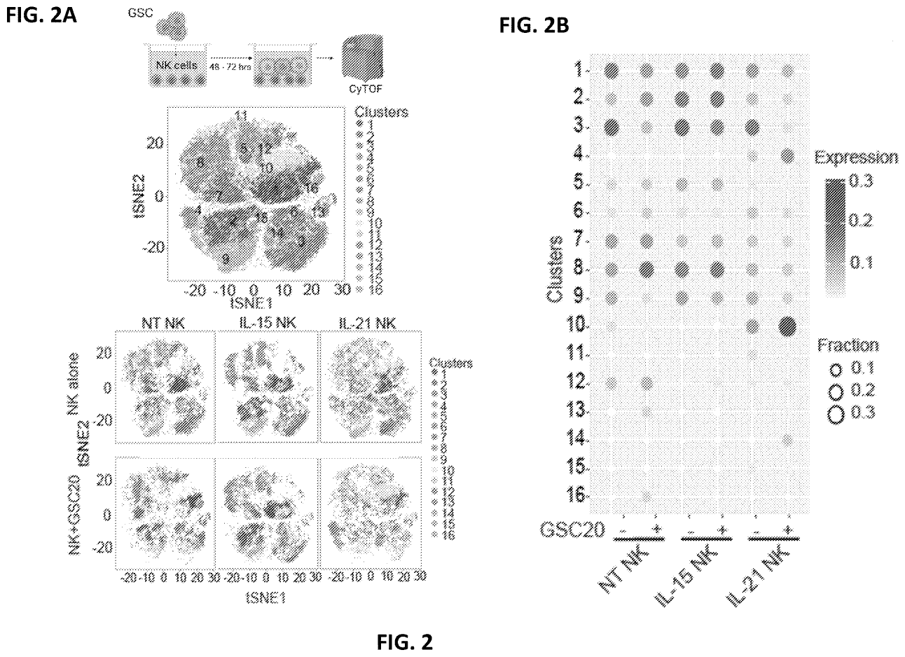

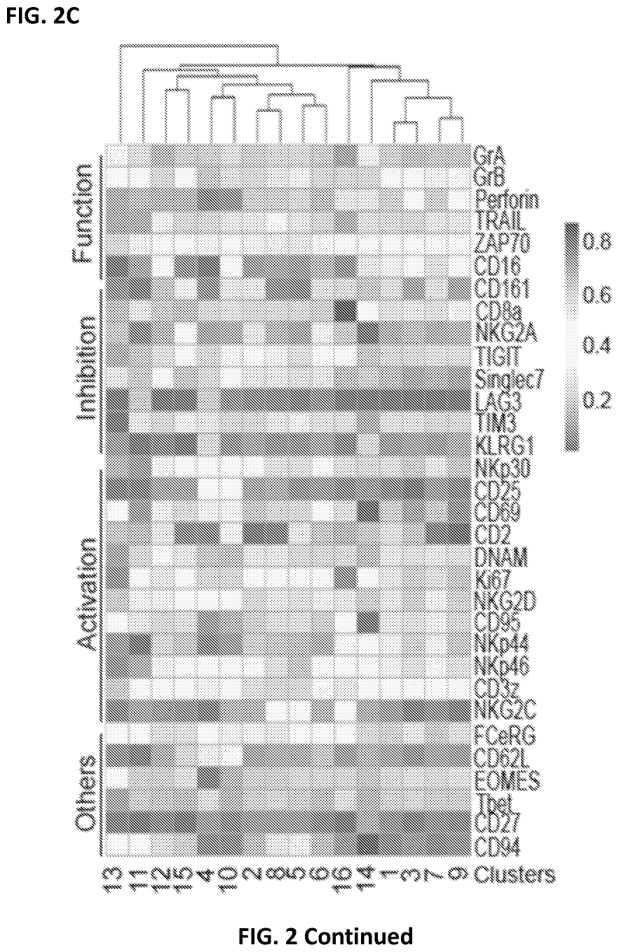

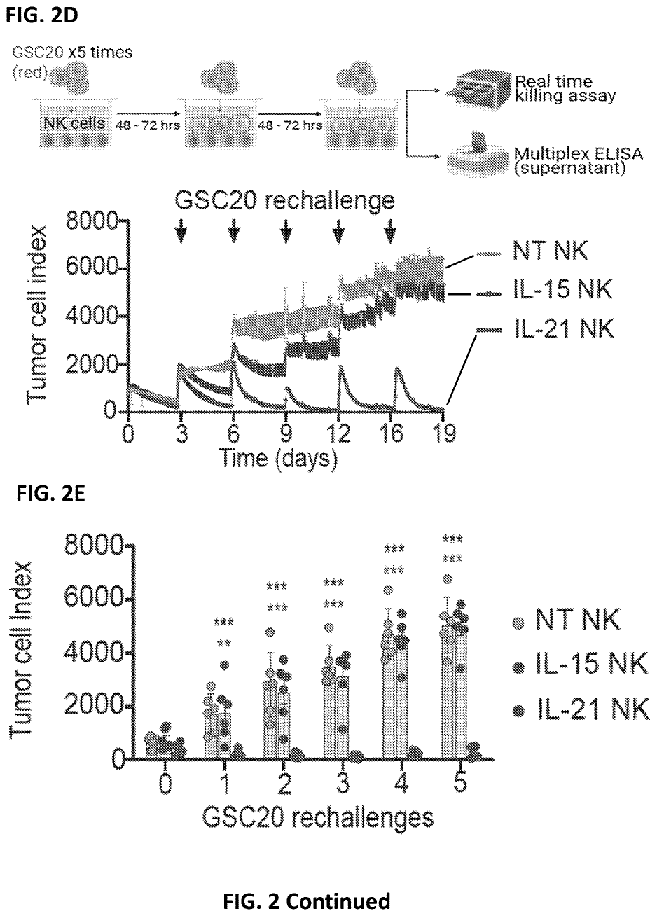

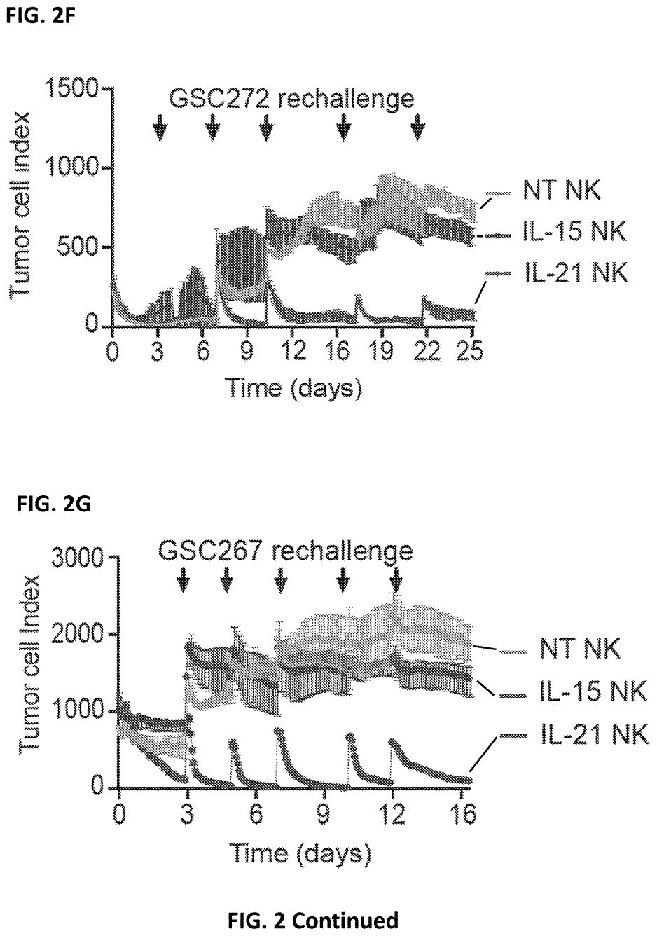

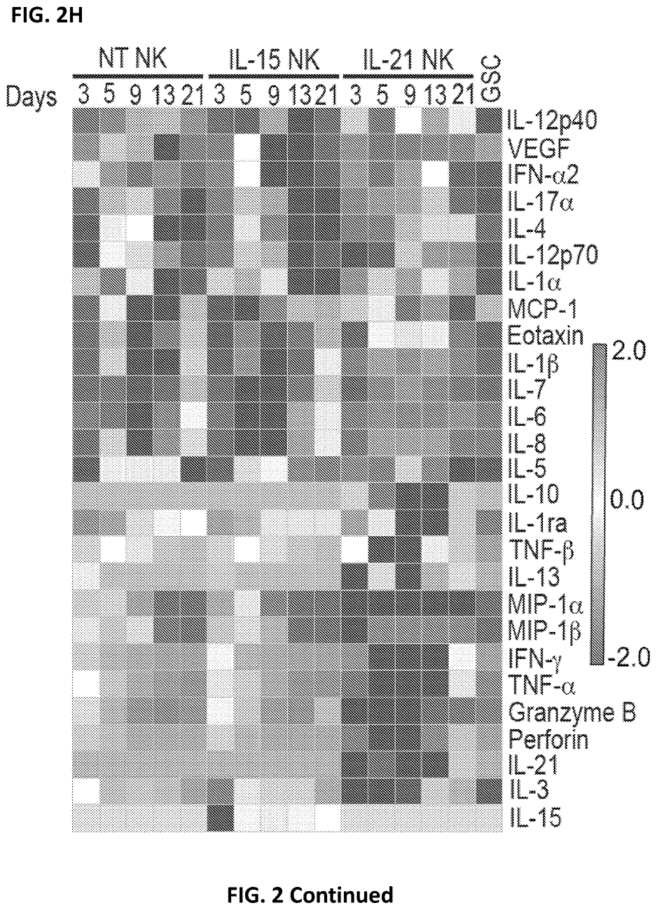

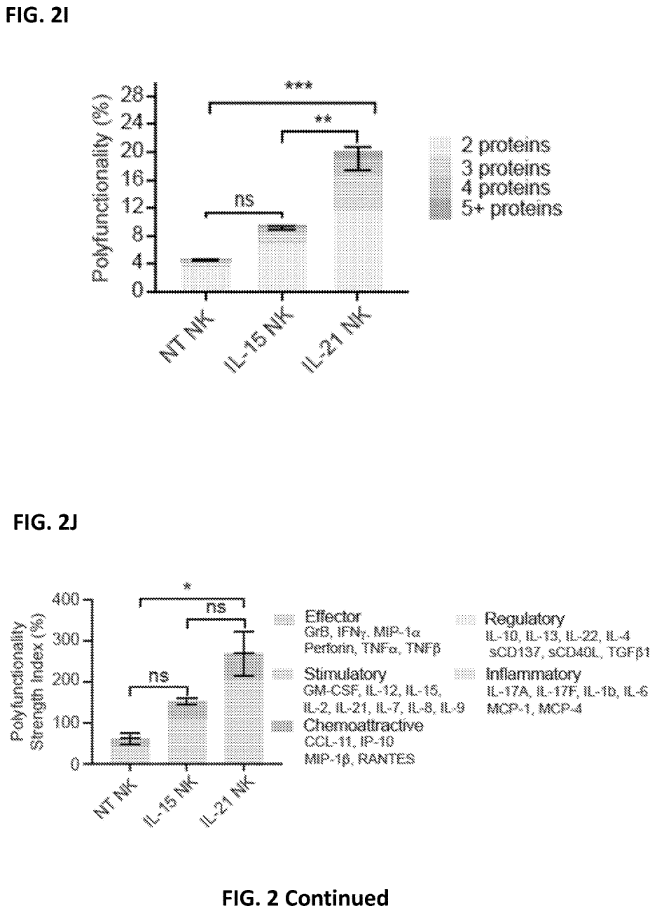

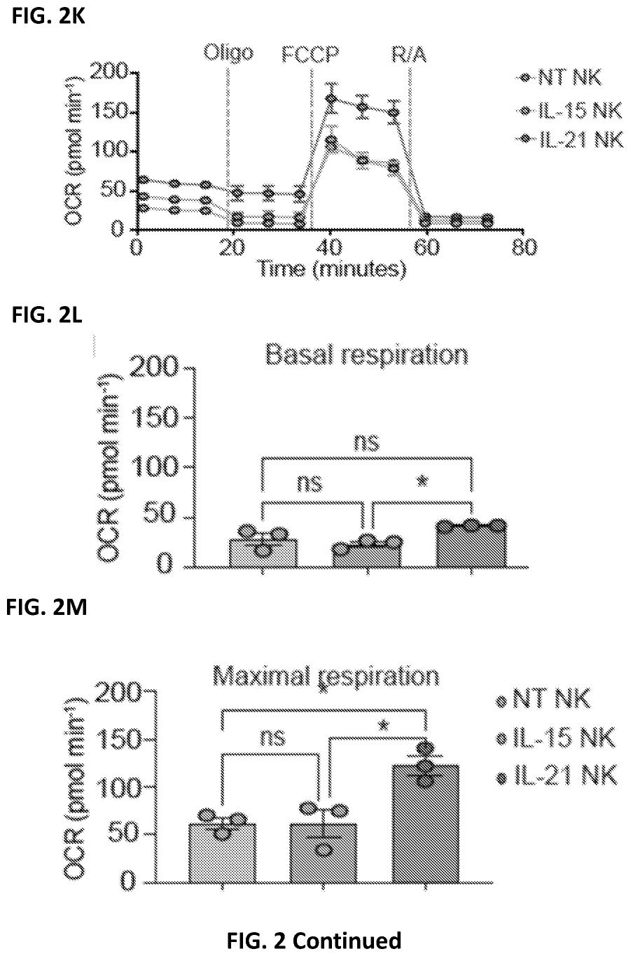

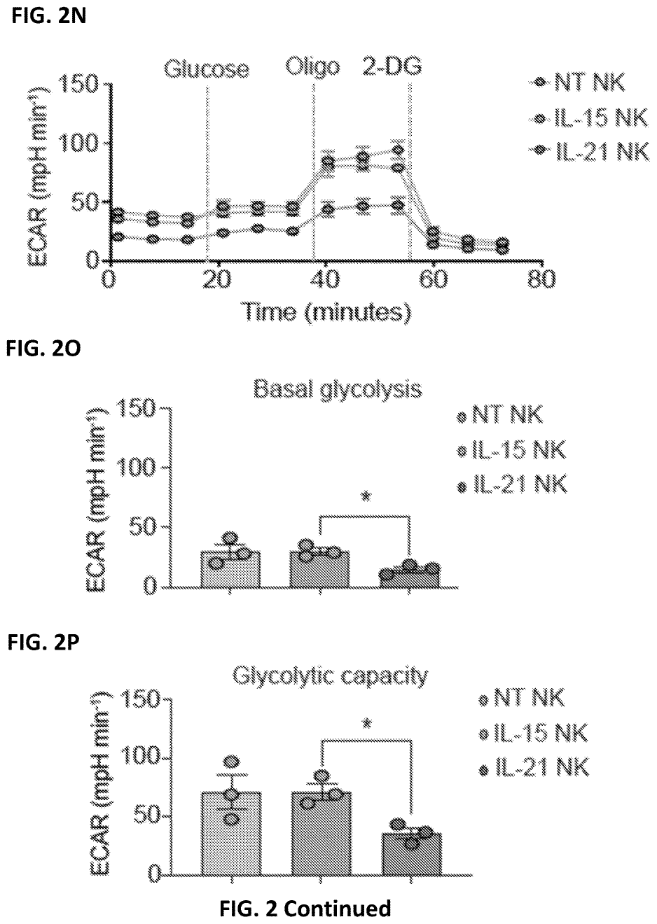

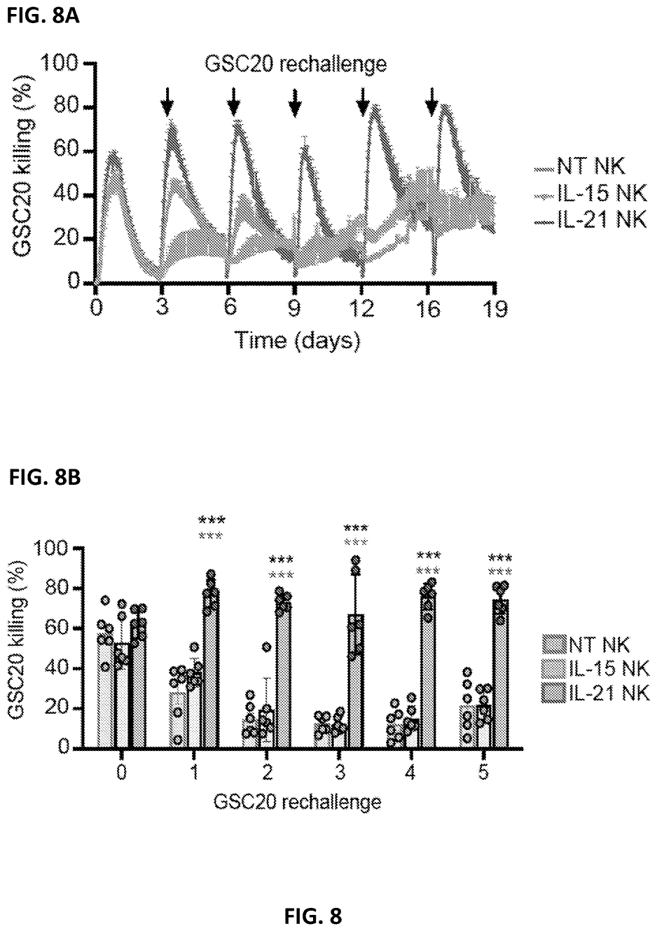

The short-term cytotoxicity assays described in Example 1 confirmed that, when compared to NT NK cells, IL-15 NK and IL-21 NK cells exerted greater cytotoxicity against GSCs. The long-term cytotoxicity of NT NK, IL-15 NK, and IL-21 NK cells was then evaluated using in vitro tumor rechallenge assays using multiple different GSCs, where the NK cells were challenged at a 1:1 E:T ratio with mCherry-transduced GSCs (e.g., GSC20, GSC272, GSC267, or GSC8-11) (red), and then rechallenged (200,000 GSC for each rechallenge) with additional mCherry-transduced GSCs every 2-3 days for at least five tumor rechallenges. Both IL-15 NK and IL-21 NK cells were equally effective at eliminating GSCs during the initial 3 days of co-culture after a single GSC rechallenge at an E:T ratio of 1:1 (FIGS. 2D-2G, FIG. 7C, and FIGS. 8A-8B). However, unlike IL-21 NK cells, IL-15 NK cells lost their ability to respond to additional GSC rechallenges, despite excellent viability (FIGS. 2D-2G, and FIGS. 7C-7E). Moreover, short-term priming of NT NK cells with exogenous human IL-21 (3 ng/ml) for 48 hours prior to co-culture with GSCs did not effectively control tumor growth (FIG. 7F), supporting a strategy to genetically modify NK cells to continuously secrete IL-21 at stable levels as shown in FIG. 7G. The stable autonomous IL-21 expression allowed the IL-21-transduced NK cells to maintain their ability to produce inflammatory cytokines (e.g., IFNγ and TNFα) and cytotoxicity molecules such as Granzyme B and Perforin throughout the various rechallenges (FIG. 2H and FIGS. 7H-7K). In contrast, both NT and IL-15 NK cells lost their ability to secrete these critical effector cytokines over time. Moreover, NK cells expressing IL-21 produced substantially lower levels of cytokines and chemokines associated with neurotoxicity (e.g., IL-6, IL-1β, MCP-1), when compared to their IL-15 transduced counterparts (FIG. 2H).

Polyfunctionality, cell diversity and metabolic fitness are important determinants of effective antitumor NK cell responses 25. Thus, the inventors utilized the single-cell IsoPlexis® platform to measure cytokine secretion by IL-15 NK, IL-21 NK, and NT NK cells in response to GSC20. It was observed that IL-21 NK cells were significantly more polyfunctional (e.g., significantly more NK cells exhibiting 2 protein, 3 protein, 4 protein, and/or 5+ protein secretion profiles) when compared to IL-15 NK or NT-NK cells (n=3 donors; FIGS. 2I-2J, and FIG. 7L). Furthermore, IL-21 NK cells were shown to exhibit increased polyfunctionality strength index (%), where IL-21 NK cells exhibited increased effector (e.g., GrB, IFNγ, MIP-1α, Perforin, TNFα, and/or TNFβ) and chemoattractive (e.g., CCL-11, IP-10, MIP-1β, and/or RANTES) expression profiles when compared to NT NK cells (FIG. 2H).

Mass cytometry was utilized to interrogate single-cell proteomic profiles of IL-15 NK, IL-21 NK, and NT NK cells cultured for 48 hours in the presence or absence of GSC20 cells (n=3 CB donors) (FIGS. 2A-2C). A number of clusters were observed that appeared to be exclusive to IL-21 NK cells (e.g., cluster 4, cluster 10, cluster 11, and cluster 14). A number of these clusters (e.g., cluster 4, cluster 10) were observed to further expand upon co-culture with GSC cells (FIGS. 2A-2C). These clusters included clusters 4 and 10, which were characterized by high or higher expression of functional/cytotoxicity markers such as Granzyme A (GrA), Granzyme B (GrB), Perforin, and Zap70; low or lower expression of inhibitory markers including LAG3, CD95, and KLRG1; and upregulation of markers associated with activation and proliferation such as NKp30, CD25, DNAM, Ki67, CD3ζ, EOMES, T-bet, and/or FCεRG (FIG. 2C). Cluster 14 was exclusive to IL-21 NK cells after co-culture with GSCs and had high levels of cytotoxicity markers (e.g., including GrA, GrB, Perforin, TRAIL, CD95), as well as activation markers/receptors (e.g., CD25, CD69, DNAM, NKG2D, NKp44 and NKp46).

Arming NK cells with cytokines may also modulate their intrinsic metabolic fitness; thus, the mitochondrial metabolism and glycolytic potential of NT NK, IL-15 NK, and IL-21 NK cells was analyzed in cells that had been cultured for 48 hours with GSC20 cells (FIGS. 2K-2P). IL-21 NK cells showed significantly higher basal and maximal oxygen consumption rate (OCR) compared to IL-15 NK cells (FIGS. 2K-2M). Furthermore, IL-21 NK cells showed a reciprocal significant reduction in extracellular acidification rate (ECAR), a measure of glycolysis, when compared to IL-15 NK cells (FIGS. 2N-2P). These results indicated that IL-21 NK cells produced ATP primarily by oxidative phosphorylation, a feature associated with long-term persistence and anti-tumor activity²⁶.

### Example 3

### IL-21 NK Cells Displayed Cytotoxicity to GSC Cells In Vivo, and Displayed Memory Response to GSC Rechallenge In Vivo

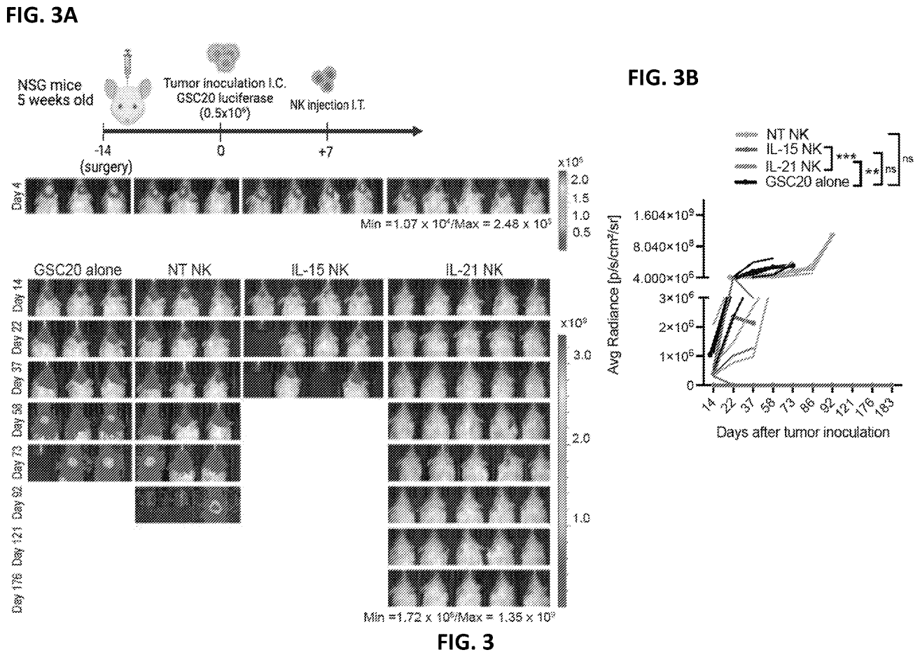

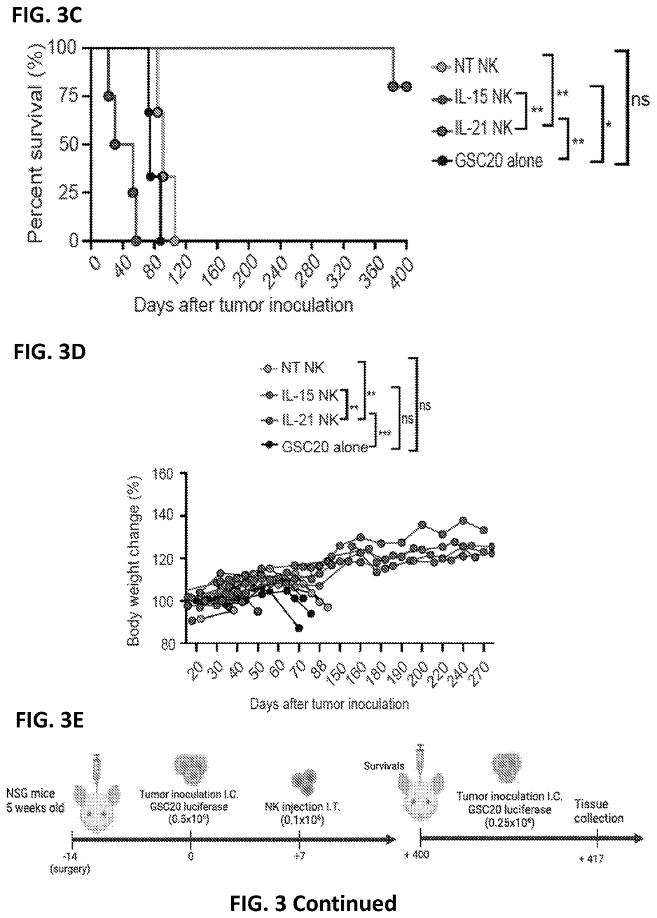

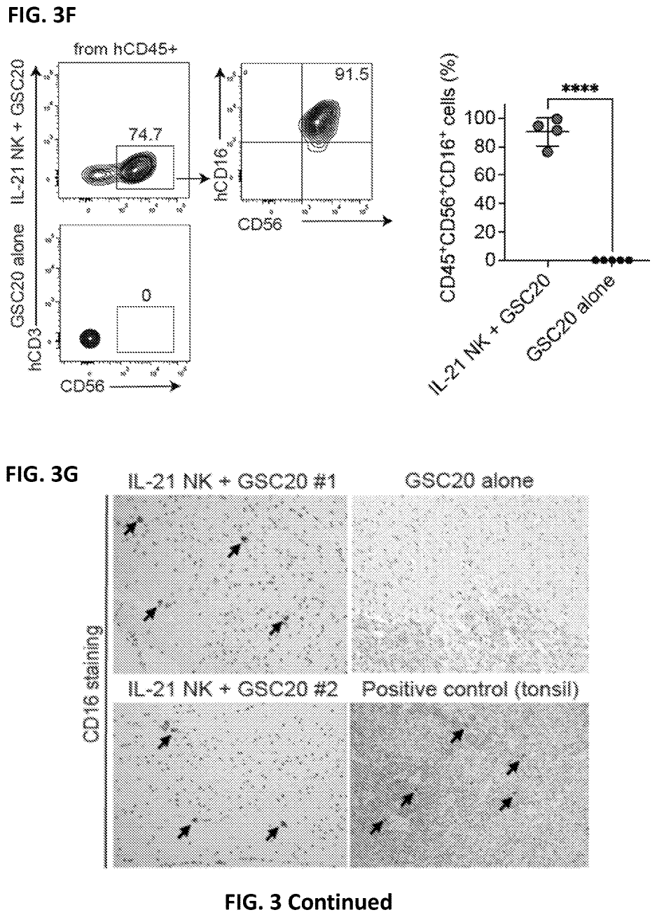

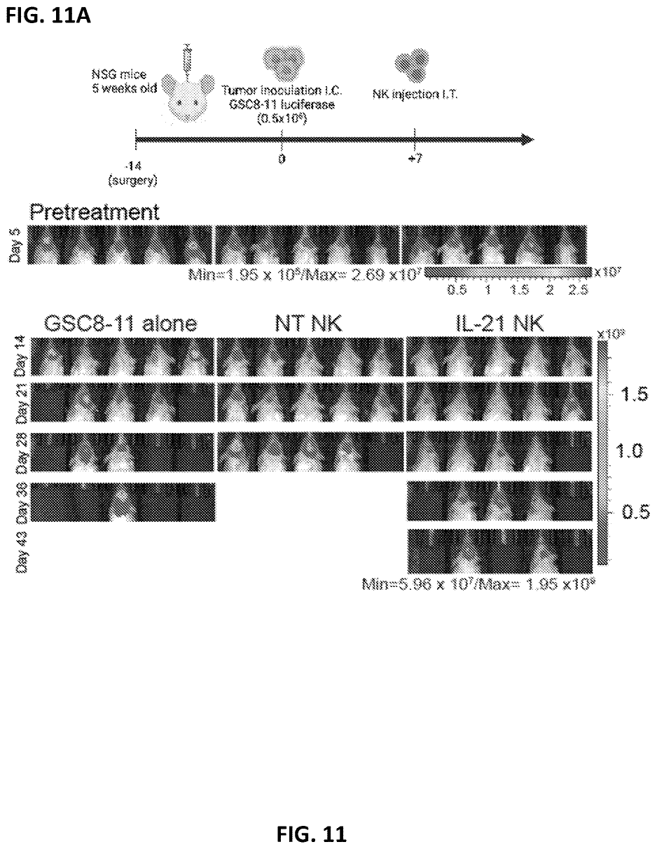

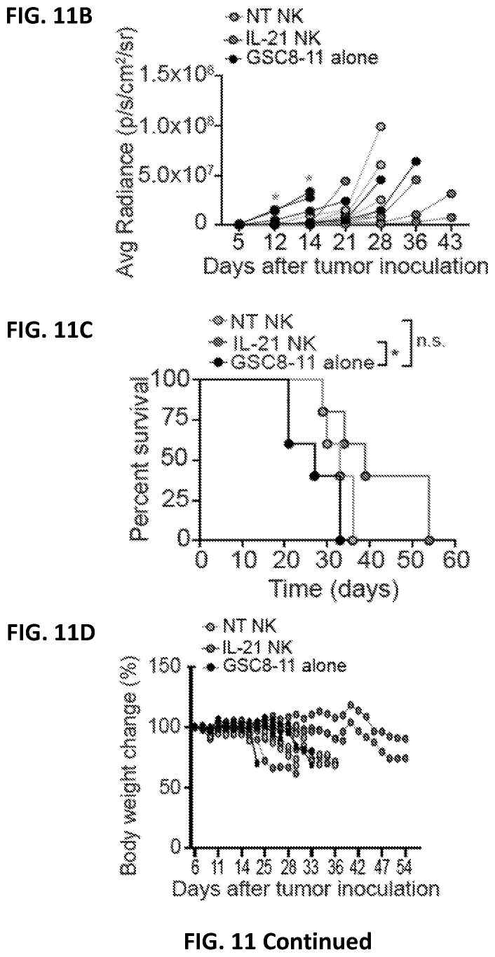

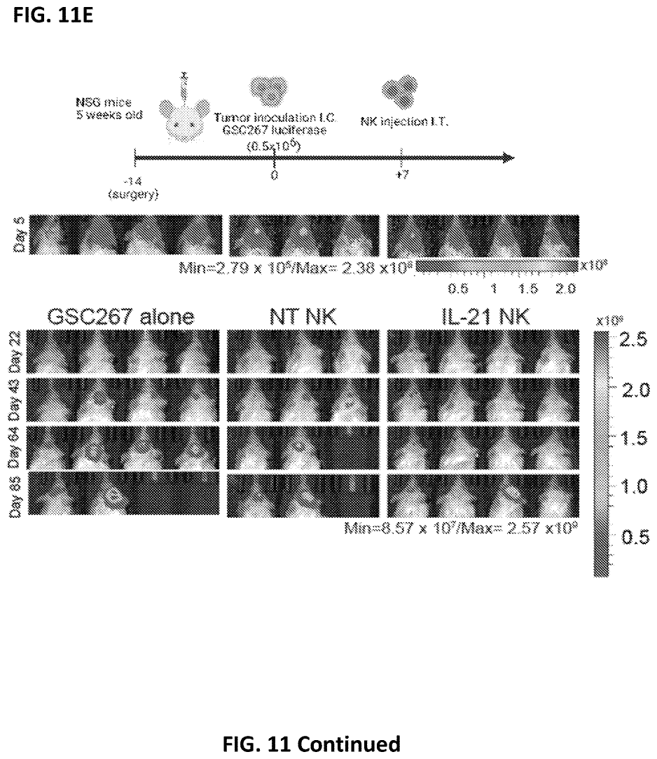

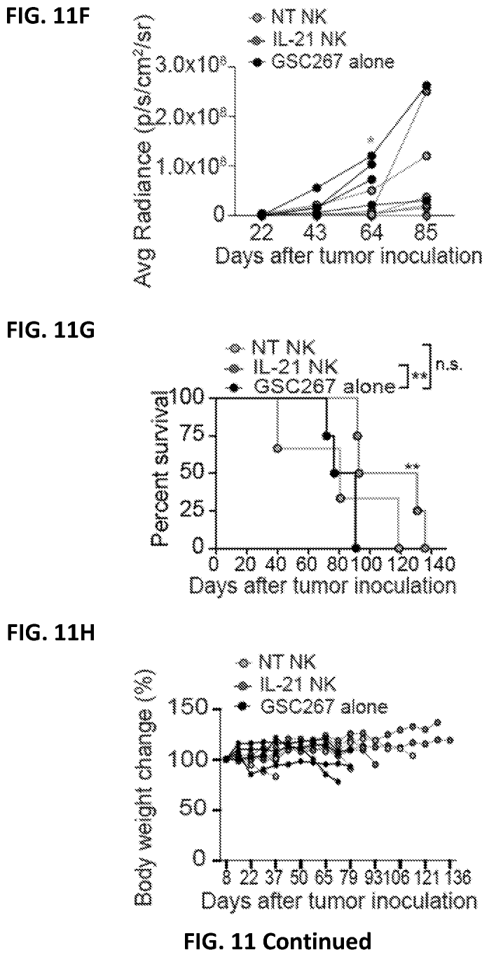

To investigate and compare the in vivo antitumor function of IL-21 NK, IL-15 NK cells, and NT NK cells, three orthotopic PDX mouse models of patient-derived GSCs were utilized (GSCs described herein in the Materials and Methods, and as previously reported⁵). In brief, at day 0, 5 week old NSG mice were inoculated (intracranial, I.C.) with 0.5×10⁶ GSC20 luciferase cells. Seven days after tumor implantation, mice with established tumor were treated intratumorally (IT) with a single infusion of 0.1×10⁶ IL-15 NK, IL-21 NK, or NT NK cells, or mock treatment. Similar to the in vitro results described above, good short-term tumor control was observed with both IL-15 NK and IL-21 NK cell groups and to a lesser extent with NT NK cells (FIGS. 3A-3B) relative to GSC20 alone animals. The best tumor control and survival were observed in mice treated with IL-21 NK cells, with a single IT administration of IL-21 NK cells resulting in long-term eradication of tumor in the GSC20 model and significant prolongation of survival in the GSC8-11 and GSC267 models, without any evidence of toxicity or weight loss (FIGS. 3A-3D, and FIGS. 11A-11H).

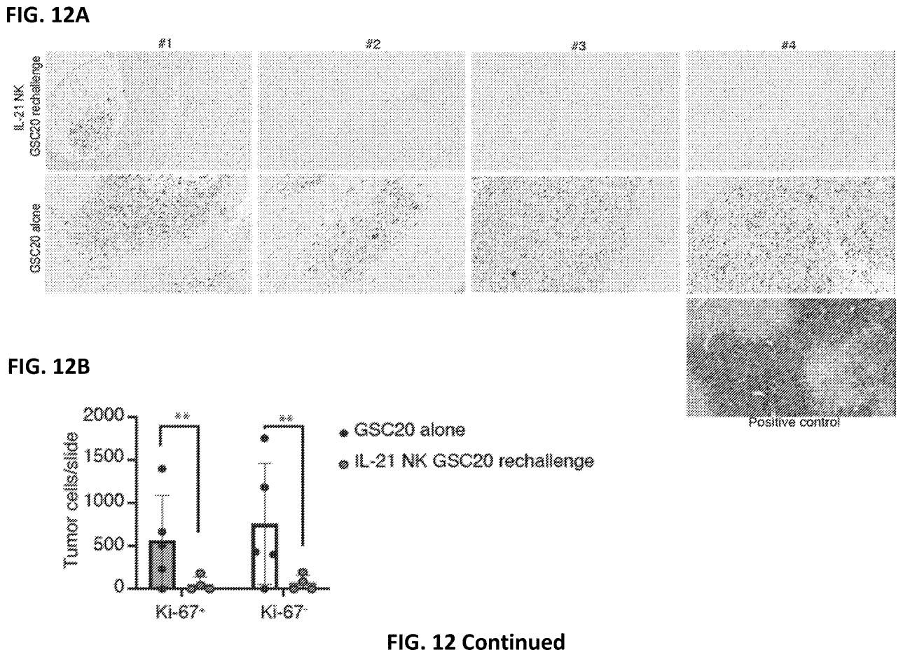

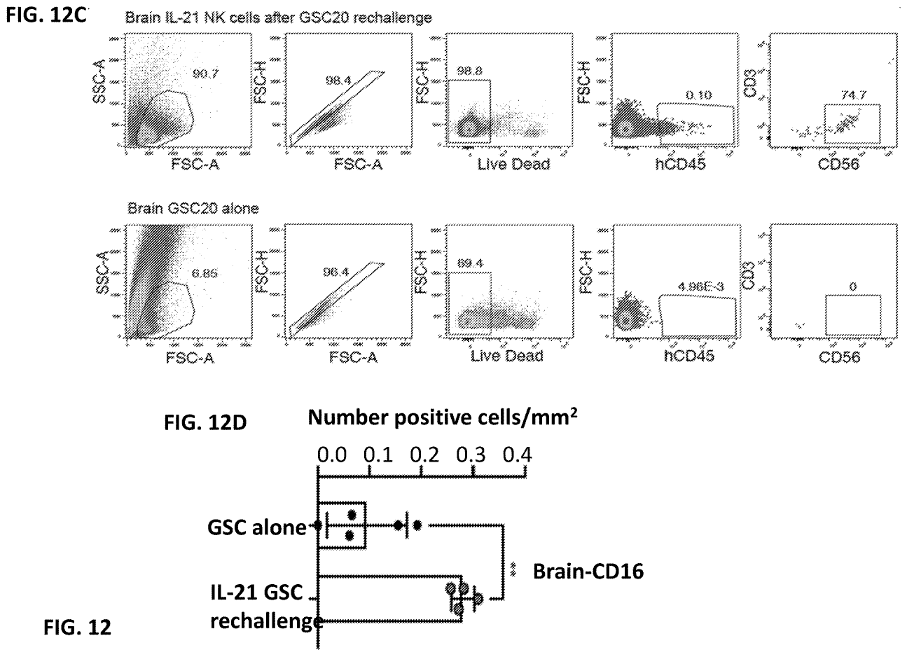

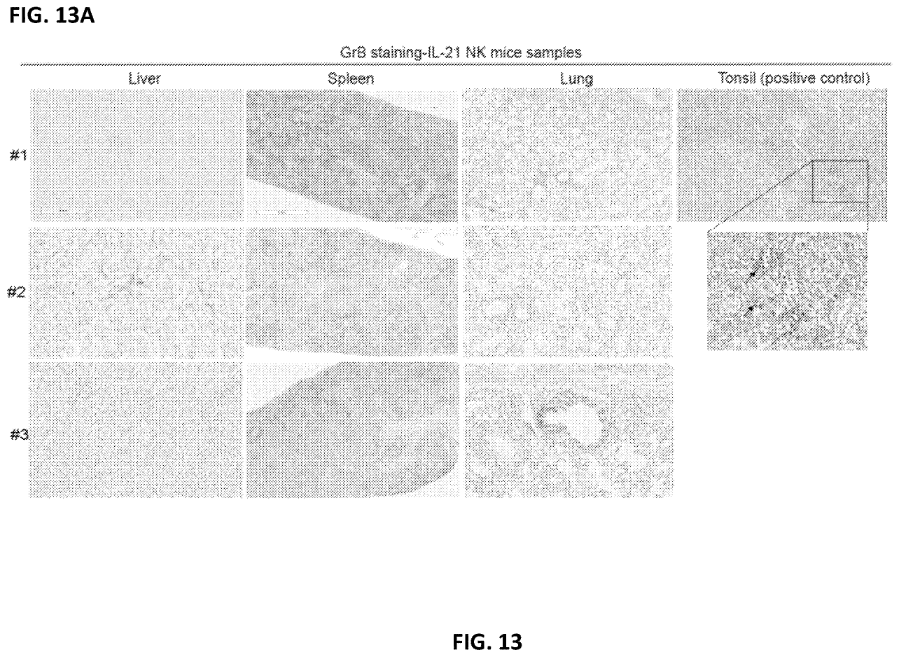

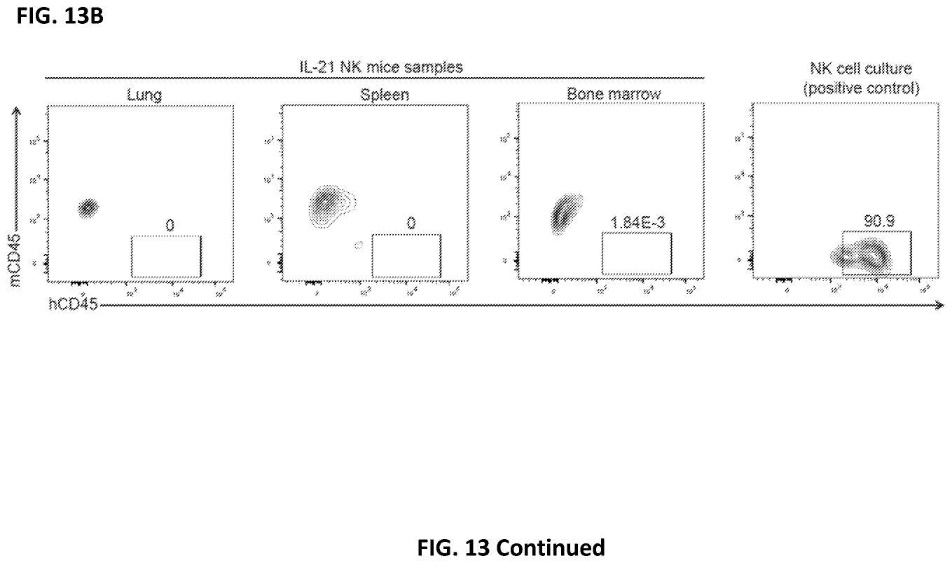

The persistence of IL-21 NK cells was analyzed using an in vivo GSC tumor rechallenge model. Mice that had previously been treated with IL-21 NK cells and had survived the initial GSC20 tumor challenge (n=4) underwent a second tumor inoculation of 0.25×10⁶ GSC20 tumor cells 400 days after the initial treatment (FIG. 3E). This group was compared to a control group of naïve non-tumor bearing mice (n=5) also injected intracranially with 0.25×10⁶ GSC20 cells. All animals were sacrificed two weeks after the tumor challenge and necropsy was performed. Examination of brain tissue from the control group confirmed tumor presence (FIG. 12A-12B). In stark contrast, brains from the IL-21 NK cell rechallenge group showed a distinct infiltration of human CD45⁺ cells, all of which were CD56⁺CD3⁻CD16+ NK cells, with only minimal tumor detection (FIGS. 3F-G; and FIGS. 12A-12D). Furthermore, analysis by flow cytometry and IHC revealed no NK cell infiltration in secondary organs such as lungs, spleen, liver and bone marrow (FIGS. 13A-13B), suggesting a potential brain-residency of these long-lived IL-21 NK cells.

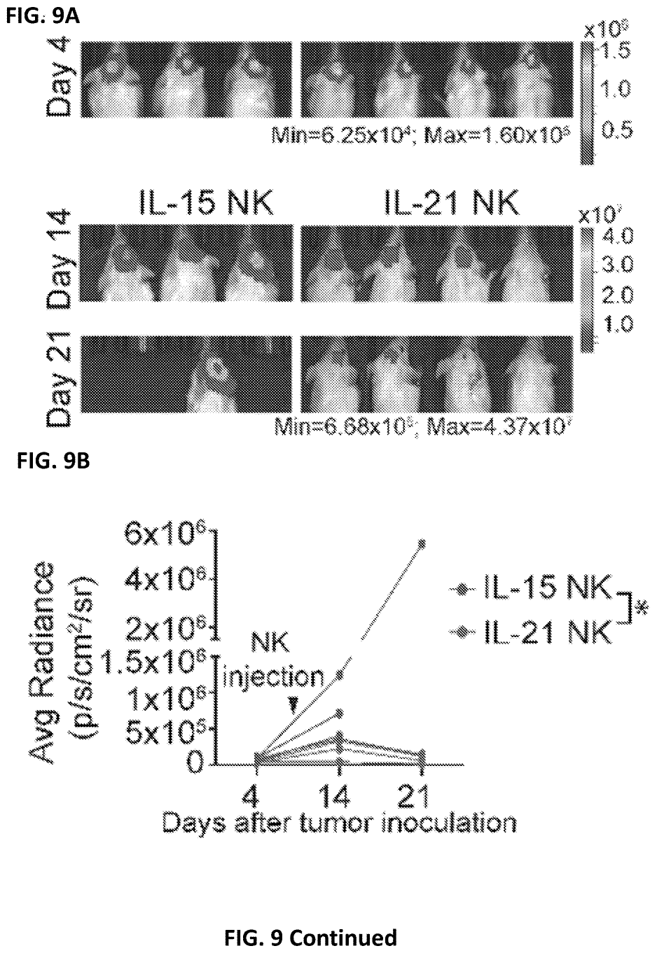

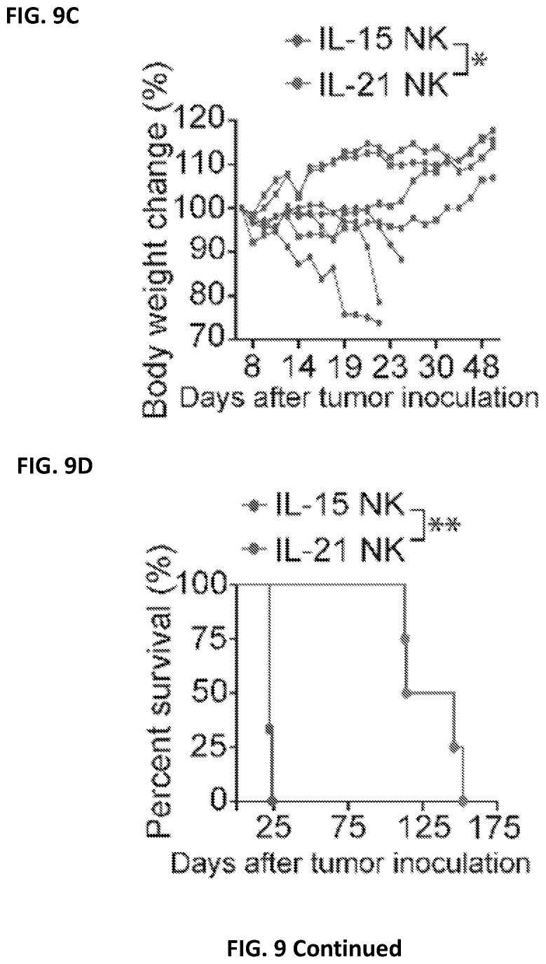

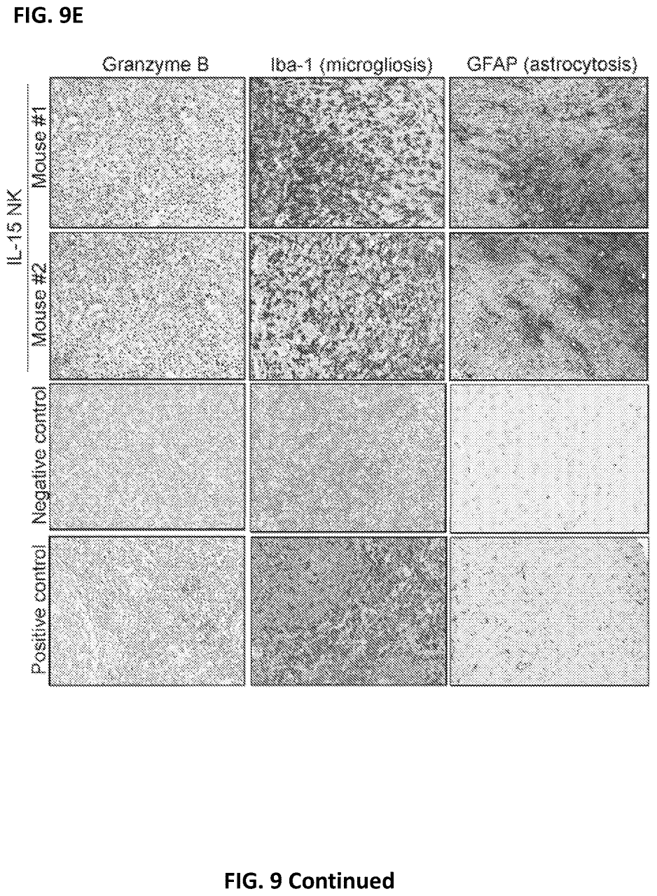

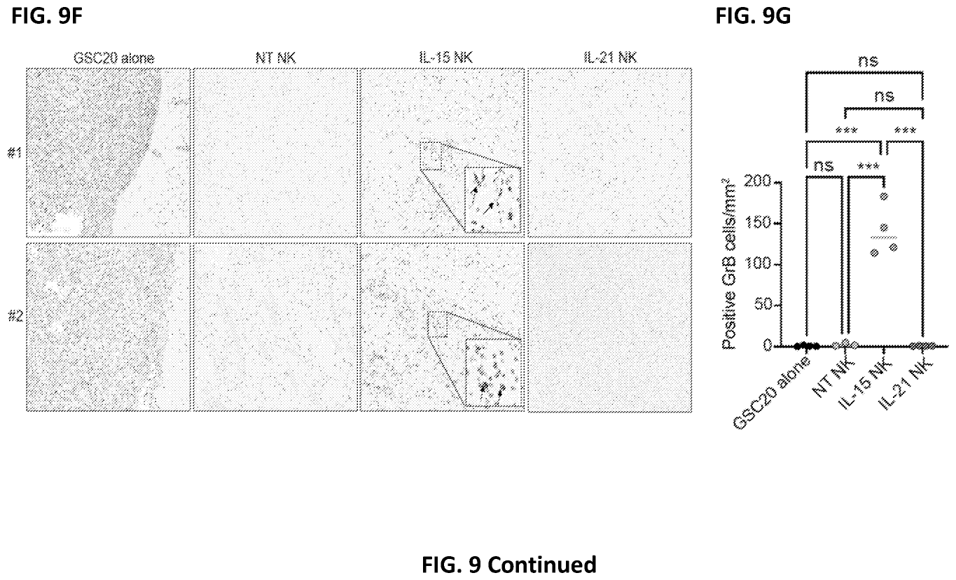

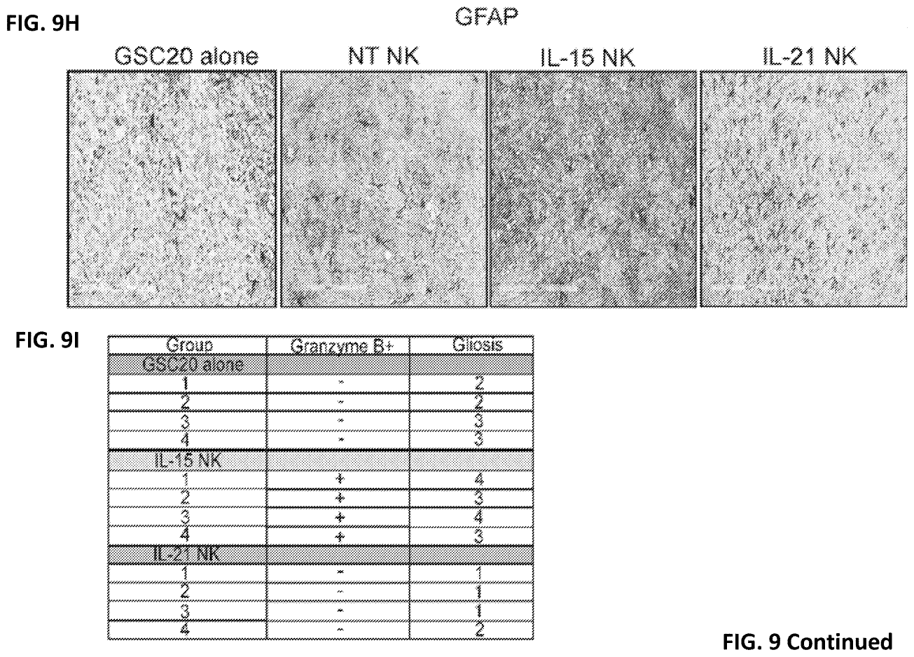

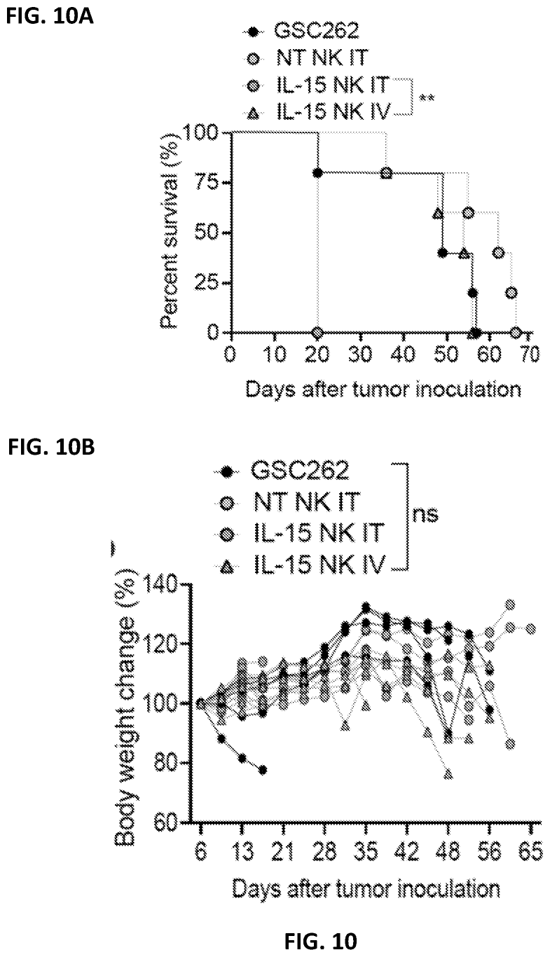

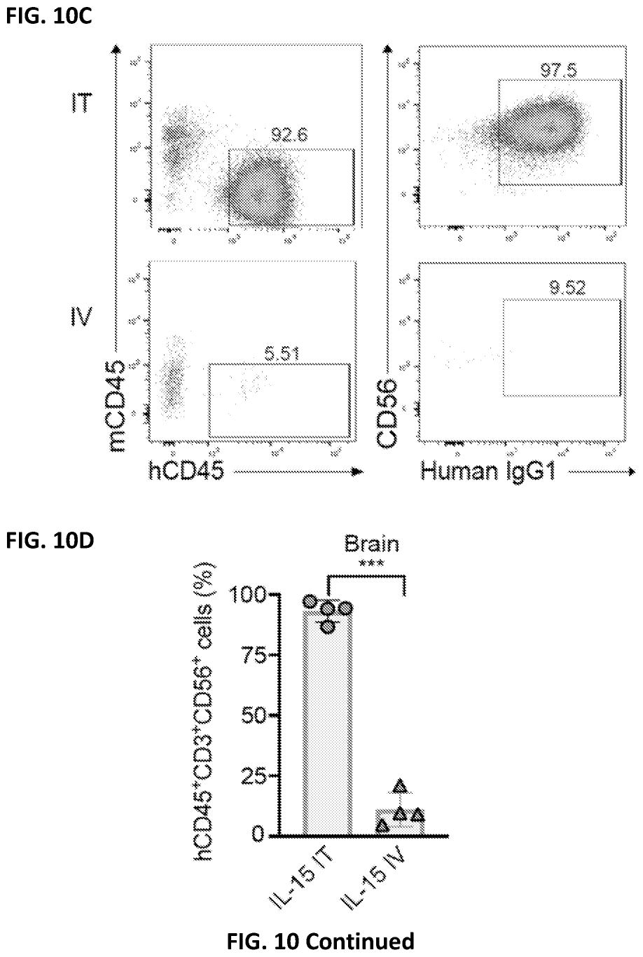

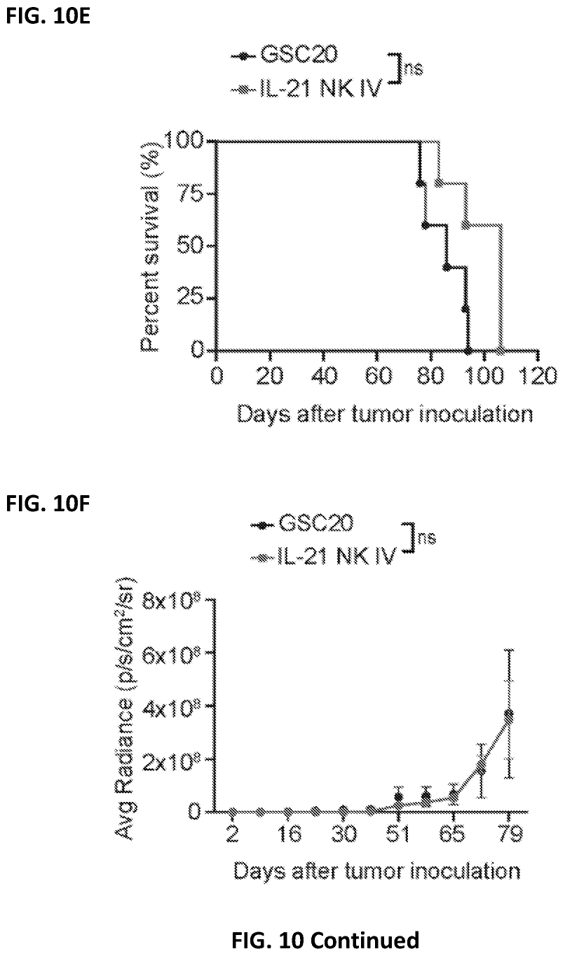

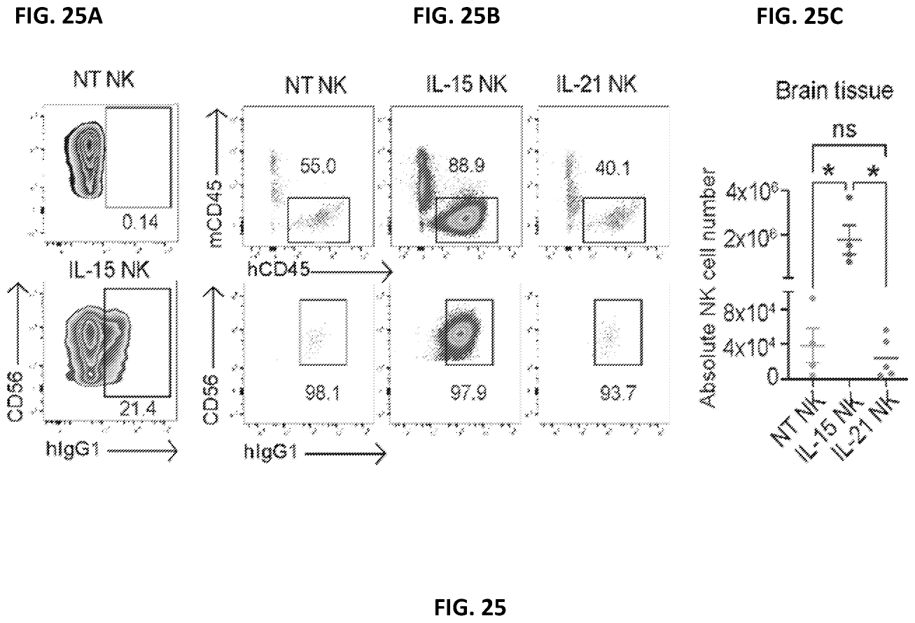

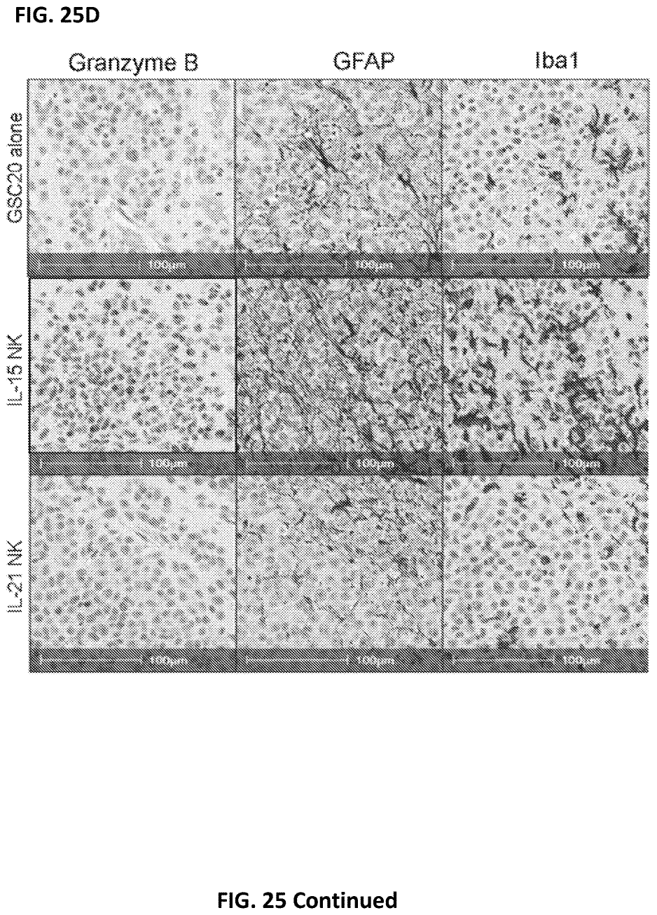

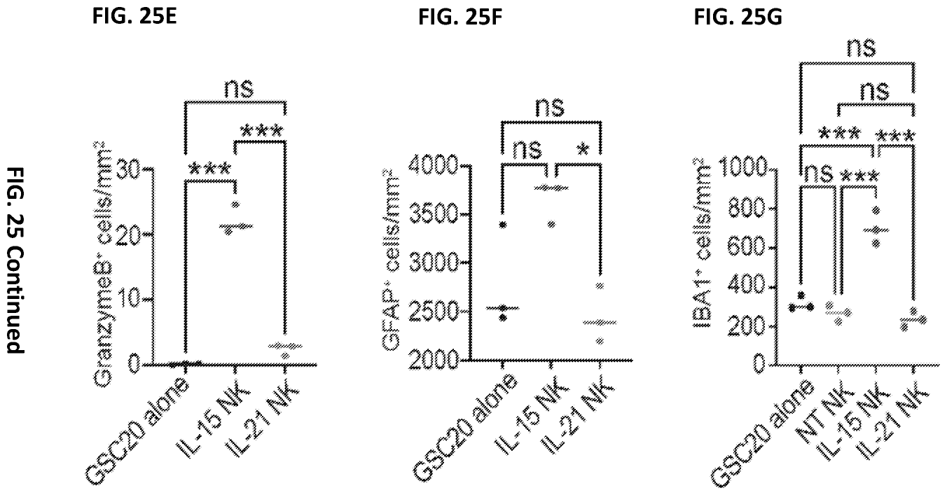

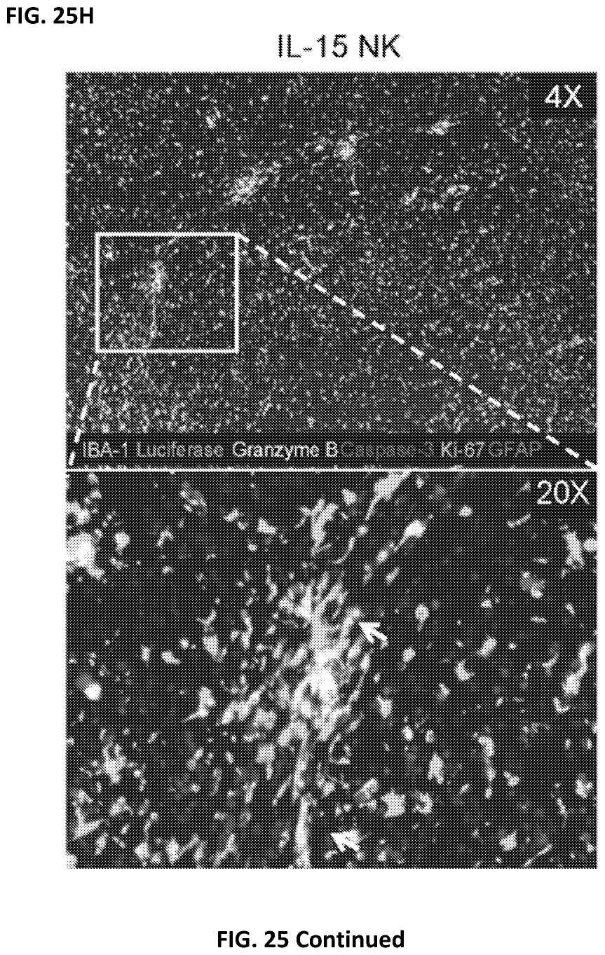

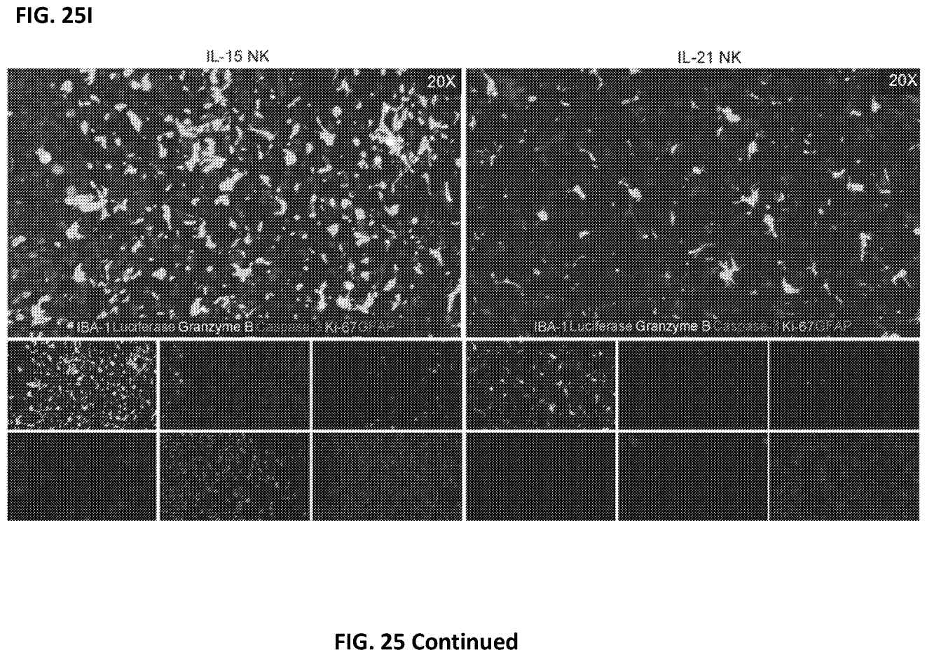

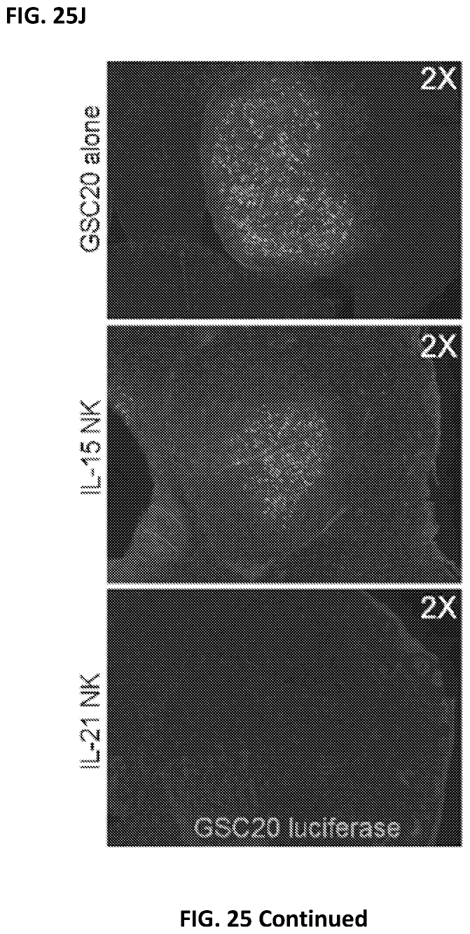

In contrast to the IL-21 NK, or NT NK cell groups, IT administration of 0.5×10⁶ IL-15 NK cells resulted in significant toxicity and suboptimal GSC20 tumor control, and early mortality in mice (FIGS. 3A-3D), associated with heavy NK cell infiltration and marked gliosis in the brain (Table 4; FIGS. 9A-9E). This was characterized by significant weight loss, heavy infiltration of activated NK cells as evidenced by Granzyme B staining, marked activation of microglia indicated by increased Iba1 staining and changes in cell morphology as previously defined²⁷, and considerable astrocytosis highlighted by glial fibrillary acidic protein (GFAP) staining (FIGS. 9A-9E). A similar level of toxicity was observed both with a lower dose of IL-15 NK cells (0.1×10\hat{ }6) (FIGS. 3A-3D, and FIGS. 9F-9I) and lower transduction efficiency of IL-15 (21.4% IL-15 transduction vs ~80% normal transduction (FIGS. 25A-25J). Notably, close proximity of NK cells to Ki67 positive microglia was observe din normal brain tissue adjacent to the tumor, but not within the tumor itself, suggesting a possible role for NK-microglia crosstalk in the proliferation of microglia and subsequent neuroinflammation (FIGS. 25H-25I). The underlying mechanism could involve the expression of IL-15 receptor (IL-15R) on microglia and the ability of human IL-15 to engage the murine IL-15R as reported in the literature²⁸, supporting the notion that microglia activation and proliferation are a consequence of IL-15 released by NK cells. When compared to IL-21 NK cell treated mice, the IL-15 NK cell group had inferior tumor control efficacy (FIG. 25J). The observed neurotoxicity and early mortality caused by IL-15 NK cells following IT administration were observed in a second model using GSC262 cells (FIG. 10A). To determine if the route of administration influences the degree of toxicity, IT vs intravenous (IV) administration of IL-15 NK cells was compared (FIGS. 10A-10D). When IL-15 NK cells were given IV (FIGS. 10A-10B) toxicity was not observed, although there was also minimal NK cell infiltration into the brain and no tumor control (FIGS. 10C-10D), suggesting that the lack of toxicity following IV infusion was due to the limited trafficking of IL-15 NK cells to the brain. Serum analysis from mice that died of toxicity following treatment with IT IL-15 NK cells did not show markers of cytokine release syndrome (e.g. IL-6, IL-1β, MIP1α, IFNγ, TNFα, and IL-10; Table 1), supporting localized toxicity effects of IL-15 in the brain rather than systemic inflammation. Similarly, IV administration of IL-21 NK cells was not toxic but also failed to control tumor growth, again likely attributed to limited NK cell trafficking to the brain (FIGS. 10E-10F). Collectively, these data suggest that the antitumor efficacy of IL-21 NK cells and the toxicity of IL-15 NK cells are restricted to their localized effect within the brain when administered intratumorally.

TABLE 4
Brain histopathology measuring infiltration with human granzyme
B+ cells (as a measure of NK cells) and the gliosis
score in human IL-15 NK and IL-21 NK cell-treated mice

| Treatment | Group | Granzyme B+ | Gliosis |
|---|---|---|---|
| GSC20 alone | 1 | Negative | 2 |
| | 2 | Negative | 2 |
| | 3 | Negative | 3 |
| | 4 | Negative | 3 |
| IL-15 NK cells | 1 | Positive | 4 |
| | 2 | Positive | 3 |
| | 3 | Positive | 4 |
| | 4 | Positive | 3 |
| IL-21 NK cells | 1 | Negative | 1 |
| | 2 | Negative | 1 |
| | 3 | Negative | 1 |
| | 4 | Negative | 2 |

*Brain histopathology measuring was performed to analyze infiltration with human granzyme B+ cells (as a measure of NK cells), and the gliosis score in human IL-15 NK and IL-21 NK cell-treated mice was analyzed. The frontal lobe was examined for the presence or absence of granzyme B+ cells (as a marker of human NK cells) and for the presence or absence of increased glial cells (gliosis).
*Gliosis was scored semi-quantitatively using the following scale: 0 = no apparent increase in glial cells, 1 = minimal gliosis (cellularity increased 1-10%), 2 = mild gliosis (cellularity increase 11-30%), 3 = moderate gliosis (cellularity increase 31-50%) and 4 = marked gliosis (cellularity increased greater than 50%).

TABLE 1
Human cytokine concentration (pg/ml) in mouse serum after
treatment with IL-15 NK (n = 2), IL-21 NK (n =
5) or GSC20 control (n = 3).

| Sample | IL-6 | IL-1β | MIP-1α | IFN-γ | TNF-α | IL-10 |
|---|---|---|---|---|---|---|
| GSC control | 10.9 | 0.8 | 0.9 | 0.7 | 0.3 | 1.2 |
| IL-15 NK | 0.0 | 0.0 | 1.0 | 0.1 | 0.4 | 0.0 |
| IL-21 NK | 0.0 | 0.2 | 1.0 | 0.0 | 0.8 | 0.0 |

### Example 4

### IL-21 NK Cells Displayed Transcriptomic and Epigenetic Signatures

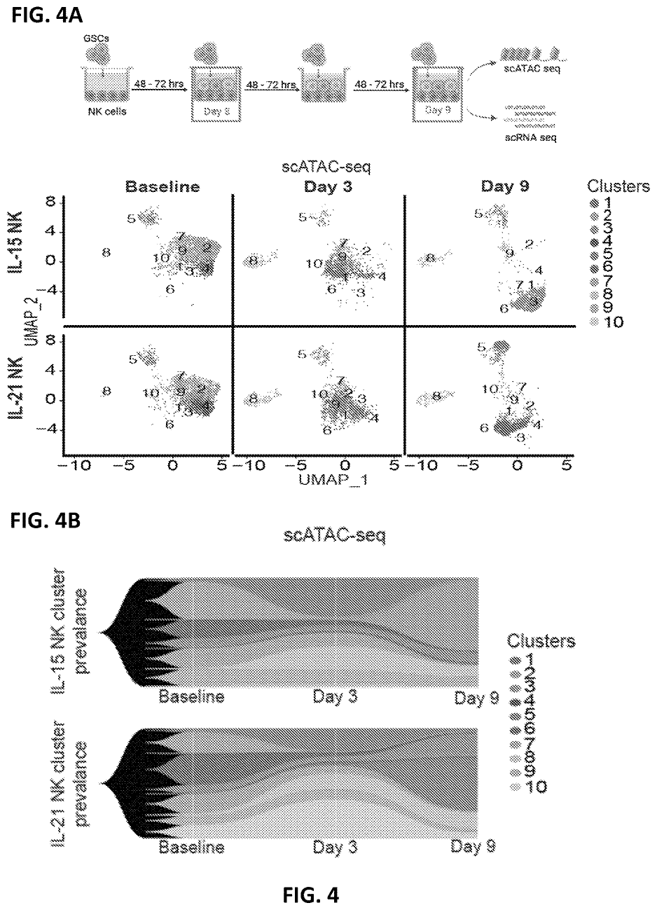

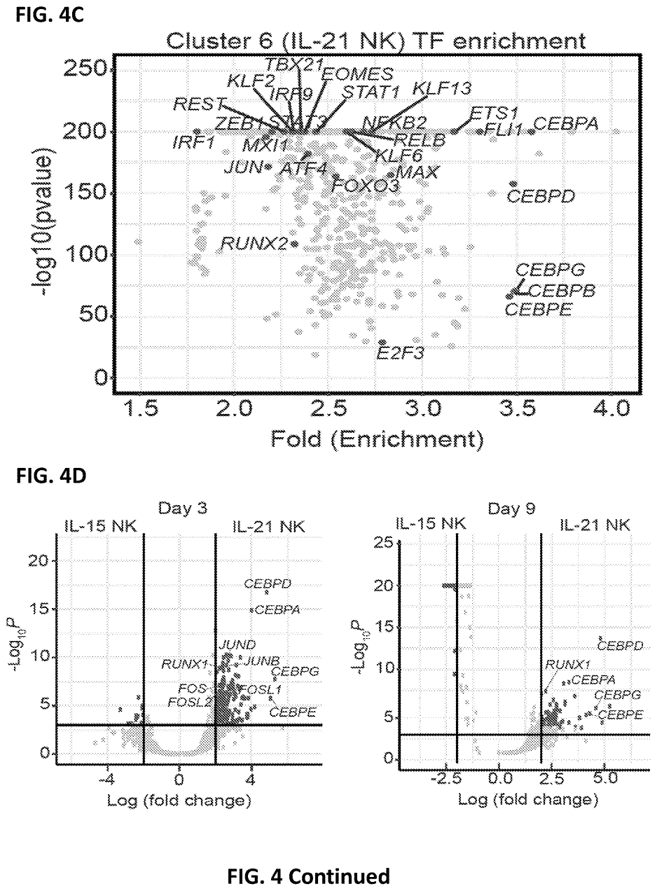

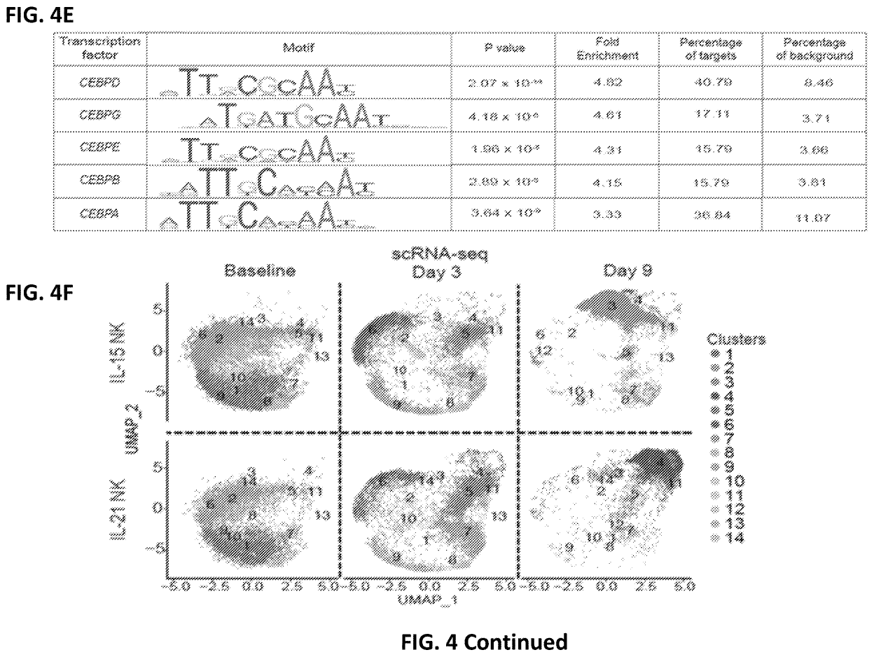

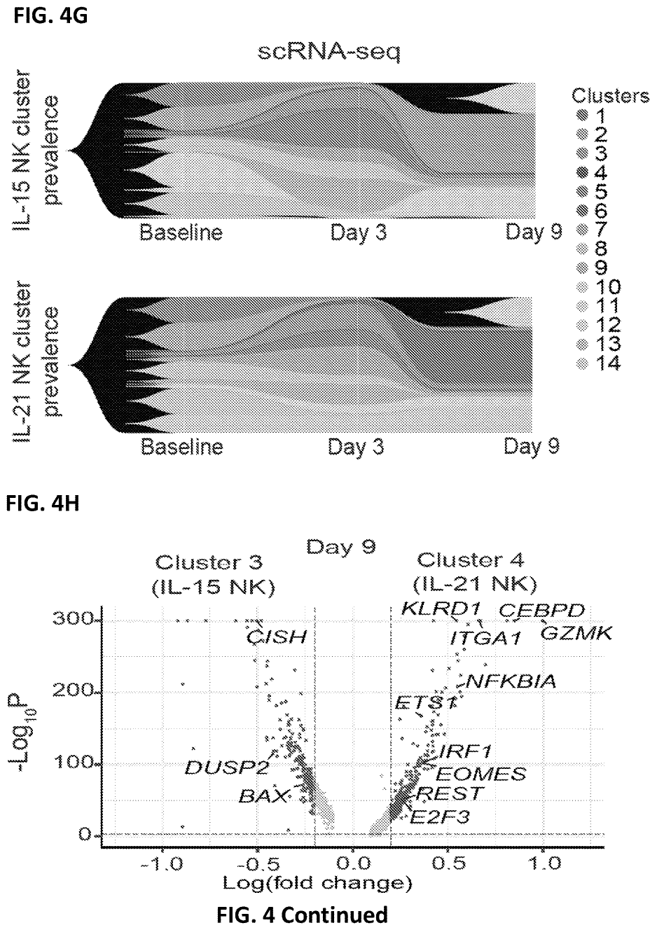

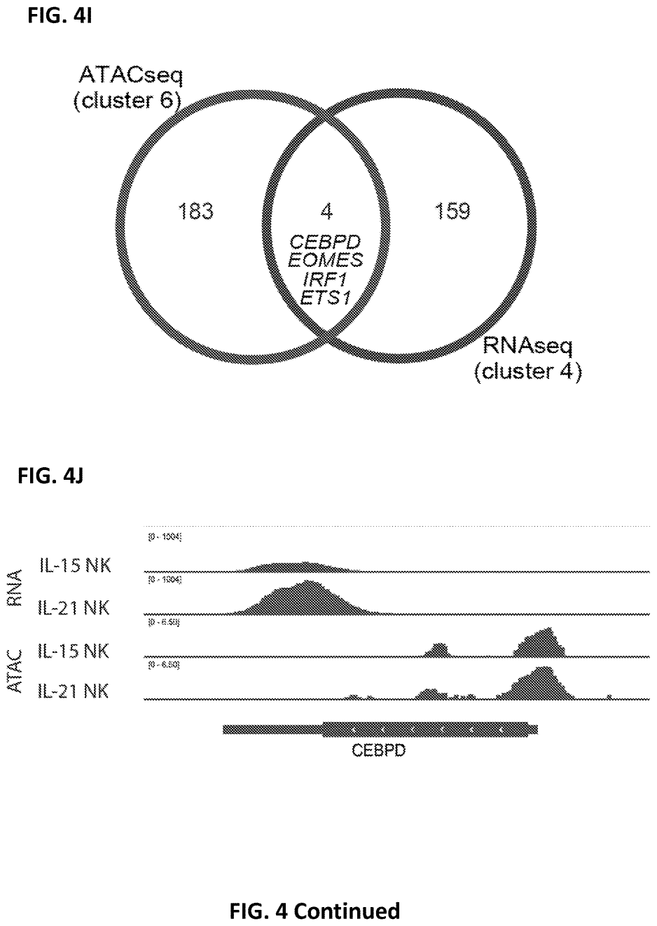

To elucidate the mechanisms underlying the persistence of IL-21 NK cells and their ability to mount a memory response (immune memory, recall) to tumor rechallenges in vitro and in vivo, the inventors studied the epigenetic and transcriptomic changes in cytokine-transduced NK cells (IL-21 NK or IL-15 NK) in the short-term (e.g., after one GSC challenge (day 3) compared to the longer-term (e.g., after three GSC rechallenges; day 9)). IL-15 NK and IL-21 NK cells cultured without GSC exposure were included as controls. Single-cell ATAC sequencing (scATAC-seq) was used to identify epigenetically different clusters between IL-15 NK and IL-21 NK cells, and between the cells at different timepoints. At baseline (prior to addition of GSCs), IL-21 NK and IL-15 NK cells had similar cluster distributions (FIGS. 4A-4B). A single GSC challenge/short-term co-culture with GSCs (day 3) resulted in contraction of some clusters (e.g., clusters 2, 4 and 7) and expansion of other clusters (e.g., clusters 1 and 8) for both IL-15 NK and IL-21 NK cells. However, upon multiple (3) rechallenges (day 9 of GSC co-culture) major/distinct epigenetic differences between IL-15 and IL-21 NK cells were observed: Cluster 3 was dominant in the IL-15 NK cell population while cluster 6 was dominant in the IL-21 NK cell population (FIGS. 4A-4B).

To further characterize the epigenetic landscape of IL-21 NK cells, 7,117 differentially accessible peaks (DAPs) were identified in cluster 6 vs all other clusters. Analysis of cluster 6-specific peaks revealed enrichment of TFs important for NK cell maturation and immune effector function such as IRF1, TBX21, EOMES IRF9 and STAT1²⁹⁻³¹, and multiple AP-1 complex TFs such as JUN, JUNB, JUND, FOS and FOSL1³². Interestingly, the CCAAT-enhancer-binding proteins (C/EBPs; CEBPs) family motifs were also found to be enriched. The CEBP family has been shown to play critical roles in cellular proliferation and differentiation in various tissues, but data on their role in NK cells was previously limited³³ (FIG. 4C and Tables 5 and 6). This enrichment was also observed following analysis of DAPs for the whole population of IL-21 NK relative to IL-15 NK cells after short-term co-culture with GSCs (day 3), specifically the data revealed enrichment in C/EBP family motifs including CEBPD (also referred to herein as CEBP delta, or CEBPδ), CEBPA (also referred to herein as CEBP alpha, or CEBPα), CEBPG (also referred to herein as CEBP gamma, or CEBPγ), and CEBPE (also referred to herein as CEBP epsilon, or CEBPε), the AP-1 family complex, and the RUNX1 TF shown to promote the adaptive behavior of NK cells against viruses 34 (FIG. 4D, left panel). Notably, after multiple tumor rechallenges (day 9), several C/EBP family TFs were significantly enriched in IL-21 NK cells compared to IL-15 NK cells, with CEBPD being the most significantly enriched (FIG. 4D, right panel and FIG. 4E), with CEBPD being enriched 4.82 fold, CEBPG enriched 4.61 fold, CEBPE enriched 4.31 fold, CEBPB (also referred to herein as CEBP beta, or CEBPβ) enriched 4.15 fold, and CEBPA enriched 3.33 fold. The consensus binding motif's for the identified CEBP family proteins are noted in FIG. 4E.

TABLE 5
Transcription factors enrichment in cluster 6

| motif | Obs | BG | % Obs | % BG | FE | PV | Name |
|---|---|---|---|---|---|---|---|
| MA0131.2 | 817 | 2150 | 21.648 | 5.375 | 4.028 | 1.00E−200 | HINFP |
| MA0028.2 | 876 | 2447 | 23.211 | 6.118 | 3.794 | 1.00E−200 | ELK1 |
| MA0506.1 | 1380 | 3863 | 36.566 | 9.658 | 3.786 | 1.00E−200 | NRF1 |
| MA0877.2 | 258 | 746 | 6.836 | 1.865 | 3.666 | 4.70E−81 | BARHL1 |
| MA0527.1 | 578 | 1693 | 15.315 | 4.233 | 3.619 | 6.57E−183 | ZBTB33 |
| MA1122.1 | 1309 | 3879 | 34.685 | 9.698 | 3.577 | 1.00E−200 | TFDP1 |
| MA1650.1 | 1532 | 4550 | 40.594 | 11.375 | 3.569 | 1.00E−200 | ZBTB14 |
| MA0632.2 | 984 | 2947 | 26.073 | 7.368 | 3.539 | 1.00E−200 | TCFL5 |
| MA0838.1 | 413 | 1756 | 10.943 | 4.390 | 3.493 | 1.39E−71 | CEBPG |
| MA0466.2 | 411 | 1751 | 10.890 | 4.378 | 3.488 | 5.90E−71 | CEBPB |
| MA0836.2 | 870 | 3717 | 23.052 | 9.293 | 3.481 | 2.79E−158 | CEBPD |
| MA0759.1 | 843 | 2574 | 22.337 | 6.435 | 3.471 | 1.00E−200 | ELK3 |
| MA0837.1 | 395 | 1701 | 10.466 | 4.253 | 3.461 | 9.78E−67 | CEBPE |
| MA0102.4 | 1084 | 4679 | 28.723 | 11.698 | 3.455 | 3.76E−200 | CEBPA |
| MA0472.2 | 1203 | 3690 | 31.876 | 9.225 | 3.455 | 1.00E−200 | EGR2 |
| MA1636.1 | 1117 | 4987 | 29.597 | 12.468 | 3.374 | 1.88E−194 | CEBPG(var.2) |
| MA0767.1 | 528 | 1661 | 13.990 | 4.153 | 3.369 | 9.04E−151 | GCM2 |
| MA1099.2 | 996 | 3146 | 26.391 | 7.865 | 3.356 | 1.00E−200 | HES1 |
| MA0475.2 | 978 | 3138 | 25.914 | 7.845 | 3.303 | 1.00E−200 | FLI1 |
| MA0156.2 | 912 | 2948 | 24.165 | 7.370 | 3.279 | 1.00E−200 | FEV |
| MA0748.2 | 868 | 2810 | 22.999 | 7.025 | 3.274 | 1.00E−200 | YY2 |
| MA0763.1 | 893 | 2900 | 23.662 | 7.250 | 3.264 | 1.00E−200 | ETV3 |
| MA0864.2 | 398 | 1296 | 10.546 | 3.240 | 3.255 | 6.88E−107 | E2F2 |
| MA0076.2 | 1603 | 5236 | 42.475 | 13.090 | 3.245 | 1.00E−200 | ELK4 |
| MA0777.1 | 216 | 710 | 5.723 | 1.775 | 3.224 | 8.22E−57 | MYBL2 |
| MA0024.3 | 253 | 840 | 6.704 | 2.100 | 3.192 | 1.50E−65 | E2F1 |
| MA1545.1 | 165 | 549 | 4.372 | 1.373 | 3.185 | 8.37E−43 | OVOL2 |
| MA1544.1 | 177 | 592 | 4.690 | 1.480 | 3.169 | 1.68E−45 | OVOL1 |
| MA0098.3 | 1046 | 3499 | 27.716 | 8.748 | 3.168 | 1.00E−200 | ETS1 |
| MA1484.1 | 1055 | 3544 | 27.954 | 8.860 | 3.155 | 1.00E−200 | ETS2 |
| MA0749.1 | 102 | 343 | 2.703 | 0.858 | 3.152 | 1.58E−26 | ZBED1 |
| MA0694.1 | 799 | 2707 | 21.171 | 6.768 | 3.128 | 1.00E−200 | ZBTB7B |
| MA0635.1 | 402 | 1366 | 10.652 | 3.415 | 3.119 | 2.10E−101 | BARHL2 |
| MA1475.1 | 420 | 1431 | 11.129 | 3.578 | 3.111 | 1.12E−105 | CREB3L4(var.2) |
| MA1483.1 | 1042 | 3557 | 27.610 | 8.893 | 3.105 | 1.00E−200 | ELF2 |
| MA0470.2 | 274 | 936 | 7.260 | 2.340 | 3.103 | 4.59E−68 | E2F4 |
| MA0474.2 | 973 | 3337 | 25.782 | 8.343 | 3.090 | 1.00E−200 | ERG |
| MA0695.1 | 956 | 3288 | 25.331 | 8.220 | 3.082 | 1.00E−200 | ZBTB7C |
| MA0637.1 | 321 | 1106 | 8.506 | 2.765 | 3.076 | 7.47E−79 | CENPB |
| MA0104.4 | 768 | 2647 | 20.350 | 6.618 | 3.075 | 2.17E−197 | MYCN |
| MA0649.1 | 736 | 2539 | 19.502 | 6.348 | 3.072 | 3.82E−188 | HEY2 |
| MA0616.2 | 677 | 2338 | 17.939 | 5.845 | 3.069 | 1.59E−171 | HES2 |
| MA0014.3 | 732 | 2528 | 19.396 | 6.320 | 3.069 | 1.03E−186 | PAX5 |
| MA0711.1 | 145 | 501 | 3.842 | 1.253 | 3.068 | 1.06E−35 | OTX1 |
| MA0750.2 | 1928 | 6700 | 51.086 | 16.750 | 3.050 | 1.00E−200 | ZBTB7A |
| MA0735.1 | 672 | 2342 | 17.806 | 5.855 | 3.041 | 8.30E−168 | GLIS1 |
| MA0732.1 | 1726 | 6021 | 45.734 | 15.053 | 3.038 | 1.00E−200 | EGR3 |
| MA0636.1 | 429 | 1500 | 11.367 | 3.750 | 3.031 | 7.71E−104 | BHLHE41 |
| MA1583.1 | 565 | 1976 | 14.971 | 4.940 | 3.031 | 1.36E−138 | ZFP57 |
| MA0823.1 | 779 | 2729 | 20.641 | 6.823 | 3.025 | 1.33E−195 | HEY1 |
| MA0831.2 | 555 | 1945 | 14.706 | 4.863 | 3.024 | 1.46E−135 | TFE3 |
| MA1513.1 | 2393 | 8399 | 63.408 | 20.998 | 3.020 | 1.00E−200 | KLF15 |
| MA0733.1 | 1638 | 5756 | 43.402 | 14.390 | 3.016 | 1.00E−200 | EGR4 |
| MA0765.2 | 1316 | 4625 | 34.870 | 11.563 | 3.016 | 1.00E−200 | ETV5 |
| MA1464.1 | 644 | 2266 | 17.064 | 5.665 | 3.012 | 6.21E−158 | ARNT2 |
| MA0641.1 | 1244 | 4396 | 32.962 | 10.990 | 2.999 | 1.00E−200 | ELF4 |
| MA1106.1 | 544 | 1926 | 14.414 | 4.815 | 2.994 | 1.24E−130 | HIF1A |
| MA0686.1 | 599 | 2128 | 15.872 | 5.320 | 2.983 | 5.84E−144 | SPDEF |
| MA0891.1 | 664 | 2360 | 17.594 | 5.900 | 2.982 | 1.05E−160 | GSC2 |
| MA0760.1 | 1055 | 3771 | 27.954 | 9.428 | 2.965 | 1.00E−200 | ERF |
| MA0594.2 | 134 | 479 | 3.551 | 1.198 | 2.965 | 2.01E−31 | HOXA9 |
| MA0873.1 | 427 | 1527 | 11.314 | 3.818 | 2.964 | 9.04E−100 | HOXD12 |
| MA0908.1 | 516 | 1848 | 13.672 | 4.620 | 2.959 | 2.48E−121 | HOXD11 |
| MA0779.1 | 465 | 1671 | 12.321 | 4.178 | 2.949 | 3.39E−108 | PAX1 |
| MA0147.3 | 965 | 3468 | 25.570 | 8.670 | 2.949 | 1.00E−200 | MYC |
| MA0259.1 | 299 | 1076 | 7.923 | 2.690 | 2.945 | 1.39E−68 | ARNT::HIF1A |
| MA0906.1 | 456 | 1649 | 12.083 | 4.123 | 2.931 | 5.87E−105 | HOXC12 |
| MA0647.1 | 560 | 2027 | 14.838 | 5.068 | 2.928 | 4.22E−130 | GRHL1 |
| MA0628.1 | 472 | 1712 | 12.507 | 4.280 | 2.922 | 2.75E−108 | POU6F1 |
| MA0736.1 | 1141 | 4146 | 30.233 | 10.365 | 2.917 | 1.00E−200 | GLIS2 |
| MA0781.1 | 527 | 1920 | 13.964 | 4.800 | 2.909 | 9.86E−121 | PAX9 |
| MA0865.1 | 1057 | 3859 | 28.007 | 9.648 | 2.903 | 1.00E−200 | E2F8 |
| MA0074.1 | 906 | 3310 | 24.006 | 8.275 | 2.901 | 1.00E−200 | RXRA::VDR |
| MA0740.1 | 2073 | 7581 | 54.928 | 18.953 | 2.898 | 1.00E−200 | KLF14 |
| MA0871.2 | 752 | 2753 | 19.926 | 6.883 | 2.895 | 1.19E−175 | TFEC |
| MA0682.2 | 742 | 2717 | 19.661 | 6.793 | 2.894 | 4.99E−173 | PITX1 |
| MA0780.1 | 438 | 1605 | 11.606 | 4.013 | 2.892 | 1.55E−98 | PAX3 |
| MA1533.1 | 967 | 3546 | 25.623 | 8.865 | 2.890 | 1.00E−200 | NR1I2 |
| MA0648.1 | 620 | 2276 | 16.428 | 5.690 | 2.887 | 8.16E−142 | GSC |
| MA0794.1 | 547 | 2009 | 14.494 | 5.023 | 2.886 | 5.70E−124 | PROX1 |
| MA0758.1 | 762 | 2800 | 20.191 | 7.000 | 2.884 | 3.64E−177 | E2F7 |
| MA0646.1 | 626 | 2301 | 16.587 | 5.753 | 2.883 | 5.42E−143 | GCM1 |
| MA0822.1 | 830 | 3057 | 21.993 | 7.643 | 2.878 | 5.12E−194 | HES7 |
| MA1560.1 | 553 | 2037 | 14.653 | 5.093 | 2.877 | 8.03E−125 | SOHLH2 |
| MA0145.3 | 381 | 1404 | 10.095 | 3.510 | 2.876 | 1.56E−84 | TFCP2 |
| MA0663.1 | 484 | 1784 | 12.825 | 4.460 | 2.875 | 2.48E−108 | MLX |
| MA0067.1 | 484 | 1785 | 12.825 | 4.463 | 2.874 | 3.10E−108 | Pax2 |
| MA1505.1 | 305 | 1126 | 8.082 | 2.815 | 2.871 | 3.60E−67 | HOXC8 |
| MA1493.1 | 773 | 2856 | 20.482 | 7.140 | 2.869 | 2.07E−178 | HES6 |
| MA0872.1 | 1417 | 5236 | 37.546 | 13.090 | 2.868 | 1.00E−200 | TFAP2A(var.3) |
| MA0755.1 | 321 | 1187 | 8.506 | 2.968 | 2.866 | 1.48E−70 | CUX2 |
| MA1474.1 | 683 | 2526 | 18.098 | 6.315 | 2.866 | 1.47E−155 | CREB3L4 |
| MA0603.1 | 666 | 2464 | 17.647 | 6.160 | 2.865 | 2.80E−151 | Arntl |
| MA0714.1 | 875 | 3247 | 23.185 | 8.118 | 2.856 | 1.00E−200 | PITX3 |
| MA1509.1 | 267 | 992 | 7.075 | 2.480 | 2.853 | 3.86E−58 | IRF6 |
| MA1516.1 | 1988 | 7389 | 52.676 | 18.473 | 2.852 | 1.00E−200 | KLF3 |
| MA0664.1 | 440 | 1637 | 11.659 | 4.093 | 2.849 | 1.37E−96 | MLXIPL |
| MA0821.1 | 748 | 2784 | 19.820 | 6.960 | 2.848 | 4.84E−170 | HES5 |
| MA1143.1 | 459 | 1712 | 12.162 | 4.280 | 2.842 | 1.59E−100 | FOSL1::JUND(var.2) |
| MA0883.1 | 640 | 2388 | 16.958 | 5.970 | 2.841 | 6.40E−143 | Dmbx1 |
| MA0464.2 | 476 | 1779 | 12.613 | 4.448 | 2.836 | 4.47E−104 | BHLHE40 |
| MA0815.1 | 1358 | 5085 | 35.983 | 12.713 | 2.831 | 1.00E−200 | TFAP2C(var.3) |
| MA0058.3 | 737 | 2760 | 19.528 | 6.900 | 2.830 | 1.25E−165 | MAX |
| MA0828.1 | 494 | 1850 | 13.090 | 4.625 | 2.830 | 7.51E−108 | SREBF2(var.2) |
| MA1569.1 | 1236 | 4629 | 32.750 | 11.573 | 2.830 | 1.00E−200 | TFAP2E |
| MA1506.1 | 502 | 1888 | 13.302 | 4.720 | 2.818 | 6.42E−109 | HOXD10 |
| MA0825.1 | 736 | 2781 | 19.502 | 6.953 | 2.805 | 5.62E−163 | MNT |
| MA1147.1 | 787 | 2974 | 20.853 | 7.435 | 2.805 | 1.96E−175 | NR4A2::RXRA |
| MA0651.1 | 404 | 1527 | 10.705 | 3.818 | 2.804 | 3.89E−86 | HOXC11 |
| MA1466.1 | 515 | 1949 | 13.646 | 4.873 | 2.801 | 9.61E−111 | ATF6 |
| MA0680.1 | 481 | 1826 | 12.745 | 4.565 | 2.792 | 1.56E−102 | PAX7 |
| MA0610.1 | 689 | 2616 | 18.256 | 6.540 | 2.792 | 1.96E−150 | DMRT3 |
| MA0645.1 | 1614 | 6136 | 42.766 | 15.340 | 2.788 | 1.00E−200 | ETV6 |
| MA0819.1 | 456 | 1734 | 12.083 | 4.335 | 2.787 | 1.07E−96 | CLOCK |
| MA0608.1 | 725 | 2757 | 19.210 | 6.893 | 2.787 | 1.27E−158 | Creb3l2 |
| MA0469.3 | 139 | 529 | 3.683 | 1.323 | 2.785 | 1.78E−29 | E2F3 |
| MA0754.1 | 284 | 1081 | 7.525 | 2.703 | 2.785 | 1.93E−59 | CUX1 |
| MA1602.1 | 515 | 1964 | 13.646 | 4.910 | 2.779 | 2.46E−109 | ZSCAN29 |
| MA0638.1 | 463 | 1768 | 12.268 | 4.420 | 2.776 | 1.47E−97 | CREB3 |
| MA0762.1 | 1063 | 4063 | 28.166 | 10.158 | 2.773 | 1.00E−200 | ETV2 |
| MA0162.4 | 2303 | 8804 | 61.023 | 22.010 | 2.773 | 1.00E−200 | EGR1 |
| MA0007.3 | 698 | 2669 | 18.495 | 6.673 | 2.772 | 8.66E−151 | Ar |
| MA0108.2 | 728 | 2789 | 19.290 | 6.973 | 2.767 | 2.18E−157 | TBP |
| MA0105.4 | 1417 | 5433 | 37.546 | 13.583 | 2.764 | 1.00E−200 | NFKB1 |
| MA0622.1 | 686 | 2633 | 18.177 | 6.583 | 2.761 | 4.87E−147 | Mlxip |
| MA0153.2 | 841 | 3229 | 22.284 | 8.073 | 2.760 | 5.76E−184 | HNF1B |
| MA0697.1 | 1472 | 5653 | 39.004 | 14.133 | 2.760 | 1.00E−200 | ZIC3 |
| MA1578.1 | 1099 | 4226 | 29.120 | 10.565 | 2.756 | 1.00E−200 | VEZF1 |
| MA1496.1 | 571 | 2200 | 15.130 | 5.500 | 2.751 | 6.70E−120 | HOXA4 |
| MA1504.1 | 571 | 2200 | 15.130 | 5.500 | 2.751 | 6.70E−120 | HOXC4 |
| MA1507.1 | 571 | 2200 | 15.130 | 5.500 | 2.751 | 6.70E−120 | HOXD4 |
| MA0804.1 | 530 | 2045 | 14.043 | 5.113 | 2.747 | 1.67E−110 | TBX19 |
| MA0083.3 | 538 | 2079 | 14.255 | 5.198 | 2.743 | 5.52E−112 | SRF |
| MA1494.1 | 1130 | 4368 | 29.942 | 10.920 | 2.742 | 1.00E−200 | HNF4A(var.2) |
| MA0069.1 | 581 | 2246 | 15.395 | 5.615 | 2.742 | 1.87E−121 | PAX6 |
| MA0626.1 | 812 | 3139 | 21.516 | 7.848 | 2.742 | 6.79E−175 | Npas2 |
| MA0704.1 | 428 | 1656 | 11.341 | 4.140 | 2.739 | 6.48E−88 | Lhx4 |
| MA1584.1 | 1490 | 5771 | 39.481 | 14.428 | 2.736 | 1.00E−200 | ZIC5 |
| MA0631.1 | 760 | 2944 | 20.138 | 7.360 | 2.736 | 5.69E−162 | Six3 |
| MA0756.1 | 795 | 3084 | 21.065 | 7.710 | 2.732 | 7.86E−170 | ONECUT2 |
| MA0657.1 | 1140 | 4426 | 30.207 | 11.065 | 2.730 | 1.00E−200 | KLF13 |
| MA0905.1 | 622 | 2417 | 16.481 | 6.043 | 2.728 | 1.60E−129 | HOXC10 |
| MA0839.1 | 649 | 2522 | 17.197 | 6.305 | 2.727 | 1.37E−135 | CREB3L1 |
| MA0604.1 | 202 | 785 | 5.352 | 1.963 | 2.727 | 6.48E−41 | Atf1 |
| MA1140.2 | 464 | 1804 | 12.295 | 4.510 | 2.726 | 7.62E−95 | JUNB(var.2) |
| MA0813.1 | 1501 | 5838 | 39.772 | 14.595 | 2.725 | 1.00E−200 | TFAP2B(var.3) |
| MA0874.1 | 519 | 2020 | 13.752 | 5.050 | 2.723 | 1.70E−106 | Arx |
| MA1503.1 | 450 | 1753 | 11.924 | 4.383 | 2.721 | 1.49E−91 | HOXB9 |
| MA1495.1 | 232 | 904 | 6.147 | 2.260 | 2.720 | 1.08E−46 | HOXA1 |
| MA0904.2 | 232 | 904 | 6.147 | 2.260 | 2.720 | 1.08E−46 | HOXB5 |
| MA0612.2 | 319 | 1245 | 8.453 | 3.113 | 2.716 | 4.47E−64 | EMX1 |
| MA0900.2 | 319 | 1245 | 8.453 | 3.113 | 2.716 | 4.47E−64 | HOXA2 |
| MA1478.1 | 775 | 3025 | 20.535 | 7.563 | 2.715 | 2.18E−163 | DMRTA2 |
| MA0639.1 | 303 | 1183 | 8.029 | 2.958 | 2.715 | 8.73E−61 | DBP |
| MA1547.1 | 853 | 3332 | 22.602 | 8.330 | 2.713 | 1.43E−181 | PITX2 |
| MA0109.1 | 709 | 2770 | 18.786 | 6.925 | 2.713 | 6.47E−148 | HLTF |
| MA0113.3 | 592 | 2314 | 15.686 | 5.785 | 2.712 | 1.34E−121 | NR3C1 |
| MA1538.1 | 887 | 3469 | 23.503 | 8.673 | 2.710 | 2.41E−189 | NR2F1(var.3) |
| MA1575.1 | 902 | 3533 | 23.900 | 8.833 | 2.706 | 1.79E−192 | THRB(var.2) |
| MA0757.1 | 1213 | 4757 | 32.141 | 11.893 | 2.703 | 1.00E−200 | ONECUT3 |
| MA0827.1 | 436 | 1710 | 11.553 | 4.275 | 2.702 | 1.45E−87 | OLIG3 |
| MA1548.1 | 1546 | 6068 | 40.964 | 15.170 | 2.700 | 1.00E−200 | PLAGL2 |
| MA0615.1 | 151 | 593 | 4.001 | 1.483 | 2.699 | 2.68E−30 | Gmeb1 |
| MA0812.1 | 1471 | 5779 | 38.977 | 14.448 | 2.698 | 1.00E−200 | TFAP2B(var.2) |
| MA0898.1 | 945 | 3718 | 25.040 | 9.295 | 2.694 | 1.00E−200 | Hmx3 |
| MA0751.1 | 1468 | 5781 | 38.898 | 14.453 | 2.691 | 1.00E−200 | ZIC4 |
| MA0887.1 | 402 | 1584 | 10.652 | 3.960 | 2.690 | 7.44E−80 | EVX1 |
| MA1497.1 | 538 | 2120 | 14.255 | 5.300 | 2.690 | 2.68E−108 | HOXA6 |
| MA0124.2 | 858 | 3381 | 22.734 | 8.453 | 2.690 | 4.64E−180 | Nkx3-1 |
| MA0844.1 | 407 | 1605 | 10.784 | 4.013 | 2.688 | 9.09E−81 | XBP1 |
| MA0132.2 | 447 | 1763 | 11.844 | 4.408 | 2.687 | 5.36E−89 | PDX1 |
| MA1481.1 | 447 | 1763 | 11.844 | 4.408 | 2.687 | 5.36E−89 | DRGX |
| MA0829.2 | 1302 | 5137 | 34.499 | 12.843 | 2.686 | 1.00E−200 | SREBF1(var.2) |
| MA0853.1 | 518 | 2044 | 13.725 | 5.110 | 2.686 | 8.54E−104 | Alx4 |
| MA0778.1 | 1065 | 4203 | 28.219 | 10.508 | 2.686 | 1.00E−200 | NFKB2 |
| MA0914.1 | 694 | 2739 | 18.389 | 6.848 | 2.686 | 5.20E−142 | ISL2 |
| MA0854.1 | 589 | 2325 | 15.607 | 5.813 | 2.685 | 6.55E−119 | Alx1 |
| MA0907.1 | 655 | 2586 | 17.356 | 6.465 | 2.685 | 2.83E−133 | HOXC13 |
| MA0727.1 | 584 | 2307 | 15.474 | 5.768 | 2.683 | 1.12E−117 | NR3C2 |
| MA0046.2 | 1217 | 4810 | 32.247 | 12.025 | 2.682 | 1.00E−200 | HNF1A |
| MA0059.1 | 821 | 3245 | 21.754 | 8.113 | 2.682 | 1.68E−170 | MAX::MYC |
| MA0885.1 | 507 | 2006 | 13.434 | 5.015 | 2.679 | 5.37E−101 | Dlx2 |
| MA0702.2 | 610 | 2415 | 16.163 | 6.038 | 2.677 | 7.81E−123 | LMX1A |
| MA0897.1 | 920 | 3643 | 24.377 | 9.108 | 2.677 | 3.30E−193 | Hmx2 |
| MA0720.1 | 444 | 1759 | 11.765 | 4.398 | 2.675 | 1.08E−87 | Shox2 |
| MA1499.1 | 749 | 2968 | 19.846 | 7.420 | 2.675 | 2.52E−153 | HOXB4 |
| MA0685.1 | 2335 | 9254 | 61.871 | 23.135 | 2.674 | 1.00E−200 | SP4 |
| MA0722.1 | 548 | 2172 | 14.520 | 5.430 | 2.674 | 2.51E−109 | VAX1 |
| MA0723.1 | 548 | 2172 | 14.520 | 5.430 | 2.674 | 2.51E−109 | VAX2 |
| MA0799.1 | 757 | 3002 | 20.058 | 7.505 | 2.673 | 6.31E−155 | RFX4 |
| MA0855.1 | 1415 | 5614 | 37.493 | 14.035 | 2.671 | 1.00E−200 | RXRB |
| MA0068.2 | 352 | 1397 | 9.327 | 3.493 | 2.671 | 8.30E−69 | PAX4 |
| MA0678.1 | 384 | 1524 | 10.175 | 3.810 | 2.671 | 3.17E−75 | OLIG2 |
| MA0621.1 | 832 | 3302 | 22.046 | 8.255 | 2.671 | 7.75E−172 | mix-a |
| MA0136.2 | 1534 | 6093 | 40.647 | 15.233 | 2.668 | 1.00E−200 | ELF5 |
| MA0861.1 | 742 | 2949 | 19.661 | 7.373 | 2.667 | 5.72E−151 | TP73 |
| MA0703.2 | 824 | 3277 | 21.834 | 8.193 | 2.665 | 2.19E−169 | LMX1B |
| MA1564.1 | 2603 | 10364 | 68.972 | 25.910 | 2.662 | 1.00E−200 | SP9 |
| MA1420.1 | 821 | 3269 | 21.754 | 8.173 | 2.662 | 2.40E−168 | IRF5 |
| MA1535.1 | 1114 | 4438 | 29.518 | 11.095 | 2.660 | 1.00E−200 | NR2C1 |
| MA0112.3 | 1368 | 5451 | 36.248 | 13.628 | 2.660 | 1.00E−200 | ESR1 |
| MA1146.1 | 777 | 3100 | 20.588 | 7.750 | 2.657 | 8.56E−158 | NR1H4::RXRA |
| MA1539.1 | 850 | 3392 | 22.523 | 8.480 | 2.656 | 2.41E−174 | NR2F6(var.3) |
| MA0029.1 | 1293 | 5161 | 34.261 | 12.903 | 2.655 | 1.00E−200 | Mecom |
| MA0721.1 | 672 | 2683 | 17.806 | 6.708 | 2.655 | 2.17E−134 | UNCX |
| MA1500.1 | 563 | 2248 | 14.918 | 5.620 | 2.654 | 4.53E−111 | HOXB6 |
| MA1603.1 | 992 | 3961 | 26.285 | 9.903 | 2.654 | 1.00E−200 | Dmrt1 |
| MA1556.1 | 1001 | 3998 | 26.524 | 9.995 | 2.654 | 1.00E−200 | RXRG(var.2) |
| MA1471.1 | 1255 | 5013 | 33.254 | 12.533 | 2.653 | 1.00E−200 | BARX2 |
| MA0909.2 | 903 | 3608 | 23.927 | 9.020 | 2.653 | 2.99E−186 | HOXD13 |
| MA0811.1 | 1469 | 5873 | 38.924 | 14.683 | 2.651 | 1.00E−200 | TFAP2B |
| MA0731.1 | 1140 | 4558 | 30.207 | 11.395 | 2.651 | 1.00E−200 | BCL6B |
| MA0843.1 | 326 | 1304 | 8.638 | 3.260 | 2.650 | 9.08E−63 | TEF |
| MA0463.2 | 1022 | 4091 | 27.080 | 10.228 | 2.648 | 1.00E−200 | BCL6 |
| MA0719.1 | 782 | 3133 | 20.721 | 7.833 | 2.645 | 9.10E−158 | RHOXF1 |
| MA1574.1 | 1488 | 5963 | 39.428 | 14.908 | 2.645 | 1.00E−200 | THRB |
| MA0661.1 | 556 | 2229 | 14.732 | 5.573 | 2.644 | 7.91E−109 | MEOX1 |
| MA0893.2 | 420 | 1684 | 11.129 | 4.210 | 2.643 | 4.74E−81 | GSX2 |
| MA0912.2 | 420 | 1684 | 11.129 | 4.210 | 2.643 | 4.74E−81 | HOXD3 |
| MA0810.1 | 1701 | 6822 | 45.072 | 17.055 | 2.643 | 1.00E−200 | TFAP2A(var.2) |
| MA1555.1 | 735 | 2948 | 19.475 | 7.370 | 2.643 | 4.86E−147 | RXRB(var.2) |
| MA0122.3 | 1033 | 4144 | 27.371 | 10.360 | 2.642 | 1.00E−200 | Nkx3-2 |
| MA0658.1 | 457 | 1834 | 12.109 | 4.585 | 2.641 | 2.38E−88 | LHX6 |
| MA1463.1 | 708 | 2843 | 18.760 | 7.108 | 2.639 | 7.74E−141 | ARGFX |
| MA0485.2 | 438 | 1759 | 11.606 | 4.398 | 2.639 | 2.03E−84 | HOXC9 |
| MA0889.1 | 450 | 1812 | 11.924 | 4.530 | 2.632 | 2.02E−86 | GBX1 |
| MA0879.1 | 553 | 2227 | 14.653 | 5.568 | 2.632 | 2.41E−107 | Dlx1 |
| MA0776.1 | 184 | 741 | 4.875 | 1.853 | 2.632 | 3.92E−35 | MYBL1 |
| MA0600.2 | 597 | 2406 | 15.819 | 6.015 | 2.630 | 2.45E−116 | RFX2 |
| MA0683.1 | 1336 | 5385 | 35.400 | 13.463 | 2.630 | 1.00E−200 | POU4F2 |
| MA1519.1 | 496 | 2001 | 13.143 | 5.003 | 2.627 | 2.24E−95 | LHX5 |
| MA0035.4 | 816 | 3292 | 21.622 | 8.230 | 2.627 | 2.04E−163 | GATA1 |
| MA0027.2 | 396 | 1598 | 10.493 | 3.995 | 2.626 | 2.09E−75 | EN1 |
| MA0629.1 | 668 | 2696 | 17.700 | 6.740 | 2.626 | 5.23E−131 | Rhox11 |
| MA0601.1 | 1281 | 5172 | 33.943 | 12.930 | 2.625 | 1.00E−200 | Arid3b |
| MA1117.1 | 1008 | 4073 | 26.709 | 10.183 | 2.623 | 1.00E−200 | RELB |
| MA0725.1 | 429 | 1734 | 11.367 | 4.335 | 2.622 | 1.12E−81 | VSX1 |
| MA0726.1 | 429 | 1734 | 11.367 | 4.335 | 2.622 | 1.12E−81 | VSX2 |
| MA0706.1 | 744 | 3008 | 19.714 | 7.520 | 2.622 | 6.04E−147 | MEOX2 |
| MA0609.2 | 557 | 2252 | 14.759 | 5.630 | 2.621 | 2.05E−107 | CREM |
| MA0715.1 | 1104 | 4468 | 29.253 | 11.170 | 2.619 | 1.00E−200 | PROP1 |
| MA0875.1 | 479 | 1940 | 12.692 | 4.850 | 2.617 | 2.63E−91 | BARX1 |
| MA0718.1 | 597 | 2419 | 15.819 | 6.048 | 2.616 | 3.14E−115 | RAX |
| MA0737.1 | 954 | 3870 | 25.278 | 9.675 | 2.613 | 4.19E−193 | GLIS3 |
| MA0644.1 | 565 | 2292 | 14.971 | 5.730 | 2.613 | 2.07E−108 | ESX1 |
| MA0712.2 | 1086 | 4406 | 28.776 | 11.015 | 2.612 | 1.00E−200 | OTX2 |
| MA0115.1 | 1261 | 5120 | 33.413 | 12.800 | 2.610 | 1.00E−200 | NR1H2::RXRA |
| MA0840.1 | 501 | 2035 | 13.275 | 5.088 | 2.609 | 3.18E−95 | Creb5 |
| MA1126.1 | 689 | 2799 | 18.256 | 6.998 | 2.609 | 6.18E−134 | FOS::JUN(var.2) |
| MA0164.1 | 943 | 3833 | 24.987 | 9.583 | 2.608 | 6.83E−190 | Nr2e3 |
| MA0486.2 | 1049 | 4265 | 27.795 | 10.663 | 2.607 | 1.00E−200 | HSF1 |
| MA0009.2 | 529 | 2151 | 14.017 | 5.378 | 2.607 | 1.16E−100 | TBXT |
| MA0674.1 | 614 | 2497 | 16.269 | 6.243 | 2.606 | 5.50E−118 | NKX6-1 |
| MA0524.2 | 1461 | 5944 | 38.712 | 14.860 | 2.605 | 1.00E−200 | TFAP2C |
| MA1550.1 | 1457 | 5931 | 38.606 | 14.828 | 2.604 | 1.00E−200 | PPARD |
| MA1113.2 | 1095 | 4458 | 29.014 | 11.145 | 2.603 | 1.00E−200 | PBX2 |
| MA0911.1 | 414 | 1686 | 10.970 | 4.215 | 2.603 | 1.14E−77 | Hoxa11 |
| MA0650.2 | 1067 | 4346 | 28.272 | 10.865 | 2.602 | 1.00E−200 | HOXA13 |
| MA0512.2 | 1343 | 5478 | 35.586 | 13.695 | 2.598 | 1.00E−200 | Rxra |
| MA1133.1 | 528 | 2154 | 13.990 | 5.385 | 2.598 | 7.15E−100 | JUN::JUNB(var.2) |
| MA0692.1 | 702 | 2865 | 18.601 | 7.163 | 2.597 | 1.41E−135 | TFEB |
| MA0624.1 | 1481 | 6046 | 39.242 | 15.115 | 2.596 | 1.00E−200 | NFATC1 |
| MA1129.1 | 349 | 1425 | 9.247 | 3.563 | 2.596 | 7.27E−65 | FOSL1::JUN(var.2) |
| MA0135.1 | 1273 | 5199 | 33.731 | 12.998 | 2.595 | 1.00E−200 | Lhx3 |
| MA0625.1 | 1472 | 6012 | 39.004 | 15.030 | 2.595 | 1.00E−200 | NFATC3 |
| MA1517.1 | 2211 | 9031 | 58.585 | 22.578 | 2.595 | 1.00E−200 | KLF6 |
| MA0856.1 | 1346 | 5498 | 35.665 | 13.745 | 2.595 | 1.00E−200 | RXRG |
| MA1553.1 | 651 | 2661 | 17.250 | 6.653 | 2.593 | 1.53E−124 | RARG(var.3) |
| MA1111.1 | 1550 | 6340 | 41.070 | 15.850 | 2.591 | 1.00E−200 | NR2F2 |
| MA1154.1 | 1283 | 5251 | 33.996 | 13.128 | 2.590 | 1.00E−200 | ZNF282 |
| MA0699.1 | 569 | 2329 | 15.077 | 5.823 | 2.589 | 1.72E−107 | LBX2 |
| MA1524.1 | 664 | 2718 | 17.594 | 6.795 | 2.589 | 6.44E−127 | MSGN1 |
| MA0882.1 | 481 | 1971 | 12.745 | 4.928 | 2.587 | 8.37E−90 | DLX6 |
| MA0716.1 | 491 | 2012 | 13.010 | 5.030 | 2.586 | 9.18E−92 | PRRX1 |
| MA0701.2 | 491 | 2012 | 13.010 | 5.030 | 2.586 | 9.18E−92 | LHX9 |
| MA0125.1 | 539 | 2209 | 14.282 | 5.523 | 2.586 | 3.08E−101 | Nobox |
| MA1537.1 | 1521 | 6236 | 40.302 | 15.590 | 2.585 | 1.00E−200 | NR2F1(var.2) |
| MA1479.1 | 820 | 3367 | 21.728 | 8.418 | 2.581 | 3.01E−159 | DMRTC2 |
| MA0679.2 | 1388 | 5700 | 36.778 | 14.250 | 2.581 | 1.00E−200 | ONECUT1 |
| MA0602.1 | 907 | 3725 | 24.033 | 9.313 | 2.581 | 2.37E−178 | Arid5a |
| MA1585.1 | 756 | 3105 | 20.032 | 7.763 | 2.581 | 1.99E−145 | ZKSCAN1 |
| MA1532.1 | 1202 | 4938 | 31.849 | 12.345 | 2.580 | 1.00E−200 | NR1D2 |
| MA0899.1 | 1319 | 5420 | 34.950 | 13.550 | 2.579 | 1.00E−200 | HOXA10 |
| MA0888.1 | 413 | 1698 | 10.943 | 4.245 | 2.578 | 3.70E−76 | EVX2 |
| MA1470.1 | 457 | 1879 | 12.109 | 4.698 | 2.578 | 1.34E−84 | BACH2(var.2) |
| MA0700.2 | 719 | 2957 | 19.051 | 7.393 | 2.577 | 2.83E−137 | LHX2 |
| MA0036.3 | 1019 | 4191 | 27.001 | 10.478 | 2.577 | 1.00E−200 | GATA2 |
| MA0660.1 | 1873 | 7708 | 49.629 | 19.270 | 2.575 | 1.00E−200 | MEF2B |
| MA1552.1 | 664 | 2733 | 17.594 | 6.833 | 2.575 | 1.15E−125 | RARB(var.3) |
| MA1561.1 | 404 | 1663 | 10.705 | 4.158 | 2.575 | 2.79E−74 | SOX12 |
| MA0770.1 | 1103 | 4541 | 29.226 | 11.353 | 2.574 | 1.00E−200 | HSF2 |
| MA0713.1 | 903 | 3719 | 23.927 | 9.298 | 2.573 | 1.44E−176 | PHOX2A |
| MA0895.1 | 476 | 1962 | 12.613 | 4.905 | 2.571 | 6.93E−88 | HMBOX1 |
| MA1570.1 | 480 | 1979 | 12.719 | 4.948 | 2.571 | 1.28E−88 | TFAP4(var.2) |
| MA0896.1 | 677 | 2792 | 17.939 | 6.980 | 2.570 | 6.89E−128 | Hmx1 |
| MA0079.4 | 2480 | 10231 | 65.713 | 25.578 | 2.569 | 1.00E−200 | SP1 |
| MA0913.2 | 1065 | 4394 | 28.219 | 10.985 | 2.569 | 1.00E−200 | HOXD9 |
| MA0771.1 | 1025 | 4229 | 27.160 | 10.573 | 2.569 | 1.00E−200 | HSF4 |
| MA0525.2 | 478 | 1973 | 12.666 | 4.933 | 2.568 | 4.76E−88 | TP63 |
| MA1651.1 | 1375 | 5679 | 36.433 | 14.198 | 2.566 | 1.00E−200 | ZFP42 |
| MA0892.1 | 690 | 2851 | 18.283 | 7.128 | 2.565 | 4.02E−130 | GSX1 |
| MA0471.2 | 2482 | 10257 | 65.766 | 25.643 | 2.565 | 1.00E−200 | E2F6 |
| MA0662.1 | 656 | 2711 | 17.382 | 6.778 | 2.565 | 4.06E−123 | MIXL1 |
| MA0107.1 | 1350 | 5581 | 35.771 | 13.953 | 2.564 | 1.00E−200 | RELA |
| MA0742.1 | 1980 | 8189 | 52.464 | 20.473 | 2.563 | 1.00E−200 | Klf12 |
| MA0791.1 | 1382 | 5716 | 36.619 | 14.290 | 2.563 | 1.00E−200 | POU4F3 |
| MA0643.1 | 1279 | 5290 | 33.890 | 13.225 | 2.563 | 1.00E−200 | Esrrg |
| MA0084.1 | 1090 | 4509 | 28.882 | 11.273 | 2.562 | 1.00E−200 | SRY |
| MA0863.1 | 785 | 3248 | 20.800 | 8.120 | 2.562 | 1.29E−149 | MTF1 |
| MA0746.2 | 2616 | 10824 | 69.316 | 27.060 | 2.562 | 1.00E−200 | SP3 |
| MA0666.1 | 484 | 2003 | 12.825 | 5.008 | 2.561 | 8.76E−89 | MSX1 |
| MA0708.1 | 484 | 2003 | 12.825 | 5.008 | 2.561 | 8.76E−89 | MSX2 |
| MA0876.1 | 484 | 2003 | 12.825 | 5.008 | 2.561 | 8.76E−89 | BSX |
| MA1127.1 | 584 | 2417 | 15.474 | 6.043 | 2.561 | 2.50E−108 | FOSB::JUN |
| MA0101.1 | 1207 | 4996 | 31.982 | 12.490 | 2.561 | 1.00E−200 | REL |
| MA0630.1 | 557 | 2306 | 14.759 | 5.765 | 2.560 | 6.28E−103 | SHOX |
| MA0654.1 | 557 | 2306 | 14.759 | 5.765 | 2.560 | 6.28E−103 | ISX |
| MA0717.1 | 557 | 2306 | 14.759 | 5.765 | 2.560 | 6.28E−103 | RAX2 |
| MA0705.1 | 446 | 1847 | 11.818 | 4.618 | 2.559 | 2.09E−81 | Lhx8 |
| MA0075.3 | 446 | 1847 | 11.818 | 4.618 | 2.559 | 2.09E−81 | PRRX2 |
| MA0095.2 | 1567 | 6492 | 41.521 | 16.230 | 2.558 | 1.00E−200 | YY1 |
| MA1149.1 | 1489 | 6169 | 39.454 | 15.423 | 2.558 | 1.00E−200 | RARA::RXRG |
| MA1562.1 | 614 | 2545 | 16.269 | 6.363 | 2.557 | 5.33E−114 | SOX14 |
| MA0858.1 | 1200 | 4974 | 31.797 | 12.435 | 2.557 | 1.00E−200 | Rarb(var.2) |
| MA0111.1 | 1317 | 5462 | 34.897 | 13.655 | 2.556 | 1.00E−200 | Spz1 |
| MA0467.1 | 1059 | 4394 | 28.060 | 10.985 | 2.554 | 1.00E−200 | Crx |
| MA0729.1 | 1746 | 7250 | 46.264 | 18.125 | 2.552 | 1.00E−200 | RARA |
| MA0710.1 | 518 | 2151 | 13.725 | 5.378 | 2.552 | 8.92E−95 | NOTO |
| MA0057.1 | 1779 | 7391 | 47.138 | 18.478 | 2.551 | 1.00E−200 | MZF1(var.2) |
| MA1525.1 | 1435 | 5962 | 38.023 | 14.905 | 2.551 | 1.00E−200 | NFATC4 |
| MA1480.1 | 562 | 2336 | 14.891 | 5.840 | 2.550 | 3.70E−103 | DPRX |
| MA1148.1 | 1489 | 6191 | 39.454 | 15.478 | 2.549 | 1.00E−200 | PPARA::RXRA |
| MA0004.1 | 1719 | 7148 | 45.548 | 17.870 | 2.549 | 1.00E−200 | Arnt |
| MA0618.1 | 581 | 2416 | 15.395 | 6.040 | 2.549 | 8.35E−107 | LBX1 |
| MA1489.1 | 322 | 1340 | 8.532 | 3.350 | 2.547 | 9.67E−58 | FOXN3 |
| MA0693.2 | 1295 | 5390 | 34.314 | 13.475 | 2.546 | 1.00E−200 | VDR |
| MA0724.1 | 357 | 1486 | 9.459 | 3.715 | 2.546 | 4.19E−64 | VENTX |
| MA0886.1 | 392 | 1632 | 10.387 | 4.080 | 2.546 | 1.56E−70 | EMX2 |
| MA0903.1 | 653 | 2719 | 17.303 | 6.798 | 2.545 | 7.76E−121 | HOXB3 |
| MA0677.1 | 1450 | 6043 | 38.421 | 15.108 | 2.543 | 1.00E−200 | Nr2f6 |
| MA0141.3 | 1262 | 5261 | 33.439 | 13.153 | 2.542 | 1.00E−200 | ESRRB |
| MA0878.2 | 937 | 3909 | 24.828 | 9.773 | 2.541 | 6.05E−180 | CDX1 |
| MA0031.1 | 866 | 3616 | 22.946 | 9.040 | 2.538 | 2.88E−164 | FOXD1 |
| MA0157.2 | 866 | 3616 | 22.946 | 9.040 | 2.538 | 2.88E−164 | FOXO3 |
| MA1110.1 | 871 | 3637 | 23.079 | 9.093 | 2.538 | 2.50E−165 | NR1H4 |
| MA0623.2 | 380 | 1589 | 10.069 | 3.973 | 2.535 | 9.16E−68 | NEUROG1 |
| MA1657.1 | 1082 | 4525 | 28.670 | 11.313 | 2.534 | 1.00E−200 | ZNF652 |
| MA0890.1 | 520 | 2175 | 13.778 | 5.438 | 2.534 | 6.64E−94 | GBX2 |
| MA1136.1 | 445 | 1862 | 11.791 | 4.655 | 2.533 | 1.09E−79 | FOSB::JUNB(var.2) |
| MA0817.1 | 361 | 1511 | 9.565 | 3.778 | 2.532 | 3.51E−64 | BHLHE23 |
| MA0019.1 | 934 | 3913 | 24.748 | 9.783 | 2.530 | 6.52E−178 | Ddit3::Cebpa |
| MA1554.1 | 446 | 1869 | 11.818 | 4.673 | 2.529 | 1.18E−79 | RFX7 |
| MA1118.1 | 984 | 4125 | 26.073 | 10.313 | 2.528 | 1.09E−188 | SIX1 |
| MA0752.1 | 758 | 3178 | 20.085 | 7.945 | 2.528 | 1.71E−140 | ZNF410 |
| MA1501.1 | 502 | 2105 | 13.302 | 5.263 | 2.528 | 4.67E−90 | HOXB7 |
| MA1530.1 | 782 | 3280 | 20.721 | 8.200 | 2.527 | 2.53E−145 | NKX6-3 |
| MA0482.2 | 1128 | 4735 | 29.889 | 11.838 | 2.525 | 1.00E−200 | GATA4 |
| MA1577.1 | 367 | 1542 | 9.724 | 3.855 | 2.523 | 8.20E−65 | TLX2 |
| MA0676.1 | 1229 | 5169 | 32.565 | 12.923 | 2.520 | 1.00E−200 | Nr2e1 |
| MA0696.1 | 1476 | 6209 | 39.110 | 15.523 | 2.520 | 1.00E−200 | ZIC1 |
| MA0744.2 | 1227 | 5164 | 32.512 | 12.910 | 2.518 | 1.00E−200 | SCRT2 |
| MA1592.1 | 1146 | 4825 | 30.366 | 12.063 | 2.517 | 1.00E−200 | ZNF274 |
| MA0158.2 | 365 | 1537 | 9.671 | 3.843 | 2.517 | 3.47E−64 | HOXA5 |
| MA1105.2 | 1209 | 5093 | 32.035 | 12.733 | 2.516 | 1.00E−200 | GRHL2 |
| MA1639.1 | 897 | 3780 | 23.768 | 9.450 | 2.515 | 4.29E−168 | MEIS1(var.2) |
| MA0884.1 | 836 | 3523 | 22.152 | 8.808 | 2.515 | 3.11E−155 | DUXA |
| MA0025.2 | 852 | 3591 | 22.576 | 8.978 | 2.515 | 1.56E−158 | NFIL3 |
| MA0484.2 | 1445 | 6091 | 38.288 | 15.228 | 2.514 | 1.00E−200 | HNF4G |
| MA0672.1 | 909 | 3832 | 24.086 | 9.580 | 2.514 | 1.55E−170 | NKX2-3 |
| MA1473.1 | 1001 | 4224 | 26.524 | 10.560 | 2.512 | 3.66E−190 | CDX4 |
| MA0516.2 | 2555 | 10803 | 67.700 | 27.008 | 2.507 | 1.00E−200 | SP2 |
| MA0709.1 | 639 | 2703 | 16.932 | 6.758 | 2.506 | 1.22E−114 | Msx3 |
| MA0675.1 | 823 | 3483 | 21.807 | 8.708 | 2.504 | 2.47E−151 | NKX6-2 |
| MA0798.2 | 1136 | 4809 | 30.101 | 12.023 | 2.504 | 1.00E−200 | RFX3 |
| MA1546.1 | 302 | 1279 | 8.002 | 3.198 | 2.503 | 2.15E−52 | PAX3(var.2) |
| MA0509.2 | 1103 | 4673 | 29.226 | 11.683 | 2.502 | 1.00E−200 | RFX1 |
| MA0140.2 | 928 | 3936 | 24.589 | 9.840 | 2.499 | 1.16E−172 | GATA1::TAL1 |
| MA1523.1 | 1263 | 5358 | 33.466 | 13.395 | 2.498 | 1.00E−200 | MSANTD3 |
| MA0613.1 | 951 | 4042 | 25.199 | 10.105 | 2.494 | 6.91E−177 | FOXG1 |
| MA1491.1 | 1622 | 6894 | 42.978 | 17.235 | 2.494 | 1.00E−200 | GLI3 |
| MA1541.1 | 1336 | 5680 | 35.400 | 14.200 | 2.493 | 1.00E−200 | NR6A1 |
| MA0461.2 | 427 | 1816 | 11.314 | 4.540 | 2.492 | 4.63E−74 | Atoh1 |
| MA0730.1 | 1357 | 5772 | 35.957 | 14.430 | 2.492 | 1.00E−200 | RARA(var.2) |
| MA1534.1 | 869 | 3697 | 23.026 | 9.243 | 2.491 | 2.49E−159 | NR1I3 |
| MA0106.3 | 603 | 2566 | 15.978 | 6.415 | 2.491 | 1.71E−106 | TP53 |
| MA1594.1 | 1143 | 4865 | 30.286 | 12.163 | 2.490 | 1.00E−200 | ZNF382 |
| MA0066.1 | 1161 | 4942 | 30.763 | 12.355 | 2.490 | 1.00E−200 | PPARG |
| MA0707.1 | 948 | 4037 | 25.119 | 10.093 | 2.489 | 1.28E−175 | MNX1 |
| MA0774.1 | 947 | 4033 | 25.093 | 10.083 | 2.489 | 2.19E−175 | MEIS2 |
| MA1549.1 | 838 | 3571 | 22.205 | 8.928 | 2.487 | 1.82E−152 | POU6F1(var.2) |
| MA0037.3 | 1093 | 4658 | 28.961 | 11.645 | 2.487 | 1.00E−200 | GATA3 |
| MA0634.1 | 729 | 3108 | 19.316 | 7.770 | 2.486 | 1.88E−130 | ALX3 |
| MA0880.1 | 467 | 1991 | 12.374 | 4.978 | 2.486 | 6.30E−81 | Dlx3 |
| MA0881.1 | 467 | 1991 | 12.374 | 4.978 | 2.486 | 6.30E−81 | Dlx4 |
| MA0790.1 | 1393 | 5942 | 36.910 | 14.855 | 2.485 | 1.00E−200 | POU4F1 |
| MA1573.1 | 1740 | 7423 | 46.105 | 18.558 | 2.484 | 1.00E−200 | THAP11 |
| MA1520.1 | 772 | 3295 | 20.456 | 8.238 | 2.483 | 1.07E−138 | MAF |
| MA0063.2 | 973 | 4153 | 25.782 | 10.383 | 2.483 | 3.45E−180 | NKX2-5 |
| MA1589.1 | 1480 | 6321 | 39.216 | 15.803 | 2.482 | 1.00E−200 | ZNF140 |
| MA0818.1 | 298 | 1273 | 7.896 | 3.183 | 2.481 | 7.23E−51 | BHLHE22 |
| MA0773.1 | 2026 | 8656 | 53.683 | 21.640 | 2.481 | 1.00E−200 | MEF2D |
| MA1151.1 | 910 | 3888 | 24.112 | 9.720 | 2.481 | 1.46E−166 | RORC |
| MA0902.2 | 363 | 1551 | 9.618 | 3.878 | 2.481 | 4.13E−62 | HOXB2 |
| MA1476.1 | 438 | 1872 | 11.606 | 4.680 | 2.480 | 2.44E−75 | DLX5 |
| MA1580.1 | 826 | 3531 | 21.887 | 8.828 | 2.479 | 3.92E−149 | ZBTB32 |
| MA1531.1 | 1520 | 6498 | 40.276 | 16.245 | 2.479 | 1.00E−200 | NR1D1 |
| MA1616.1 | 1738 | 7430 | 46.052 | 18.575 | 2.479 | 1.00E−200 | Prdm15 |
| MA0834.1 | 432 | 1848 | 11.447 | 4.620 | 2.478 | 3.84E−74 | ATF7 |
| MA1640.1 | 1016 | 4349 | 26.921 | 10.873 | 2.476 | 1.94E−188 | MEIS2(var.2) |
| MA1625.1 | 1783 | 7633 | 47.244 | 19.083 | 2.476 | 1.00E−200 | Stat5b |
| MA0614.1 | 901 | 3858 | 23.874 | 9.645 | 2.475 | 5.17E−164 | Foxj2 |
| MA0114.4 | 1446 | 6201 | 38.315 | 15.503 | 2.472 | 1.00E−200 | HNF4A |
| MA1571.1 | 728 | 3122 | 19.290 | 7.805 | 2.471 | 7.99E−129 | TGIF2LX |
| MA1588.1 | 1111 | 4766 | 29.438 | 11.915 | 2.471 | 1.00E−200 | ZNF136 |
| MA0592.3 | 1544 | 6627 | 40.911 | 16.568 | 2.469 | 1.00E−200 | ESRRA |
| MA0043.3 | 712 | 3056 | 18.866 | 7.640 | 2.469 | 1.65E−125 | HLF |
| MA0826.1 | 321 | 1378 | 8.506 | 3.445 | 2.469 | 2.53E−54 | OLIG1 |
| MA0673.1 | 1005 | 4320 | 26.630 | 10.800 | 2.466 | 1.28E−184 | NKX2-8 |
| MA1139.1 | 418 | 1799 | 11.076 | 4.498 | 2.463 | 7.87E−71 | FOSL2::JUNB(var.2) |
| MA1104.2 | 1148 | 4942 | 30.419 | 12.355 | 2.462 | 1.00E−200 | GATA6 |
| MA0611.1 | 554 | 2385 | 14.679 | 5.963 | 2.462 | 3.80E−95 | Dux |
| MA1498.1 | 365 | 1572 | 9.671 | 3.930 | 2.461 | 1.59E−61 | HOXA7 |
| MA1502.1 | 365 | 1572 | 9.671 | 3.930 | 2.461 | 1.59E−61 | HOXB8 |
| MA0910.2 | 365 | 1572 | 9.671 | 3.930 | 2.461 | 1.59E−61 | HOXD8 |
| MA0894.1 | 501 | 2158 | 13.275 | 5.395 | 2.461 | 1.91E−85 | HESX1 |
| MA0159.1 | 1532 | 6600 | 40.594 | 16.500 | 2.460 | 1.00E−200 | RARA::RXRA |
| MA0681.2 | 1807 | 7785 | 47.880 | 19.463 | 2.460 | 1.00E−200 | PHOX2B |
| MA1624.1 | 1625 | 7001 | 43.058 | 17.503 | 2.460 | 1.00E−200 | Stat5a |
| MA1649.1 | 1479 | 6374 | 39.189 | 15.935 | 2.459 | 1.00E−200 | ZBTB12 |
| MA0743.2 | 1187 | 5116 | 31.452 | 12.790 | 2.459 | 1.00E−200 | SCRT1 |
| MA0734.2 | 1654 | 7129 | 43.826 | 17.823 | 2.459 | 1.00E−200 | GLI2 |
| MA0040.1 | 1274 | 5493 | 33.757 | 13.733 | 2.458 | 1.00E−200 | Foxq1 |
| MA0160.1 | 1355 | 5843 | 35.904 | 14.608 | 2.458 | 1.00E−200 | NR4A2 |
| MA0006.1 | 2099 | 9061 | 55.617 | 22.653 | 2.455 | 1.00E−200 | Ahr::Arnt |
| MA0520.1 | 1459 | 6307 | 38.659 | 15.768 | 2.452 | 1.00E−200 | Stat6 |
| MA0017.2 | 2120 | 9166 | 56.174 | 22.915 | 2.451 | 1.00E−200 | NR2F1 |
| MA0607.1 | 570 | 2467 | 15.103 | 6.168 | 2.449 | 4.36E−97 | Bhlha15 |
| MA1521.1 | 798 | 3454 | 21.145 | 8.635 | 2.449 | 3.75E−140 | MAFA |
| MA0859.1 | 1978 | 8565 | 52.411 | 21.413 | 2.448 | 1.00E−200 | Rarg |
| MA0606.1 | 1637 | 7091 | 43.376 | 17.728 | 2.447 | 1.00E−200 | NFAT5 |
| MA0739.1 | 1024 | 4436 | 27.133 | 11.090 | 2.447 | 5.93E−186 | Hic1 |
| MA1542.1 | 674 | 2920 | 17.859 | 7.300 | 2.446 | 4.06E−116 | OSR1 |
| MA0143.4 | 948 | 4108 | 25.119 | 10.270 | 2.446 | 5.68E−170 | SOX2 |
| MA0860.1 | 1618 | 7014 | 42.872 | 17.535 | 2.445 | 1.00E−200 | Rarg(var.2) |
| MA1145.1 | 618 | 2680 | 16.375 | 6.700 | 2.444 | 1.71E−105 | FOSL2::JUND(var.2) |
| MA0077.1 | 775 | 3362 | 20.535 | 8.405 | 2.443 | 4.46E−135 | SOX9 |
| MA1512.1 | 2510 | 10890 | 66.508 | 27.225 | 2.443 | 1.00E−200 | KLF11 |
| MA1468.1 | 708 | 3073 | 18.760 | 7.683 | 2.442 | 4.15E−122 | ATOH7 |
| MA0671.1 | 1022 | 4436 | 27.080 | 11.090 | 2.442 | 7.60E−185 | NFIX |
| MA0652.1 | 1159 | 5031 | 30.710 | 12.578 | 2.442 | 1.00E−200 | IRF8 |
| MA1119.1 | 1287 | 5589 | 34.102 | 13.973 | 2.441 | 1.00E−200 | SIX2 |
| MA0070.1 | 700 | 3040 | 18.548 | 7.600 | 2.441 | 1.83E−120 | PBX1 |
| MA0030.1 | 1014 | 4405 | 26.868 | 11.013 | 2.440 | 7.22E−183 | FOXF2 |
| MA0857.1 | 1914 | 8319 | 50.715 | 20.798 | 2.439 | 1.00E−200 | Rarb |
| MA0769.2 | 1202 | 5230 | 31.849 | 13.075 | 2.436 | 1.00E−200 | TCF7 |
| MA0766.2 | 816 | 3553 | 21.622 | 8.883 | 2.434 | 4.54E−142 | GATA5 |
| MA0137.3 | 1609 | 7006 | 42.634 | 17.515 | 2.434 | 1.00E−200 | STAT1 |
| MA0479.1 | 1043 | 4544 | 27.636 | 11.360 | 2.433 | 5.76E−188 | FOXH1 |
| MA0814.2 | 2282 | 9943 | 60.466 | 24.858 | 2.433 | 1.00E−200 | TFAP2C(var.2) |
| MA0862.1 | 114 | 497 | 3.021 | 1.243 | 2.431 | 2.49E−19 | GMEB2 |
| MA0042.2 | 1061 | 4628 | 28.113 | 11.570 | 2.430 | 2.44E−191 | FOXI1 |
| MA0033.2 | 1061 | 4628 | 28.113 | 11.570 | 2.430 | 2.44E−191 | FOXL1 |
| MA0848.1 | 1061 | 4628 | 28.113 | 11.570 | 2.430 | 2.44E−191 | FOXO4 |
| MA0849.1 | 1061 | 4628 | 28.113 | 11.570 | 2.430 | 2.44E−191 | FOXO6 |
| MA0850.1 | 1061 | 4628 | 28.113 | 11.570 | 2.430 | 2.44E−191 | FOXP3 |
| MA0668.1 | 267 | 1165 | 7.075 | 2.913 | 2.429 | 8.99E−44 | NEUROD2 |
| MA0642.1 | 397 | 1733 | 10.519 | 4.333 | 2.428 | 2.35E−65 | EN2 |
| MA1572.1 | 668 | 2921 | 17.700 | 7.303 | 2.424 | 5.91E−113 | TGIF2LY |
| MA1421.1 | 1482 | 6482 | 39.269 | 16.205 | 2.423 | 1.00E−200 | TCF7L1 |
| MA0510.2 | 627 | 2744 | 16.614 | 6.860 | 2.422 | 2.79E−105 | RFX5 |
| MA1529.1 | 1491 | 6529 | 39.507 | 16.323 | 2.420 | 1.00E−200 | NHLH2 |
| MA0656.1 | 538 | 2356 | 14.255 | 5.890 | 2.420 | 3.02E−89 | JDP2(var.2) |
| MA1124.1 | 1566 | 6866 | 41.494 | 17.165 | 2.417 | 1.00E−200 | ZNF24 |
| MA0869.1 | 1167 | 5119 | 30.922 | 12.798 | 2.416 | 1.00E−200 | Sox11 |
| MA1103.2 | 1067 | 4697 | 28.272 | 11.743 | 2.408 | 2.82E−189 | FOXK2 |
| MA0728.1 | 2087 | 9188 | 55.299 | 22.970 | 2.407 | 1.00E−200 | Nr2f6(var.2) |
| MA0792.1 | 1068 | 4702 | 28.299 | 11.755 | 2.407 | 1.94E−189 | POU5F1B |
| MA0627.2 | 1445 | 6366 | 38.288 | 15.915 | 2.406 | 1.00E−200 | POU2F3 |
| MA1540.1 | 1561 | 6878 | 41.362 | 17.195 | 2.405 | 1.00E−200 | NR5A1 |
| MA0619.1 | 1430 | 6301 | 37.891 | 15.753 | 2.405 | 1.00E−200 | LIN54 |
| MA0519.1 | 1733 | 7638 | 45.919 | 19.095 | 2.405 | 1.00E−200 | Stat5a::Stat5b |
| MA1115.1 | 1394 | 6146 | 36.937 | 15.365 | 2.404 | 1.00E−200 | POU5F1 |
| MA0795.1 | 342 | 1508 | 9.062 | 3.770 | 2.404 | 5.08E−55 | SMAD3 |
| MA0092.1 | 1129 | 4980 | 29.915 | 12.450 | 2.403 | 1.00E−200 | Hand1::Tcf3 |
| MA0747.1 | 2558 | 11287 | 67.780 | 28.218 | 2.402 | 1.00E−200 | SP8 |
| MA0504.1 | 2182 | 9631 | 57.817 | 24.078 | 2.401 | 1.00E−200 | NR2C2 |
| MA0100.3 | 905 | 3997 | 23.980 | 9.993 | 2.400 | 2.15E−155 | MYB |
| MA0669.1 | 517 | 2284 | 13.699 | 5.710 | 2.399 | 4.19E−84 | NEUROG2 |
| MA0789.1 | 1033 | 4565 | 27.371 | 11.413 | 2.398 | 7.38E−181 | POU3F4 |
| MA0468.1 | 951 | 4204 | 25.199 | 10.510 | 2.398 | 3.41E−164 | DUX4 |
| MA0667.1 | 914 | 4043 | 24.218 | 10.108 | 2.396 | 1.09E−156 | MYF6 |
| MA0148.4 | 1293 | 5724 | 34.261 | 14.310 | 2.394 | 1.00E−200 | FOXA1 |
| MA0605.2 | 494 | 2187 | 13.090 | 5.468 | 2.394 | 7.87E−80 | ATF3 |
| MA0670.1 | 969 | 4290 | 25.676 | 10.725 | 2.394 | 2.68E−167 | NFIA |
| MA1112.2 | 1645 | 7283 | 43.588 | 18.208 | 2.394 | 1.00E−200 | NR4A1 |
| MA0868.2 | 983 | 4354 | 26.047 | 10.885 | 2.393 | 6.02E−170 | SOX8 |
| MA0056.2 | 1393 | 6172 | 36.910 | 15.430 | 2.392 | 1.00E−200 | MZF1 |
| MA0072.1 | 910 | 4034 | 24.112 | 10.085 | 2.391 | 2.98E−155 | RORA(var.2) |
| MA0155.1 | 1974 | 8751 | 52.305 | 21.878 | 2.391 | 1.00E−200 | INSM1 |
| MA0833.2 | 1048 | 4646 | 27.769 | 11.615 | 2.391 | 8.12E−183 | ATF4 |
| MA0146.2 | 2727 | 12092 | 72.258 | 30.230 | 2.390 | 1.00E−200 | Zfx |
| MA0785.1 | 1190 | 5277 | 31.532 | 13.193 | 2.390 | 1.00E−200 | POU2F1 |
| MA1153.1 | 936 | 4154 | 24.801 | 10.385 | 2.388 | 5.46E−160 | Smad4 |
| MA1599.1 | 1955 | 8679 | 51.802 | 21.698 | 2.387 | 1.00E−200 | ZNF682 |
| MA0738.1 | 1567 | 6960 | 41.521 | 17.400 | 2.386 | 1.00E−200 | HIC2 |
| MA1131.1 | 645 | 2866 | 17.091 | 7.165 | 2.385 | 2.23E−105 | FOSL2::JUN(var.2) |
| MA1576.1 | 1420 | 6311 | 37.626 | 15.778 | 2.385 | 1.00E−200 | THRB(var.3) |
| MA0845.1 | 1501 | 6677 | 39.772 | 16.693 | 2.383 | 1.00E−200 | FOXB1 |
| MA0152.1 | 2043 | 9090 | 54.134 | 22.725 | 2.382 | 1.00E−200 | NFATC2 |
| MA0116.1 | 1327 | 5906 | 35.162 | 14.765 | 2.381 | 1.00E−200 | Znf423 |
| MA0498.2 | 1045 | 4651 | 27.689 | 11.628 | 2.381 | 8.19E−181 | MEIS1 |
| MA0465.2 | 1317 | 5862 | 34.897 | 14.655 | 2.381 | 1.00E−200 | CDX2 |
| MA1150.1 | 923 | 4109 | 24.457 | 10.273 | 2.381 | 1.71E−156 | RORB |
| MA0796.1 | 1028 | 4580 | 27.239 | 11.450 | 2.379 | 5.11E−177 | TGIF1 |
| MA0753.2 | 2184 | 9733 | 57.870 | 24.333 | 2.378 | 1.00E−200 | ZNF740 |
| MA0048.2 | 1511 | 6735 | 40.037 | 16.838 | 2.378 | 1.00E−200 | NHLH1 |
| MA0481.3 | 1251 | 5579 | 33.148 | 13.948 | 2.377 | 1.00E−200 | FOXP1 |
| MA0620.3 | 561 | 2503 | 14.865 | 6.258 | 2.376 | 5.93E−90 | MITF |
| MA0800.1 | 1210 | 5402 | 32.061 | 13.505 | 2.374 | 1.00E−200 | EOMES |
| MA0060.3 | 880 | 3930 | 23.317 | 9.825 | 2.373 | 2.80E−147 | NFYA |
| MA0901.2 | 1930 | 8624 | 51.139 | 21.560 | 2.372 | 1.00E−200 | HOXB13 |
| MA1528.1 | 1270 | 5675 | 33.651 | 14.188 | 2.372 | 1.00E−200 | NFIX(var.2) |
| MA0078.1 | 863 | 3859 | 22.867 | 9.648 | 2.370 | 1.08E−143 | Sox17 |
| MA1606.1 | 1365 | 6105 | 36.169 | 15.263 | 2.370 | 1.00E−200 | Foxf1 |
| MA0797.1 | 1010 | 4519 | 26.762 | 11.298 | 2.369 | 5.78E−172 | TGIF2 |
| MA0851.1 | 1719 | 7693 | 45.548 | 19.233 | 2.368 | 1.00E−200 | Foxj3 |
| MA0117.2 | 1083 | 4856 | 28.696 | 12.140 | 2.364 | 7.06E−186 | Mafb |
| MA0689.1 | 1315 | 5904 | 34.844 | 14.760 | 2.361 | 1.00E−200 | TBX20 |
| MA1597.1 | 1754 | 7876 | 46.476 | 19.690 | 2.360 | 1.00E−200 | ZNF528 |
| MA0595.1 | 1414 | 6350 | 37.467 | 15.875 | 2.360 | 1.00E−200 | SREBF1 |
| MA0032.2 | 1609 | 7231 | 42.634 | 18.078 | 2.358 | 1.00E−200 | FOXC1 |
| MA0784.1 | 1365 | 6140 | 36.169 | 15.350 | 2.356 | 1.00E−200 | POU1F1 |
| MA0494.1 | 1658 | 7458 | 43.932 | 18.645 | 2.356 | 1.00E−200 | Nr1h3::Rxra |
| MA0793.1 | 774 | 3485 | 20.509 | 8.713 | 2.354 | 2.02E−125 | POU6F2 |
| MA0690.1 | 1236 | 5569 | 32.750 | 13.923 | 2.352 | 1.00E−200 | TBX21 |
| MA0119.1 | 1435 | 6469 | 38.023 | 16.173 | 2.351 | 1.00E−200 | NFIC::TLX1 |
| MA1608.1 | 547 | 2468 | 14.494 | 6.170 | 2.349 | 1.26E−85 | Isl1 |
| MA1419.1 | 971 | 4382 | 25.729 | 10.955 | 2.349 | 1.66E−161 | IRF4 |
| MA1120.1 | 934 | 4217 | 24.748 | 10.543 | 2.347 | 2.79E−154 | SOX13 |
| MA0802.1 | 1146 | 5176 | 30.366 | 12.940 | 2.347 | 5.19E−196 | TBR1 |
| MA1102.2 | 2161 | 9761 | 57.260 | 24.403 | 2.346 | 1.00E−200 | CTCFL |
| MA0087.1 | 1220 | 5511 | 32.326 | 13.778 | 2.346 | 1.00E−200 | Sox5 |
| MA0047.3 | 1237 | 5590 | 32.777 | 13.975 | 2.345 | 1.00E−200 | FOXA2 |
| MA0783.1 | 1282 | 5794 | 33.969 | 14.485 | 2.345 | 1.00E−200 | PKNOX2 |
| MA0163.1 | 2285 | 10330 | 60.546 | 25.825 | 2.344 | 1.00E−200 | PLAG1 |
| MA0507.1 | 1467 | 6632 | 38.871 | 16.580 | 2.344 | 1.00E−200 | POU2F2 |
| MA1579.1 | 1923 | 8696 | 50.954 | 21.740 | 2.344 | 1.00E−200 | ZBTB26 |
| MA1683.1 | 1126 | 5095 | 29.836 | 12.738 | 2.342 | 3.15E−191 | FOXA3 |
| MA0596.1 | 1376 | 6227 | 36.460 | 15.568 | 2.342 | 1.00E−200 | SREBF2 |
| MA1563.1 | 807 | 3653 | 21.383 | 9.133 | 2.341 | 5.10E−130 | SOX18 |
| MA1557.1 | 250 | 1132 | 6.624 | 2.830 | 2.341 | 3.89E−38 | SMAD5 |
| MA0866.1 | 895 | 4056 | 23.715 | 10.140 | 2.339 | 7.83E−146 | SOX21 |
| MA0051.1 | 1521 | 6896 | 40.302 | 17.240 | 2.338 | 1.00E−200 | IRF2 |
| MA0842.2 | 863 | 3914 | 22.867 | 9.785 | 2.337 | 1.07E−139 | NRL |
| MA0775.1 | 1106 | 5018 | 29.306 | 12.545 | 2.336 | 3.47E−186 | MEIS3 |
| MA0847.2 | 1527 | 6930 | 40.461 | 17.325 | 2.335 | 1.00E−200 | FOXD2 |
| MA0788.1 | 1335 | 6059 | 35.374 | 15.148 | 2.335 | 1.00E−200 | POU3F3 |
| MA0801.1 | 1474 | 6700 | 39.057 | 16.750 | 2.332 | 1.00E−200 | MGA |
| MA0803.1 | 1474 | 6700 | 39.057 | 16.750 | 2.332 | 1.00E−200 | TBX15 |
| MA0495.3 | 1392 | 6331 | 36.884 | 15.828 | 2.330 | 1.00E−200 | MAFF |
| MA0688.1 | 1247 | 5672 | 33.042 | 14.180 | 2.330 | 1.00E−200 | TBX2 |
| MA1644.1 | 924 | 4204 | 24.483 | 10.510 | 2.330 | 4.64E−150 | NFYC |
| MA1485.1 | 1231 | 5601 | 32.618 | 14.003 | 2.329 | 1.00E−200 | FERD3L |
| MA1536.1 | 1236 | 5624 | 32.750 | 14.060 | 2.329 | 1.00E−200 | NR2C2(var.2) |
| MA1655.1 | 1406 | 6405 | 37.255 | 16.013 | 2.327 | 1.00E−200 | ZNF341 |
| MA0870.1 | 697 | 3184 | 18.468 | 7.960 | 2.320 | 1.73E−108 | Sox1 |
| MA0511.2 | 704 | 3218 | 18.654 | 8.045 | 2.319 | 1.52E−109 | RUNX2 |
| MA0806.1 | 1365 | 6244 | 36.169 | 15.610 | 2.317 | 1.00E−200 | TBX4 |
| MA0807.1 | 1365 | 6244 | 36.169 | 15.610 | 2.317 | 1.00E−200 | TBX5 |
| MA1656.1 | 1810 | 8283 | 47.960 | 20.708 | 2.316 | 1.00E−200 | ZNF449 |
| MA0653.1 | 1536 | 7032 | 40.700 | 17.580 | 2.315 | 1.00E−200 | IRF9 |
| MA0786.1 | 1297 | 5943 | 34.367 | 14.858 | 2.313 | 1.00E−200 | POU3F1 |
| MA0852.2 | 1092 | 5007 | 28.935 | 12.518 | 2.312 | 1.47E−179 | FOXK1 |
| MA0071.1 | 1078 | 4943 | 28.564 | 12.358 | 2.311 | 8.15E−177 | RORA |
| MA0144.2 | 1954 | 8960 | 51.775 | 22.400 | 2.311 | 1.00E−200 | STAT3 |
| MA0633.1 | 763 | 3500 | 20.217 | 8.750 | 2.311 | 6.98E−119 | Twist2 |
| MA0659.2 | 1415 | 6492 | 37.493 | 16.230 | 2.310 | 1.00E−200 | MAFG |
| MA1515.1 | 2800 | 12849 | 74.192 | 32.123 | 2.310 | 1.00E−200 | KLF2 |
| MA0787.1 | 1241 | 5696 | 32.883 | 14.240 | 2.309 | 1.00E−200 | POU3F2 |
| MA1418.1 | 1980 | 9091 | 52.464 | 22.728 | 2.308 | 1.00E−200 | IRF3 |
| MA0518.1 | 1833 | 8418 | 48.569 | 21.045 | 2.308 | 1.00E−200 | Stat4 |
| MA0832.1 | 1016 | 4669 | 26.921 | 11.673 | 2.306 | 3.64E−164 | Tcf21 |
| MA0088.2 | 1217 | 5593 | 32.247 | 13.983 | 2.306 | 1.00E−200 | ZNF143 |
| MA0741.1 | 2707 | 12450 | 71.728 | 31.125 | 2.305 | 1.00E−200 | KLF16 |
| MA0258.2 | 1599 | 7356 | 42.369 | 18.390 | 2.304 | 1.00E−200 | ESR2 |
| MA0593.1 | 1518 | 7009 | 40.223 | 17.523 | 2.295 | 1.00E−200 | FOXP2 |
| MA0523.1 | 1393 | 6432 | 36.910 | 16.080 | 2.295 | 1.00E−200 | TCF7L2 |
| MA1632.1 | 1202 | 5552 | 31.849 | 13.880 | 2.295 | 1.48E−198 | ATF2 |
| MA1121.1 | 1148 | 5303 | 30.419 | 13.258 | 2.294 | 8.77E−188 | TEAD2 |
| MA0505.1 | 1786 | 8257 | 47.324 | 20.643 | 2.293 | 1.00E−200 | Nr5a2 |
| MA0502.2 | 1052 | 4864 | 27.875 | 12.160 | 2.292 | 6.49E−169 | NFYB |
| MA1641.1 | 1179 | 5456 | 31.240 | 13.640 | 2.290 | 3.24E−193 | MYF5 |
| MA1607.1 | 1449 | 6707 | 38.394 | 16.768 | 2.290 | 1.00E−200 | Foxl2 |
| MA0503.1 | 1432 | 6629 | 37.944 | 16.573 | 2.290 | 1.00E−200 | Nkx2-5(var.2) |
| MA0768.1 | 1422 | 6583 | 37.679 | 16.458 | 2.289 | 1.00E−200 | LEF1 |
| MA0492.1 | 1114 | 5162 | 29.518 | 12.905 | 2.287 | 5.45E−180 | JUND(var.2) |
| MA1511.1 | 2329 | 10802 | 61.712 | 27.005 | 2.285 | 1.00E−200 | KLF10 |
| MA1123.2 | 1409 | 6542 | 37.334 | 16.355 | 2.283 | 1.00E−200 | TWIST1 |
| MA0500.2 | 1327 | 6162 | 35.162 | 15.405 | 2.282 | 1.00E−200 | MYOG |
| MA0003.4 | 2103 | 9769 | 55.723 | 24.423 | 2.282 | 1.00E−200 | TFAP2A |
| MA1116.1 | 2064 | 9607 | 54.690 | 24.018 | 2.277 | 1.00E−200 | RBPJ |
| MA0038.2 | 1235 | 5756 | 32.724 | 14.390 | 2.274 | 1.00E−200 | GFI1 |
| MA1635.1 | 1586 | 7393 | 42.024 | 18.483 | 2.274 | 1.00E−200 | BHLHE22(var.2) |
| MA1567.1 | 1466 | 6836 | 38.845 | 17.090 | 2.273 | 1.00E−200 | TBX6 |
| MA1645.1 | 1357 | 6332 | 35.957 | 15.830 | 2.271 | 1.00E−200 | NKX2-2 |
| MA1619.1 | 1235 | 5767 | 32.724 | 14.418 | 2.270 | 1.00E−200 | Ptf1a(var.2) |
| MA0138.2 | 1761 | 8238 | 46.661 | 20.595 | 2.266 | 1.00E−200 | REST |
| MA0820.1 | 1556 | 7290 | 41.229 | 18.225 | 2.262 | 1.00E−200 | FIGLA |
| MA0665.1 | 1182 | 5547 | 31.320 | 13.868 | 2.258 | 2.46E−188 | MSC |
| MA1637.1 | 1799 | 8443 | 47.668 | 21.108 | 2.258 | 1.00E−200 | EBF3 |
| MA0772.1 | 1765 | 8286 | 46.767 | 20.715 | 2.258 | 1.00E−200 | IRF7 |
| MA0805.1 | 1571 | 7377 | 41.627 | 18.443 | 2.257 | 1.00E−200 | TBX1 |
| MA0018.4 | 1125 | 5291 | 29.809 | 13.228 | 2.254 | 1.24E−176 | CREB1 |
| MA1581.1 | 2153 | 10129 | 57.048 | 25.323 | 2.253 | 1.00E−200 | ZBTB6 |
| MA1621.1 | 1893 | 8907 | 50.159 | 22.268 | 2.253 | 1.00E−200 | Rbpjl |
| MA1600.1 | 1598 | 7523 | 42.342 | 18.808 | 2.251 | 1.00E−200 | ZNF684 |
| MA1618.1 | 1403 | 6605 | 37.175 | 16.513 | 2.251 | 1.00E−200 | Ptf1a |
| MA1615.1 | 2263 | 10679 | 59.963 | 26.698 | 2.246 | 1.00E−200 | Plagl1 |
| MA1604.1 | 1733 | 8185 | 45.919 | 20.463 | 2.244 | 1.00E−200 | Ebf2 |
| MA0480.1 | 1628 | 7691 | 43.137 | 19.228 | 2.244 | 1.00E−200 | Foxo1 |
| MA1514.1 | 2284 | 10791 | 60.519 | 26.978 | 2.243 | 1.00E−200 | KLF17 |
| MA1527.1 | 1246 | 5887 | 33.015 | 14.718 | 2.243 | 4.39E−198 | NFIC(var.2) |
| MA0809.2 | 1252 | 5918 | 33.174 | 14.795 | 2.242 | 4.49E−199 | TEAD4 |
| MA0745.2 | 1907 | 9020 | 50.530 | 22.550 | 2.241 | 1.00E−200 | SNAI2 |
| MA0808.1 | 1055 | 4991 | 27.954 | 12.478 | 2.240 | 1.26E−161 | TEAD3 |
| MA1643.1 | 1743 | 8247 | 46.184 | 20.618 | 2.240 | 1.00E−200 | NFIB |
| MA0090.3 | 1293 | 6129 | 34.261 | 15.323 | 2.236 | 1.00E−200 | TEAD1 |
| MA0497.1 | 2473 | 11723 | 65.527 | 29.308 | 2.236 | 1.00E−200 | MEF2C |
| MA1566.1 | 1575 | 7468 | 41.733 | 18.670 | 2.235 | 1.00E−200 | TBX3 |
| MA0684.2 | 1411 | 6695 | 37.387 | 16.738 | 2.234 | 1.00E−200 | RUNX3 |
| MA0093.3 | 1079 | 5120 | 28.590 | 12.800 | 2.234 | 6.09E−165 | USF1 |
| MA1630.1 | 2304 | 10957 | 61.049 | 27.393 | 2.229 | 1.00E−200 | Znf281 |
| MA1487.1 | 1550 | 7376 | 41.070 | 18.440 | 2.227 | 1.00E−200 | FOXE1 |
| MA0687.1 | 1846 | 8786 | 48.914 | 21.965 | 2.227 | 1.00E−200 | SPIC |
| MA0052.4 | 2581 | 12285 | 68.389 | 30.713 | 2.227 | 1.00E−200 | MEF2A |
| MA0597.1 | 1569 | 7469 | 41.574 | 18.673 | 2.226 | 1.00E−200 | THAP1 |
| MA0091.1 | 1033 | 4925 | 27.371 | 12.313 | 2.223 | 4.36E−155 | TAL1::TCF3 |
| MA1654.1 | 1929 | 9204 | 51.113 | 23.010 | 2.221 | 1.00E−200 | ZNF16 |
| MA1518.1 | 382 | 1824 | 10.122 | 4.560 | 2.220 | 2.61E−52 | LHX1 |
| MA0846.1 | 1972 | 9424 | 52.252 | 23.560 | 2.218 | 1.00E−200 | FOXC2 |
| MA1100.2 | 1556 | 7437 | 41.229 | 18.593 | 2.218 | 1.00E−200 | ASCL1 |
| MA0142.1 | 1464 | 6998 | 38.792 | 17.495 | 2.217 | 1.00E−200 | Pou5f1::Sox2 |
| MA0515.1 | 1634 | 7813 | 43.296 | 19.533 | 2.217 | 1.00E−200 | Sox6 |
| MA1601.1 | 1946 | 9307 | 51.563 | 23.268 | 2.216 | 1.00E−200 | ZNF75D |
| MA1472.1 | 1355 | 6487 | 35.904 | 16.218 | 2.214 | 1.00E−200 | BHLHA15(var.2) |
| MA0526.3 | 1036 | 4974 | 27.451 | 12.435 | 2.208 | 2.62E−153 | USF2 |
| MA0154.4 | 1905 | 9157 | 50.477 | 22.893 | 2.205 | 1.00E−200 | EBF1 |
| MA0103.3 | 2056 | 9905 | 54.478 | 24.763 | 2.200 | 1.00E−200 | ZEB1 |
| MA0499.2 | 1827 | 8805 | 48.410 | 22.013 | 2.199 | 1.00E−200 | MYOD1 |
| MA0867.2 | 1513 | 7304 | 40.090 | 18.260 | 2.196 | 1.00E−200 | SOX4 |
| MA1647.1 | 1819 | 8803 | 48.198 | 22.008 | 2.190 | 1.00E−200 | PRDM4 |
| MA0161.2 | 1306 | 6332 | 34.605 | 15.830 | 2.186 | 7.47E−199 | NFIC |
| MA1152.1 | 1686 | 8181 | 44.674 | 20.453 | 2.184 | 1.00E−200 | SOX15 |
| MA0488.1 | 1168 | 5679 | 30.949 | 14.198 | 2.180 | 3.21E−172 | JUN |
| MA0065.2 | 2316 | 11291 | 61.367 | 28.228 | 2.174 | 1.00E−200 | Pparg::Rxra |
| MA1593.1 | 1531 | 7464 | 40.567 | 18.660 | 2.174 | 1.00E−200 | ZNF317 |
| MA1568.1 | 374 | 1824 | 9.910 | 4.560 | 2.173 | 7.06E−49 | TCF21(var.2) |
| MA0698.1 | 1641 | 8004 | 43.482 | 20.010 | 2.173 | 1.00E−200 | ZBTB18 |
| MA1646.1 | 1837 | 8960 | 48.675 | 22.400 | 2.173 | 1.00E−200 | OSR2 |
| MA1620.1 | 1857 | 9061 | 49.205 | 22.653 | 2.172 | 1.00E−200 | Ptf1a(var.3) |
| MA0782.2 | 1693 | 8270 | 44.860 | 20.675 | 2.170 | 1.00E−200 | PKNOX1 |
| MA0691.1 | 978 | 4779 | 25.914 | 11.948 | 2.169 | 6.70E−138 | TFAP4 |
| MA1108.2 | 1312 | 6415 | 34.764 | 16.038 | 2.168 | 2.15E−196 | MXI1 |
| MA0522.3 | 2094 | 10245 | 55.485 | 25.613 | 2.166 | 1.00E−200 | TCF3 |
| MA0041.1 | 2235 | 10970 | 59.221 | 27.425 | 2.159 | 1.00E−200 | Foxd3 |
| MA1559.1 | 1987 | 9757 | 52.650 | 24.393 | 2.158 | 1.00E−200 | SNAI3 |
| MA0830.2 | 2132 | 10488 | 56.492 | 26.220 | 2.155 | 1.00E−200 | TCF4 |
| MA0483.1 | 1947 | 9595 | 51.590 | 23.988 | 2.151 | 1.00E−200 | Gfi1b |
| MA1648.1 | 2094 | 10333 | 55.485 | 25.833 | 2.148 | 1.00E−200 | TCF12(var.2) |
| MA1565.1 | 1982 | 9785 | 52.517 | 24.463 | 2.147 | 1.00E−200 | TBX18 |
| MA0591.1 | 1394 | 6883 | 36.937 | 17.208 | 2.147 | 1.00E−200 | Bach1::Mafk |
| MA0816.1 | 1570 | 7782 | 41.600 | 19.455 | 2.138 | 1.00E−200 | Ascl2 |
| MA1638.1 | 1330 | 6602 | 35.241 | 16.505 | 2.135 | 2.71E−193 | HAND2 |
| MA0508.3 | 2242 | 11140 | 59.406 | 27.850 | 2.133 | 1.00E−200 | PRDM1 |
| MA1558.1 | 2087 | 10381 | 55.299 | 25.953 | 2.131 | 1.00E−200 | SNAI1 |
| MA1109.1 | 1536 | 7649 | 40.700 | 19.123 | 2.128 | 1.00E−200 | NEUROD1 |
| MA1467.1 | 1492 | 7440 | 39.534 | 18.600 | 2.125 | 1.00E−200 | ATOH1(var.2) |
| MA1114.1 | 1901 | 9499 | 50.371 | 23.748 | 2.121 | 1.00E−200 | PBX3 |
| MA1155.1 | 1508 | 7540 | 39.958 | 18.850 | 2.120 | 1.00E−200 | ZSCAN4 |
| MA1627.1 | 2781 | 13954 | 73.688 | 34.885 | 2.112 | 1.00E−200 | Wt1 |
| MA1653.1 | 2992 | 15020 | 79.279 | 37.550 | 2.111 | 1.00E−200 | ZNF148 |
| MA1522.1 | 2685 | 13507 | 71.145 | 33.768 | 2.107 | 1.00E−200 | MAZ |
| MA0442.2 | 1629 | 8195 | 43.164 | 20.488 | 2.107 | 1.00E−200 | SOX10 |
| MA1642.1 | 1478 | 7436 | 39.163 | 18.590 | 2.107 | 1.00E−200 | NEUROG2(var.2) |
| MA0493.1 | 2340 | 11849 | 62.003 | 29.623 | 2.093 | 1.00E−200 | Klf1 |
| MA0150.2 | 1404 | 7131 | 37.202 | 17.828 | 2.087 | 9.94E−197 | Nfe2l2 |
| MA1631.1 | 2215 | 11292 | 58.691 | 28.230 | 2.079 | 1.00E−200 | ASCL1(var.2) |
| MA0514.1 | 2034 | 10373 | 53.895 | 25.933 | 2.078 | 1.00E−200 | Sox3 |
| MA0521.1 | 2029 | 10366 | 53.763 | 25.915 | 2.075 | 1.00E−200 | Tcf12 |
| MA0598.3 | 2496 | 12798 | 66.137 | 31.995 | 2.067 | 1.00E−200 | EHF |
| MA0517.1 | 2609 | 13426 | 69.131 | 33.565 | 2.060 | 1.00E−200 | STAT1::STAT2 |
| MA0473.3 | 2620 | 13512 | 69.422 | 33.780 | 2.055 | 1.00E−200 | ELF1 |
| MA0599.1 | 3034 | 15706 | 80.392 | 39.265 | 2.047 | 1.00E−200 | KLF5 |
| MA0513.1 | 1983 | 10306 | 52.544 | 25.765 | 2.039 | 1.00E−200 | SMAD2::SMAD3::SMAD4 |
| MA0501.1 | 1341 | 6970 | 35.533 | 17.425 | 2.039 | 7.89E−176 | MAF::NFE2 |
| MA1587.1 | 2899 | 15113 | 76.815 | 37.783 | 2.033 | 1.00E−200 | ZNF135 |
| MA1652.1 | 2677 | 13995 | 70.933 | 34.988 | 2.027 | 1.00E−200 | ZKSCAN5 |
| MA0039.4 | 3106 | 16359 | 82.300 | 40.898 | 2.012 | 1.00E−200 | KLF4 |
| MA0496.3 | 2195 | 11579 | 58.161 | 28.948 | 2.009 | 1.00E−200 | MAFK |
| MA0151.1 | 2713 | 14341 | 71.887 | 35.853 | 2.005 | 1.00E−200 | Arid3a |
| MA0062.3 | 2611 | 13864 | 69.184 | 34.660 | 1.996 | 1.00E−200 | GABPA |
| MA0640.2 | 2615 | 13921 | 69.290 | 34.803 | 1.991 | 1.00E−200 | ELF3 |
| MA0761.2 | 2549 | 13660 | 67.541 | 34.150 | 1.978 | 1.00E−200 | ETV1 |
| MA1629.1 | 2135 | 11450 | 56.571 | 28.625 | 1.976 | 1.00E−200 | Zic2 |
| MA0130.1 | 2604 | 13973 | 68.998 | 34.933 | 1.975 | 1.00E−200 | ZNF354C |
| MA1508.1 | 2635 | 14174 | 69.820 | 35.435 | 1.970 | 1.00E−200 | IKZF1 |
| MA1107.2 | 2877 | 15586 | 76.232 | 38.965 | 1.956 | 1.00E−200 | KLF9 |
| MA1633.1 | 1502 | 8140 | 39.799 | 20.350 | 1.956 | 3.87E−184 | BACH1 |
| MA0764.2 | 2707 | 14686 | 71.728 | 36.715 | 1.954 | 1.00E−200 | ETV4 |
| MA0528.2 | 2693 | 14612 | 71.357 | 36.530 | 1.953 | 1.00E−200 | ZNF263 |
| MA0089.2 | 1277 | 6934 | 33.837 | 17.335 | 1.952 | 1.77E−148 | NFE2L1 |
| MA0073.1 | 2810 | 15272 | 74.457 | 38.180 | 1.950 | 1.00E−200 | RREB1 |
| MA0081.2 | 2804 | 15303 | 74.298 | 38.258 | 1.942 | 1.00E−200 | SPIB |
| MA0002.2 | 1984 | 10842 | 52.570 | 27.105 | 1.940 | 1.00E−200 | RUNX1 |
| MA1125.1 | 2988 | 16339 | 79.173 | 40.848 | 1.938 | 1.00E−200 | ZNF384 |
| MA1628.1 | 2246 | 12369 | 59.512 | 30.923 | 1.925 | 1.00E−200 | Zic1::Zic2 |
| MA0841.1 | 984 | 5433 | 26.073 | 13.583 | 1.920 | 1.73E−103 | NFE2 |
| MA0476.1 | 1373 | 7750 | 36.380 | 19.375 | 1.878 | 3.19E−147 | FOS |
| MA1623.1 | 2853 | 16148 | 75.596 | 40.370 | 1.873 | 1.00E−200 | Stat2 |
| MA1596.1 | 3287 | 18707 | 87.096 | 46.768 | 1.862 | 1.00E−200 | ZNF460 |
| MA0478.1 | 1306 | 7439 | 34.605 | 18.598 | 1.861 | 1.16E−134 | FOSL2 |
| MA0149.1 | 2976 | 16998 | 78.855 | 42.495 | 1.856 | 1.00E−200 | EWSR1-FLI1 |
| MA1132.1 | 934 | 5360 | 24.748 | 13.400 | 1.847 | 1.32E−87 | JUN::JUNB |
| MA0489.1 | 1401 | 8042 | 37.122 | 20.105 | 1.846 | 2.20E−144 | JUN(var.2) |
| MA1101.2 | 918 | 5279 | 24.324 | 13.198 | 1.843 | 2.33E−85 | BACH2 |
| MA1622.1 | 1460 | 8416 | 38.686 | 21.040 | 1.839 | 1.02E−150 | Smad2::Smad3 |
| MA0099.3 | 1157 | 6694 | 30.657 | 16.735 | 1.832 | 1.04E−110 | FOS::JUN |
| MA1128.1 | 1283 | 7423 | 33.996 | 18.558 | 1.832 | 4.82E−126 | FOSL1::JUN |
| MA1142.1 | 1186 | 6864 | 31.426 | 17.160 | 1.831 | 4.68E−114 | FOSL1::JUND |
| MA1144.1 | 1142 | 6613 | 30.260 | 16.533 | 1.830 | 1.13E−108 | FOSL2::JUND |
| MA0655.1 | 981 | 5687 | 25.994 | 14.218 | 1.828 | 3.51E−90 | JDP2 |
| MA1138.1 | 1122 | 6505 | 29.730 | 16.263 | 1.828 | 5.64E−106 | FOSL2::JUNB |
| MA0477.2 | 1291 | 7509 | 34.208 | 18.773 | 1.822 | 3.75E−125 | FOSL1 |
| MA1130.1 | 1201 | 7009 | 31.823 | 17.523 | 1.816 | 3.92E−113 | FOSL2::JUN |
| MA1137.1 | 1176 | 6893 | 31.161 | 17.233 | 1.808 | 7.89E−109 | FOSL1::JUNB |
| MA0080.5 | 2996 | 17568 | 79.385 | 43.920 | 1.807 | 1.00E−200 | SPI1 |
| MA1135.1 | 1104 | 6496 | 29.253 | 16.240 | 1.801 | 1.49E−99 | FOSB::JUNB |
| MA0050.2 | 3155 | 18571 | 83.598 | 46.428 | 1.801 | 1.00E−200 | IRF1 |
| MA0491.2 | 1093 | 6437 | 28.961 | 16.093 | 1.800 | 4.36E−98 | JUND |
| MA1141.1 | 1288 | 7590 | 34.128 | 18.975 | 1.799 | 3.57E−120 | FOS::JUND |
| MA0462.2 | 1433 | 8445 | 37.970 | 21.113 | 1.798 | 4.39E−138 | BATF::JUN |
| MA1634.1 | 1424 | 8396 | 37.732 | 20.990 | 1.798 | 9.62E−137 | BATF |
| MA0835.2 | 1447 | 8539 | 38.341 | 21.348 | 1.796 | 2.44E−139 | BATF3 |
| MA1134.1 | 1068 | 6327 | 28.299 | 15.818 | 1.789 | 1.01E−93 | FOS::JUNB |
| MA0490.2 | 1105 | 6617 | 29.279 | 16.543 | 1.770 | 1.37E−94 | JUNB |
| MA0139.1 | 1942 | 13821 | 51.457 | 34.553 | 1.489 | 2.31E−111 | CTCF |

Abbreviations: Observed = Obs; Background = BG; Percent = %; Fold Enrichment = FE; P Value = PV; Motif Name = Name.

TABLE 6
Transcription factors involved in immune effector, metabolic
reprogramming, and memory formation enriched in cluster
6 (IL-21 NK cells) from scATAC-seq data

| Function | TF | Reference |
|---|---|---|
| Maturation and | EOMES | {Gordon, 2012 #778} |
| Immune effector | TBX21 | {Huang, 2021 #780} |
| function | RELB | {Caamano, 1999 #751} |
| | IRF1 | {Duncan, 1996 #782} |
| | IRF9 | {Huber, 2017 #745 } |
| Metabolic | CEBPD | {Banerjee, 2016 #739} |
| reprogramming | EOMES | {Istaces, 2019 #727} |
| | STAT3 | {Poli, 2015 #738} |
| | ATF4 | {Yang, 2018 #728} |
| | ZEB1 | {Jiang, 2022 #747} |
| | KLF2 | {Kuo, 1997 #648; |
| | | Schober, 1999 #649} |
| | MAX | {Carroll, 2018 #788; |
| | | Gnanaprakasam, 2017 #742} |
| | MXI1 | {Corn, 2005 #741} |
| | REST | {Cavadas, 2016 #749} |
| | E2F3 | {Araki, 2019 #743 } |
| Memory formation | CEBPB | {de Laval, 2020 #737} |
| | AP-1 Complex | {Larsen, 2021 #713 } |
| | EOMES | {Istaces, 2019 #727} |
| | STAT1 | {Quigley, 2008 #731 } |
| | ETS1 | [Grenningloh, 2011 #787} |
| | KLF13 | {Zhou, 2007 #736} |
| | JUN | {Larsen, 2021 #707} |
| | RUNX2 | {Olesin, 2018 #748} |
| | TBX21 | {Munoz, 2021 #750} |

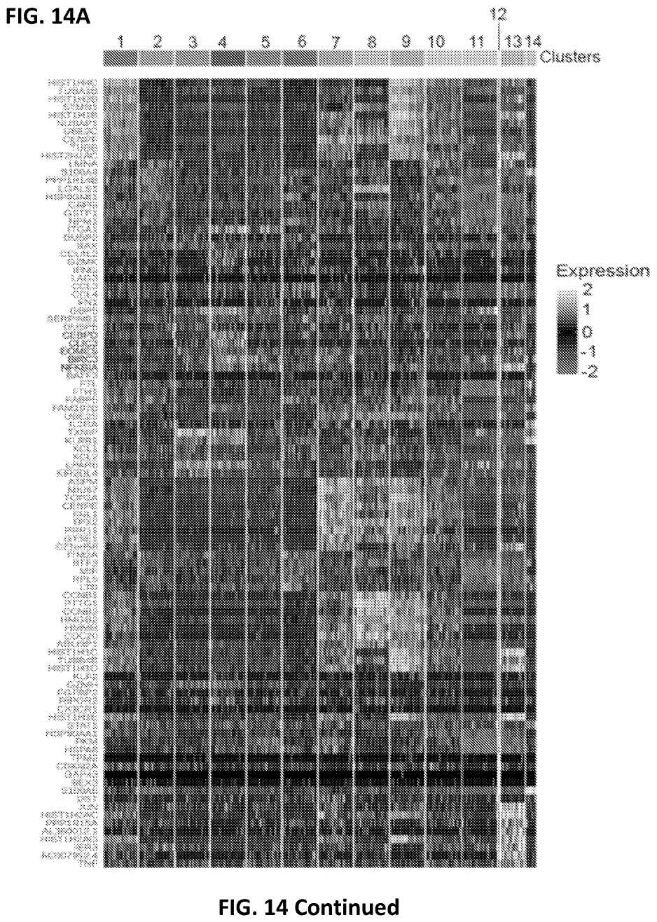

To further characterize the transcriptional landscape/heterogeneity in IL-21 NK cells, unbiased single-cell RNA (scRNA) profiling was performed. Clustering of the scRNA profiles revealed increasing differences over time between IL-21 NK and IL-15 NK cells cultured with or without GSCs, similar to the epigenetic profiles, with the most striking differences/divergences between the engineered cells being observed at day 9 after multiple GSC rechallenges (FIG. 4F and FIG. 14). Differential gene expression analysis comparing the dominant cluster in IL-15 (cluster 3) vs IL-21 NK cells (cluster 4) (FIGS. 4F-4G and FIG. 14) identified 176 genes upregulated in cluster 3, and 163 genes upregulated in cluster 4 (e.g., CEBPD, CLIC3, EOMES, BIRC3, and NFκBIA). A major feature of cluster 4 in the IL-21 NK cell product was upregulation of TFs previously identified in the scATAC-seq dataset such as CEBPD, ETS1, IRF1 and EOMES (FIGS. 4H-4J). We also identified genes important for T-cell memory and NK cell survival such as KLRD1, ITGA1 and GZMK³⁵⁻³⁷ Additional genes such as NFKBIA, REST, and E2F3 were also upregulated. In contrast, cluster 3 (IL-15 NK cells) showed upregulation of genes related to suppression of antitumor immunity and cell exhaustion such as DUSP2³⁸, CISH³⁹ and BAX⁴⁰ (FIG. 4H).

### Example 5

### C/EBP Regulated IL-21 NK Cells at the Transcriptional and Epigenetic Levels

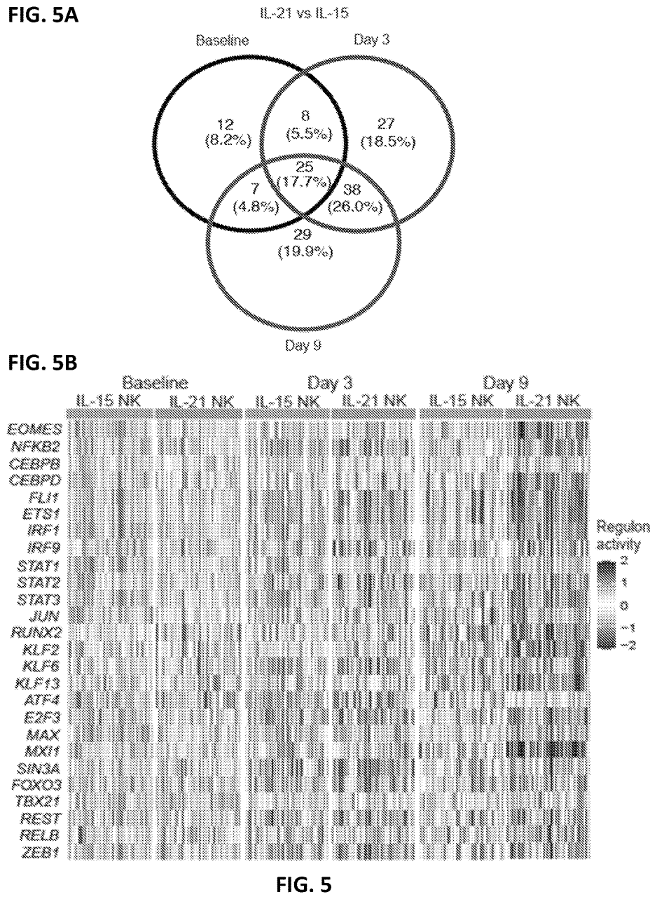

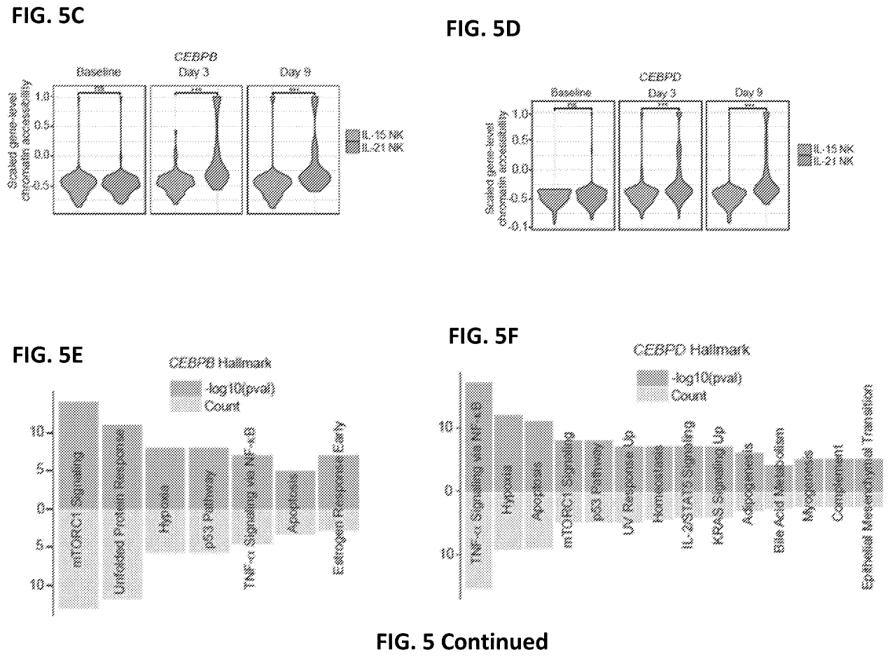

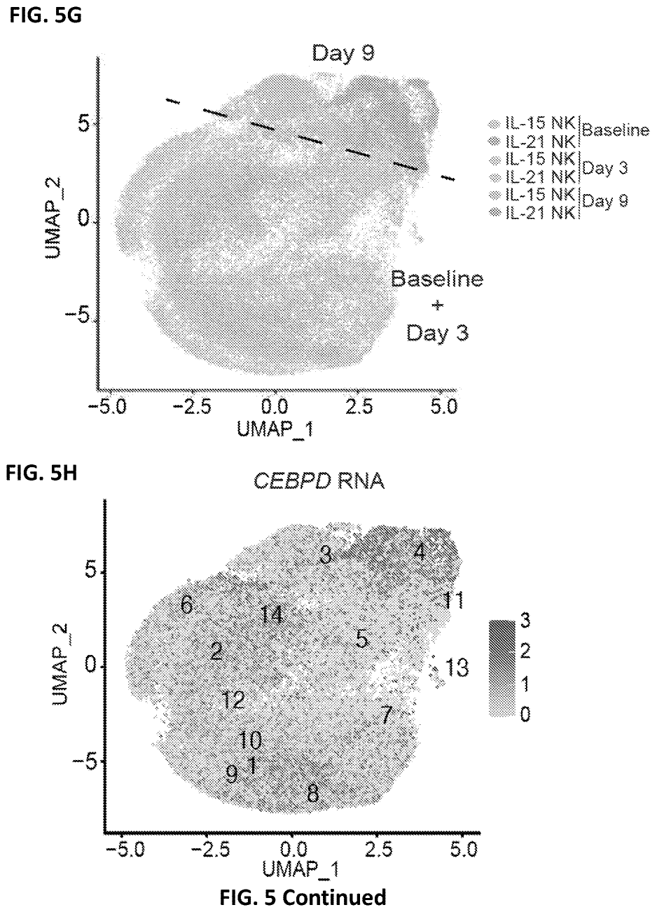

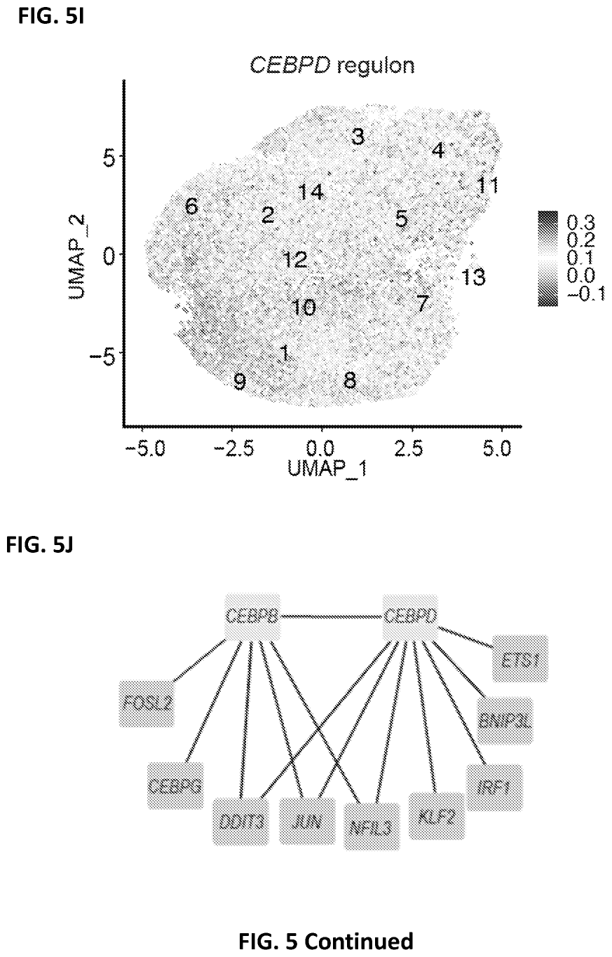

To validate the scATAC-seq TF findings reported above, the pySCENIC pipeline was utilized to infer gene regulatory networks from the scRNA-seq data. The results identified 146 differentially active regulons between IL-21 and IL-15 NK samples across all time points (FIG. 5A). Notably, most of the TF motifs enriched in the DAPs (differentially accessible peaks) in IL-21 NK vs IL-15 NK cells from the scATAC-seq data were also observed in this analysis (FIG. 5B). Both CEBPD and CEBPB were identified among the top 50 differentially active regulons, supporting the potential importance of these TFs in the modulation of IL-21 NK cell activity at the transcriptomic and epigenetic levels.

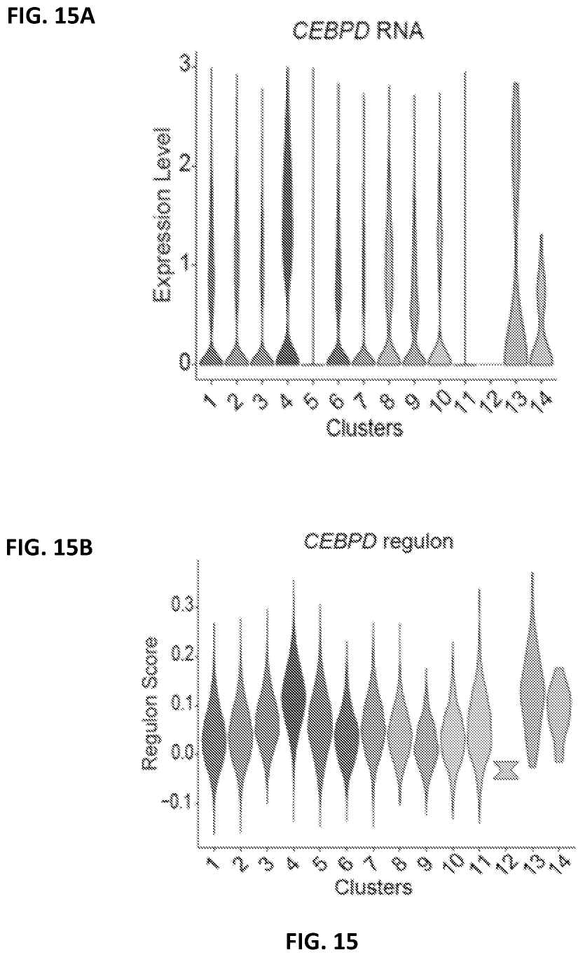

The gene level chromatin accessibility profiles of CEBPD (106 target genes, including target gene CEBPB) and CEBPB (85 targeted genes) were significantly higher in IL-21 NK cells after challenge or rechallenge with GSCs at day 3 and/or day 9 when compared to IL-15 NK cells (FIGS. 5C-5D). Among the pathways enriched for CEBPD and CEBPB regulons were TNF-α signaling via NF-κB, mTORC1 signaling, and hypoxia, pathways previously known to be implicated in memory formation/long-term persistence in T cells^{41,42} (FIGS. 5E-5F). Genes involved in CEBPB hallmark pathways such as mTORC1 signaling, unfolded protein response, hypoxia, p53 pathway, TNFα signaling via NF-κB, apoptosis, and estrogen response were significantly more accessible (FIG. 5E). Genes involved in CEBPD hallmark pathways such as TNFα signaling via NF-κB, hypoxia, apoptosis, mTORC1 signaling, p53 pathway, UV response up, homeostasis, IL-2/STAT5 signaling, KRAS signaling up, adipogenesis, bile acid metabolism, myogenesis, complement, and epithelial mesenchymal transition were significantly more accessible (FIG. 5F). Interestingly, cluster 4 which defined these IL-21 NK cells at day 9 of GSC co-culture (FIG. 5G) was enriched in CEBPD at both the transcriptomic level (FIG. 5H and FIG. 15A) and in the target genes of the CEBPD regulons (FIG. 5I and FIG. 15B). Taken together these data validated the importance of CEBPD as a TF that regulated multiple downstream genes such as CEBPβ, JUN, FOSL2, KLF2, ETS1, NFIL3 and BNIP3L^{32,33,43-46} and that could be implicated in the memory/cell persistence and mitochondrial fitness phenotypes observed for IL-21 NK cells (FIG. 5I).

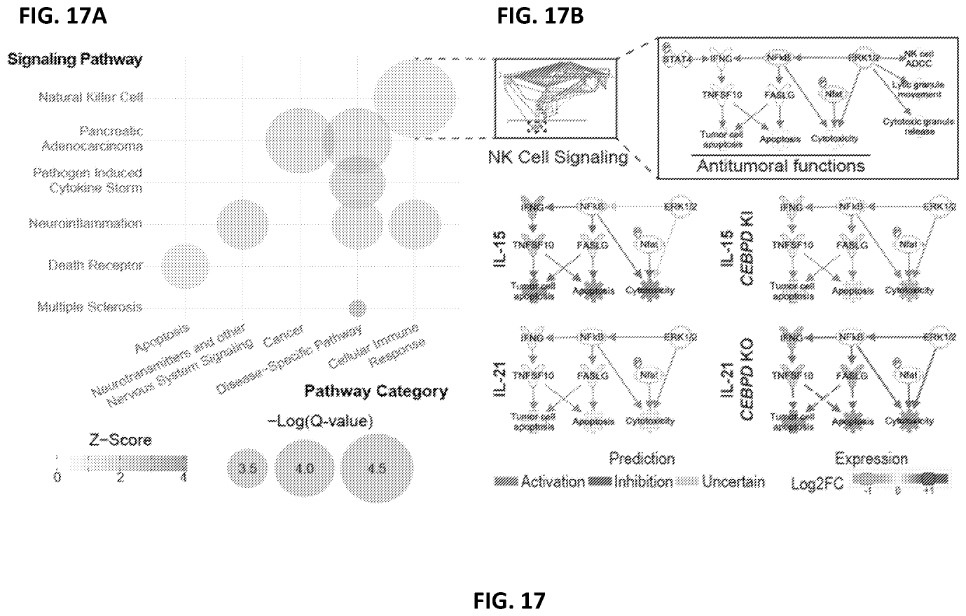

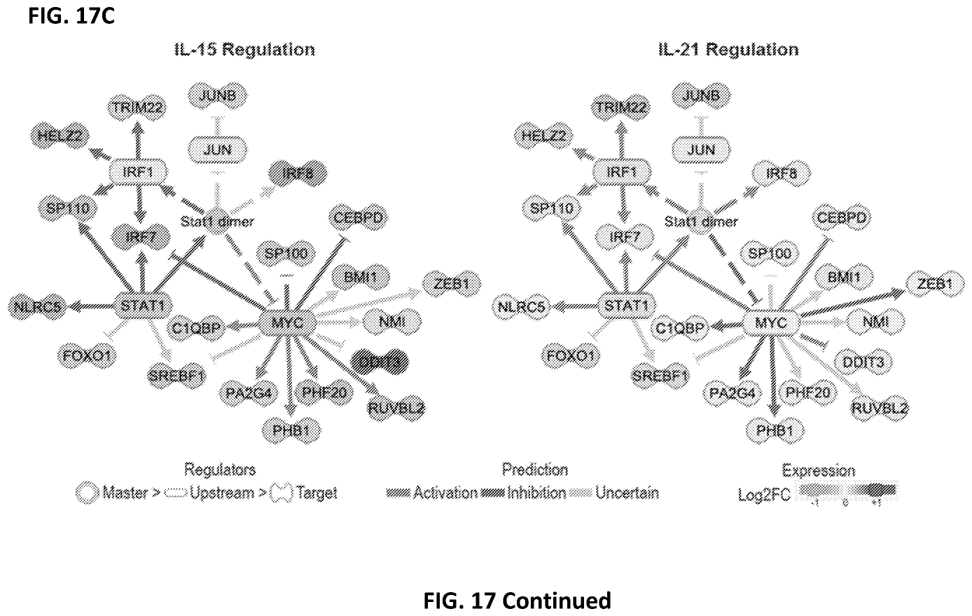

In addition, integrated analysis of bulk RNA-seq and ATAC-seq⁴⁷ from another set of 4 CB donors of NK cells engineered to express IL-15 or IL-21 revealed 1,146 DEGs and identified NK cell signaling as the top canonical pathway activated in IL-21 NK cells (FIG. 17A-17B). Further, analysis of TF motif activity revealed distinct regulatory network activity in IL-21 vs IL-15 NK cells (FIG. 17C). Specifically, the STAT1 gene network was upregulated in IL-21 NK cells and downregulated in IL-15 NK cells, while the MYC gene network was upregulated in IL-15 compared to IL-21 NK cells. The data also independently validated JUN as a top upstream regulator and CEBPD as a top target regulator activated in IL-21 but not IL-15 NK cells.

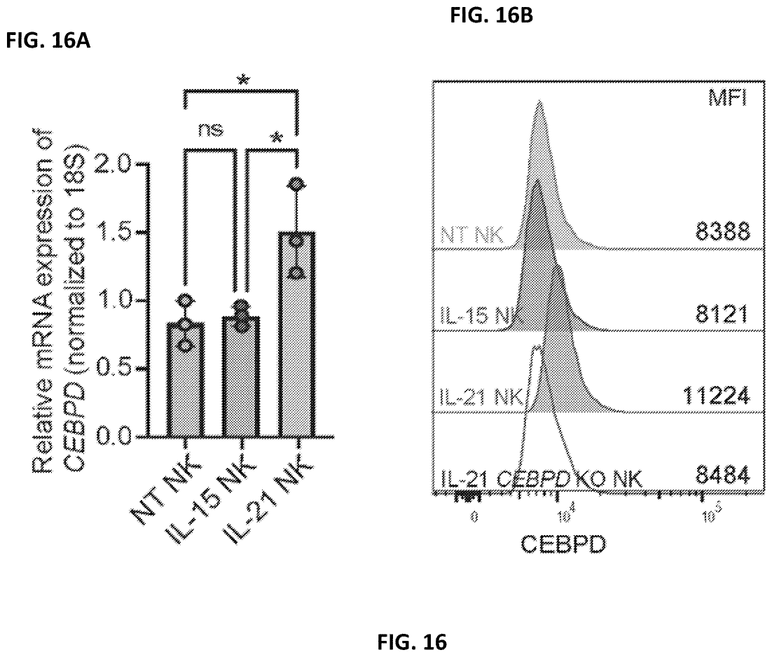

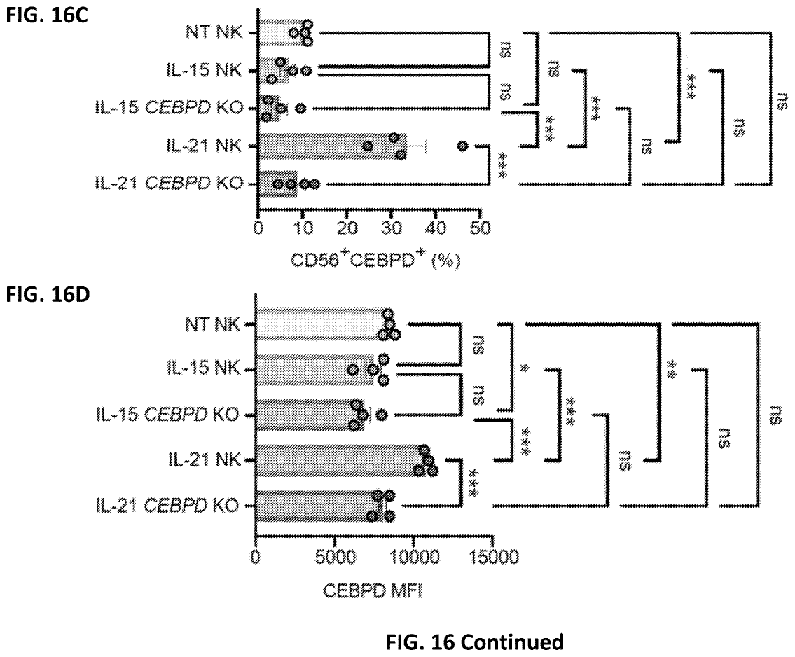

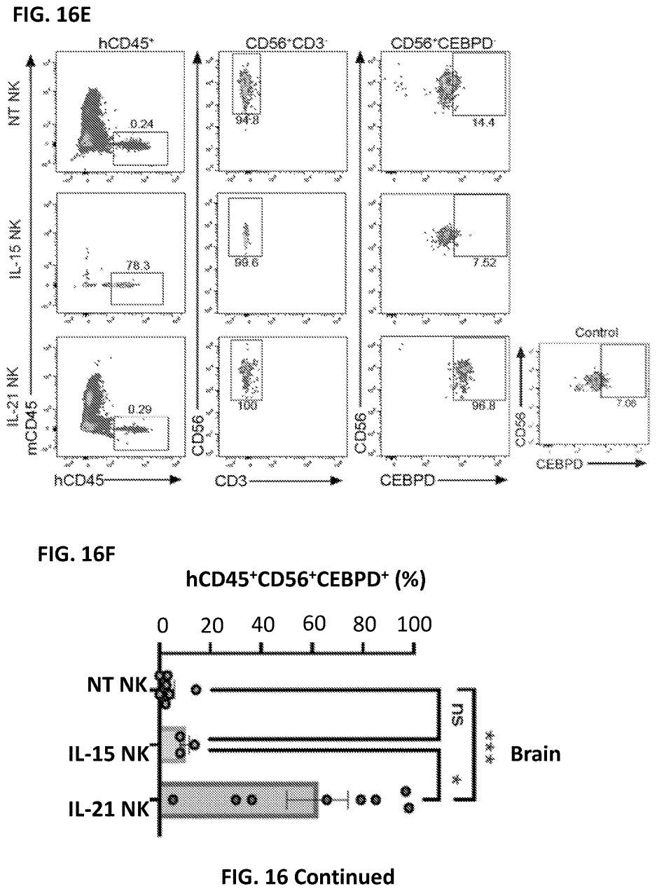

Finally, the inventors confirmed upregulation of CEBPD at the transcriptomic and proteomic levels in IL-21 NK cells after multiple GSC rechallenges, but not in NT NK cells or IL-15 NK cells (FIGS. 16A-D). This upregulation was also noted at the proteomic level in NK cells extracted from the brains of GSC-implanted mice treated with IL-21 NK cells but not with NT NK or IL-15 NK cells, reinforcing CEBPD's potentially important role in mediating the strong antitumor activity of IL-21 NK cells in vivo (FIGS. 16E-16F).

These results further supported the key role of CEBP family proteins, such as CEBPD, in mediating a strong antitumor NK cell response in vivo. Taken together, these data supported the role of CEBPD as a key TF mediating effector functions of IL-21 NK cells against GBM.

### Example 6

### CEBPD KO Reversed the Observed Advantages Conferred by IL-21

C/EBPs are a family of six structurally homologous TFs that can promote the expression of genes involved in various cellular process, such as cell proliferation, differentiation and death⁴⁸. In cancer, C/EBPs have been shown to serve both pro-oncogenic and co-suppressor roles⁴⁹, but their specific role in NK cell function at the molecular level was unclear and/or was previously unknown⁵⁰. Since the results reported herein suggested that CEBPD is a regulator of CEBPB and identified CEBPD as the most differentially enriched TF in IL-21 NK cells, the inventors first examined the impact of CEBPD deletion (CEBPD-knock out, CEBPD-KO) or insertion/overexpression (CEBPD-knock in, CEBPD-KI, CEBPD-Overexpression, CEBPD-OE) in IL-21 NK cells and IL-15 NK cells, respectively.

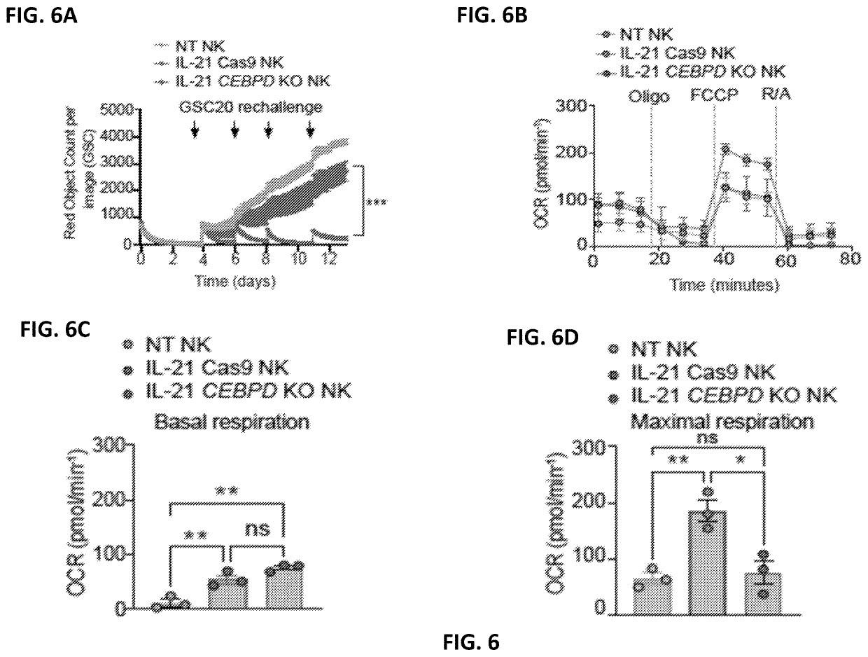

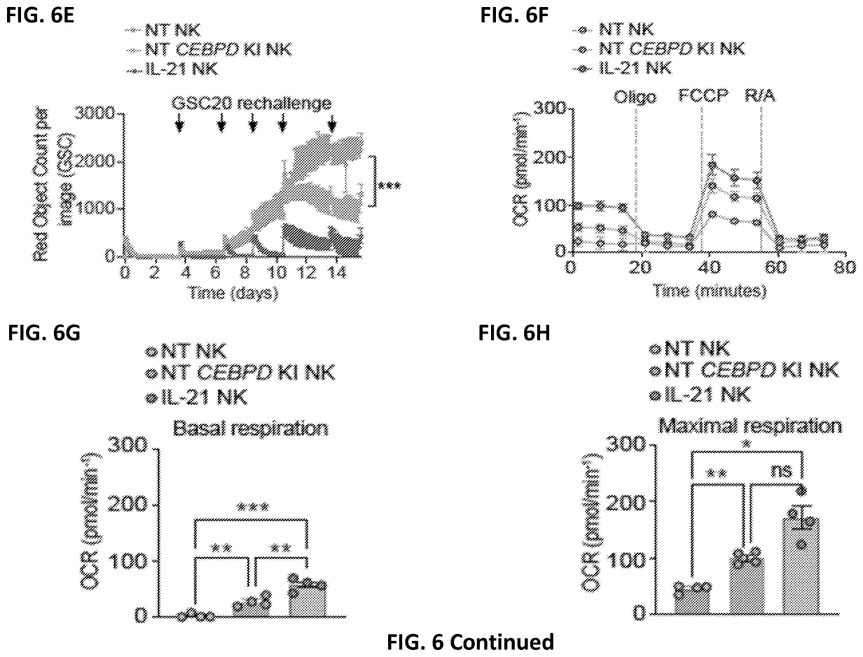

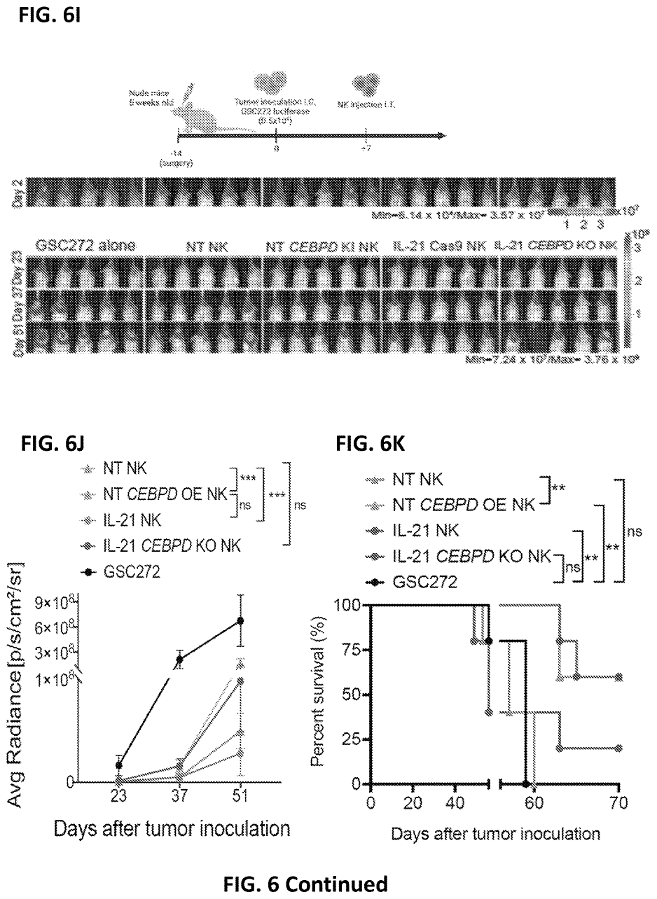

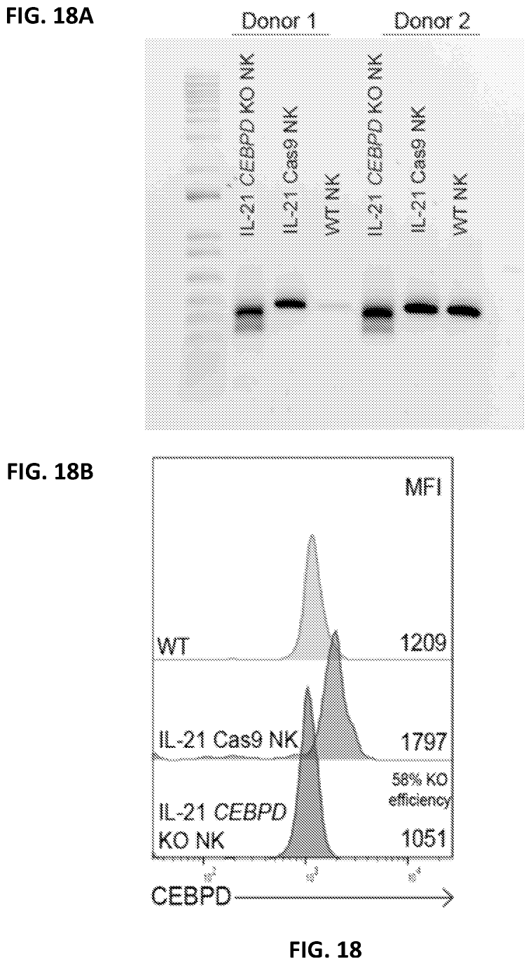

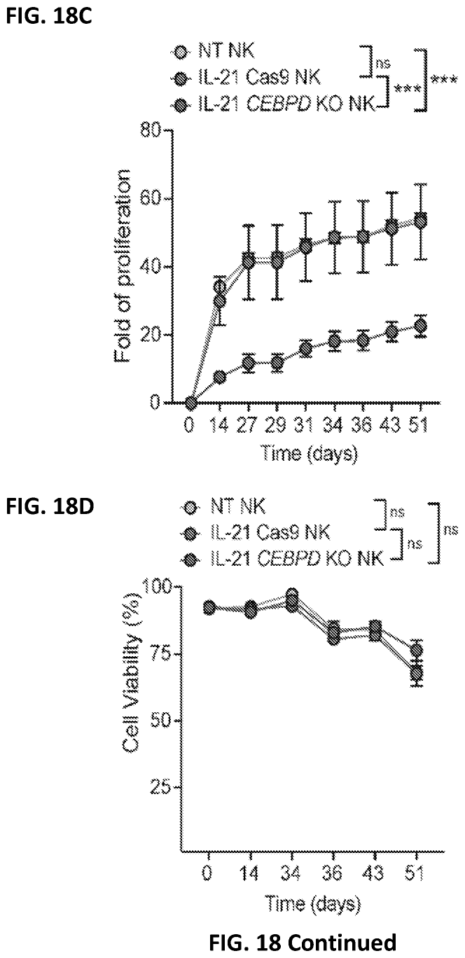

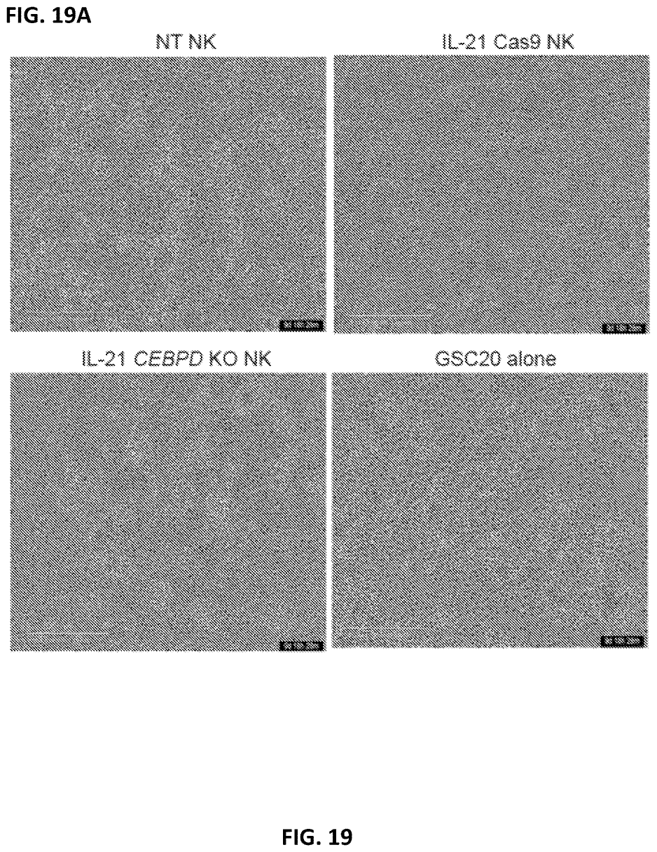

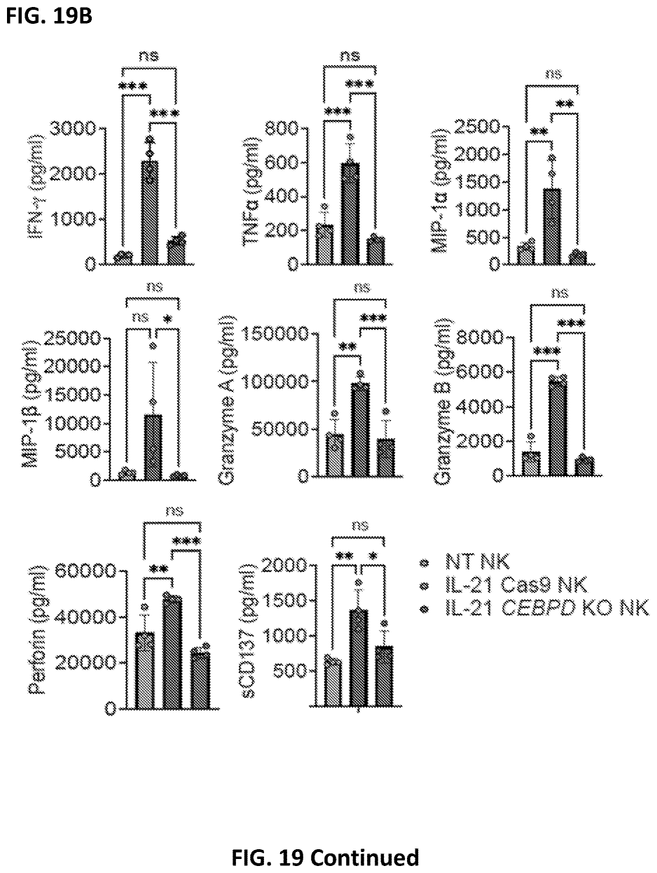

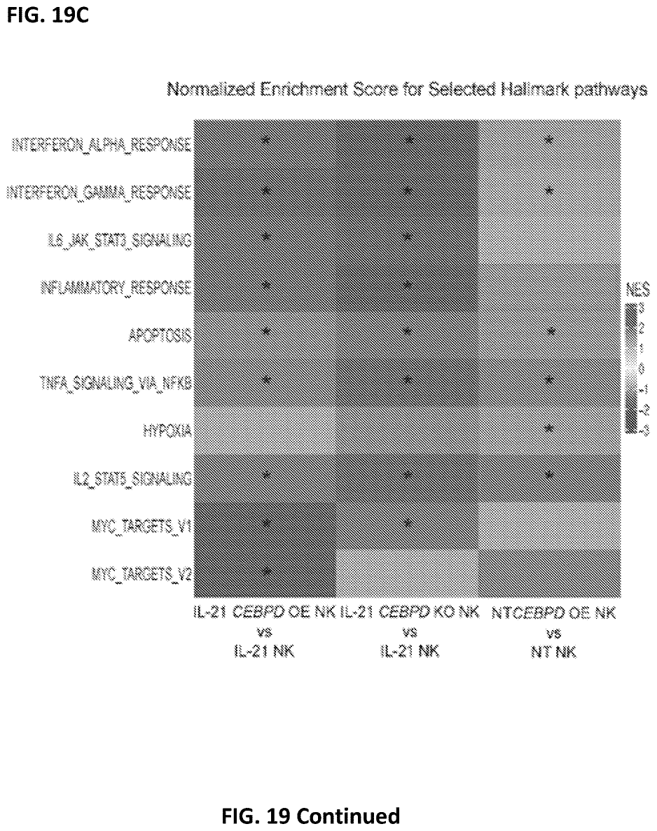

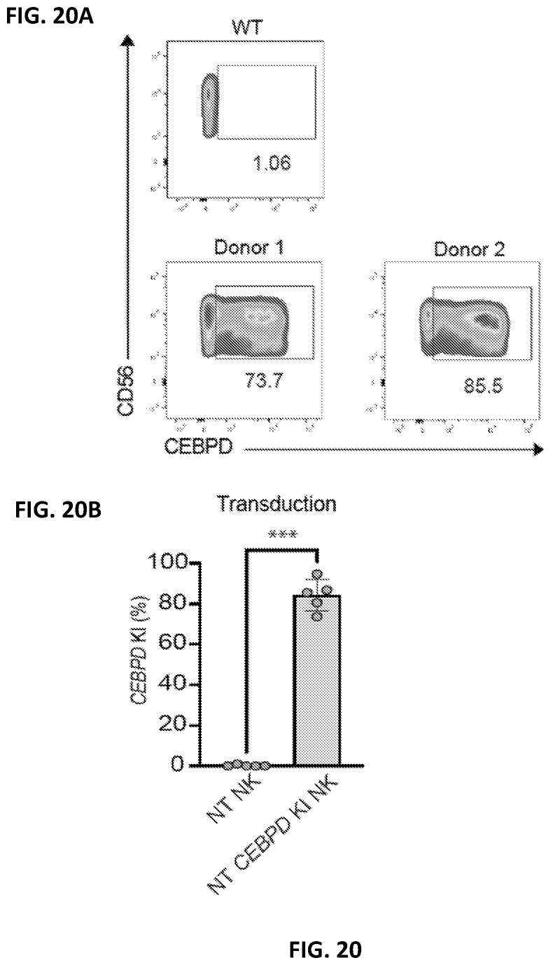

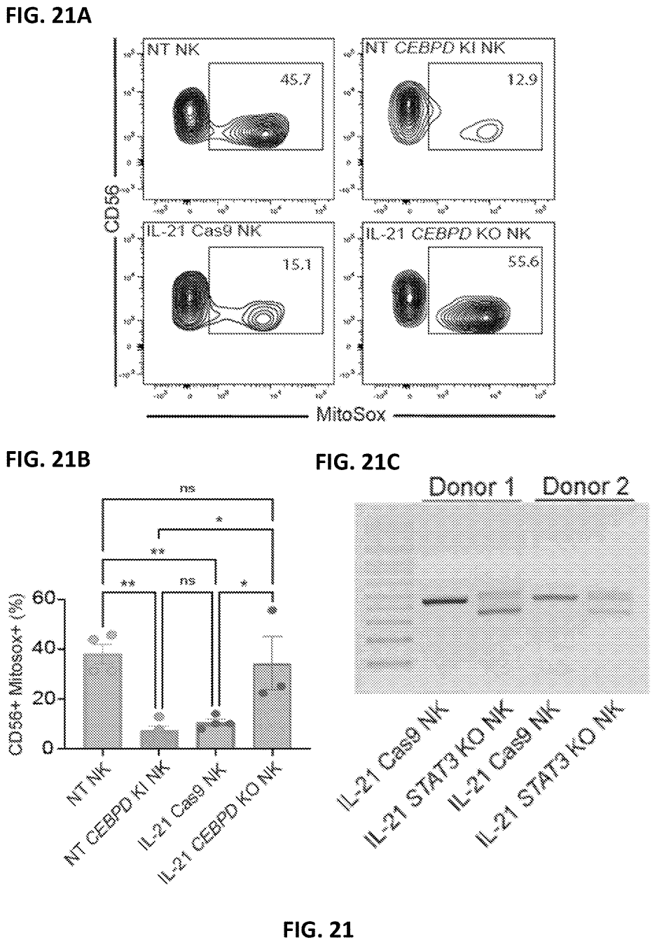

Genetic deletion of CEBPD reduced the in vitro proliferative capacity of IL-21 NK cells, without compromising their cell viability when compared with IL-21 Cas9 NK cell controls (FIGS. 18A-18D). Notably, CEBPD KO IL-21 NK cells rapidly lost their cytotoxic and effector response to multiple in vitro GSC rechallenges and displayed significant impairment in their metabolic fitness compared to control IL-21 Cas9 NK cells (FIGS. 6A-D and FIGS. 19A-19C). Transcriptomic analysis following CEBPD KO revealed downregulation of IFN signaling, JAK/STAT pathways and hypoxia response, with a reciprocal upregulation of MYC target genes (FIG. 19C). In contrast, CEBPD overexpression (CEBPD OE) in NT-NK cells with a vector encoding full-length CEBPD (FIGS. 20A-20B) substantially increased their cytotoxic response to GSC rechallenge, associated with enhanced mitochondrial fitness and lower mitochondrial reactive oxygen species (ROS) compared to NT NK cells (FIGS. 6E-H and FIGS. 21A-21B). RNA-seq analysis confirmed upregulation of the same pathways that were downregulated following CEBPD KO (FIG. 19C).

Bulk RNA-seq and ATAC-seq analyses from IL-15 NK or IL-21 NK cells revealed 1,146 differentially expressed genes (DEGs), and identified NK cell signaling as the top canonical pathway activated with IL-21 (FIG. 17A). Activity prediction in silico of this pathway revealed that both IL-15 (1,122 DEGs) and IL-21 (248 DEGs) activated basic cytotoxic NK functions, while antitumoral functions of NK cells (e.g., tumor cell apoptosis, apoptosis, and/or cytotoxicity, e.g., through ERK1/2, NFκB, IFNG, TNFSF10, FASLG, and/or Nfat activity) were activated predominantly with IL-21 stimulation (FIG. 17B). Furthermore, CEBPD-KI (SEQ ID NO: 20, translating as SEQ ID NO: 19) (1,832 DEGs) in IL-15 NK cells increased the NK cell's antitumoral functions, while CEBPD-KO (1,032 DEGs) in IL-21 NK cells abrogated the NK cell's antitumoral functions (FIG. 17B). Transcriptional differences found in network analysis were associated with corresponding changes in chromatin accessibility patterns of the NK anti-tumor response, where TF motif activity from bulk ATAC-seq revealed that STAT1 worked as a master regulator; the data also independently validated JUN and CEBPD as the top upstream and target regulators activated with IL-21, but not with IL-15.

The inventors determined whether genetic deletion of CEBPD was sufficient to abrogate the IL-21 NK cell memory response to GSC rechallenge in vitro. CRISPR/Cas9 mediated CEBPD-KO did not significantly negatively impact the short term in vitro proliferation of IL-21 NK cells (FIGS. 18A-18D) or their short-term cytotoxicity against GSCs (FIG. 6A). However, the in vitro longer term proliferation and killing capacity of IL-21 CEBPD-KO NK cells to tumor rechallenge was significantly impaired (FIG. 6A, FIGS. 18A-18D, and FIG. 19A) relative to control IL-21 Cas9 NK cells. Furthermore, IL-21 CEBPD-KO NK cells displayed a significant reduction in the release of effector molecules (e.g., IFNγ, TNFα, MIP-1α, MIP-1β, Granzyme A, Granzyme B, Perforin, and sCD137) when compared to control IL-21 Cas9 NK cells (FIG. 19B). Similarly, mitochondrial fitness was significantly reduced (e.g., reduction in maximal respiration) in IL-21 CEBPD-KO NK cells when compared to control IL-21 Cas9 NK cells (FIGS. 6B-6D). Next, to determine if overexpression of CEBPD in NK cells was sufficient to endow them with long-term GSC killing ability, the inventors generated CEBPD-KI (“CEBPD-OE”) NK cells using a retroviral vector encoding full-length codon optimized CEBPD (SEQ ID NO: 20, translating as SEQ ID NO: 19) (FIGS. 20A-20B). CEBPD-KI transduction efficiency was measured using flow cytometry, and was ~80% when averaged across 5 independent donor samples at day 5 following transduction. A significant improvement in GSC killing in response to multiple tumor rechallenge was observed for CEBPD-KINT NK when compared to NT NK cells (FIG. 6E) Additionally, when compared to NT NK cells, CEBPD-KI NT NK displayed significantly enhanced mitochondrial fitness (e.g., significantly increased basal and maximal respiration) (FIGS. 6F-6H). After 20 days of co-culture and multiple GSC rechallenges, mitochondrial reactive oxygen species (ROS) were significantly lower in CEBPD-KINK cells when compared with control NK cells (FIGS. 21A-21B), and significantly higher in IL-21 CEBPD-KO NK cells compared with control IL-21 Cas9 NK cells.

Next, the in vitro results were validated in an aggressive orthotopic GBM model (using GSC272 cells). NSG mice were surgically prepared for the assay (e.g., intracranial bolt implantation). Following recovery, NSG mice were inoculated with tumor cells intracranially on day 0 with 0.5×10⁶ GSC272 luciferase cells. Tumor bearing mice were either left untreated, or treated intratumorally with 2×10⁶ NT NK cells with or without CEBPD-KI (NT NK, or CEBPD-KI NT NK), or with IL-21 NK cells with or without CEBPD-KO (IL-21 NK, or IL-21 CEBPD-KO NK) (FIG. 6I). As described above, IL-15 NK cells were shown to cause severe toxicity and mortality in multiple orthotopic mouse models, as such, they were not included as controls in this in vivo experiment. Strong and significant antitumor activity was observed in mice treated with IL-21 NK cells (FIGS. 6I-6K) when compared to GSC272 alone (tumor only) or NT NK cells. In contrast, mice treated with IL-21 CEBPD-KO NK cells did not show significant improvement in survival when compared to the tumor only control group. Furthermore, CEBPD-KI NT-NK cells improved in vivo antitumor activity (FIG. 6J) and animal survival (FIG. 6K) compared to NT-NK cells.

Together, these data indicated that arming NK cells with IL-21 induced stable epigenetic alterations in NK cells, with CEBPD playing a critical and essential role in establishing an enhanced and sustained antitumor memory response, particularly against GBM. Furthermore, that overexpression of CEBP family proteins, such as CEBPD, was sufficient to recapitulate IL-21 induced phenotypes in NK cells.

### Example 7

### TI-NK Cells Expressed Abundant CEBPB, CEBPD, and Ap-1 Complex Associated Transcription Factors

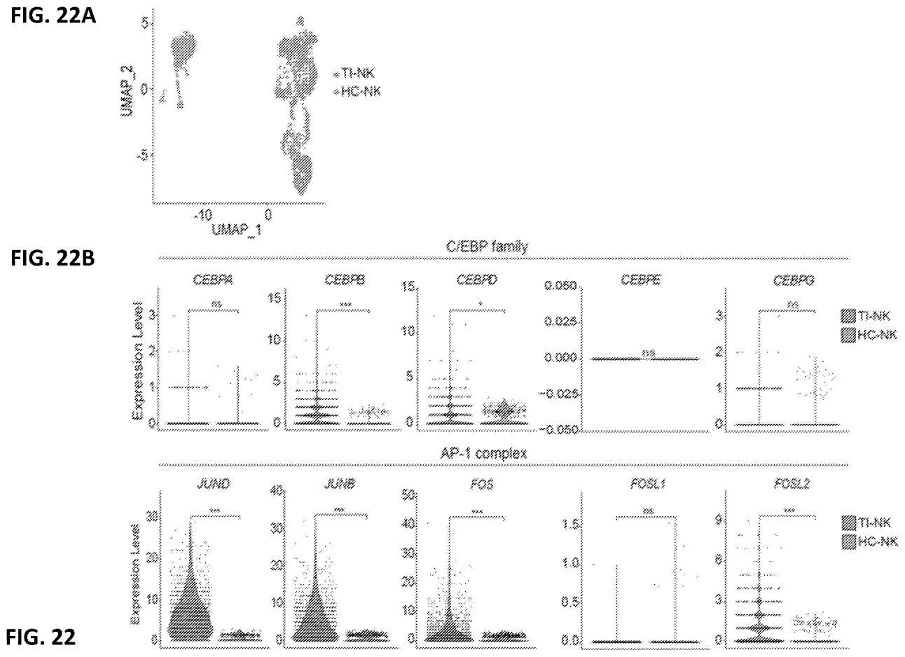

In a dataset of scRNA from patients with GBM (obtained from, Shaim, H., et al., Targeting the alphav integrin/TGF-beta axis improves natural killer cell function against glioblastoma stem cells. J. Clin Invest 131 (2021))., the expression of CEBPD, CEBPB and AP-1 complex genes in tumor-infiltrated NK cells (TI-NK) was analyzed, and found to be significantly higher when compared with peripheral blood mononuclear cell-derived NK cells from healthy donors (HC-NK) (FIGS. 22A-22B). These results supported the clinical relevance of CEBPD as an important transcriptional and epigenetic coordinator of NK cell memory to cancer, and support advancing IL-21 engineered NK cells for future vetting in clinical trials for GBM immunotherapy.

### Example 8

### Stat3 Signaling Induced CEBPD Target Gene Expression in IL-21 NK Cells

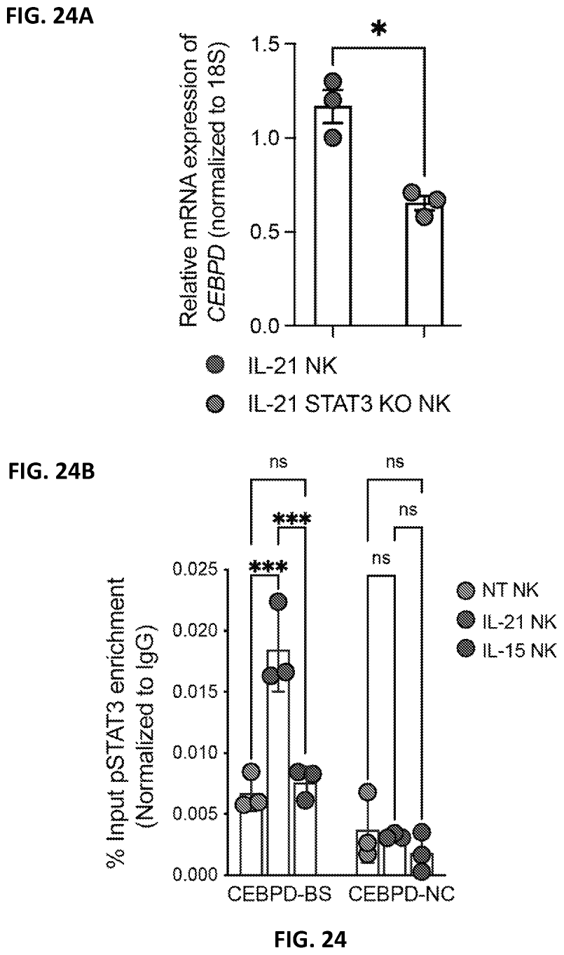

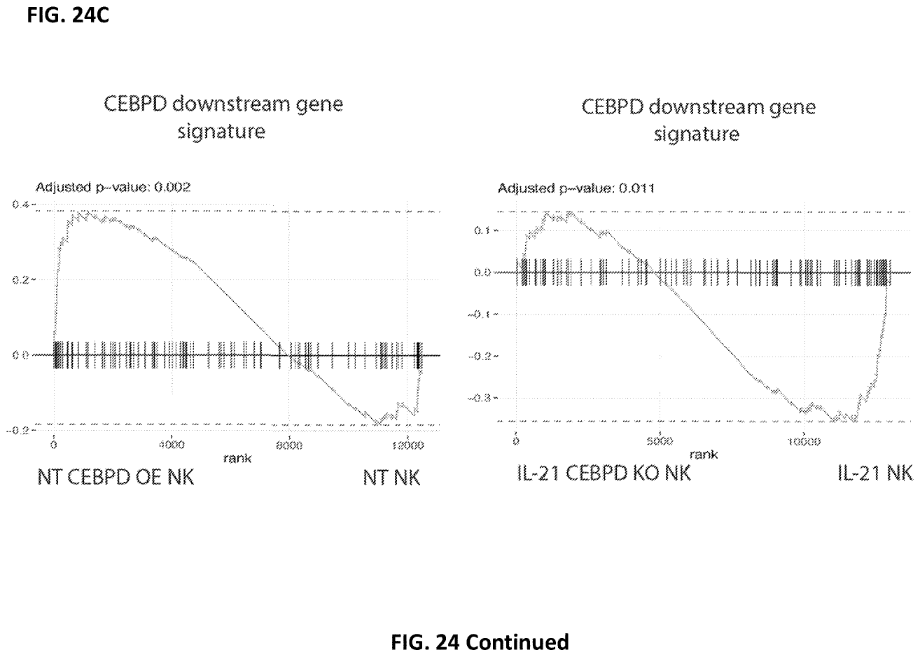

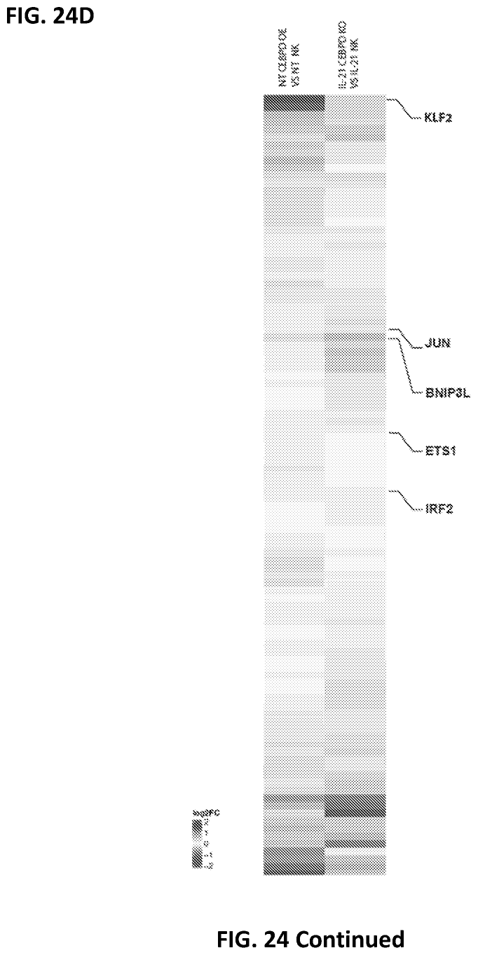

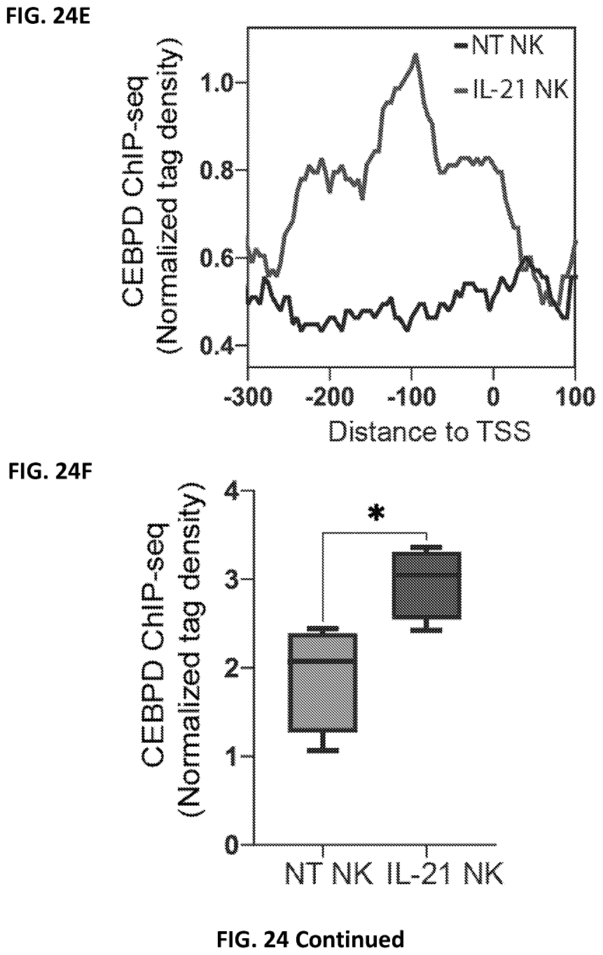

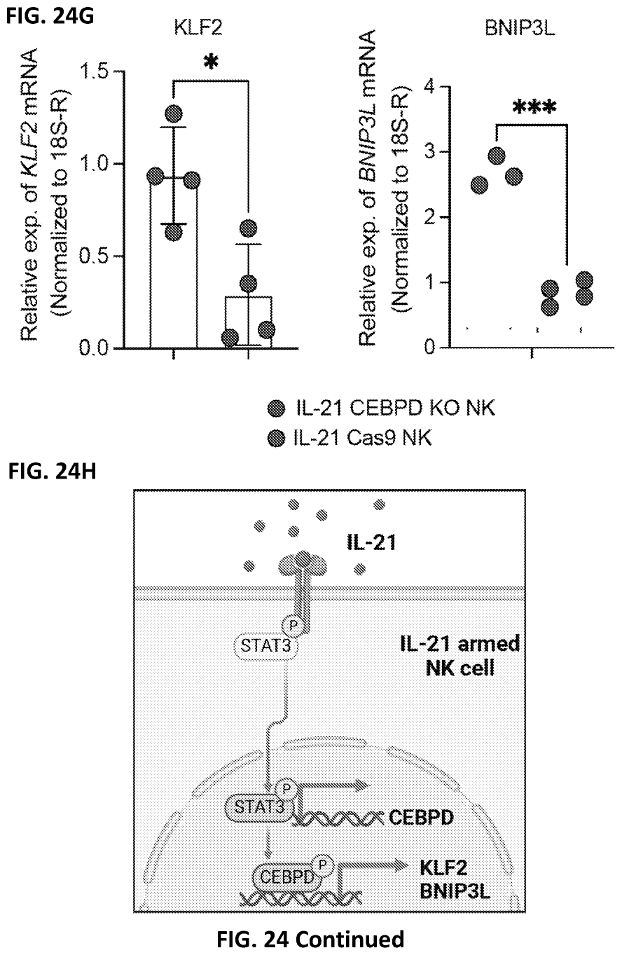

IL-21 contributes to the activation of the JAK3/STAT3 pathway, which is important for transmitting signals related to cell growth and survival⁵¹. Thus, the inventors investigated if IL-21 regulated CEBPD levels through STAT3. Deletion of STAT3 in IL-21 NK cells resulted in a significant reduction in CEBPD expression (FIG. 21C, FIG. 24A). Furthermore, chromatin immunoprecipitation (ChIP-qPCR) confirmed pSTAT3 binding to the promoter region of CEBPD in IL-21 NK cells (FIG. 24B), indicating regulation of CEBPD levels by STAT3 in IL-21 NK cells.

Next, to validate the target genes activated by CEBPD predicted by pySCENIC, the inventors analyzed bulk RNA-seq data from IL-21 NK with deletion of CEBPD or NT-NK cells following overexpression of CEBPD. The data confirmed that the predicted CEBPD downstream genes were indeed downregulated following CEBPD KO in IL-21 NK cells and upregulated following CEBPD OE in NT-NK cells, both at the signature level by gene set enrichment analysis (FIG. 24C) and at the individual gene level (FIG. 24D). Chromatin immunoprecipitation sequencing of CEBPD (ChIP-seq) analysis revealed significant peak enrichment in the promoter regions of KLF2, BNIP3L, IRF1 and ETS1 in IL-21 NK compared with NT NK cells, supporting their downstream regulation by CEBPD (FIGS. 24E-24F). Furthermore, qPCR analysis comparing IL-21 Cas9 NK cells with IL-21 CEBPD KO cells confirmed KLF2 and BNIP3L as CEBPD target genes (FIG. 24G). Taken together, these data demonstrate that IL-21 increased expression of CEBPD, most likely through pSTAT3, and that CEBPD in turn induced upregulation of its target genes including KLF2 and BNIP3L (FIG. 24H). As KLF2 and BNIP3L were known to contribute to NK/T cell survival and metabolic fitness, the expression of these and other related CEBPD target genes likely contributed to the effective and sustained antitumor activity of IL-21 NK cells against GSCs^{44,52,53}.

### Example 9

### Discussion

The data and analyses presented herein showed that arming NK cells with constitutively and/or autonomously expressed and secreted IL-21 endowed them with enhanced and long-lasting antitumor activity. Furthermore, the data and analyses provided mechanistic insights into IL-21 NK cell's unexpected memory-like features. More specifically, the inventors have demonstrated that IL-21 NK cells maintained a robust effector function against tumor rechallenge, while IL-15 NK cells more readily acquired an exhausted phenotype associated with a greater loss of effector potency. The enhanced IL-21 NK cell response was mediated through superior metabolic fitness and memory-like features. The inventors have also identified the TF family CEBP, and in particular CEBPD, as an important mediator of this phenotype. Indeed, CEBPD deletion in IL-21 NK cells strongly impaired their effector function against tumor rechallenge and reduced their antitumor activity in an orthotopic mouse model of human GBM, showing the necessity of CEBPD in mediating these IL-21 NK phenotypes. Surprisingly, the inventors have also shown that ectopic expression of CEBP family proteins, and in particular CEBPD, in non-cytokine-armed (e.g., non-transduced) NT NK cells enhanced their metabolic fitness and long-term antitumor potency, showing the sufficiency of CEBPD in mediating these IL-21 NK phenotypes.

While NK memory against viruses have been described, little was previously known of epigenetic and transcriptomic mechanisms that regulated NK cell memory to cancer. In an elegant paper, Lau et al. reported dynamic changes in the chromatin architecture during murine CMV infection, and showed an important role for IL-12-induced STAT4 and STAT1 in promoting epigenetic and transcriptional changes that could play a role in NK memory response to infection. Similarly, Ruckert et al. reported that NK cells from individuals with human cytomegalovirus (HCMV) undergo clonal expansion with a memory signature, enrichment of AP-1 motifs and maintenance of specific epigenetic states over time.

As described herein, the inventors have explored dynamic molecular changes in NK cells during the acquisition of memory-like features in response to cancer. The inventors depicted the single cell chromatin accessibility landscape of cytokine-engineered NK cells following in vitro co-culture with tumor cells by scATAC-seq. The inventors found that NK cells expressing IL-21 vs IL-15 showed rapid, comprehensive, and dramatic changes in their chromatin accessibility in vitro, associated with different and distinct responses to tumor challenge. While IL-21-armed NK cells showed enhanced short-term and long-term activity against GSCs, IL-15-armed NK cells were more prone to exhaustion after tumor rechallenge. Previous studies have shown that continuous exposure to higher doses of IL-15 (e.g., in the nanogram range), but not lower doses (e.g., picogram range), can potentially drive NK cell exhaustion. Indeed, in this case, the engineered IL-15 NK cells released IL-15 in the nanogram range, which may explain their cytotoxicity against GCSs in short-term cultures (comparable to IL-21 cells), but their eventual exhaustion in long-term cultures (significantly more exhausted and less cytotoxic when compared to IL-21 NK cells).

In addition, the inventors have identified key TFs that were upregulated in IL-21 NK cells after multiple rounds of tumor rechallenge. While several of the TFs were part of the AP-1 network, reported to play a key role in memory formation, members of the previously non-memory formation associated C/EBP family of TFs were also significantly upregulated in IL-21 NK cells exposed to GSCs. CEBPD, which showed the highest upregulation, was pursued as a pivotal regulator of the IL-21 NK cell memory response. The inventors have confirmed the importance of CEBPD in NK cell memory through multiple lines of evidence: 1) deletion of CEBPD significantly inhibited the long-term antitumor efficacy of IL-21 NK cells (necessary) and overexpression of CEBPD in wild-type NK cells enhanced their cytotoxicity and metabolic fitness (sufficiency). Furthermore, the inventors have confirmed higher expression of CEBPB, CEBPD, and AP-1 complex genes in tumor-infiltrating NK cells from patients with GBM when compared to PB NK cells.

Differential expression of CEBPD between the engineered IL-21 NK and IL-15 NK cells was observed even at baseline (before tumor challenge) and was further magnified after multiple tumor rechallenges during extended in vitro culture. IL-21 has been shown to induce memory in T cells but its role in NK cell memory was not previously identified or appreciated. Fuch and colleagues unveiled the epigenetic mechanisms of memory formation in an inflammatory state to be driven by an obligatory cooperation between STAT3 and the TFs FOS and JUN (members of the AP-1 complex). IL-21 is an important mediator of STAT3 activation, and STAT3 has been reported to play a role in CEBPD expression. Similar to STAT3, STAT1 has also been implicated in the regulation of IL-21-mediated genes such as IFNG and TBX21 in T cells. The interplay between STATs and CEBPD in memory formation warrants further investigation.

As described above, in these studies the inventors showed that arming NK cells with the ability to autonomously express and secrete IL-21 endowed them with enhanced and long-lasting antitumor activity and metabolic fitness. Importantly, the inventors identified the transcription factor CEBPD as an important mediator of this functional phenotype.

The inventors discovered that IL-21-armored NK cells sustained their antitumor activity against GSCs over time, while IL-15 NK cells were more prone to becoming exhausted after multiple tumor rechallenges. This is consistent with previous studies reporting that continuous exposure to higher doses of IL-15 (in the nanogram range)⁵⁴ but not lower doses (picogram range)^{10,11} can drive NK cell exhaustion. Indeed, in this case, the IL-15-engineered NK cells released IL-15 in the nanogram range, which may explain their potent cytotoxicity in short-term cultures but their eventual exhaustion in long-term cultures. In addition, IL-15 NK cells demonstrated limited antitumor efficacy in vivo, and their direct injection into the orthotopically implanted tumor resulted in significant NK cell proliferation and neurotoxicity, associated with marked astrocytosis and microgliosis. These effects were observed in multiple murine models of GSCs, regardless of the dose of IL-15 NK cells administered or IL-15 expression levels used in the immediate studies. In contrast, systemic delivery of IL-15 NK cells did not produce such neurotoxicity, in keeping with the established safety profile of IL-15-engineered CAR-NK cells currently under clinical evaluation 10,12. These findings indicated that local effects of IL-15 in the brain microenvironment were principally responsible for the observed neurotoxicity. Notably, IL-15 is known to promote interactions between astrocytes and microglia, contributing to neuroinflammation by propagating cellular immunity in conditions such as intracranial hemorrhage^{55,56}. The immediate study highlights the critical need to consider the secondary effects of cytokines like IL-15 on non-target cells such as microglia, which may become activated due to IL-15's pleiotropic effects. In contrast, IL-21 does not affect cells of the myeloid lineage⁵⁷ and may be less toxic for local administration into the brain. Indeed, using multiple orthotopic models of GSCs, the inventors showed that IL-21 NK cells were safe and result in long-term tumor control.

The experiments designed and performed as described herein also explored the molecular evolution of cytokine-engineered NK cells in response to tumor rechallenge. While epigenetic and transcriptional changes associated with long-term NK cell responses against viruses have been described^{58,59}, little was known of the molecular mechanisms that regulated long-term NK cell responses to cancer. The data provided herein showed significant and dramatic changes in the chromatin accessibility landscape of NK cells after tumor rechallenge. Notably, IL-21 NK cells showed significant upregulation of key TFs such as the AP-1 network, reported to play a key role in cellular persistence^{32,59}, and the C/EBP family of TFs, with CEBPD being the most prominent. CEBPD was pursued as a pivotal regulator of the IL-21 NK cell enhanced response through multiple lines of evidence: deletion of CEBPD significantly inhibited the long-term antitumor efficacy of IL-21 NK cells while its overexpression in wild-type NK cells improved their cytotoxicity and metabolic fitness. These effects were associated with moderate upregulation of IFN response genes and the STAT3/STAT5 pathways, suggesting that additional factors could also play a role in the enhanced IL-21 NK cell response. Fuch and colleagues showed that memory in an inflammatory state is driven by an obligatory cooperation between STAT3 and AP1 complex members, FOS and JUN³². STAT3, a known target of IL-21⁶⁰, has been implicated in regulating CEBPD expression⁶¹. The data supported a possible role for STAT3 in the regulation of CEBPD by IL-21 in NK cells.

Taken together, the findings discussed herein supported arming of NK cells with IL-21, and treatment of solid tumors, particularly brain tumors such as glioblastoma, with NK cells that autonomously express and secrete IL-21. Furthermore, that such arming induced NK cell epigenetic reprogramming, strongly mediated by CEBP family proteins, particularly requiring CEBPD. The resulting reprogramming elicited beneficial phenotypes such as enhanced mitochondrial fitness and memory-like features in NK cells. In this study, deletion of CEBPD significantly impaired IL-21 NK cell potency and recall responses against tumor rechallenge, while CEBPD overexpression in NK cells increased their functional capacity, metabolic fitness and anti-GSC potency. These data support the development of IL-21-armed NK cells as a novel immunotherapeutic approach for GBM. These data support the development of CEBP protein family overexpression in NK cells as a novel immunotherapeutic approach for treatment of cancer.

All of the methods disclosed and claimed herein can be made and executed without undue experimentation in light of the present disclosure. While the compositions and methods of this inventions have been described in terms of preferred embodiments, it will be apparent to those of skill in the art that variations may be applied to the methods and in the steps or in the sequence of steps of the method described herein without departing from the concept, spirit and scope of the inventions. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the inventions as defined by the appended claims.

What is claimed is:

1. An engineered Natural Killer (NK) cell modified to overexpress a CCAAT/Enhancer-binding protein (CEBP) transcription factor family protein.

2. The engineered NK cell of claim 1, wherein CEBP transcription factor expression is relative to a non-engineered NK cell and/or relative to an NK cell engineered to express IL-15.

3. The engineered NK cell of claim 1, wherein the CEBP protein is CEBPD (CEBP-delta, CEBPδ), CEBPA (CEBP-alpha, CEBPα), CEBPB (CEBP-beta, CEBPβ), CEBPG (CEBP-gamma, CEBPγ), CEBPE (CEBP-epsilon, CEBPε) and/or CEBP Homologous Protein (CHOP).

4. The engineered NK cell of claim 1, wherein the CEBP protein is CEBPD and/or CEBPB.

5. The engineered NK cell of claim 1, wherein the NK cell overexpresses a transgenic CEBPD and/or CEBPB protein.

6. The engineered NK cell of claim 1, wherein the NK cell transgenically expresses and/or are subjected to a CEBP protein transcriptional and/or translational activator.

7. The engineered NK cell of claim 1, wherein the NK cell transgenically expresses and/or are subjected to an inhibitor of a CEBP protein transcriptional and/or translational inhibitor.

8. The engineered NK cell of claim 1, wherein the CEBP protein comprises a sequence at least 80%, 85%, 90%, 95%, 99%, or 100% identical to SEQ ID NO: 19.

9. The engineered NK cell of claim 1, wherein the CEBP protein is encoded by a sequence comprising a sequence at least 80%, 85%, 90%, 95%, 99%, or 100% identical to SEQ ID NOs: 20 or 21.

10. The engineered NK cell of claim 1, wherein the CEBP protein comprises a sequence at least 80%, 85%, 90%, 95%, 99%, or 100% identical to any one of SEQ ID NOs: 23, 25, or 27.

11. The engineered NK cell of claim 1, wherein the CEBP protein is encoded by a sequence comprising a sequence at least 80%, 85%, 90%, 95%, 99%, or 100% identical to any one of SEQ ID NOs: 24, 26, or 28.

12. The engineered NK cell of claim 1, wherein the NK cell has enhanced mitochondrial fitness and/or memory-like features relative to a non-engineered NK cell and/or relative to an NK cell engineered to express IL-15.

13. The engineered NK cell of claim 12, wherein the enhanced mitochondrial fitness comprises increased basal and/or maximal oxygen consumption rate (OCR) compared to a non-engineered NK cell and/or an NK cell engineered to express IL-15, and/or a reduction in glycolysis (e.g., wherein glycolysis is measured by extracellular acidification rate (ECAR)) compared to a non-engineered NK cell and/or an NK cell engineered to express IL-15.

14. The engineered NK cell of claim 1, wherein the NK cell has enhanced anti-tumor cytotoxicity relative to a non-engineered NK cell and/or relative to an NK cell engineered to express IL-15.

15. The engineered NK cell of claim 1, wherein the NK cell has enhanced anti-tumor memory relative to a non-engineered NK cell and/or relative to an NK cell engineered to express IL-15.

16. The engineered NK cell of claim 1, wherein the NK cell has high expression of functional markers, low expression of inhibitory markers, high expression of survival genes, low expression of exhaustion genes, and/or upregulation of activation receptors and markers, relative to a non-engineered NK cell and/or relative to an NK cell engineered to express IL-15.

17. The engineered NK cell of claim 16, wherein the functional markers comprises Granzyme A (GrA), Granzyme B (GrB), Perforin, and/or Zap70.

18. The engineered NK cell of claim 16, wherein the inhibitory markers comprises LAG3 and/or KLRG1.

19. The engineered NK cell of claim 16, wherein the activation receptors and markers comprises NKp30, CD25, DNAM, Ki67, CD3ζ, T-bet, and/or FCεRG.

20. The engineered NK cell of claim 16, wherein the survival genes comprises KLRD1, ITGA1 and/or GZMK.

21. The engineered NK cell of claim 16, wherein the exhaustion genes comprises DUSP2, CISH, and/or BAX.

22. The engineered NK cell of claim 1, wherein the NK cells comprise high expression levels of cytotoxicity markers GrA, GrB, Perforin, TRAIL, and/or CD95, and/or high expression levels of activation markers/receptors CD25, CD69, DNAM, NKG2D, NKp44 and/or NKp46, relative to a non-engineered NK cell and/or relative to an NK cell engineered to express IL-15.

23. The engineered NK cell of claim 1, wherein the NK cells comprise high expression levels of transcription factors important for NK cell maturation, NK cell immune function, NK cell cytotoxicity, IFN-γ response, memory formation, and/or AP-1 complex members, relative to a non-engineered NK cell and/or relative to an NK cell engineered to express IL-15.

24. The engineered NK cell of claim 23, wherein the transcription factors comprise CEBPD, CEBPB, ETS1, IRF1, TBX21, EOMES, IRF9, STAT1, ETS1, JUN, JUNB, JUND, FOS, and/or FOSL1.

25. The engineered NK cell of claim 1, wherein the cell is further engineered to provide one or more interleukins (IL).

26. The engineered NK cell of claim 25, wherein the IL is IL-2, IL-7, IL-12, IL-15, IL-17, IL-18, IL-21, and/or the p35 and p40 subunits of IL-12 artificially linked together.

27. The engineered NK cell of claim 26, wherein the IL is IL-21 and/or IL-15.

28. The engineered NK cell of claim 26, wherein the NK cell gains increased tumor cell apoptosis, apoptosis, and/or cytotoxicity capacity.

29. The engineered NK cell of claim 26, wherein the NK cell has increased levels and/or activity of ERK1/2, NFκB, IFNG, TNFSF10, FASLG, and/or Nfat.

30. The engineered NK cell of claim 25, wherein the IL is secreted, tethered, or membrane bound in the cell.

31. The engineered NK cell of claim 1, wherein the NK cells are derived from cord blood (CB), peripheral blood (PB), bone marrow, stem cells, NK cell lines, or a combination thereof.

32. The engineered NK cell of claim 1, wherein the NK cells are primary NK cells, and are not derived from stem cells and/or induced pluripotent stem cells (iPSCs).

33. The engineered NK cell of claim 1, wherein the NK cells are complexed to one or more monospecific, bispecific, and/or multi-specific antibodies.

34. The engineered NK cell of claim 33, wherein the NK cell expresses one or more antibody.

35. The engineered NK cell of claim 1, wherein the NK cell is further modified to express one or more additional heterologous proteins selected from the group consisting of an antigen receptor, a cytokine, a homing receptor, a chemokine receptor, and a combination thereof.

36. The engineered NK cell of claim 35, wherein the engineered receptor is an engineered antigen receptor.

37. The engineered NK cell of claim 33, wherein a target antigen is a cancer antigen.

38. The engineered NK cell of claim 1, wherein the NK cell comprises a suicide gene.

39. The engineered NK cell of claim 1, wherein the NK cell further comprises one or more engineered mutations in an endogenous gene.

40. The engineered NK cell of claim 39, wherein the endogenous gene is TGFBR2, CISH, GR, and/or CD38.

41. The engineered NK cell of claim 1, wherein the NK cells are pre-activated with one or more cytokines.

42. The engineered NK cell of claim 41, wherein the one or more cytokines comprises IL-2.

43. A composition comprising the engineered NK cell of claim 1.

44. The composition of claim 43, further comprising a pharmaceutically acceptable excipient.

45. The composition of claim 43, wherein the composition is comprised in a delivery device.

46. A method of treating a disease in an individual, the method comprising the step of administering to the individual a therapeutically effective amount of any one of the engineered NK cells or compositions of any one of the preceding claims.

47. The method of claim 46, wherein the disease is an autoimmune disease, infection, and/or cancer.

48. The method of claim 46, wherein the disease is cancer.

49. The method of claim 48, wherein the cancer is of the lung, brain, breast, blood, skin, pancreas, liver, colon, head and neck, kidney, thyroid, stomach, spleen, gallbladder, bone, ovary, testes, endometrium, prostate, rectum, anus, cervix, or is hematological.

50. The method of claim 48, wherein the cancer is glioblastoma.

51. The method of claim 50, where the glioblastoma is a TCGA mesenchymal, neural, classical, or Proneural subtype.

52. The method of claim 51, where the glioblastoma is a mesenchymal, or Proneural subtype.

53. The method of claim 50, wherein the glioblastoma has an MGMT unmethylated, methylated, or indeterminate status.

54. The method of claim 50, wherein the glioblastoma is primary or recurrent.

55. The method of claim 48, wherein the engineered NK cells display increased in vivo antitumor functionality relative to a non-engineered NK cell and/or an NK cell engineered to express IL-15.

56. The method of claim 55, wherein the increased in vivo antitumor functionality comprises long-term tumor eradication, significant prolongation of subject survival, and/or reduced toxicity or weight loss.

57. The method of claim 50, wherein the administering is through intracranial injection.

58. The method of claim 50, wherein the administering is through intratumoral injection.

59. The method of claim 50, wherein the method provides immune memory against glioblastoma.

60. The method of claim 50, wherein the method provides immune memory against glioblastoma stem cells.

61. A method of providing a subject with immune memory against cancer, the method comprising administering to an individual a therapeutically effective amount of the engineered NK cells of any one of claims 1 to 42, or compositions of any one of claims 43 to 45.

62. An isolated nucleic acid encoding a CEBP protein fused to a heterologous transcriptional regulatory element.

63. The isolated nucleic acid of claim 62, wherein the CEBP protein comprises a sequence at least 80%, 85%, 90%, 95%, 99%, or 100% identical to SEQ ID NO: 19.

64. The isolated nucleic acid of claim 62, wherein the CEBP protein is encoded by a sequence comprising a sequence at least 80%, 85%, 90%, 95%, 99%, or 100% identical to SEQ ID NO: 20 or 21.

65. The isolated nucleic acid of claim 62, wherein the heterologous transcriptional regulatory element is a promoter.

66. A method of treating glioblastoma, the method comprising administering to an individual a therapeutically effective amount of an NK cell engineered to autonomously and/or constitutively express secreted IL-21.

67. The method of claim 66, wherein the administering is through intracranial injection.

68. The method of claim 66, wherein the administering is through intratumoral injection.

69. The method of claim 66, wherein the NK cell engineered to constitutively and/or autonomously express secreted IL-21 comprises a transgenic polynucleotide sequence encoding and/or comprising a sequence at least 80%, 85%, 90%, 95%, or 100% identical to SEQ ID NOs: 31-32.

70. The method of claim 66, wherein the method provides immune memory against glioblastoma.

71. The method of claim 66, wherein the method provides immune memory against glioblastoma stem cells.

72. The method of claim 66, wherein the NK cells are engineered to stably secrete IL-21 at rate suitable for reaching an extracellular concentration of greater than or equal to 200, 250, 300, 350, 400, 450, 500, 550, 600, 650, 700, or 750 pg/mL.

73. The method of claim 66, wherein the NK cells are engineered to stably secrete IL-21 at rate suitable for reaching an extracellular concentration of greater than or equal to or about 200-800 pg/mL, 250-750 pg/mL, 300-700 pg/mL, or 350-650 pg/mL.

74. The method of claim 66, wherein the glioblastoma is a TCGA mesenchymal, neural, classical, or Proneural subtype.

75. The method of claim 66, wherein the glioblastoma is a mesenchymal, or Proneural subtype.

76. The method of claim 66, wherein the glioblastoma has an MGMT unmethylated, methylated, or indeterminate status.

77. The method of claim 66, wherein the glioblastoma is primary or recurrent.

78. The method of claim 66, wherein the engineered NK cells display increased in vivo antitumor functionality relative to a non-engineered NK cell and/or an NK cell engineered to express IL-15.

79. The method of claim 78, wherein the increased in vivo antitumor functionality comprises long-term tumor eradication, significant prolongation of subject survival, and/or reduced toxicity or weight loss.
